(19) United States
(12) Patent Application Publication
(10) Pub. No.: US 2016/0002343 A1
(43) Pub. Date: Jan. 7, 2016

(54) BLOOD-BRAIN BARRIER (BBB) PENETRATING DUAL SPECIFIC BINDING PROTEINS FOR TREATING BRAIN AND NEUROLOGICAL DISEASES

(71) Applicant: AbbVie Inc., North Chicago, IL (US)

(72) Inventors: Denise Karaoglu Hanzatian, Natick, MA (US); Andrew D. Goodearl, Sherborn, MA (US); Bernhard Klaus Mueller, Neustadt-gimmelding (DE); Reinhold Mueller, Schifferstadt (DE); Corinna Klein, Mainz (DE)

(21) Appl. No.: 14/737,474

(22) Filed: Jun. 11, 2015

Related U.S. Application Data
(60) Provisional application No. 62/011,010, filed on Jun. 11, 2014.
(60) Provisional application No. 62/148,623, filed on Apr. 16, 2015.

(51) Int. Cl. C07K 16/28; A61K 39/395

(52) U.S. Cl. CPC C07K 16/2881; C07K 16/2875; A61K 39/3955; C07K 2319/00; C07K 2317/24; C07K 2317/31; C07K 2317/76; A61K 2039/505

(57) ABSTRACT

Engineered agents or multivalent and multispecific binding proteins capable of penetrating the cells or tissue of the blood-brain barrier (BBB) are provided, along with methods of making and uses in the prevention, diagnosis, and/or treatment of disease.

### RELATED APPLICATIONS

This application claims priority to U.S. provisional application Ser. No. 62/148,623, filed Apr. 16, 2015, and U.S. provisional application Ser. No. 62/011,010, filed Jun. 11, 2014, each of which is incorporated herein by reference in its entirety.

### SEQUENCE LISTING

The instant application contains a Sequence Listing which has been submitted electronically in ASCII format and is hereby incorporated by reference in its entirety. Said ASCII copy, created on Aug. 19, 2015, is named 569553-347_SL.txt and is 184,169 bytes in size.

### FIELD OF THE INVENTION

Multivalent and multispecific binding proteins that bind receptors on the brain endothelial cells that form the Blood-Brain Barrier (BBB), and comprise domains that bind protein targets inside the brain or ligands for delivery into the brain, methods of making, in vivo distribution thereof in brain, and uses thereof in the treatment of acute and chronic neurological diseases, including multiple sclerosis, Parkinson's disease and Alzheimer's disease, are provided.

### BACKGROUND OF THE INVENTION

Alzheimer's disease (AD) is a neurodegenerative disorder associated with progressive memory loss and cognitive dysfunction. The disease is characterized by the presence in the brain of amyloid plaques, comprised of amyloid beta (Aβ) protein, and neurofibrillary tangles (NFTs), comprised of hyperphosphorylated tau protein. Several different Aβ species have been identified that are 36-43 amino acids in length, including Aβ38, Aβ40, and Aβ42. Aβ40 peptide is the most abundant (˜80-90%), followed by Aβ42 (˜5-10%). The longer forms of Aβ, particularly Aβ42, are more hydrophobic and fibrillogenic, and are the principal species deposited in the brain in AD patients (Selkoe (2001) Neuron 32(2):177-80). An estimated 4 million Americans suffer from AD and with approximately 1 in every 80 persons in the U.S. projected to have the disease by 2030.

Parkinson's disease (PD) is a progressive neurodegenerative disease affecting 1-2% of the population over 65 years of age. It has been estimated that the number of cases of PD worldwide will double by the year 2030. Currently, there is no cure, early detection mechanism, preventative treatment, or effective way to slow disease progression. Classic neuronal pathological features of PD include the loss of dopaminergic (DA) neurons in the substantia nigra (SN) and the presence of cytoplasmic inclusions, known as Lewy bodies. Classic clinical features of PD include resting tremor, bradykinesia and rigidity, but the disease also leads to a wide variety of non-motor features such as autonomic dysfunction and dementia. The majority of PD patients suffer from idiopathic disease with no clear etiology, and approximately 5% of patients present with familial PD. Although the pattern of neuronal loss in PD is well characterized, the molecular mechanisms that lead to cell death are still unknown.

Multiple Sclerosis (MS) is a neurological disease affecting more than 1 million people worldwide. It is the most common cause of neurological disability in young and middle-aged adults and has major physical, psychological, social and financial impacts on subjects and their families, friends and health care providers. It is generally assumed that MS is mediated by an autoimmune process possibly triggered by infection and superimposed upon a genetic predisposition. It is a chronic inflammatory condition that damages the myelin of the central nervous system (CNS). The pathogenesis of MS is characterized by the infiltration of autoreactive T-cells from the circulation directed against myelin antigens into the CNS. In addition to the inflammatory phase of MS, axonal loss occurs early in the course of the disease and over time can lead to the development of progressive, permanent, neurologic impairment and, frequently, severe disability. Symptoms associated with the disease include fatigue, spasticity, ataxia, weakness, bladder and bowel disturbances, sexual dysfunction, pain, tremor, paroxysmal manifestations, visual impairment, psychological problems and cognitive dysfunction.

AD, PD and MS are complex and multifactorial neurological diseases with the possible involvement of age, genetics and environmental factors. Existing treatments for DA, PD, and MS cannot stop their progression, let alone cure the disease. A major hurdle to treating these diseases is the ability to delivery therapies into the brain, which requires effectively traversing the blood brain barrier (BBB).

A number of strategies have been employed to deliver therapeutics across the BBB. Mechanisms for drug targeting into the brain by passing through the BBB have entailed its disruption by osmotic means, by vasoactive substances such as bradykinin, or by localized exposure to high-intensity focused ultrasound (HIFU). Other methods for permeating the BBB have entailed the use of endogenous transport systems, such as glucose and amino acid carriers, receptor-mediated transcytosis for insulin or transferrin, and the blocking of active efflux transporters such as p-glycoprotein. Methods for drug delivery behind the BBB also include intracerebral implantation and convection-enhanced distribution.

Biopharmaceuticals offer the advantages of high specificity, potency and lower off-target toxicity, however, the delivery of these drugs through natural protective barriers such as the BBB poses substantial challenges owing in part to their large size and susceptibility to degradation. Cells and tissues that make up natural barriers, for example cells in the brain, stomach, and colon, work effectively to protect the organism, for example by preventing entrance of agents that cause infection or disorder. Unfortunately, many therapeutic molecules do not cross these cells and tissues in adequate amounts, such that they serve as highly selective or impermeable barriers that limit effective treatment of disorders and conditions.

Effective methods and agents that pass natural biological barriers, and especially the BBB, are needed. These agents would allow for more effective treatment of neurological diseases that affect millions of patients.

### SUMMARY OF THE INVENTION

The instant disclosure improves upon the art by providing high molecular weight (HMW) binding proteins capable of binding a BBB antigen, e.g., an extracellular receptor, a surface protein, an intracellular receptor, an intracellular protein, a carbohydrate, a target, and a ligand receptor, that is expressed on a cell or tissue of the barrier (e.g., the brain vascular endothelium) and traversing the BBB of a subject. In certain aspects, the disclosure provides HMW multivalent binding proteins (e.g., a DVD binding protein) comprising at least one binding domain or binding site targeting a BBB antigen combined with one or more second binding domains directed against a therapeutically relevant target. In an embodiment, the binding proteins of the disclosure have one or more binding domains that are unoccupied upon BBB uptake such that they remain available for binding to a therapeutically relevant target molecule (e.g., present in or on the brain). Alternatively, one or more of the binding domains of the DVD binding protein may be pre-loaded with a therapeutic agent (e.g., an endogenous or exogenous therapeutic protein) to facilitate delivery of the agent to the brain. Accordingly, the binding proteins of the invention are well-suited for the treatment of various neurological disorders or conditions. For example, the binding proteins of the invention are effective for treating brain and CNS diseases including, but not limited to, Alzheimer's disease (AD), Parkinson's disease (PD) or multiple sclerosis (MS). In various embodiments, the binding protein is effective for treating metastic or cancerous conditions (e.g., brain tumors or metastases). For example, the binding protein binds a target associated with a metastatic condition (e.g., HER2).

In various embodiments, the invention provides a DVD binding protein comprising at least a first and a second binding domain, wherein the first binding domain specifically binds a target that facilitates entrance of the binding protein into a brain, and the second binding domain specifically binds to amyloid precursor protein (APP), wherein administration of the binding protein to a subject is effective for modulating the concentration of APP within the brain of the subject.

In various embodiments, the invention provides a DVD binding protein comprising at least a first and a second binding domain, wherein the first binding domain specifically binds a target that facilitates entrance of the binding protein into a brain, and the second binding domain specifically binds to HER2 wherein administration of the binding protein to a subject is effective for modulating the concentration of HER2 within the brain of the subject.

In various embodiments, the invention provides a DVD binding protein comprising at least a first and a second binding domain, wherein the first binding domain specifically binds a target that facilitates entrance of the binding protein into a brain, and the second binding domain specifically binds to BACE1 wherein administration of the binding protein to a subject is effective for modulating the concentration of BACE1 within the brain of the subject.

In various embodiments, the invention provides a DVD binding protein comprising at least a first and a second binding domain, wherein the first binding domain specifically binds a target that facilitates entrance of the binding protein into a brain, and the second binding domain specifically binds to Repulsive guidance molecule A (RGMa or RGMA) wherein administration of the binding protein to a subject is effective for modulating the concentration of RGMa within the brain of the subject.

In various embodiments, the invention provides a DVD binding protein comprising at least a first and a second binding domain, wherein the first binding domain specifically binds a target that facilitates entrance of the binding protein into a brain, and the second binding domain specifically binds to amyloid β (Abeta or Aβ) wherein administration of the binding protein to a subject is effective for modulating the concentration of Abeta within the brain of the subject.

In various embodiments, the invention provides a DVD binding protein comprising at least a first and a second binding domain, wherein the first binding domain specifically binds a target that facilitates entrance of the binding protein into a brain, and the second binding domain specifically binds to TNF wherein administration of the binding protein to a subject is effective for modulating the concentration of TNF within the brain of the subject.

In various embodiments, the DVD binding proteins described herein comprises any of the amino acid sequences (e.g., APP, HER2, BACE1, Abeta, RGMa, and TNF) described in the Tables and Examples herein.

In various embodiments, the invention provides a DVD binding protein comprising at least a first and a second binding domain, such that the first binding domain specifically binds a target that facilitates entrance or passage of the binding protein across a natural BBB biological barrier, and the second binding domain specifically binds to BACE1, APP, HER2, Abeta, RGMa or TNF, such that administration of the binding protein to a subject is effective for modulating the concentration of BACE1, APP, HER2, Abeta, RGMa, or TNF within the brain of the subject. In various embodiments, the subject is a mammal. For example, the mammal is a human or a rodent.

In various embodiments, the binding protein binds an Abeta that is extracellular, intracellular, or membrane-associated. In various embodiments, the binding protein is effective for treating a condition, disease or disorder of the brain such as, for example, AD, PD or MS.

In various embodiments, the DVD binding protein is a DVD-Ig protein. In various embodiments, the DVD binding protein is a half-DVD-Ig, a scDVD-Ig, an fDVD-Ig, an rDVD-Ig, a pDVD-Ig, an mDVD-Ig or a coDVD-Ig.

In various embodiments, the DVD binding protein localizes to brain parenchyma, a neuronal cell, or a neuronal tissue. The DVD binding protein in various embodiments is formulated in a composition for in vivo administration to a subject. For example, the composition is formulated for parenteral administration. In various embodiments, the composition is formulated for subcutaneous administration or intravenous administration.

In various embodiments of the binding protein, the target that facilitates entrance into brain comprises a receptor, for example, a receptor that is expressed on brain vascular endothelial cells. In various embodiments of the binding protein, the receptor is selected from the group consisting of the insulin receptor, the transferrin receptor, LRP, melanocortin receptor, nicotinic acetylcholine receptor, VACM-1 receptor, IGFR, EPCR, EGFR, TNFR, Leptin receptor, M6PR, Lipoprotein receptor, NCAM, LIFR, LfR, MRP1, AchR, DTr, Glutathione transporter, SR-B1, MYOF, TFRC, ECE1, LDLR, PVR, CDC50A, SCARF1, MRC1, HLA-DRA, RAMP2, VLDLR, STAB1, TLR9, CXCL16, NTRK1, CD74, DPP4, endothelial growth factor receptors 1, 2 and 3, glucocorticoid receptor, ionotropic glutamate receptor, M3 receptor, aryl hydrocarbon receptor, GLUT-1, inositol-1,4,5-trisphosphate (IP3) receptor, N-methyl-D-aspartate receptor, S1P1, P2Y receptor, TMEM30A, and RAGE.

In particular embodiments, the DVD binding protein specifically binds transferrin receptor (TfR) and Abeta or RGMa.

In various embodiments, the binding protein is effective for modulating the concentration, activity, or amount of the Abeta or RGMa. In various embodiments, the binding protein is effective for modulating the extent of the solubility of the Abeta or RGMa in serum or the brain. In various embodiments, the DVD binding protein causes Abeta or RGMa levels to increase. Alternatively, in various embodiments, the DVD binding protein causes Abeta or RGMa levels to decrease.

The binding protein in various embodiments further comprises a composition and/or additional agent. In various embodiments, the composition or additional agent is co-administered with the DVD binding protein. Alternatively, the composition and/or the additional agent are administered prior to administering the binding protein. In various embodiments, the composition and/or the additional agent is administered subsequent to administering the binding protein.

In various embodiments, the binding protein binds BACE1 and comprises a VD1 or VD2 in the heavy chain that comprises an amino acid sequence selected from the group consisting of SEQ ID NOs: 93-98. In various embodiments, the binding protein binds BACE1 and comprises at least one CDR in the heavy chain that comprises an amino acid sequence selected from the group consisting of SEQ ID NOs: 136-141. In various embodiments, the binding protein binds BACE1 and comprises at least one CDR in the light chain that comprises an amino acid sequence selected from the group consisting of SEQ ID NOs: 136-141. In various embodiments, the binding protein that binds BACE1 comprises an amino acid sequence found in any Table herein, e.g., Table 3. In various embodiments, the binding protein binds BACE1 and comprises a VD1 or VD2 in the light chain that comprises an amino acid sequence selected from the group consisting of SEQ ID NOs: 87-92. In various embodiments, the binding protein binds BACE1 and comprises at least one CDR in the light chain that comprises an amino acid sequence selected from the group consisting of SEQ ID NOs: 127-134. In various embodiments, the binding protein that binds BACE1 comprises a CDR having an amino acid sequence found in any Table herein.

In various embodiments, the binding protein binds APP and comprises a VD1 or VD2 in the heavy chain that comprises an amino acid sequence of SEQ ID NO: 38. In various embodiments, the binding protein that binds APP comprises a VD1 or VD2 in the light chain that comprises an amino acid sequence of SEQ ID NO: 39. In various embodiments, the binding protein that binds APP comprises an amino acid sequence found in any Table herein. In various embodiments, the binding protein CDR that binds APP comprises an amino acid sequence found in any Table herein.

In various embodiments, the binding protein binds HER2 and comprises a VD1 or VD2 in the heavy chain that comprises an amino acid sequence of SEQ ID NO: 58. In various embodiments, the binding protein binds HER2 and a VD1 or VD2 in the light chain comprises an amino acid sequence of SEQ ID NO: 59. In various embodiments, the binding protein that binds HER2 comprises a CDR having an amino acid sequence found in any Table herein. In various embodiments, the binding protein includes a heavy chain variable domain that comprises the amino acid sequence of SEQ ID NO: 192. In various embodiments, the binding protein includes a heavy chain variable domain that comprises the amino acid sequence of SEQ ID NO: 193.

In various embodiments, the binding protein binds TNF (TNFα) and comprises a VD1 or VD2 in the heavy chain that comprises an amino acid sequence of SEQ ID NO: 162. In various embodiments, the binding protein that binds TNF comprises a VD1 or VD2 in the light chain that comprises an amino acid sequence of SEQ ID NO: 163. In various embodiments, the binding protein or component CDR that binds TNF comprises an amino acid sequence found in any Table herein (e.g., Table 2).

In various embodiments, the binding protein binds Abeta and comprises a VD1 or VD2 in the heavy chain that comprises an amino acid sequence of SEQ ID NO: 99 or SEQ ID NO: 101. In various embodiments, the binding protein that binds APP comprises a VD1 or VD2 in the light chain that comprises an amino acid sequence of SEQ ID NO:100 or SEQ ID NO: 102. In various embodiments, the binding protein that binds Abeta comprises an amino acid sequence found in any Table herein. In various embodiments, the binding protein CDR that binds TNF comprises an amino acid sequence found in any Table herein (e.g., Tables 1 and 2). In various embodiments, the binding protein that binds Abeta comprises at least one amino acid sequence selected from the group consisting of SEQ ID NOs: 196-208.

In various embodiments, the binding protein binds RGMA and comprises a VD1 or VD2 in the heavy chain that comprises an amino acid sequence selected of SEQ ID NO: 170. In various embodiments, the binding protein binds RGMA and comprises at least one CDR in the light chain that comprises an amino acid sequence of SEQ ID NO: 171. In various embodiments, the binding protein (e.g., DVD-Ig binding protein) that binds RGMA comprises a CDR having an amino acid sequence found in any Table herein.

In various embodiments, the binding protein binds TfR and comprises a VD1 or VD2 in the heavy chain that comprises an amino acid sequence selected from the group consisting of SEQ ID NOs: 30, 32, 34, 36, and 56. In various embodiments, the binding protein that binds TfR comprises a VD1 or VD2 in the light chain that comprises an amino acid sequence of SEQ ID NOs: 31, 33, 35, 37 and 57. In various embodiments, the binding protein that binds TfR comprises a CDR having an amino acid sequence found in any Table herein, e.g., Table 2.

In various embodiments, the binding protein binds TMEM30A and comprises a VD1 or VD2 in the heavy chain that comprises an amino acid sequence of SEQ ID NO: 103. In various embodiments, the binding protein binds TMEM30A and comprises a VD1 or VD2 in the light chain that comprises an amino acid sequence of SEQ ID NO: 103. In In various embodiments, the binding protein (e.g., DVD-Ig binding protein) that binds TMEM30A comprises a CDR having an amino acid sequence found in any Table herein.

In various embodiments, the binding protein binds human insulin receptor (HIR) and comprises a VD1 or VD2 in the heavy chain that comprises an amino acid sequence of SEQ ID NO: 104. In various embodiments, the binding protein that binds HIR comprises a VD1 or VD2 in the light chain that comprises an amino acid sequence selected from the group consisting of SEQ ID NOs: 105-108. In various embodiments, the binding protein or component CDR that binds HIR comprises an amino acid sequence found in any Table herein.

In various embodiments, the DVD-Ig comprises a polypeptide chain comprising the formula VD1-(X1)n-VD2-C-(X2)n, wherein

VD1 is a first heavy chain variable domain;

VD2 is a second heavy chain variable domain;

C is a heavy chain constant domain;

X1 is a linker with the proviso that it is not CH1;

X2 is an Fc region;

(X1)n is (X1)0 or (X1)1;

(X2)n is (X2)0 or (X2)1.

In various embodiments, the VD1 or VD2 comprises three CDRs, wherein at least one CDR comprises an amino acid sequence selected from the group consisting of SEQ ID NOs: 141, 142, 143, 147, 148, 149, 172, 173, and 174; the VD1 or VD2 comprises three CDRs, wherein at least one CDR comprises an amino acid sequence selected from the group consisting of SEQ ID NOs: 76, 77, 78, 82, 83, 115, 116, and 117; the VD1 or VD2 comprises three CDRs, wherein at least one CDR comprises an amino acid sequence selected from the group consisting of SEQ ID NOs: 109, 110, and 111; the VD1 or VD2 comprises three CDRs, wherein at least one CDR comprises an amino acid sequence selected from the group consisting of SEQ ID NOs: 121, 122, and 123; the VD1 or VD2 comprises three CDRs, wherein at least one CDR comprises an amino acid sequence selected from the group consisting of SEQ ID NOs: 136, 137, 138, 139, and 140; the VD1 or VD2 comprises three CDRs, wherein at least one CDR comprises an amino acid sequence selected from the group consisting of SEQ ID NOs: 153, 154, and 155; the VD1 or VD2 comprises three CDRs, wherein at least one CDR comprises an amino acid sequence selected from the group consisting of SEQ ID NOs: 156, 157, and 158; the VD1 or VD2 comprises three CDRs, wherein at least one CDR comprises an amino acid sequence selected from the group consisting of SEQ ID NOs: 164, 165, and 166; the VD1 or VD2 comprises three CDRs, wherein at least one CDR comprises an amino acid sequence selected from the group consisting of SEQ ID NOs: 172, 173, and 174; the VD1 or VD2 independently comprises an amino acid sequence selected from the group consisting of SEQ ID NOs: 99 and 101; the VD1 or VD2 independently comprises an amino acid sequence of SEQ ID NO: 38; the VD1 or VD2 independently comprises an amino acid sequence of SEQ ID NO: 58; the VD1 or VD2 independently comprises an amino acid sequence selected from the group consisting of SEQ ID NOs: 93, 94, 95, 96, 97 and 98; the VD1 or VD2 independently comprises an amino acid sequence of SEQ ID NO: 103; the VD1 or VD2 independently comprises an amino acid sequence of SEQ ID NO: 104; the VD1 or VD2 independently comprises an amino acid sequence of SEQ ID NO: 162; the VD1 or VD2 independently comprises an amino acid sequence of SEQ ID NO: 170; the VD1 or VD2 independently comprises an amino acid sequence selected from the group consisting of SEQ ID NOs: 30, 32, 34, 36, and 56; the VD1 comprises an amino acid sequence selected from the group consisting of SEQ ID NOs: 30, 32, 34, 38, 56, 103, and 104, and VD2 comprises an amino acid sequence selected from the group consisting of SEQ ID NOs: 38, 58, 93, 94, 95, 96, 97, 98, 99, 101, 162, and 170; the VD1 comprises an amino acid sequence selected from the group consisting of SEQ ID NOs: 38, 58, 93, 94, 95, 96, 97, 98, 99, 101, 162, and 170, and VD2 comprises an amino acid sequence selected from the group consisting of SEQ ID NOs: 30, 32, 34, 36, 38, 56, 103, and 104; and/or the VD1 and VD2 comprise an amino acid sequence selected from SEQ ID NOs: 40, 42, 44, 46, 48, 50, 52, 54, 60, 62, 64, 66, 68, 74, 180, 182, 184, 188, 190 and 192. In various embodiments, VD1 or VD2 binds to Abeta and comprises three CDRs, wherein at least one CDR comprises an amino acid sequence selected from the group consisting of SEQ ID NOs: 141, 142, 143, 147, 148, 149, 172, 173, and 174.

In various embodiments, the VD1 or VD2 binds transferrin receptor (TfR) and comprises three CDRs, wherein at least one CDR comprises an amino acid sequence selected from the group consisting of SEQ ID NOs: 76, 77, 78, 82, 83, 115, 116, and 117.

In various embodiments, the VD1 or VD2 binds APP and comprises three CDRs, wherein at least one CDR comprises an amino acid sequence selected from the group consisting of SEQ ID NOs: 109, 110, and 111;

In various embodiments, the VD1 or VD2 binds Her2 and comprises three CDRs, wherein at least one CDR comprises an amino acid sequence selected from the group consisting of SEQ ID NOs: 121, 122, and 123;

In various embodiments, the VD1 or VD2 binds BACE1 and comprises three CDRs, wherein at least one CDR comprises an amino acid sequence selected from the group consisting of SEQ ID NOs: 136, 137, 138, 139, and 140;

In various embodiments, the VD1 or VD2 binds TMEM30A and comprises three CDRs, wherein at least one CDR comprises an amino acid sequence selected from the group consisting of SEQ ID NOs: 153, 154, and 155;

In various embodiments, the VD1 or VD2 binds human insulin receptor (HIR) comprises three CDRs, wherein at least one CDR comprises an amino acid sequence selected from the group consisting of SEQ ID NOs: 156, 157, and 158;

In various embodiments, the VD1 or VD2 binds TNF and comprises three CDRs, wherein at least one CDR comprises an amino acid sequence selected from the group consisting of SEQ ID NOs: 164, 165, and 166;

In various embodiments, the VD1 or VD2 binds RGMA comprises three CDRs, wherein at least one CDR comprises an amino acid sequence selected from the group consisting of SEQ ID NOs: 172, 173, and 174;

In various embodiments, the VD1 or VD2 Abeta and independently comprises an amino acid sequence selected from the group consisting of SEQ ID NOs: 99 and 101;

In various embodiments, the VD1 or VD2 binds APP and independently comprises an amino acid sequence of SEQ ID NO: 38;

In various embodiments, the VD1 or VD2 binds HER2 and independently comprises an amino acid sequence of SEQ ID NO: 58;

In various embodiments, the VD1 or VD2 binds BACE1 and independently comprises an amino acid sequence selected from the group consisting of SEQ ID NOs: 93, 94, 95, 96, 97 and 98;

In various embodiments, the VD1 or VD2 binds TMEM30A and independently comprises an amino acid sequence of SEQ ID NO: 103;

In various embodiments, the VD1 or VD2 binds HIR and independently comprises an amino acid sequence of SEQ ID NO: 104;

In various embodiments, the VD1 or VD2 binds TNF and independently comprises an amino acid sequence of SEQ ID NO: 162;

In various embodiments, the VD1 or VD2 binds RGMA and independently comprises an amino acid sequence of SEQ ID NO: 170

In various embodiments, the VD1 or VD2 binds TNF and independently comprises an amino acid sequence selected from the group consisting of SEQ ID NOs: 30, 32, 34, 36, and 56;

In various embodiments, the VD1 binds TfR, HIR, or TMEM30A, and comprises an amino acid sequence selected from the group consisting of SEQ ID NOs: 30, 32, 34, 38, 56, 103, and 104, and the VD2 binds Abeta, APP, Her2, BAC1, TNF or RGMA, and comprises an amino acid sequence selected from the group consisting of SEQ ID NOs: 38, 58, 93, 94, 95, 96, 97, 98, 99, 101, 162, and 170;

In various embodiments, the VD1 binds Abeta, APP, Her2, BAC1, TNF, or RGMA, and comprises an amino acid sequence selected from the group consisting of SEQ ID NOs: 38, 58, 93, 94, 95, 96, 97, 98, 99, 101, 162, and 170, and the VD2 binds TfR, HIR, or TMEM30A, and comprises an amino acid sequence selected from the group consisting of SEQ ID NOs: 30, 32, 34, 36, 38, 56, 103, and 104; and/or

In various embodiments, the VD1 and VD2 comprise an amino acid sequence selected from SEQ ID NOs: 40, 42, 44, 46, 48, 50, 52, 54, 60, 62, 64, 66, 68, 74, 180, 182, 184, 188, 190 and 192.

In various embodiments, the DVD-Ig comprises a polypeptide chain comprising the formula VD1-(X1)n-VD2-C-(X2)n, wherein

VD1 is a first light chain variable domain;

VD2 is a second light chain variable domain;

C is a light chain constant domain;

X1 is a linker with the proviso that it is not CH1;

X2 is an Fc region;

(X1)n is (X1)0 or (X1)1;

(X2)n is (X2)0 or (X2)1.

In various embodiments, the VD1 or VD2 comprises three CDRs, wherein at least one CDR comprises an amino acid sequence selected from the group consisting of SEQ ID NOs: 144, 145, 146, 150, 151, and 152; the VD1 or VD2 comprises three CDRs, wherein at least one CDR comprises an amino acid sequence selected from the group consisting of SEQ ID NOs: 79, 80, 81, 84, 85, 86, 112, 113, 114, 118, 119, and 120; the VD1 or VD2 comprises three CDRs, wherein at least one CDR comprises an amino acid sequence selected from the group consisting of SEQ ID NOs: 112, 113, and 114; the VD1 or VD2 comprises three CDRs, wherein at least one CDR comprises an amino acid sequence selected from the group consisting of SEQ ID NOs: 124, 125, and 126; the VD1 or VD2 comprises three CDRs, wherein at least one CDR comprises an amino acid sequence selected from the group consisting of SEQ ID NOs: 127, 128, 129, 130, 131, 132, 133, 134, and 135; the VD1 or VD2 comprises three CDRs, wherein at least one CDR comprises an amino acid sequence selected from the group consisting of SEQ ID NOs: 153, 154, and 155; the VD1 or VD2 comprises three CDRs, wherein at least one CDR comprises an amino acid sequence selected from the group consisting of SEQ ID NOs: 159, 160, 161; the VD1 or VD2 comprises three CDRs, wherein at least one CDR comprises an amino acid sequence selected from the group consisting of SEQ ID NOs: 167, 168, and 169; the VD1 or VD2 comprises three CDRs, wherein at least one CDR comprises an amino acid sequence selected from the group consisting of SEQ ID NOs: 175, 176, and 177; the VD1 or VD2 independently comprises an amino acid sequence selected from the group consisting of SEQ ID NOs: 100 and 102; the VD1 or VD2 independently comprises an amino acid sequence of SEQ ID NO: 39; the VD1 or VD2 independently comprises an amino acid sequence of SEQ ID NO: 59; the VD1 or VD2 independently comprises an amino acid sequence selected from the group consisting of SEQ ID NOs: 87, 88, 89, 90, 91, and 92; the VD1 or VD2 independently comprises an amino acid sequence of SEQ ID NO: 103; the VD1 or VD2 independently comprises an amino acid sequence selected from the group consisting of SEQ ID NOs: 105, 106, 107, and 108; the VD1 or VD2 independently comprises an amino acid sequence of SEQ ID NO: 163; the VD1 or VD2 independently comprises an amino acid sequence of SEQ ID NO: 171; the VD1 or VD2 independently comprises an amino acid sequence selected from the group consisting of SEQ ID NOs: 31, 33, 35, 37, 39, and 57; the VD1 comprises an amino acid sequence selected from the group consisting of SEQ ID NOs: 31, 33, 35, 37, 39, 57, 59, 103, 105, 106, 107, and 108, and VD2 comprises an amino acid sequence selected from the group consisting of SEQ ID NOs: 39, 87, 88, 89, 90, 91, 92, 100, 102, 163, and 171; the VD1 comprises an amino acid sequence selected from the group consisting of SEQ ID NOs: 39, 87, 88, 89, 90, 91, 92, 100, 102, 163 and 171, and VD2 comprises an amino acid sequence selected from the group consisting of SEQ ID NO: 31, 33, 35, 37, 39, 57, 59, 103, 105, 106, 107, and 108; and/or the VD1 and VD2 comprise an amino acid sequence selected from SEQ ID NOs: 41, 43, 45, 47, 49, 51, 53, 55, 61, 63, 65, 67, 69, 75, 181, 193, 185, 189, 191, and 193.

In various embodiments, the VD1 or VD2 binds Abeta and comprises three CDRs, wherein at least one CDR comprises an amino acid sequence selected from the group consisting of SEQ ID NOs: 144, 145, 146, 150, 151, and 152.

In various embodiments, the VD1 or VD2 binds TfR and comprises three CDRs, wherein at least one CDR comprises an amino acid sequence selected from the group consisting of SEQ ID NOs: 79, 80, 81, 84, 85, 86, 112, 113, 114, 118, 119, and 120.

In various embodiments, the VD1 or VD2 binds APP and comprises three CDRs, wherein at least one CDR comprises an amino acid sequence selected from the group consisting of SEQ ID NOs: 112, 113, and 114.

In various embodiments, the VD1 or VD2 binds Her2 and comprises three CDRs, wherein at least one CDR comprises an amino acid sequence selected from the group consisting of SEQ ID NOs: 124, 125, and 126.

In various embodiments, the VD1 or VD2 binds BACE1 and comprises three CDRs, wherein at least one CDR comprises an amino acid sequence selected from the group consisting of SEQ ID NOs: 127, 128, 129, 130, 131, 132, 133, 134, and 135.

In various embodiments, the VD1 or VD2 binds TMEM30A and comprises three CDRs, wherein at least one CDR comprises an amino acid sequence selected from the group consisting of SEQ ID NOs: 153, 154, and 155.

In various embodiments, the VD1 or VD2 binds HIR2 and comprises three CDRs, wherein at least one CDR comprises an amino acid sequence selected from the group consisting of SEQ ID NOs: 159, 160, and 161.

In various embodiments, the VD1 or VD2 binds TNF and comprises three CDRs, wherein at least one CDR comprises an amino acid sequence selected from the group consisting of SEQ ID NOs: 167, 168, and 169.

In various embodiments, the VD1 or VD2 binds RGMA and comprises three CDRs, wherein at least one CDR comprises an amino acid sequence selected from the group consisting of SEQ ID NOs: 175, 176, and 177.

In various embodiments, the VD1 or VD2 binds Abeta and independently comprises an amino acid sequence selected from the group consisting of SEQ ID NOs: 100 and 102;

In various embodiments, the VD1 or VD2 binds APP and independently comprises an amino acid sequence of SEQ ID NO: 39.

In various embodiments, the VD1 or VD2 binds Her2 and independently comprises an amino acid sequence of SEQ ID NO: 59.

In various embodiments, the VD1 or VD2 binds BACE1 and independently comprises an amino acid sequence selected from the group consisting of SEQ ID NOs: 87, 88, 89, 90, 91, and 92.

In various embodiments, the VD1 or VD2 binds TMEM30A and independently comprises an amino acid sequence of SEQ ID NO: 103.

In various embodiments, the VD1 or VD2 binds HIR and independently comprises an amino acid sequence selected from the group consisting of SEQ ID NOs: 105, 106, 107, and 108.

In various embodiments, the VD1 or VD2 binds TNF and independently comprises an amino acid sequence of SEQ ID NO: 163.

In various embodiments, the VD1 or VD2 binds RGMA and independently comprises an amino acid sequence of SEQ ID NO: 171.

In various embodiments, the VD1 or VD2 binds TfR and independently comprises an amino acid sequence selected from the group consisting of SEQ ID NOs: 31, 33, 35, 37, 39, and 57.

In various embodiments, the VD1 binds to TfR, HIR or TMEM30A, and comprises an amino acid sequence selected from the group consisting of SEQ ID NOs: 31, 33, 35, 37, 39, 57, 59, 103, 105, 106, 107, and 108, and the VD2 binds to APP, BACE1, Abeta, TNF or RGMA, and comprises an amino acid sequence selected from the group consisting of SEQ ID NOs: 39, 87, 88, 89, 90, 91, 92, 100, 102, 163, and 171.

In various embodiments, the VD1 binds to APP, BACE1, Abeta, TNF or RGMA, and comprises an amino acid sequence selected from the group consisting of SEQ ID NOs: 39, 87, 88, 89, 90, 91, 92, 100, 102, 163 and 171, and the VD2 binds TfR, HIR, or TMEM30A; and comprises an amino acid sequence selected from the group consisting of SEQ ID NO: 31, 33, 35, 37, 39, 57, 59, 103, 105, 106, 107, and 108; or

In various embodiments, the VD1 and VD2 comprise an amino acid sequence selected from SEQ ID NOs: 41, 43, 45, 47, 49, 51, 53, 55, 61, 63, 65, 67, 69, 75, 181, 193, 185, 189, 191, and 193.

In various embodiments, the VD1, VD2, or VD1-X1-VD2, VD1-X2-VD2 is any of the sequences shown in Tables and Working Examples herein.

In various embodiments, (X1)n is (X1)0.

In various embodiments, the linker comprises an amino acid sequence selected from the group consisting of AKTTPKLEEGEFSEAR (SEQ ID NO: 1); AKTTPKLEEGEFSEARV (SEQ ID NO: 2); AKTTPKLGG (SEQ ID NO: 3); SAKTTPKLGG (SEQ ID NO: 4); SAKTTP (SEQ ID NO: 5); RADAAP (SEQ ID NO: 6); RADAAPTVS (SEQ ID NO: 7); RADAAAAGGPGS (SEQ ID NO: 8); RADAAAA (G₄S)₄ (SEQ ID NO: 9); SAKTTPKLEEGEFSEARV (SEQ ID NO: 10); ADAAP (SEQ ID NO: 11); ADAAPTVSIFPP (SEQ ID NO: 12); TVAAP (SEQ ID NO: 13); TVAAPSVFIFPP (SEQ ID NO: 14); QPKAAP (SEQ ID NO: 15); QPKAAPSVTLFPP (SEQ ID NO: 16); AKTTPP (SEQ ID NO: 17); AKTTPPSVTPLAP (SEQ ID NO: 18); AKTTAP (SEQ ID NO: 19); AKTTAPSVYPLAP (SEQ ID NO: 20); ASTKGP (SEQ ID NO: 21); ASTKGPSVFPLAP (SEQ ID NO: 22), GGGGSGGGGSGGGGS (SEQ ID NO: 23); GENKVEYAPALMALS (SEQ ID NO: 24); GPAKELTPLKEAKVS (SEQ ID NO: 25); or GHEAAAVMQVQYPAS (SEQ ID NO: 26); TVAAPSVFIFPPTVAAPSVFIFPP (SEQ ID NO: 27); ASTKGPSVFPLAPASTKGPSVFPLAP (SEQ ID NO: 28); G/S based sequences (e.g., G4S repeats; SEQ ID NO: 29); GGGGSGGGGS (SEQ ID NO:178); GGSGGGGSG (SEQ ID NO:179), GSGSGNGS (SEQ ID NO: 209), GSGSGSGS (SEQ ID NO: 210), GGSGSGSG (SEQ ID NO: 211), GGSGSG (SEQ ID NO: 212), GGSG (SEQ ID NO: 213), GGSGNGSG (SEQ ID:214), or GSG (SEQ ID NO: 215). In various embodiments, the linker comprises a portion of the linkers described above.

In various embodiments, the binding protein comprises at least one disulfide bond.

For example, the DVD-Ig binding protein comprises an antibody or portion thereof described herein, e.g., Tables 1-20. In various embodiments, the binding protein comprises an amino acid sequence found in Tables 1-6. In various embodiments, the binding protein comprises a Fc constant region. In various embodiments, the Fc constant region comprises at least one mutation compared to a wild-type Fc constant region. For example, the binding protein comprises at least one amino acid sequence selected from the group consisting of SEQ ID NOs: 70, 71, 72, and 73. In various embodiments, the Fc region comprises at least one mutation compared to SEQ ID NOs: 70, 71, 72 and 73. In various embodiments, the binding protein comprises a CH domain and/or a CL domain.

In various embodiments of the binding protein, (X1)n is (X1)0.

An aspect of the invention provides an isolated nucleic acid encoding any one of the DVD binding proteins described herein.

An aspect of the invention provides a vector comprising any isolated nucleic acid described herein.

An aspect of the invention provides a host cell comprising any vector described herein.

Host cells useful in various embodiments are prokaryotic, e.g., Escherichia coli. Host cells useful in various embodiments are eukaryotic, e.g., a protist cell, an animal cell, a plant cell, and a fungal cell.

An aspect of the invention provides methods of producing a DVD binding protein, the method including the step of culturing a host cell described herein in culture medium under conditions sufficient to produce the DVD binding protein. The method in various embodiments produces the binding protein using various compositions, buffers, and/or materials described herein. In various embodiments, the protein produced by the method described herein is isolated and purified.

An aspect of the invention provides a pharmaceutical composition comprising any of the DVD binding proteins described herein, and a pharmaceutically acceptable carrier. For example, the pharmaceutical composition includes a DVD binding protein that specifically binds a receptor. For example, the DVD binding protein binds a receptor (e.g., a transferrin receptor). In various embodiments, the binding protein modulates the concentration, amount or activity of Abeta or RGMa. For example, the pharmaceutical composition is effective for treating or ameliorating the severity of AD, PD or and MS. For example, the pharmaceutical composition reduces at least one symptom and/or improves a metric associated with AD, PD, or MS.

In various embodiments, the pharmaceutical composition further comprises at least one additional therapeutic agent. In various embodiments, the additional therapeutic agent is selected from the group consisting of an imaging agent, a cytotoxic agent, an angiogenesis inhibitor, a kinase inhibitor, a co-stimulation molecule blocker, an adhesion molecule blocker, an anti-cytokine antibody or functional fragment thereof, a detectable label or reporter, an antirheumatic, a muscle relaxant, a narcotic, a non-steroid anti-inflammatory drug (NSAID), an analgesic, an anesthetic, a sedative, a neuromuscular blocker, an antimicrobial, an antipsoriatic, a corticosteriod, an anabolic steroid, an erythropoietin, an immunoglobulin, an immunosuppressive, a growth hormone, a hormone replacement drug, a radiopharmaceutical, an antidepressant, an antipsychotic, a stimulant, an asthma medication, a beta agonist, an inhaled steroid, an epinephrine or analog, a cytokine, and a cytokine antagonist.

In an embodiment, the additional therapeutic agent is selected from the group consisting of budenoside, epidermal growth factor, a corticosteroid, cyclosporin, sulfasalazine, an aminosalicylate, 6-mercaptopurine, azathioprine, metronidazole, a lipoxygenase inhibitor, mesalamine, olsalazine, balsalazide, an antioxidant, a thromboxane inhibitor, a growth factor, an elastase inhibitor, a pyridinyl-imidazole compound, an antibody, antagonist or agonist of TNF, LT, IL-1, IL-1R, IL-2, IL-4, IL-6, IL-6R, IL-7, IL-8, IL-10, IL-11, IL-12, IL-13, IL-15, IL-16, IL-18, IL-23, TGF-β, EMAP-II, GM-CSF, FGF, PDGF, CD2, CD3, CD4, CD8, CD-19, CD25, CD28, CD30, CD40, CD45, CD69, CD90 or a ligand thereof, methotrexate, FK506, rapamycin, mycophenolate mofetil, leflunomide, ibuprofen, prednisolone, a phosphodiesterase inhibitor, an adenosine agonist, an antithrombotic agent, a complement inhibitor, an adrenergic agent, IRAK, NIK, IKK, p38, a MAP kinase inhibitor, an IL-1β converting enzyme inhibitor, a TNFα-converting enzyme inhibitor, a T-cell signaling inhibitor, a metalloproteinase inhibitor, an angiotensin converting enzyme inhibitor, a soluble cytokine receptor, a soluble p55 TNF receptor, a soluble p75 TNF receptor, sIL-1RI, sIL-1RII, sIL-6R and combinations thereof.

An aspect of the invention provides a kit comprising: a DVD binding protein described herein or the pharmaceutical composition described herein; and, a container.

An aspect of the invention provides a method for treating Alzheimer's disease (AD), in a subject in need thereof by administering to the subject a DVD binding protein comprising at least a first and a second binding domain, wherein the first binding domain specifically binds a target that facilitates entrance of the binding protein into a brain, and the second binding domain specifically binds to Abeta, wherein administration of the binding protein to a subject is effective for modulating the concentration of Abeta within the brain of the subject.

An aspect of the invention provides a method for modulating Abeta levels within a cell in a subject's central nervous system comprising administering to the subject a dual variable domain (DVD) binding protein comprising at least a first and a second binding domain, wherein the first binding domain specifically binds a target that facilitates entrance of the binding protein into a brain, and the second binding domain specifically binds to Abeta, and administration of the binding protein to a subject is effective for modulating the concentration of Abeta within the brain of the subject. In various embodiments, the binding protein that binds Abeta comprises at least one amino acid sequence found in Tables herein. For example, the binding protein that binds Abeta comprises at least one amino acid sequence selected from the group consisting of SEQ ID NOs: 196-208.

In various embodiments of the method, the Abeta level within cells is at least: about 1%-5%, about 5%-10%, about 10%-15%, about 15%-20%, about 20%-25%, or about 25%-30% greater than the Abeta level within cells in the absence of the DVD binding protein. In various embodiments of the method, the Abeta level within cells is about 20% greater than the Abeta level within cells in the absence of the DVD binding protein.

In various embodiments of the method the modulating the concentration of Abeta occurs between 4 and 24 hours after administration of the DVD binding protein to the subject. For example, modulating occurs after at least about 4 hours to about 8 hours, about 8 hours to about 12 hours, about 12 hours to about 16 hours, about 16 hours to about 20 hours, about 20 hours to about 24 hour, or about 24 hours to about 30 hours after administration of the DVD binding protein to the subject.

In various embodiments of the method, the Abeta is intracellular. In various embodiments, the Abeta is cell membrane bound. In various embodiments of the method, the Abeta is extracellular.

In various embodiments of the method, administering the binding protein to the subject is effective for modulating Abeta levels and reducing the formation of insoluble brain plaques associated with AD, PD and/or MS.

An aspect of the invention provides a method for treating multiple sclerosis in a subject in need thereof by administering to the subject a DVD binding protein described herein, comprising at least a first and a second binding domain, wherein the first binding domain specifically binds a target that facilitates entrance of the binding protein into a brain, and the second binding domain specifically binds to RGMa, wherein administration of the binding protein to a subject is effective for modulating the RGMa levels within the brain of the subject.

An aspect of the invention provides a method for modulating myelin basic protein levels within a cell in a subject's central nervous system comprising administering to the subject a DVD binding protein described herein, comprising at least a first and a second binding domain, wherein the first binding domain specifically binds a target that facilitates entrance of the binding protein into a brain, and the second binding domain specifically binds to RGMa, wherein administration of the binding protein to a subject is effective for modulating the myelin basic protein levels within the brain of the subject. In various embodiments, the binding protein increases myelin basic protein level compared to the level observed in the absence of the DVD binding protein or pharmaceutical composition. In various embodiments, the modulating occurs between 4 and 24 hours after administration of the DVD binding protein or pharmaceutical composition to the subject.

An aspect of the invention provides a method for treating Parkinson's disease in a subject in need thereof by administering to the subject a DVD binding protein described herein comprising at least a first and a second binding domain, wherein the first binding domain specifically binds a target that facilitates entrance of TNF into the brain, wherein administration of the binding protein to a subject is effective for modulating the concentration of TNF within the brain and/or cerebrospinal fluid of the subject. In various embodiments, the method modulates the concentration of TNF such that improvement of the Parkinson's disease is observed. In various embodiments, the binding protein reduces the loss of dopaminergic neurons in the subject.

An aspect of the invention provides a method for treating a disease or condition in a subject in need thereof by administering to the subject a DVD binding protein described herein comprising at least a first and a second binding domain, wherein the first binding domain specifically binds a target that facilitates entrance into the brain, and the second binding domain specifically binds to BACE1, HER2, or APP, wherein administration of the binding protein to a subject is effective for modulating the concentration of BACE1, HER2, or APP in the brain or cerebrospinal fluid of the subject. In various embodiments, the disease or condition is neurological or associated with cancer. In various embodiments, the disease or condition is selected from the group consisting of: Alzheimer's disease, Parkinson's disease, and multiple sclerosis.

In various embodiments, the DVD binding protein is a DVD-Ig protein. In various embodiments, the DVD binding protein is selected from the group consisting of a half-DVD-Ig, a scDVD-Ig, an fDVD-Ig, an rDVD-Ig, a pDVD-Ig, an mDVD-Ig and a coDVD-Ig.

In various embodiments, the DVD-Ig comprises a polypeptide chain comprising the formula VD1-(X1)n-VD2-C-(X2)n, wherein

VD1 is a first heavy chain variable domain;

VD2 is a second heavy chain variable domain;

C is a heavy chain constant domain;

X1 is a linker (for example a linker with the proviso that it is not CH1);

X2 is an Fc region;

(X1)n is (X1)0 or (X1)1;

(X2)n is (X2)0 or (X2)1.

In various embodiments, the VD1 or VD2 binds APP and comprises three CDRs, wherein at least one CDR comprises an amino acid sequence selected from the group consisting of SEQ ID NOs: 109, 110, and 111. In various embodiments, the VD1 or VD2 binds APP and independently comprises an amino acid sequence of SEQ ID NO: 38. In various embodiments, the binding protein that binds APP comprises an amino acid sequence described in a Table herein.

In various embodiments, the VD1 or VD2 binds BACE1 and comprises three CDRs, wherein at least one CDR comprises an amino acid sequence selected from the group consisting of SEQ ID NOs: 136, 137, 138, 139, and 140. In various embodiments, the VD1 or VD2 binds BACE1 and independently comprises an amino acid sequence selected from the group consisting of SEQ ID NOs: 93, 94, 95, 96, 97 and 98. In various embodiments, the binding protein that binds BAC1 comprises an amino acid sequence described in a Table herein.

In various embodiments, the VD1 or VD2 binds Her2 and comprises three CDRs, wherein at least one CDR comprises an amino acid sequence selected from the group consisting of SEQ ID NOs: 121, 122, and 123. In various embodiments, the VD1 or VD2 that binds Her2 independently comprises an amino acid sequence of SEQ ID NO: 58. In various embodiments, the binding protein that binds Her2 comprises an amino acid sequence described in a Table herein.

In various embodiments, the DVD-Ig comprises a polypeptide chain comprising the formula VD1-(X1)n-VD2-C-(X2)n, wherein

VD1 is a first light chain variable domain;

VD2 is a second light chain variable domain;

C is a light chain constant domain;

X1 is a linker (for example a linker with the proviso that it is not CH1);

X2 is an Fc region;

(X1)n is (X1)0 or (X1)1;

(X2)n is (X2)0 or (X2)1.

In various embodiments, the VD1 or VD2 binds APP and comprises three CDRs, wherein at least one CDR comprises an amino acid sequence selected from the group consisting of SEQ ID NOs: 112, 113, and 114. In various embodiments, the VD1 or VD2 binds APP and independently comprises an amino acid sequence of SEQ ID NO: 39. In various embodiments, the binding protein that binds APP comprises an amino acid sequence described in a Table herein.

In various embodiments, the VD1 or VD2 that binds BACE1 comprises three CDRs, wherein at least one CDR comprises an amino acid sequence selected from the group consisting of SEQ ID NOs: 127, 128, 129, 130, 131, 132, 133, 134, and 135. In various embodiments, the VD1 or VD2 that binds BACE1 independently comprises an amino acid sequence selected from the group consisting of SEQ ID NOs: 87, 88, 89, 90, 91, and 92.

In various embodiments, the VD1 or VD2 that binds Her2 comprises three CDRs, wherein at least one CDR comprises an amino acid sequence selected from the group consisting of SEQ ID NOs: 124, 125, and 126. In various embodiments, the VD1 or VD2 that binds Her2 independently comprises an amino acid sequence of SEQ ID NO: 59.

In various embodiments of the method, the administration of the binding protein is parenteral, subcutaneous, intramuscular, intravenous, intrarticular, intrabronchial, intraabdominal, intracapsular, intracartilaginous, intracavitary, intracelial, intracerebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrathecal, epidural, intrasynovial, intrathoracic, intrauterine, intravesical, bolus, vaginal, rectal, buccal, sublingual, intranasal, or transdermal.

### BRIEF DESCRIPTION OF THE DRAWINGS

FIG. 1 is a schematic representation of Dual Variable Domain (DVD) binding protein constructs.

FIG. 2 is a set of micrographs of brain tissues showing elevated levels of AB221 TfR mAb in brain extracts using Meso Scale Discovery-electrochemiluminescence (MSD-ECL) localized to parenchyma and neuronal cell bodies using immunohistochemistry (IHC) after therapeutic dosing.

FIG. 3 is a set of micrographs of brain tissues showing elevated DVD-Ig protein levels localized to Purkinje cells in cerebellum and cranial nerve cell bodies by IHC.

FIG. 4 is a flow chart representation of methods and systems used for the generation and in vitro/in vivo screening of receptor-mediated transcytosis domain DVD-Ig proteins.

FIG. 5 is a schematic representation of an exemplary DVD-Ig protein. The DVD-Ig protein includes at least one variable domain that specifically binds a BBB antigen (anti-BBB antigen), and at least one variable domain that specifically binds a different target X. For example, the DVD-Ig protein in various embodiments is a TNFα/TfR, RGMA/TfR, Abeta/TfR, APP/TfR, BACE1/TfR or Her2/TfR DVD-Ig protein.

FIG. 6, panel A is a graph showing signal for protein binding to Abeta (ordinate) as a function of concentration of binding protein (abscissa; ng/nL). The binding proteins used were an Abeta monoclonal antibody, a TfR-Abeta DVD-Ig binding protein, an Abeta-TfR DVD-Ig binding protein, a control DVD-Ig binding protein, or a mouse immunoglobulin.

FIG. 6, panel B is a graph showing signal for protein binding to TfR (ordinate) as a function of concentration of binding protein (abscissa; ng/nL). The binding proteins used were AB221 TfR antibody, AB405-SL-Abeta (3D6) DVD-Ig binding protein, or Abeta (3D6)-GS-AB403 DVD-Ig binding protein.

FIG. 7 is a schematic representation of an in vivo tissue distribution protocol used for analyzing characteristics of an antibody or a DVD-Ig protein. Tissues were homogenized and Ab/DVD-Ig amounts were quantified. Tissues were snap frozen in liquid nitrogen. Hard tissues (e.g., colon, skin, etc.) were pulverized while frozen either manually or using a Cryoprep™ system, Homogenization of the tissues involved using beads in lysis buffer with protease inhibitors, and a blender. The homogenate material was rotated for an hour, then spun, aliquoted and frozen. The antibody concentration was measured using a MSD assay with an anti-huFc, or hu-/mu TNF capture antibody.

FIG. 8 is a set of micrographs of stained brain tissue from subjects administered with either (1) 40 mpk of control human IgG at 24 hours; (2) 20 mpK of a 8C11-hFc binding protein at 48 hours; (3) 30 mpk of a non-specific DVD-Ig protein control at 48 hours; (4) 20 mpk of TNFα-GS-AB221 DVD-Ig protein at 24 hours; or (5) 20 mpk of TfR (AB405)-SL-TNF DVD-Ig protein at 24 hours.

FIG. 9 is a bar graph showing the percent maximum possible effect (% MPE; ordinate) at day 1 and day 5 for BALB-C mice 15 days after Bennett surgery and after intrathecal injection (abscissa) with (1) control IgG (48 μg/10 μl dose per injection); (2) 8C11-GS-AB221 DVD-Ig protein (anti-TNFα/anti-TfR; 55 μg/10 μl dose per injection); or (3) morphine (10 μg/10 μl dose per injection). The injections were performed daily for five days after the Bennett surgery. Mechanical allodynia was assessed in the Bennett model 120 minutes post-injection at day 1 and day 5. IgG (IgG Fc mut, 48 μg/10 μl/mouse) anti-TNFα/TfR DVD (8C11-GS-AB221, 55 μg/10 μl/mouse dosed i.t. QD for 5 days. Mechanical anodynia to be assessed 120 minutes post-drug administration at day 1 & 5. Morphine (10 μg/10 μl/mouse in saline) i.t. acute on day 1 & day 5 testing. Experiments done in BALB/c male mice approximately 15 days post-Bennett surgery. Plasma, *p<0.05; **p<0.01; ***p<0.001 vs vehicle (n=10).

FIG. 10 is a bar graph showing the percent efficacy (ordinate) at day 1 and day 5 for BALB-C mice 15 days after intravenous injection (abscissa) with (1) control IgG (48 μg/10 μl dose per injection); (2) 8C11-GS-AB221 DVD-Ig protein (anti-TNFα/anti-TfR; 55 μg/10 μl dose per injection); or (3) an acute post-operation dose of gabapentin (10 μg/10 μl dose per injection). The injections were performed daily for five days after the Bennett surgery. Mechanical allodynia was assessed in the above Bennett model 120 minutes post-injection at day 1 and at day 5.

FIG. 11, panel A is a set of micrographs of stained brain tissue at 24 hours and 48 hours from mice administered with either (1) 40 mpk of RGMA (AE12-1)-hFc; (2) 30 mpk human IgG control; (3) 20 mpk of RGMA (AE12-1)-GS-AB403 DVD-Ig protein; or (4) 30 mpk of RGMA (AE12-1)-GS-AB403 DVD-Ig protein.

FIG. 11, panel B is a table showing route of administration (IV), doses (40 mg/kg), time (24 hours) and brain concentration (nM±SD) in the brain at 24 hours for mice intravenously administered (40 mg/kg) anti-RGMA antibody, or a TfR/RGMA DVD-Ig binding protein.

FIG. 12, panel A and FIG. 12, panel B are a set of bar graphs showing the amount of 3D6 anti-amyloid beta antibody and TfR/Abeta DVD-Ig protein in serum and brain samples, respectively (ordinate), of Tg2576 mice intravenously injected with 5 mpk 3D6 or 20 mpk AB405-SL-AB(3D6), a TfR/Abeta DVD-Ig protein. Control subjects were intravenously injected with control IgG1 (anti-IL-18 antibody). FIG. 12, panel A shows amounts of anti-amyloid beta antibody and TfR-SL-Aβ (3D6) DVD-Ig protein, respectively (ordinate; nanomolar, nM), in serum as a function of time (2 hours, 4 hours and 14 day). FIG. 12, panel B shows amounts of anti-amyloid beta antibody and TfR-SL-Aβ (3D6) DVD-Ig protein, respectively (ordinate; nanomolar, nM), in brain cerebellum as a function of time (2 hours, 4 hours and 14 days). Data show greater amounts of TfR/Abeta DVD-Ig protein in the serum and brain of mice compared to 3D6 antibody. The homogenates were prepared using different mixtures/buffers: phosphate buffered saline (PBS), nonyl phenoxypolyethoxylethanol (NP-40); or sodium dodecyl sulphate (SDS).

FIG. 13 is a bar graph showing the amount of Aβ (ordinate; ng/mL) in serum samples from Tg2576 mice at different time points (abscissa; 4 hours and 24 hours) after being intravenously injected with either 3D6 antibody (5 mpk) or AB405-SL-AB (3D6), which is a TfR/Abeta DVD-Ig protein (20 mpk). Control subjects were intravenously injected with control IgG.

FIG. 14, panels A-D are a set of bar graphs showing the amount (pg/mg) of Aβ in brain samples from Tg2576 mice at different time points (abscissa; 4 hours, 24 hours and 14 days) after being intravenously injected with either 3D6 antibody (5 mpk) or TfR/Abeta DVD-Ig protein AB405-SL-AB (3D6) (20 mpk). Control subjects were intravenously injected with control IgG. FIG. 14, panel A shows the total amount of Aβ¹⁻⁴⁰ (Aβ40; Aβ peptides 1-40); Aβ¹⁻³⁸ (Aβ38; Aβ peptides 1-38); and Aβ¹⁻⁴² (Aβ42; Aβ peptides 1-42) in each brain sample. FIG. 14, panel B shows the (pg/mg) amount of Aβ42. FIG. 14, panel C shows the (pg/mg) amount of Aβ38. FIG. 14, panel D shows the amount of Aβ40.

FIG. 15, panels A-D are a set of a bar graphs showing the amount (ordinate; pg/mg) of Aβ40 in brain homogenates from Tg2576 subjects at different time points (abscissa; 4 hours and 24 hours) after being intravenously injected with either 3D6 antibody (5 mpk) or AB405-SL-AB (3D6), which is a TfR/Abeta DVD-Ig protein (20 mpk). Control subjects were intravenously injected with control IgG. FIG. 15, panel A shows the total amount of Aβ40 in each type of brain homogenate (i.e., PBS, NP-40 and SDS). FIG. 15, panel B shows the amount of Aβ40 in NP-40 homogenates. FIG. 15, panel C shows the amount of Aβ40 in PBS homogenates. FIG. 15, panel D shows the amount of Aβ40 in SDS homogenates.

FIG. 16, panels A-D are a set of a bar graphs showing the amount (ordinate; pg/mg) of Aβ38 in brain homogenates from Tg2576 mice at different time points (abscissa; 4 hours and 24 hours) after being intravenously injected with either 3D6 antibody (5 mpk) or TfR/Abeta DVD-Ig protein AB405-SL-AB (3D6) (20 mpk). Control subjects were intravenously injected with control IgG. The homogenates were prepared using different mixtures/buffers: PBS, NP-40; or SDS. FIG. 16, panel A shows the total amount of Aβ38 in each type of brain homogenate (i.e., PBS, NP-40 and SDS). FIG. 16, panel B shows the amount of Aβ38 in NP-40 homogenates. FIG. 16, panel C shows the amount of Aβ38 in PBS homogenates. FIG. 16, panel D shows the amount of Aβ38 in SDS homogenates.

FIG. 17, panels A-D are a set of a bar graphs showing the amount (ordinate; pg/mg) of Aβ42 in brain homogenates from Tg2576 mice at different time points (abscissa; 4 hours and 24 hours) after being intravenously injected with either 3D6 antibody (5 mpk) or AB405-SL-AB (3D6) DVD-Ig protein (20 mpk). Control subjects were intravenously injected with control IgG. The homogenates were prepared using different mixtures/buffers: PBS, NP-40; or SDS. FIG. 17, panel A shows the total amount of Aβ42 in each type of brain homogenate (i.e., PBS, NP-40 and SDS). FIG. 17, panel B shows the amount of Aβ42 in NP-40 homogenates. FIG. 17, panel C shows the amount of Aβ42 in PBS homogenates. FIG. 17, panel D shows the amount of Aβ42 in SDS homogenates.

FIG. 18, panels A-I are a series of photographs of murine central nervous system tissues stained for myelin basic protein for efficacy evaluation for an in vivo model. Data was generated using a “covered area” measurement, involving determining the number of pixel/section that show a minimum grey-scale value in relation to all pixels in that section—e.g., are positive for MBP staining. FIG. 18, panels A-C show representative photographs. FIG. 18, panel D corresponds a score of 4 showing normal myelin; FIG. 18, panel E corresponds to a score of zero with demyelination; FIG. 18, panel F corresponds to a score of 1 with low re-myelination; FIG. 18, panel G corresponds to a score of 2 showing medium re-myelination; FIG. 18, panel H corresponds to a score of 3 showing my re-myelination; and FIG. 18, panel I corresponds to a score of 4 showing complete myelination.

FIG. 19, panels A-H show photographs of mouse brain stained for myelin basic protein. Subjects were previously administered either: control food and human IgG; cuprizone and human IgG; cuprizone and RGMA (AE12-1)-GS-TfR(AB403) DVD-Ig protein; or cuprizone and anti-RGMA AE12-1).

FIG. 20, panels A-D are a graph and set of photomicrographs related to brain tissues from subjects administered anti-RGMA antibody or anti-RGMA.anti-TfR DVD-Ig binding protein in a MS study using cuprizone.

FIG. 20, panel A is a bar graph showing the relative decrease in myelin basic protein positive areas compared to human IgG treatment only in mouse brain from subjects administered control, RGMA (AE12-1)-GS-TfR (AB403) DVD-Ig protein or RGMa monoclonal antibody after cuprizone challenge. Control subjects were administered human IgG only, viz. no cuprizone treatment.

FIG. 20, panel B is a set of exemplary photomicrographs of mouse brain tissue from subjects (described in FIG. 20, panel A) administered control, RGMA (AE12-1)-GS-TfR (AB403) DVD-Ig protein or RGMa monoclonal antibody after cuprizone challenge. Control subjects were administered human IgG only.

FIG. 20, panel C is a bar graph and representative photomicrographs showing the relative percent of binding protein in the brain as compared to serum (ordinate) for subjects administered human IgG1/k mutant protein with cuprizone; human IgG1/k RGMA (AE12-1)-GS-TfR (AB403) DVD-Ig protein with cuprizone; or RGMa monoclonal antibody (Ae12-1) with cuprizone. Control subjects were administered human IgG1/k mutant protein only.

FIG. 20, panel D is a graph showing antibody/DVD-Ig protein concentration in the brain (ordinate) from 4 wk Cuprizone studies (Neuro2014-17 & Neuro2013-38) and 24 h PK study using RGMA antibody and RGMA/TfR DVD-Ig binding protein. Subjects received intraperitoneal injections of RGMA or RGMA/TfR antibody on days 0, 2, 4, 7, 9, 11, 14, 16, 18, 21, 23, and 25. During the treatment period of four weeks, 20 μl of whole blood was collected every Thursday, always 24 hours after the last shot of antibody/DVD. Day 28 and Day 29 involved perfusion of the murine subjects and brains were collected 72 hours/96 hours post last dose.

FIG. 21 is a bar graph showing the accumulation of antibody in serum of subjects administered RGMA (AE12-1)-GS-TfR (AB403) DVD-Ig protein, RGMa monoclonal antibody (AE12-1), human IgG1/k, or human IgG1/k and cuprizone.

FIG. 22, panel A is a bar graph showing the amount of human IgG (no cuprizone, human IgG, RGMA (AE12-1)-GS-TfR (AB403) DVD-Ig protein, and RGMa monoclonal antibody in the brain of mouse subjects.

FIG. 22, panel B shows the relative percentage of human IgG (no cuprizone, human IgG, RGMA (AE12-1)-GS-TfR (AB403) DVD-Ig protein, and RGMa monoclonal antibody in the brain of mouse subjects compared to the amount present in serum of the mouse subjects.

FIG. 23 is a bar graph showing the relative decrease in myelin basic protein positive areas in mouse brain when equal amounts of administered control, RGMA(AE12-1)-GS-TfR(AB403) DVD-Ig protein or RGMa monoclonal antibody after cuprizone challenge.

FIG. 24 is a graph showing experimental autoimmune encephalomyelitis (EAE) scores (ordinate) for murine C57Bl/6J subjects administered myelin oligodendrocyte glycoprotein (MOG) days earlier and then injected (5 mpk) RGMA antibody (AE12-1Y) or RGMA/TfR (AE12.1/AB403; 20 mpk) DVD-Ig binding protein) (abscissa). Control subjects were administered MOG and then a control human immunoglobulin (5 mpk).

FIG. 25 is a schematic of a two week PK study using 9 wk CB-17/Icr-Fox Chase SCID female mice intraperitoneally administered either Herceptin (5 mpk) or Her2-GS-TfR (AB403) DVD-Ig binding protein. A total of five doses were administered over 2 weeks. Tail nick bleeds (day zero and at day 2, day 4, day 7, day 9, and day 11), terminal serum (MSD analysis) and brain (MSD & IHC analysis) were performed.

FIG. 26 panel A is a graph showing the concentration in the brain (nM; ordinate) of the Herceptin (5 mpk) and Her2-GS-TfR (AB403) DVD-Ig binding protein (20 mpk) (abscissa) administered to SCID mice.

FIG. 26 panel B is a graph showing the serum concentration (nM; ordinate) of the Herceptin (5 mpk) and Her2-GS-TfR (AB403) DVD-Ig binding protein (20 mpk) (abscissa) administered to SCID mice.

FIG. 26 panel C is a graph showing the percentage of brain concentration divided by serum concentration (%; ordinate) of the Herceptin (5 mpk) and Her2-GS-TfR DVD-Ig binding protein (20 mpk) (abscissa) administered to SCID mice.

FIG. 27 concentration (nM, ordinate) of Herceptin and Her2-Gs-Tfr (AB403) DVD-Ig binding protein in SCID mice tail bleed samples (abscissa).

FIG. 28 shows concentration (nM, ordinate) of Herceptin and Her2-Gs-Tfr DVD-Ig binding protein in SCID mice tail bleed samples and serum samples (abscissa).

FIG. 29 is a set of representative photomicrographs of brain purkinje samples and cranial samples (first and second column respectively) from SCID subjects administered either Herceptin (first row) or Her2-Gs-Tfr DVD-Ig binding protein.

### DETAILED DESCRIPTION

The invention in various embodiments involves binding proteins that penetrate the Blood-Brain Barrier (BBB). While naturally protective, the BBB provides a challenge for drug development as more than 98% of all small organic molecule drugs do not cross the BBB to therapeutically-relevant concentrations; nearly all large molecules do not cross the BBB. Examples herein include data showing the brain uptake, retention and in vivo PD/efficacy of HMW binding proteins, e.g., DVD-Ig binding proteins. Proprietary binding proteins described herein showed significant uptake into brain and spinal cord.

The invention in various embodiments involves applying the binding proteins and methods described herein to overcome BBB. Examples herein describe the design and formulation of biologics for delivery into the BBB.

Accordingly, the binding proteins described herein are useful to cross the BBB and/or difficult natural neurological barriers so as to deliver drugs, biologics, etc., to a subject and to treat disorders and conditions including Alzheimer's disease (AD), Parkinson's disease (PD) and Multiple Sclerosis (MS).

In certain aspects, the invention provides multivalent and/or multispecific binding proteins capable of binding receptors expressed on the brain vascular endothelium used in treating Alzheimer's disease (AD), Parkinson's disease (PD) and/or multiple sclerosis (MS).

The binding protein in various embodiments comprises an amino acid sequence that specifically binds to an epitope, antigen, receptor or target, such that the binding protein is effective for transport to or across the BBB. For example, the amino acid sequence includes at least about three amino acids, at least about five amino acids, at least about seven amino acids, at least about ten amino acids, at least about 15 amino acids, or at least 20 amino acids that binds to an epitope, antigen, receptor or target, such that the binding protein is effective for transport to or across the BBB.

In various embodiments, the receptor is selected from the group consisting of insulin receptor (e.g., human insulin receptor), transferrin receptor, LRP (e.g., LRP1, LRP6 and LRP8), melanocortin receptor, nicotinic acetylcholine receptor, VACM-1 receptor, IGFR, EPCR, EGFR, TNFR, Leptin receptor, M6PR, Lipoprotein receptor, NCAM, LIFR, LfR, MRP1, AchR, DTr, Glutathione transporter, SR-B1, MYOF, TFRC, ECE1, LDLR, PVR, CDC50A, SCARF1, MRC1, HLA-DRA, RAMP2, VLDLR, STAB1, TLR9, CXCL16, NTRK1, CD74, DPP4, endothelial growth factor receptors 1, 2 and 3, glucocorticoid receptor, ionotropic glutamate receptor, M3 receptor, aryl hydrocarbon receptor, GLUT-1, inositol-1,4,5-trisphosphate (IP3) receptor, N-methyl-D-aspartate receptor, S1P1, P2Y receptor, TMEM30A, and RAGE

The epitope, antigen, receptor or target includes for example is an insulin receptor (e.g., human insulin receptor), a transferrin receptor, a low density lipoprotein receptor-related protein (LRP), for example LRP-1 and LRP-8, a melanocortin receptor, a nicotinic acetylcholine receptor, a VACM-1 receptor, a vascular endothelial growth factor receptor 1, 2 or 3, a glucocorticoid receptor, an ionotropic glutamate receptor, a M3 receptor, an aryl hydrocarbon receptor, GLUT-1, an inositol-1,4,5-trisphosphate (IP3) receptor, a N-methyl-D-aspartate receptor, S1P1, a P2Y receptor, and RAGE. The receptor in various embodiments allows for transport of a compound, drug, peptide or protein across the BBB.

In other embodiments, the binding protein is also capable of modulating a biological function of one or more targets associated with AD, PS, MS or other neurological disease. In certain aspects of this embodiment, the binding protein comprises an amino acid sequence that specifically binds to an epitope, antigen, receptor or target, such that a biological function is modulated. In certain embodiments, multivalent and/or multispecific binding proteins bind to the binding receptors expressed on the brain vascular endothelium as well as a therapeutic target. In these embodiments, the epitope, antigen, receptor or target can be selected from CGRP, TNFα, RGMA, Substance P, Bradykinin, Nav1.7, LPA, P2X3, NGF, Abeta; APP, BACE1; IL-1β; IGF1, or 2; IL-18; IL-6; RAGE; NGF; EGFR; cMet; Her2; RGMA, and CD-20. In various embodiments, the epitope, antigen, receptor or target is Amigo or RPTP sigma. In various embodiments, the epitope, antigen, receptor or target is selected from Lingo, NogoR, a semaphoring, a neuroplexin, a plexin, an ephrin, an ephrin receptor, and portions thereof.

In various embodiments, the binding protein comprises a Fv; a Fab; a Fab′; a F(ab′)₂, or a bivalent fragment comprising two Fab fragments linked by a disulfide bridge at the hinge region. In other embodiments, the binding protein includes at least one heavy variable region (VH) and at least one light variable region (VL). In certain embodiments the binding protein includes VH and CH1 domains; VL and VH domains; or an isolated complementarity determining region (CDR). In various embodiments, the binding protein comprises at least one VH, at least one VL, or at least one hypervariable (hv) site. In various embodiments, the binding includes a constant region. For example, the constant region is from a mammal, e.g., a human and a mouse. In various embodiments, the constant region has at least one mutation.

In certain embodiments, the binding protein is monospecific for an epitope or antigen. In these embodiments, the binding protein is effective for transport to or across the BBB. In other embodiments, two or more distinct binding regions are combined to construct a chimeric binding protein. For example the chimeric protein includes at least two non-identical binding regions. For example, the binding protein comprises a DVD-Ig protein as described herein.

The binding protein includes at least one binding region that specifically binds an epitope, an antigen, a receptor or a target. In various embodiments, the binding protein comprises a single chain. In various embodiments, the binding protein comprises a plurality of chains, i.e., at least two polypeptide chains. In various embodiments, the binding protein comprises a plurality of binding regions which are ordered or orientated such that each binds to the same or different portion of an epitope, an antigen, a receptor or a target. For example, the at least one binding region is positioned proximally and/or distally to another binding region, such that each is present on the same or different variable region/domain. In various embodiments, the binding regions are positioned parallel to one another, for example on a VH and a VL. In various embodiments, the binding regions are positioned opposite or facing one another, for example a first binding region is within a first VH or first VL, and a second binding region is within a second VH or a second VL. In various embodiments, the multiple binding regions are each bound to the same separate/third portion (e.g., a constant domain or linker), such that each binding region may interact or alternatively does not interact with one another.

In various embodiments, the binding protein is a molecule with the ability to monospecifically bind a receptor, antigen or target and cross the BBB. In various embodiments, the binding protein is formulated, compounded or administered in a form (e.g., nanoparticle; liposome, mixture, or solution) and is delivered along with an agent to treat a disease (e.g., cancer, AD, PD and MS) in the brain. For example, the binding protein is administered in a composition including the agent (e.g., a peptide or protein). In various embodiments, the binding protein in bound or attached to the agent. For example, the binding protein and agent are administered within the composition. Alternatively, the agent or binding protein is administered before or after one another over a period of seconds, minutes, hours or days of one another.

In various embodiments, the binding protein is bi-specific and binds two different antigens (or epitopes). For example, the binding protein specifically binds a receptor, antigen or target for crossing the BBB, and also specifically binds another AD-associated target in the brain. In various embodiments, the binding protein comprises at least one VH and at least one VL. For example, the binding protein comprises a DVD-Ig protein (or portion thereof) as described herein.

In certain embodiments, the binding protein includes at least two VH domains. In some embodiments, one VH domain specifically binds a receptor, antigen or target for crossing the BBB and another VH domain specifically binds another target in the brain. In other embodiments, the binding protein includes at least two VL domains. In some embodiments, one VL domain specifically binds a receptor, antigen or target for crossing the BBB and another VL domain specifically binds another target in the brain. In other embodiments, the binding protein includes at least two VH and at least two VL domains. In some embodiments, one VL domain specifically binds a receptor, antigen or target for crossing the BBB and another VH domain specifically binds another target in the brain while one VH domain specifically binds a receptor, antigen or target for crossing the BBB and another VH domain specifically binds another target in the brain.

In certain embodiments, the other target is a disease-associated target, for example, an AD-associated target, a PD-associated target, or an MS-associated target.

According to other embodiments, the binding protein is made up of two polypeptides or arms. Each of the arms can have one or more VH and VL domains. In certain embodiments, each arm has two VH and two VL domains. In other embodiments, the arms have only two VH or two VL domains. In certain embodiments, the binding protein comprises two arms/regions and each arm binds the same target or binds at least two different targets. For example, one arm binds the receptor, antigen or target for crossing the BBB, and the other arm upon crossing the BBB binds a different target on or in the brain (brain target). For example, a VH or VL on one arm binds the receptor for crossing the BBB, and a VH or VL on the other arm binds to the target. Alternatively, in various embodiments the binding protein has two identical antigen binding arms, in which each arm contains a VH/VL that binds the receptor for crossing the BBB, and a VHNL that binds to a target found inside of the brain upon crossing the BBB that is effective for treating AD, PD or MS. For example, each arm has identical specificity and identical CDR sequences. In various embodiments, the binding protein is a DVD-Ig protein that contains a VH1 or VH2 that binds to the BBB receptor or binds to the target on or in the brain. For example, the VH1 or VL1 binds to the BBB receptor, and the VH2 or VL2 binds to the target on or in the brain. Alternatively, the VH2 or VL2 binds to the BBB receptor, and the VH1 or VL2 binds to the target on or in the brain.

In various embodiments, the binding protein comprises a DVD-Ig protein as described herein that binds at least two different targets. In various embodiments, the binding protein is a DVD-Ig protein contains a VH1 that binds to the BBB receptor, and a VH2 that binds to the target on or in the brain. Alternatively, the binding protein is a DVD-Ig protein contains a VH2 that binds to the BBB receptor, and a VH1 that binds to the target on or in the brain. In various embodiments, the VL1 binds to the BBB receptor, and the VL2 binds to the target on or in the brain. In various embodiments, the VL2 binds to the BBB receptor, and the VL1 binds to the target on or in the brain.

In various embodiments, the binding protein comprises a variable binding region. For example, the variable binding region comprises a VH or VL. In various embodiments, the VL is located proximally or distally to the VL. For example, the VH is adjacent, bound or connected to the VL. In various embodiments, the VH is directly contacted to the VL, or the VH is connected to the VL by a linker. In various embodiments, the VH is parallel or adjacent to the VL. For example, the VH is separated from the VL by a covalent bond that maintains the VH and the VL in a confirmation or orientation.

In various embodiments, the binding protein comprises a polypeptide chain having a structure VD1-(X1)n-VD2-C-(X2)n, such that VD1 is a first variable domain, VD2 is a second variable domain, C is a constant domain, X1 represents an amino acid or polypeptide, X2 represents an Fc region and n is 0 or 1. In an embodiment, the VD1 and VD2 in the binding protein are heavy chain variable domains. In another embodiment, VD1 and VD2 are capable of binding the same antigen. In another embodiment, VD1 and VD2 are capable of binding different antigens. In still another embodiment, C is a heavy chain constant domain. For example, X1 is a linker with the proviso that X1 is not CH1.

In an embodiment, the binding protein disclosed herein comprises a polypeptide chain that binds the epitope, receptor or antigen, such that the polypeptide chain comprises VD1-(X1)n-VD2-C-(X2)n, and VD1 is a first heavy chain variable domain, VD2 is a second heavy chain variable domain, C is a heavy chain constant domain, X1 is a linker, and X2 is an Fc region. In an embodiment, X1 is a linker with the proviso that it is not CH1.

In various embodiments, the binding protein is attached or linked to an agent (e.g., a therapeutic agent or diagnostic agent). For example the binding protein includes a linker that separates the binding regions and/or that separates the binding protein from the agent. The linker in a related embodiment separates the binding regions and/or subunits of the binding protein. In certain embodiments, the binding protein includes a linker that covalently joins at least one binding region (e.g., a VH or a VL) to at least one other amino acid residue or domain. In various embodiments, the linker includes at least one selected from the group of a peptide, a protein, a sugar, or a nucleic acid. In a related embodiment, the linker includes an amino acid sequence described herein or a portion thereof or multiples thereof. The linker in various embodiments stabilizes the binding protein and does not prevent the respective binding of a binding region or the peptide to the epitope, antigen, receptor or target, such that the protein is effective for transport to or across the BBB. In various embodiments, the binding protein comprises a linker that reduces steric hindrance.

In various embodiments, the binding protein peptide is recombinantly produced. In certain embodiments, the recombinant binding protein is encoded by a nucleotide sequence or the binding protein includes an amino acid sequence that is substantially identical or homologous to the sequences described herein, for example a sequence shown in any of the Examples and Tables herein. For example, recombinant binding protein is engineered and constructed using any of the sequences described herein. In a related embodiment, the binding protein is administered to a subject using a vector carrying a nucleotide sequence that encodes the binding protein. In various embodiments, the binding protein (with or without an agent) is delivered for example using a liposome, a lipid/polycation (LPD), a peptide, a nanoparticle, a gold particle, and a polymer.

In various embodiments, the binding protein includes an amino acid sequence having a conservative sequence modification from the sequences shown herein, e.g., SEQ ID NOs: 1-195 or sequences in Tables 1-19, or different combinations of these sequences. The phrase “conservative sequence modifications” refers to amino acid modifications that do not significantly affect or alter the characteristics (e.g., binding, stability, and orientation) of the binding protein, e.g., amino acid sequences of binding protein that present a side chain at the same relative position to allow for function in a manner similar to an unmodified binding protein. A conservative modification includes for example a substitution, addition, or deletion in the amino acid sequence of the binding protein. Modification of the amino acid sequence of recombinant multimeric binding protein is achieved using any known technique in the art, e.g., site-directed mutagenesis or PCR based mutagenesis. Such techniques are described in Sambrook et al., Molecular Cloning: A Laboratory Manual, Cold Spring Harbor Press, Plainview, N. Y., 1989 and Ausubel et al., Current Protocols in Molecular Biology, John Wiley & Sons, New York, N. Y., 1989. Conservative amino acid substitutions are modifications in which the amino acid residue is replaced with an amino acid residue having a similar side chain such as replacing a small amino acid with a different small amino acid or a hydrophilic amino acid with a different hydrophilic amino acid.

In certain embodiments, the DVD binding proteins can bind to an antigen (e.g., a target and a receptor) expressed on the brain vascular endothelium and have another unoccupied binding site. This unoccupied binding site can be specific for a composition (e.g., an endogenous or exogenous therapeutic protein) to be co-transported across the BBB. Accordingly, binding proteins “pre-loaded” in this fashion can be delivered to a desired target site in the brain to exert its desired therapeutic activity. Alternatively, the binding site can remain unoccupied following transport and BBB uptake via binding to the receptor expressed on the brain vascular endothelium so that it is capable of binding a desired neurological disease-associated, AD-associated, MS-associated or PD-associated target molecule on the brain side of the BBB.

In certain aspects of the disclosure, there is an inverse correlation between the binding affinity of a binding protein to an antigen (e.g., a receptor) expressed on or in the brain vascular endothelium. The binding proteins specifically bind to the receptor expressed on the brain vascular endothelium, but they can bind at the lower end of the binding affinity range for specific binding. Thus, in some embodiments, the binding protein will bind to a receptor expressed on the brain vascular endothelium with dissociation constant of between 1×10⁻⁶ M and 1×10⁻⁷M. In other embodiments, the dissociation constant is between 1×10⁻⁶ M and 1×10⁻⁸M. In some embodiments, lower affinity is achieved through the humanization of antibodies from non-human mammals.

In other embodiments, various portions of the binding protein will bind the receptor expressed on the brain vascular endothelium with different affinities. In certain embodiments, the binding protein is a DVD binding protein. In certain embodiments, the DVD binding protein comprises two arms. Each arm includes a heavy and a light chain. Each heavy and light chain includes a variable domain. Thus, DVD binding proteins can include 8 variable domains or 4 binding sites comprising 4 VH/VL pairs. Each of these domains can specifically bind a given antigen with a different dissociation constant. In some embodiments, the domain will bind to an antigen with dissociation constant of between 1×10⁻⁶ M and 1×10⁻⁷M. In other embodiments, the dissociation constant is between 1×10⁻⁶ M and 1×10⁻⁸M. In certain specific embodiments, the antigen is a receptor expressed on the brain vascular endothelium, a composition to be co-transported across the BBB or a target on the brain side of the BBB.

In certain embodiments of this disclosure, the binding protein that specifically binds to a receptor expressed on the brain vascular endothelium can have a 2 or more fold increase in uptake of a composition across the BBB compared to a control non-specific binding protein. In other embodiments, the binding protein that specifically binds to a receptor expressed on the brain vascular endothelium can have a 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95 or 100-fold or more fold increase in uptake of a composition across the BBB compared to a control non-specific binding protein. According to certain embodiments, a composition is co-administered with the binding protein that specifically binds to a brain receptor or receptor expressed on the brain vascular endothelium. This composition can be directly bound to the binding protein or it can be co-administered in an unconjugated form. In embodiments, wherein the composition is bound to the binding protein, in certain embodiments, the composition is bound through a linker. The linker can be a polypeptide linker. The linker can also be a non-polypeptide linker. Many suitable linkers are known in the art.

In certain embodiments, the composition co-administered with the binding protein can be selected from the following: an imaging agent, a cytotoxic agent, an angiogenesis inhibitor, a kinase inhibitor, a co-stimulation molecule blocker, an adhesion molecule blocker, an anti-cytokine antibody or functional fragment thereof, a detectable label or reporter, an antirheumatic, a muscle relaxant, a narcotic, a non-steroid anti-inflammatory drug (NSAID), an analgesic, an anesthetic, a sedative, a neuromuscular blocker, an antimicrobial, an antipsoriatic, a corticosteroid, an anabolic steroid, an erythropoietin, an immunoglobulin, an immunosuppressive, a growth hormone, a hormone replacement drug, a radiopharmaceutical, an antidepressant, an antipsychotic, a stimulant, an asthma medication, a beta agonist, an inhaled steroid, an epinephrine or analog, a cytokine, and a cytokine antagonist. In an embodiment, the composition co-administered with the binding protein can be selected from the following: budenoside, epidermal growth factor, a corticosteroid, cyclosporin, sulfasalazine, an aminosalicylate, 6-mercaptopurine, azathioprine, metronidazole, a lipoxygenase inhibitor, mesalamine, olsalazine, balsalazide, an antioxidant, a thromboxane inhibitor, a growth factor, an elastase inhibitor, a pyridinyl-imidazole compound, an antibody, antagonist or agonist of TNF, LT, IL-1, IL-1R, IL-2, IL-4, IL-6, IL-6R, IL-7, IL-8, IL-10, IL-11, IL-12, IL-13, IL-15, IL-16, IL-18, IL-23, TGFβ, EMAP-II, GM-CSF, FGF, PDGF, CD2, CD3, CD4, CD8, CD-19, CD25, CD28, CD30, CD40, CD45, CD69, CD90 or a ligand thereof, methotrexate, FK506, rapamycin, mycophenolate mofetil, leflunomide, ibuprofen, prednisolone, a phosphodiesterase inhibitor, an adenosine agonist, an antithrombotic agent, a complement inhibitor, an adrenergic agent, IRAK, NIK, IKK, p38, a MAP kinase inhibitor, an IL-1β converting enzyme inhibitor, a TNFα-converting enzyme inhibitor, a T-cell signaling inhibitor, a metalloproteinase inhibitor, an angiotensin converting enzyme inhibitor, a soluble cytokine receptor, a soluble p55 TNF receptor, a soluble p75 TNF receptor, sIL-1RI, sIL-1RII, sIL-6R and combinations thereof.

Unless otherwise defined herein, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including”, as well as other forms, such as “includes” and “included”, is not limiting.

Generally, nomenclatures used in connection with cell and tissue culture, molecular biology, immunology, microbiology, pathology, genetics and protein and nucleic acid chemistry and hybridization described herein are those well-known and commonly used in the art. The methods and techniques provided herein are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

That the disclosure may be more readily understood, select terms are defined below.

The term “antibody” refers to an immunoglobulin (Ig) molecule, which is generally comprised of four polypeptide chains, two heavy (H) chains and two light (L) chains, or a functional fragment, mutant, variant, or derivative thereof, that retains the epitope binding features of an Ig molecule. Such fragment, mutant, variant, or derivative antibody formats are known in the art. In an embodiment of a full-length antibody, each heavy chain is comprised of a heavy chain variable region (VH) and a heavy chain constant region (CH). The CH is comprised of three domains, CH1, CH2 and CH3. Each light chain is comprised of a light chain variable region (VL) and a light chain constant region (CL). The CL is comprised of a single CL domain. The VH and VL can be further subdivided into regions of hypervariability, termed complementarity determining regions (CDRs), interspersed with regions that are more conserved, termed framework regions (FRs). Generally, each VH and VL is composed of three CDRs and four FRs, arranged from amino-terminus to carboxy-terminus in the following order: FR1, CDR1, FR2, CDR2, FR3, CDR3, and FR4. Immunoglobulin molecules can be of any type (e.g., IgG, IgE, IgM, IgD, IgA and IgY), class (e.g., IgG1, IgG2, IgG3, IgG4, IgA1 and IgA2), or subclass.

The term “binding protein” means a protein or peptide that binds to a ligand, such as an antigen or epitope.

The term “bispecific antibody” means an antibody that binds one antigen (or epitope) on one of its two binding arms (one pair of HC/LC), and binds a different antigen (or epitope) on its second binding arm (a different pair of HC/LC). A bispecific antibody has two distinct antigen binding arms (in both specificity and CDR sequences), and is monovalent for each antigen to which it binds. Bispecific antibodies include those generated by quadroma technology (Milstein and Cuello (1983) Nature 305(5934): 537-40), by chemical conjugation of two different monoclonal antibodies (Staerz et al. (1985) Nature 314(6012): 628-31), or by knob-into-hole or similar approaches which introduces mutations in the Fc region (Holliger et al. (1993) Proc. Natl. Acad. Sci. USA 90(14): 6444-6448), among others.

An “affinity matured” antibody is an antibody with one or more alterations in one or more of its CDRs which result in an improvement in the affinity of the antibody for antigen, compared to a parent antibody that does not possess those alteration(s).

The term “CDR-grafted antibody” means an antibody that comprises heavy and light chain variable region sequences in which the sequences of one or more of the CDR regions of VH and/or VL are replaced with CDR sequences of another antibody. For example, the two antibodies can be from different species, such as antibodies having murine heavy and light chain variable regions in which one or more of the human CDRs of an antibody has been replaced with mouse CDR sequences (e.g., the mouse CDRs are placed into a human framework).

The term “humanized antibody” refers to an antibody from a non-human species that has been altered to be more “human-like”, i.e., more similar to human germline sequences. One type of humanized antibody is a CDR-grafted antibody. A “humanized antibody” is also an antibody or a variant, derivative, analog or fragment thereof that comprises framework region (FR) sequences having substantially (e.g., at least 80%, at least 85%, at least 90%, at least 95%, at least 98% or at least 99% identity to) the amino acid sequence of a human antibody and at least one CDR having substantially the amino acid sequence of a non-human antibody. A humanized antibody may comprise substantially all of at least one, and typically two, variable domains (Fab, Fab′, F(ab′) 2, FabC, Fv) in which the sequence of all or substantially all of the CDR regions correspond to those of a non-human immunoglobulin (i.e., donor antibody) and the sequence of all or substantially all of the FR regions are those of a human immunoglobulin. The humanized antibody also may include the CH1, hinge, CH2, CH3, and CH4 regions of the heavy chain. In an embodiment, a humanized antibody also comprises at least a portion of a human immunoglobulin Fc region. In some embodiments, a humanized antibody only contains a humanized light chain. In some embodiments, a humanized antibody only contains a humanized heavy chain. In some embodiments, a humanized antibody only contains a humanized variable domain of a light chain and/or humanized variable domain of a heavy chain. In some embodiments, a humanized antibody contains a light chain as well as at least the variable domain of a heavy chain. In some embodiments, a humanized antibody contains a heavy chain as well as at least the variable domain of a light chain.

The terms “dual variable domain binding protein” means a binding protein that has two variable domains in each polypeptide chain of its binding arm(s) (e.g., a pair of HC/LC) (see PCT Publication No. WO 02/02773), each of which is able to bind to an antigen. In an embodiment, each variable domain binds different antigens or epitopes. In another embodiment, each variable domain binds the same antigen or epitope. In another embodiment, a dual variable domain binding protein has two identical antigen binding arms, with identical specificity and identical CDR sequences, and is bivalent for each antigen to which it binds. In an embodiment, the DVD binding proteins may be monospecific, i.e., capable of binding one antigen target or multispecific, i.e., capable of binding two or more different antigen targets. In an embodiment, each half of a four chain DVD binding protein comprises a heavy chain DVD polypeptide, and a light chain DVD polypeptide, and two antigen binding sites. In an embodiment, each binding site comprises a heavy chain variable domain and a light chain variable domain with a total of 6 CDRs involved in antigen binding per antigen binding site. In an embodiment, a DVD binding protein comprising two heavy chain DVD polypeptides and two light chain DVD polypeptides is referred to as a DVD-Ig protein. In certain embodiments the DVD binding protein includes an amino acid sequence that specifically binds to an epitope, antigen, receptor or target, such that the binding protein is effective for transport to or across the BBB. For example, the amino acid sequence includes at least about three amino acids, at least about five amino acids, at least about seven amino acids, at least about ten amino acids, at least about 15 amino acids, or at least 20 amino acids that binds to an epitope, antigen, receptor or target, such that the binding protein is effective for transport to or across the BBB. In these embodiments, the epitope, antigen, receptor or target includes for example an insulin receptor, a transferrin receptor, a low density lipoprotein receptor-related protein (LRP) for example LRP-1 and LRP-8, a melanocortin receptor, a nicotinic acetylcholine receptor, a VACM-1 receptor, a vascular endothelial growth factor receptor 1, 2 or 3, a glucocorticoid receptor, an ionotropic glutamate receptor, a M3 receptor, an aryl hydrocarbon receptor, GLUT-1, an inositol-1,4,5-trisphosphate (IP3) receptor, a N-methyl-D-aspartate receptor, S1P1, a P2Y receptor, and RAGE.

In certain embodiments the DVD binding protein further includes at least one other region that is capable of modulating a biological function of one or more targets associated with AD, PS, MS or other neurological disease. In certain aspects of this embodiment, the DVD binding protein comprises an amino acid sequence that specifically binds to an epitope, antigen, receptor or target such that a biological function is modulated. In these embodiments, the epitope, antigen, receptor or target can be selected from CGRP, TNFα, RGMA, Substance P, Bradykinin, Nav1.7, LPA, P2X3, NGF, Abeta; APP, BACE1; IL-1β; IGF1, or 2; IL-18; IL-6; RAGE; NGF; EGFR; cMet; Her2; and CD-20.

The term “dual variable domain immunoglobulin” and “DVD-Ig” mean a DVD binding protein that comprising two first and two second polypeptide chains, each independently comprising VD1-(X1)n-VD2-C-(X2)n, wherein VD1 is a first variable domain; VD2 is a second variable domain; C is a constant domain; X1 is a linker; X2 is an Fc region; n is 0 or 1. In various embodiments of the method, the DVD-Ig comprises a first and second polypeptide chains, wherein said first polypeptide chain comprises a first VD1-(X1)n-VD2-C-(X2)n, wherein VD1 is a first heavy chain variable domain; VD2 is a second heavy chain variable domain; C is a heavy chain constant domain; X1 is a linker with the proviso that it is not CH1; X2 is an Fc region; and n is independently 0 or 1; and wherein said second polypeptide chain comprises a second VD1-(X1)n-VD2-C-(X2)n, wherein VD1 is a first light chain variable domain; VD2 is a second light chain variable domain; C is a light chain constant domain; X1 is a linker with the proviso that it is not CH1; X2 does not comprise an Fc region; and n is independently 0 or 1. The terms “single chain dual variable domain immunoglobulin protein” or “scDVD-Ig protein” or scFv DVD-Ig protein” mean the antigen binding fragment of a DVD molecule that is analogous to an antibody single chain Fv fragment. The scDVD-Ig proteins are described in U.S. Patent Publication Nos. 2014/0243228 and 2014/0221621, incorporated herein by reference in their entireties. scDVD-Ig proteins are generally of the formula VH1-(X1)n-VH2-X2-VL1-(X3)n-VL2, where VH1 is a first antibody heavy chain variable domain, X1 is a linker with the proviso that it is not a constant domain, VH2 is a second antibody heavy chain variable domain, X2 is a linker, VL1 is a first antibody light chain variable domain, X3 is a linker with the proviso that it is not a constant domain, VL2 is a second antibody light chain variable domain, and n is 0 or 1, where the VH1 and VL1, and the VH2 and VL2 respectively combine to form two functional antigen binding sites.

The terms “DVD-Fab” or fDVD-Ig protein” mean the antigen binding fragment of a DVD-Ig molecule that is analogous to an antibody Fab fragment. Exemplary fDVD-Ig proteins are described in U.S. Patent Publication Nos. 2014/0243228; and 2014/0235476, incorporated herein by reference in their entireties. In certain embodiments, fDVD-Ig proteins include a first polypeptide chain having the general formula VH1-(X1)n-VH2-C-(X2)n, wherein VH1 is a first heavy chain variable domain, X1 is a linker with the proviso that it is not a constant domain, VH2 is a second heavy chain variable domain, and C is a heavy chain constant domain, X2 is an Fc region, and n is 0 or 1, and wherein the amino acid sequences of VH1, VH2 and/or X1 independently vary. In certain embodiments, the fDVD-Ig proteins also include a second polypeptide chain having the general formula VL1-(Y1)n-VL2-C, wherein VL1 is a first light chain variable domain, Y1 is a linker with the proviso that it is not a constant domain, VL2 is a second light chain variable domain, C is a light chain constant domain, n is 0 or 1, wherein the VH1 and VH2 of the first polypeptide chain and VL1 and VL2 of second polypeptide chains of the binding protein combine form two functional antigen binding sites. In certain embodiments, the first and second polypeptide chains combine to form an fDVD-Ig protein.

In certain embodiments, the binding protein of the invention is a half-DVD-Ig” proteins derived from a DVD-Ig protein. The half-DVD-Ig protein preferably does not promote cross-linking observed with naturally occurring antibodies which can result in antigen clustering and undesirable activities. See U.S. Patent Publication No. 20120201746 and International Publication No. WO/2012/088302, each of which is incorporated by reference herein in its entirety.

The terms “receptor DVD-Ig protein” or “rDVD-Ig protein” mean DVD-Ig constructs comprising at least one receptor-like binding domain. Exemplary rDVD-Ig proteins are described in U.S. Patent Publication No. 2014/0219913, incorporated herein by reference in their entireties. Variable domains of the rDVD-Ig molecule may include one immunoglobulin variable domain and one non-immunoglobulin variable domain such as a ligand binding domain of a receptor, or an active domain of an enzyme. The rDVD-Ig molecules may also comprise two or more non-Ig domains (see PCT Publication No. WO 02/02773). In rDVD-Ig protein at least one of the variable domains comprises a ligand binding domain of a receptor, or receptor domain (RD). The term “receptor domain” (RD) means the portion of a cell surface receptor, cytoplasmic receptor, nuclear receptor, or soluble receptor that functions to bind one or more receptor ligands or signaling molecules (e.g., toxins, hormones, neurotransmitters, cytokines, growth factors, or cell recognition molecules).

The term “pDVD-Ig” protein means a multi-specific or multivalent IgG-like molecules that are capable of binding two or more proteins (e.g., antigens). Exemplary pDVD-Ig proteins are described in U.S. Patent Publication Nos. 2014/0243228 and 2014/0213771, incorporated herein by reference in their entireties. In certain embodiments, pDVD-Ig proteins are disclosed which are generated by specifically modifying and adapting several concepts. These concepts include but are not limited to: (1) forming Fc heterodimer using CH3 “knobs-into-holes” design, (2) reducing light chain missing pairing by using CH1/CL cross-over, and (3) pairing two separate half IgG molecules at protein production stage using “reduction then oxidation” approach.

In one embodiment, a pDVD-Ig construct may be created by combining two halves of different DVD-Ig molecules, or a half DVD-Ig protein and half IgG molecule. A pDVD-Ig construct may be expressed from four unique constructs to create a monovalent, multi-specific molecule through the use of heavy chain CH3 knobs-into-holes design. In another embodiment, a pDVD-Ig construct may contain two distinct light chains, and may utilize structural modifications on the Fc of one arm to ensure the proper pairing of the light chains with their respective heavy chains. In one aspect, the heavy chain constant region CH1 may be swapped with a light chain constant region hCk on one Fab. In another aspect, an entire light chain variable region, plus hCk, may be swapped with a heavy chain variable region, plus CH1. The pDVD-Ig construct vectors that accommodate these unique structural requirements are also disclosed.

In some embodiments, pDVD-Ig proteins contain four polypeptide chains, namely, first, second, third and fourth polypeptide chains. In one aspect, the first polypeptide chain may contain VD1-(X1)n-VD2-CH-(X2)n, wherein VD1 is a first heavy chain variable domain, VD2 is a second heavy chain variable domain, CH is a heavy chain constant domain, X1 is a linker with the proviso that it is not a constant domain, and X2 is an Fc region. In another aspect, the second polypeptide chain may contain VD1-(X1)n-VD2-CL-(X2)n, wherein VD1 is a first light chain variable domain, VD2 is a second light chain variable domain, CL is a light chain constant domain, X1 is a linker with the proviso that it is not a constant domain, and X2 does not comprise an Fc region. In another aspect, the third polypeptide chain may contain VD3-(X3)n-VD4-CL-(X4)n, wherein VD3 is a third heavy chain variable domain, VD4 is a fourth heavy chain variable domain, CL is a light chain constant domain, X3 is a linker with the proviso that it is not a constant domain, and X4 is an Fc region. In another aspect, the fourth polypeptide chain may contain VD3-(X3)n-VD4-CH-(X4)n, wherein VD3 is a third light chain variable domain, VD4 is a fourth light chain variable domain, CH is a heavy chain constant domain, X3 is a linker with the proviso that it is not a constant domain, and X4 does not comprise an Fc region. In another aspect, n is 0 or 1, and the VD1 domains on the first and second polypeptide chains form one functional binding site for antigen A, the VD2 domains on the first and second polypeptide chains form one functional binding site for antigen B, the VD3 domains on the third and fourth polypeptide chains form one functional binding site for antigen C, and the VD4 domains on the third and fourth polypeptide chains form one functional binding site for antigen D. In one embodiment, antigens A, B, C and D may be the same antigen, or they may each be a different antigen. In another embodiment, antigens A and B are the same antigen, and antigens C and D are the same antigen.

As used herein “monobody DVD-Ig protein” or “mDVD-Ig protein” means a class of binding molecules wherein one binding arm has been rendered non-functional. Exemplary mDVD-Ig proteins are described in U.S. Patent Publication Nos. 2014/0243228 and 2014/0221622, incorporated herein by reference in their entireties. In one aspect, an mDVD-Ig protein possesses only one functional arm capable of binding a ligand. In another aspect, the one functional arm may have one or more binding domains for binding to different ligands. The ligand may be a peptide, a polypeptide, a protein, an aptamer, a polysaccharide, a sugar molecule, a carbohydrate, a lipid, an oligonucleotide, a polynucleotide, a synthetic molecule, an inorganic molecule, an organic molecule, and combinations thereof.

In one embodiment, an mDVD-Ig protein contains four polypeptide chains, wherein two of the four polypeptide chains comprise VDH-(X1)n-C-(X2)n. In one aspect, VDH is a heavy chain variable domain, X1 is a linker with the proviso that it is not CH1, C is a heavy chain constant domain, X2 is an Fc region, and n is 0 or 1. The other two of the four polypeptide chains comprise VDL-(X3)n-C-(X4)n, wherein VDL is a light chain variable domain, X3 is a linker with the proviso that it is not CH1, C is a light chain constant domain, X4 does not comprise an Fc region, and n is 0 or 1. In another aspect, at least one of the four polypeptide chains comprises a mutation located in the variable domain, wherein the mutation inhibits the targeted binding between the specific antigen and the mutant binding domain.

The Fc regions of the two polypeptide chains that have a formula of VDH-(X1)n-C-(X2)n may each contain a mutation, wherein the mutations on the two Fc regions enhance heterodimerization of the two polypeptide chains. In one aspect, knobs-into-holes mutations may be introduced into these Fc regions to achieve heterodimerization of the Fc regions. See Atwell et al. (1997) J. Mol. Biol. 270: 26-35.

As used herein “cross-over DVD-Ig” protein or “coDVD-Ig” protein means a DVD-Ig protein wherein the cross-over of variable domains is used to resolve the issue of affinity loss in the inner antigen-binding domains of some DVD-Ig molecules. Exemplary coDVD-Ig proteins are described in U.S. Patent Publication Nos. 2014/0243228 and 2014-0213772, incorporated herein by reference in their entireties. In certain specific embodiments, coDVD-Ig” proteins are generated by crossing over light chain and the heavy chain variable domains of a DVD-Ig protein or DVD-Ig-like protein. In another aspect, the length and sequence of the linkers linking the variable domains may be optimized for each format and antibody sequence/structure (frameworks) to achieve desirable properties. The disclosed concept and methodology may also be extended to Ig or Ig-like proteins having more than two antigen binding domains.

The term “biological activity” or “biological function” means one or more biological properties of a molecule (whether present naturally as found in vivo, in vitro, or provided or enabled by recombinant means). Biological properties include, but are not limited to, binding a receptor, inducing cell proliferation, inhibiting cell growth, inducing other cytokines, inducing apoptosis, and enzymatic activity. Binding proteins may target several classes of antigens and achieve desired therapeutic outcomes through multiple mechanisms of action. Binding proteins may target soluble proteins, cell surface antigens, and extracellular protein deposits, for example. Binding proteins may agonize, antagonize, or neutralize the activity of their targets. Binding proteins may assist in the clearance of the targets to which they bind, or may result in cytotoxicity when bound to cells. Portions of two or more antibodies may be incorporated into a multivalent format to achieve distinct functions in a single binding protein molecule. The in vitro assays and in vivo models used to assess biological function are known to one skilled in the art (US Patent Publication No. 20090311253).

The term “neutralizing” means counteracting the biological activity of an antigen when a binding protein specifically binds to the antigen. In an embodiment, the neutralizing binding protein binds to an antigen (e.g., a cytokine) and reduces biologically activity of the antigen by at least about 20%, 30%, 40%, 50%, 60%, 70%, 80%, 85%, 90%, 95%, 96%, 97%, 98%, 99%, or 100%.

“Specificity” means the ability of a binding protein to selectively bind an antigen.

The term “specifically binds” means that a binding protein or fragment thereof forms a complex with an antigen that is relatively stable under physiologic conditions. Specific binding can be characterized by a dissociation constant of at least about 1×10⁻⁶ M or smaller. In other embodiments, the dissociation constant is at least about 1×10⁻⁷ M, 1×10⁻⁸ M, or 1×10⁻⁹ M. Methods for determining whether two molecules specifically bind are well known in the art and include, for example, equilibrium dialysis, surface plasmon resonance, and the like.

The term “a disorder in which antigen activity is detrimental” is intended to include diseases and other disorders in which the presence of the antigen in a subject suffering from the disorder has been shown to be or is suspected of being either responsible for the pathophysiology of the disorder or a factor that contributes to a worsening of the disorder. Accordingly, a disorder in which antigen activity is detrimental is a disorder in which reduction or neutralization of antigen activity is expected to alleviate the symptoms and/or progression of the disorder. Such disorders may be evidenced, for example, by an increase in the concentration of the antigen in a biological fluid of a subject suffering from the disorder (e.g., an increase in the concentration of antigen in serum, plasma, synovial fluid, etc., of the subject). Non-limiting examples of disorders that can be treated with the binding proteins provided herein include those disorders discussed below and in the section pertaining to pharmaceutical compositions comprising the binding proteins.

The term “mammal” means any species that is a member of the class mammalia, including rodents, primates, dogs, cats, camelids, lagomorphs and ungulates. The term “rodent” refers to any species that is a member of the order rodentia including mice, rats, hamsters, and gerbils. The term “primate” refers to any species that is a member of the order primates, including monkeys, apes and humans. The term “lagomorph” refers to any species that is a member of the order lagomorpha, including rabbits and hares. The term “ungulates” refers to any species that is a member of the superorder ungulata including cattle, horses and camelids. The term “camelid” refers to any species that is a member of the family camelidae including camels and llamas.

The term “affinity” means the strength of the interaction between a binding protein and an antigen, and is determined by the sequence of the CDRs of the binding protein as well as by the nature of the antigen, such as its size, shape, and/or charge. Binding proteins may be selected for affinities that provide desired therapeutic end-points while minimizing negative side-effects. Affinity may be measured using methods known to one skilled in the art (See US Patent Publication No. 20090311253).

The term “potency” means the ability of a binding protein to achieve a desired effect, and is a measurement of its therapeutic efficacy. Potency may be assessed using methods known to one skilled in the art (See US Patent Publication No. 20090311253).

The term “cross-reactivity” means the ability of a binding protein to bind a target other than that against which it was raised. Generally, a binding protein will bind its target tissue(s)/antigen(s) with an appropriately high affinity, but will display an appropriately low affinity for non-target normal tissues. The term “cross-reactive” generally refers the ability to bind the same target in different species or homologous proteins in the same species. Individual binding proteins are generally selected to meet two criteria: (1) tissue staining appropriate for the known expression of the antibody target; and (2) similar staining pattern between human and tox species (e.g., mouse and cynomolgus monkey) tissues from the same organ. These and other methods of assessing cross-reactivity are known to one skilled in the art (US Patent Publication No. 20090311253).

As used herein, the term “biological barrier” is meant to include a biological cell, tissue, membrane, or structure that prevents effective passage, diffusion, or localization of biological molecules. In various embodiments, the biological barrier comprises neuronal/nervous, connective, muscle, membrane, or epithelial (e.g., mucosal or vascular) cells or tissue. For example, the blood brain barrier (BBB) is a biological barrier that is a highly selectively permeable barrier that separates the circulating blood from the brain extracellular fluid (BECF) in the central nervous system (CNS). The BBB is formed by brain endothelial cells that are connected by tight junctions and surrounded by astrocytes. Other examples of biological barrier are alimentary tissue such as the esophagus, gastrointestinal tissue such as the colon or the small intestine, and skin.

A “stable” binding protein is one in which the binding protein essentially retains its physical stability, chemical stability and/or biological activity upon storage. A multivalent binding protein that is stable in vitro at various temperatures for an extended period of time is desirable. Methods of stabilizing binding proteins and assessing their stability at various temperatures are known to one skilled in the art (US Patent Publication No. 20090311253).

The term “solubility” refers to the ability of a protein to remain dispersed within an aqueous solution. The solubility of a protein in an aqueous formulation depends upon the proper distribution of hydrophobic and hydrophilic amino acid residues and, therefore, solubility can correlate with the production of correctly folded proteins. A person skilled in the art will be able to detect an increase or decrease in solubility of a binding protein using routine HPLC techniques and methods known to one skilled in the art (US Patent Publication No. 20090311253).

Binding proteins may be produced using a variety of host cells or may be produced in vitro, and the relative yield per effort determines the “production efficiency.” Factors influencing production efficiency include, but are not limited to, host cell type (prokaryotic or eukaryotic), choice of expression vector, choice of nucleotide sequence, and methods employed. The materials and methods used in binding protein production, as well as the measurement of production efficiency, are known to one skilled in the art (US Patent Publication No. 20090311253).

The term “immunogenicity” means the ability of a substance to induce an immune response. Administration of a therapeutic binding protein may result in a certain incidence of an immune response. Potential elements that might induce immunogenicity in a multivalent format may be analyzed during selection of the parental antibodies, and steps to reduce such risk can be taken to optimize the parental antibodies prior to incorporating their sequences into a multivalent binding protein format. Methods of reducing the immunogenicity of antibodies and binding proteins are known to one skilled in the art (US Patent Publication No. 20090311253).

The terms “label” and “detectable label” mean a moiety attached to a member of a specific binding pair, such as an DVD binding protein, or its analyte to render a reaction (e.g., binding) between the members of the specific binding pair, detectable. The labeled member of the specific binding pair is referred to as “detectably labeled.” Thus, the term “labeled binding protein” refers to a protein with a label incorporated that provides for the identification of the binding protein. In an embodiment, the label is a detectable marker that can produce a signal that is detectable by visual or instrumental means, e.g., incorporation of a radiolabeled amino acid or attachment to a polypeptide of biotinyl moieties that can be detected by marked avidin (e.g., streptavidin containing a fluorescent marker or enzymatic activity that can be detected by optical or colorimetric methods). Examples of labels for polypeptides include, but are not limited to, the following: radioisotopes or radionuclides (e.g., ³H, ¹⁴C, ³⁵S, ⁹⁰Y, ⁹⁹Tc, ¹¹¹In, ¹²⁵I, ¹³¹I, ¹⁷⁷Lu, ¹⁶⁶Ho, or ¹⁵³Sm); chromogens, fluorescent labels (e.g., FITC, rhodamine, lanthanide phosphors), enzymatic labels (e.g., horseradish peroxidase, luciferase, alkaline phosphatase); chemiluminescent markers; biotinyl groups; predetermined polypeptide epitopes recognized by a secondary reporter (e.g., leucine zipper pair sequences, binding sites for secondary antibodies, metal binding domains, epitope tags); and magnetic agents, such as gadolinium chelates. Representative examples of labels commonly employed for immunoassays include moieties that produce light, e.g., acridinium compounds, and moieties that produce fluorescence, e.g., fluorescein. In this regard, the moiety itself may not be detectably labeled but may become detectable upon reaction with yet another moiety.

The term “conjugate” refers to a binding protein, such as a DVD binding protein, that is chemically linked to a second chemical moiety, such as a therapeutic or cytotoxic agent. The term “agent” includes a chemical compound, a mixture of chemical compounds, a biological macromolecule, or an extract made from biological materials. In an embodiment, the therapeutic agents or cytotoxic agents include, but are not limited to, pertussis toxin, taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. When employed in the context of an immunoassay, the conjugate antibody may be a detectably labeled antibody used as the detection antibody.

The terms “crystal” and “crystallized” mean a binding protein (e.g., an antibody), or antigen binding portion thereof, that exists in the form of a crystal. Crystals are one form of the solid state of matter, which is distinct from other forms such as the amorphous solid state or the liquid crystalline state. Crystals are composed of regular, repeating, three-dimensional arrays of atoms, ions, molecules (e.g., proteins such as antibodies), or molecular assemblies (e.g., antigen/antibody complexes). These three-dimensional arrays are arranged according to specific mathematical relationships that are well-understood in the field.

The term “vector” means a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Other vectors include RNA vectors. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, other forms of expression vectors are also included, such as viral vectors (e.g., replication defective retroviruses, adenoviruses and adeno-associated viruses), which serve equivalent functions. A group of pHybE vectors (U.S. Pat. Nos. 8,187,836 and 8,455,219) were used for parental antibody and DVD-Ig binding protein cloning. V1, derived from pJP183; pHybE-hCg1,z,non-a V2, was used for cloning of antibody and DVD-Ig heavy chains with a wild-type constant region. V2, derived from pJP191; pHybE-hCk V3, was used for cloning of antibody and DVD-Ig light chains with a kappa constant region. V3, derived from pJP192; pHybE-hCl V2, was used for cloning of antibody and DVD-Ig proteins light chains with a lambda constant region. V4, built with a lambda signal peptide and a kappa constant region, was used for cloning of DVD-Ig light chains with a lambda-kappa hybrid V domain. V5, built with a kappa signal peptide and a lambda constant region, was used for cloning of DVD-Ig light chains with a kappa-lambda hybrid V domain. V7, derived from pJP183; pHybE-hCg1,z,non-a V2, was used for cloning of antibody and DVD-Ig heavy chains with a (234,235 AA) mutant constant region.

The terms “recombinant host cell” or “host cell” refer to a cell into which exogenous DNA has been introduced. Such terms refer not only to the particular subject cell, but to the progeny of such a cell. Because certain modifications may occur in succeeding generations due to either mutation or environmental influences, such progeny may not be identical to the parent cell, but are still included within the scope of the term “host cell” as used herein. In an embodiment, host cells include prokaryotic and eukaryotic cells. In an embodiment, eukaryotic cells include protist, fungal, plant and animal cells. In another embodiment, host cells include but are not limited to the prokaryotic cell line E. Coli; mammalian cell lines CHO, HEK 293, COS, NSO, SP2 and PER.C6; the insect cell line Sf9; and the fungal cell Saccharomyces cerevisiae.

The term “transfection” means introducing exogenous nucleic acid (e.g., DNA) into a host cell, e.g., electroporation, calcium-phosphate precipitation, DEAE-dextran transfection and the like.

The term “cytokine” means a protein released by one cell population that acts on another cell population as an intercellular mediator. The term “cytokine” includes proteins from natural sources or from recombinant cell culture and biologically active equivalents of the native sequence cytokines.

The term “biological sample” means a quantity of a substance from a living thing or formerly living thing. Such substances include, but are not limited to, blood, plasma, serum, urine, amniotic fluid, synovial fluid, endothelial cells, leukocytes, monocytes, other cells, organs, tissues, bone marrow, lymph nodes and spleen.

The term “control” means a composition known to contain analyte (“positive control”) or, alternatively, to not contain analyte (“negative control”). A positive control can comprise a known concentration of analyte. “Control,” “positive control,” and “calibrator” may be used interchangeably herein to refer to a composition comprising a known concentration of analyte. A “positive control” can be used to establish assay performance characteristics and is a useful indicator of the integrity of reagents (e.g., analytes).

The term “specific binding partner” means a member of a specific binding pair. A specific binding pair comprises two different molecules that specifically bind to each other through chemical or physical means. Therefore, in addition to antigen and antibody specific binding, other specific binding pairs can include biotin and avidin (or streptavidin), carbohydrates and lectins, complementary nucleotide sequences, effector and receptor molecules, cofactors and enzymes, enzyme inhibitors and enzymes, and the like. Furthermore, specific binding pairs can include members that are analogs of the original specific binding members, for example, an analyte-analog. Immunoreactive specific binding members include antigens, antigen fragments, and antibodies, including monoclonal and polyclonal antibodies as well as complexes, fragments, and variants (including fragments of variants) thereof, whether isolated or recombinantly produced.

The term “Fc region” defines the C-terminal region of an immunoglobulin heavy chain, which may be generated by papain digestion of an intact antibody. The Fc region may be a native sequence Fc region or a variant Fc region. The Fc region of an immunoglobulin generally comprises two constant domains, a CH2 domain and a CH3 domain, and optionally comprises a CH4 domain. Replacements of amino acid residues in the Fc portion to alter antibody effector function are known in the art (e.g., U.S. Pat. Nos. 5,648,260 and 5,624,821). The Fc region mediates several important effector functions, e.g., cytokine induction, antibody dependent cell mediated cytotoxicity (ADCC), phagocytosis, complement dependent cytotoxicity (CDC), and half-life/clearance rate of antibody and antigen-antibody complexes. In some cases these effector functions are desirable for a therapeutic immunoglobulin but in other cases might be unnecessary or even deleterious, depending on the therapeutic objectives.

The term “antigen-binding portion” of a binding protein refers to a fragment of a binding protein (e.g., an antibody) that retains the ability to specifically bind to an antigen. The antigen-binding portion of a binding protein can be performed by fragments of a full-length antibody, as well as bispecific, dual specific, or multi-specific formats; specifically binding to two or more different antigens. Examples of binding fragments encompassed within the term “antigen-binding portion” of an binding protein include (i) an Fab fragment, a monovalent fragment consisting of the VL, VH, CL and CH1 domains; (ii) an F(ab′)₂ fragment, a bivalent fragment comprising two Fab fragments linked by a disulfide bridge at the hinge region; (iii) an Fd fragment consisting of the VH and CH1 domains; (iv) an Fv fragment consisting of the VL and VH domains of a single arm of an antibody, (v) a dAb fragment, which comprises a single variable domain; and (vi) an isolated complementarity determining region (CDR). Furthermore, although the two domains of the Fv fragment, VL and VH, encoded by separate genes, they can be joined, using recombinant methods, by a synthetic linker that enables them to be made as a single protein chain in which the VL and VH regions pair to form monovalent molecules (known as single chain Fv (scFv). Such single chain antibodies are also intended to be encompassed within the term “antigen-binding portion” of an antibody. Other forms of single chain antibodies, such as diabodies are also encompassed. In addition, single chain antibodies also include “linear antibodies” comprising a pair of tandem Fv segments (VH-CH1-VH-CH1) which, together with complementary light chain polypeptides, form a pair of antigen binding regions.

The term “multivalent binding protein” means a binding protein comprising two or more antigen binding sites. In an embodiment, the multivalent binding protein is engineered to have three or more antigen binding sites, and is not a naturally occurring antibody. The term “multispecific binding protein” refers to a binding protein capable of binding two or more related or unrelated targets. In an embodiment, the dual variable domain (DVD) binding proteins provided herein comprise two or more antigen binding sites and are tetravalent or multivalent binding proteins.

The term “linker” means an amino acid residue or a polypeptide comprising two or more amino acid residues joined by peptide bonds that are used to link two polypeptides (e.g., two VH or two VL domains). Such linker polypeptides are well known in the art (see, e.g., Holliger et al. (1993) Proc. Natl. Acad. Sci. USA 90:6444-6448; Poljak et al. (1994) Structure 2:1121-1123).

The terms “Kabat numbering”, “Kabat definitions” and “Kabat labeling” are used interchangeably herein. These terms, which are recognized in the art, refer to a system of numbering amino acid residues which are more variable (i.e., hypervariable) than other amino acid residues in the heavy and light chain variable regions of an antibody, or an antigen binding portion thereof (Kabat et al. (1971) Ann. NY Acad. Sci. 190:382-391 and Kabat et al. (1991) Sequences of Proteins of Immunological Interest, Fifth Edition, U.S. Department of Health and Human Services, NIH Publication No. 91-3242). For the heavy chain variable region, the hypervariable region ranges from amino acid positions 31 to 35 for CDR1, amino acid positions 50 to 65 for CDR2, and amino acid positions 95 to 102 for CDR3. For the light chain variable region, the hypervariable region ranges from amino acid positions 24 to 34 for CDR1, amino acid positions 50 to 56 for CDR2, and amino acid positions 89 to 97 for CDR3.

The system described by Kabat not only provides an unambiguous residue numbering system applicable to any variable region of an antibody, but also provides precise residue boundaries defining the three CDRs. These CDRs may be referred to as Kabat CDRs. Chothia and coworkers (Chothia and Lesk (1987) J. Mol. Biol. 196:901-917; Chothia et al. (1989) Nature 342:877-883) found that certain sub-portions within Kabat CDRs adopt nearly identical peptide backbone conformations, despite having great diversity at the level of amino acid sequence. These sub-portions were designated as L1, L2 and L3 or H1, H2 and H3 where the “L” and the “H” designates the light chain and the heavy chain regions, respectively. These regions may be referred to as Chothia CDRs, which have boundaries that overlap with Kabat CDRs. Other boundaries defining CDRs overlapping with the Kabat CDRs have been described by Padlan (1995) FASEB J. 9:133-139 and MacCallum (1996) J. Mol. Biol. 262(5):732-45). Still other CDR boundary definitions may not strictly follow one of the herein systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although certain embodiments use Kabat or Chothia defined CDRs.

The term “epitope” means a region of an antigen that is bound by a binding protein, e.g., a polypeptide and/or other determinant capable of specific binding to an immunoglobulin or T-cell receptor. In certain embodiments, epitope determinants include chemically active surface groupings of molecules such as amino acids, sugar side chains, phosphoryl, or sulfonyl, and, in certain embodiments, may have specific three dimensional structural characteristics, and/or specific charge characteristics. In an embodiment, an epitope comprises the amino acid residues of a region of an antigen (or fragment thereof) known to bind to the complementary site on the specific binding partner. An antigenic fragment can contain more than one epitope. In certain embodiments, a binding protein specifically binds an antigen when it recognizes its target antigen in a complex mixture of proteins and/or macromolecules. Binding proteins “bind to the same epitope” if the antibodies cross-compete (e.g., one prevents the binding or modulating effect of the other). In addition, structural definitions of epitopes (e.g., overlapping, similar, identical) are informative; and functional definitions encompass structural (e.g., binding) and functional (e.g., modulation, competition) parameters. Different regions of proteins may perform different functions. For example specific regions of a cytokine interact with its cytokine receptor to bring about receptor activation whereas other regions of the protein may be required for stabilizing the cytokine. To abrogate the negative effects of cytokine signaling, the cytokine may be targeted with a binding protein that binds specifically to the receptor interacting region(s), thereby preventing the binding of its receptor. Alternatively, a binding protein may target the regions responsible for cytokine stabilization, thereby designating the protein for degradation. The methods of visualizing and modeling epitope recognition are known to one skilled in the art (See US Patent Publication No. 20090311253).

The terms “pharmacokinetics” or “PK” refer to the process by which a drug is absorbed, distributed, metabolized, and excreted by an organism. To generate a multivalent binding protein molecule with a desired PK profile, parent monoclonal antibodies with similarly desired PK profiles are selected. The PK profiles of the selected parental monoclonal antibodies can be easily determined in rodents using methods known to one skilled in the art (See US Patent Publication No. 20090311253).

The term “bioavailability” refers to the amount of active drug that reaches its target following administration. Bioavailability is function of several of the previously described properties, including stability, solubility, immunogenicity and pharmacokinetics, and can be assessed using methods known to one skilled in the art (See US Patent Publication No. 20090311253).

The term “surface plasmon resonance” means an optical phenomenon that allows for the analysis of real-time biospecific interactions by detection of alterations in protein concentrations within a biosensor matrix, for example using the BIAcore® system (BIAcore International AB, a GE Healthcare company, Uppsala, Sweden and Piscataway, N.J.). For further descriptions, see Jönsson et al. (1993) Ann. Biol. Clin. 51:19-26. The term “Kₒₙ” means the on rate constant for association of a binding protein (e.g., an antibody or DVD-Ig protein) to the antigen to form the, e.g., DVD-Ig protein/antigen complex. The term “Kₒₙ” also means “association rate constant”, or “ka”, as is used interchangeably herein. This value indicating the binding rate of a binding protein to its target antigen or the rate of complex formation between a binding protein, e.g., an antibody, and antigen also is shown by the equation below:

Antibody(“Ab”)+Antigen(“Ag”)→Ab-Ag

The term “K_{off}” means the off rate constant for dissociation, or “dissociation rate constant”, of a binding protein (e.g., an antibody or DVD-Ig protein) from the, e.g., DVD-Ig protein/antigen complex as is known in the art. This value indicates the dissociation rate of a binding protein, e.g., an antibody, from its target antigen or separation of Ab-Ag complex over time into free antibody and antigen as shown by the equation below:

Ab+Ag←Ab−Ag

The terms “K_{d}” and “equilibrium dissociation constant” means the value obtained in a titration measurement at equilibrium, or by dividing the dissociation rate constant (K_{off}) by the association rate constant (Kₒₙ). The association rate constant, the dissociation rate constant and the equilibrium dissociation constant, are used to represent the binding affinity of a binding protein (e.g., an antibody or DVD-Ig protein) to an antigen. Methods for determining association and dissociation rate constants are well known in the art. Using fluorescence-based techniques offers high sensitivity and the ability to examine samples in physiological buffers at equilibrium. Other experimental approaches and instruments such as a BIAcore® (biomolecular interaction analysis) assay, can be used (e.g., instrument available from BIAcore International AB, a GE Healthcare company, Uppsala, Sweden). Additionally, a KinExA® (Kinetic Exclusion Assay) assay, available from Sapidyne Instruments (Boise, Id.), can also be used.

The term “variant” means a polypeptide that differs from a given polypeptide in amino acid sequence by the addition (e.g., insertion), deletion, or conservative substitution of amino acids, but that retains the biological activity of the given polypeptide (e.g., a variant TfR antibody can compete with anti-TfR antibody for binding to TfR). A conservative substitution of an amino acid, i.e., replacing an amino acid with a different amino acid of similar properties (e.g., hydrophilicity and degree and distribution of charged regions) is recognized in the art as typically involving a minor change. These minor changes can be identified, in part, by considering the hydropathic index of amino acids, as understood in the art (see, e.g., Kyte et al. (1982) J. Mol. Biol. 157: 105-132). The hydropathic index of an amino acid is based on a consideration of its hydrophobicity and charge. It is known in the art that amino acids of similar hydropathic indexes in a protein can be substituted and the protein still retains protein function. In one aspect, amino acids having hydropathic indexes of ±2 are substituted. The hydrophilicity of amino acids also can be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity (see, e.g., U.S. Pat. No. 4,554,101). Substitution of amino acids having similar hydrophilicity values can result in peptides retaining biological activity, for example immunogenicity, as is understood in the art. In one aspect, substitutions are performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties. The term “variant” also includes a polypeptide or fragment thereof that has been differentially processed, such as by proteolysis, phosphorylation, or other post-translational modification, yet retains its biological activity or antigen reactivity, e.g., the ability to bind to TfR. The term “variant” encompasses fragments of a variant unless otherwise defined. A variant may be 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, or 75% identical to the wild-type sequence.

### I. Generation of Binding Proteins

Binding proteins capable of binding an antigen expressed on the brain that facilitates uptake of the binding protein into the brain (e.g., TfR) and methods of making the same are provided. The binding protein can be generated using various techniques. Expression vectors, host cell and methods of generating the binding protein are provided and are well known in the art.

A. Generation of Parent Monoclonal Antibodies

The variable domains of the DVD binding protein can be obtained from parent antibodies, including polyclonal Abs and mAbs capable of binding antigens of interest. These antibodies may be naturally occurring or may be generated by recombinant technology. The person of ordinary skill in the art is well familiar with many methods for producing antibodies, including, but not limited to using hybridoma techniques, selected lymphocyte antibody method (SLAM), use of a phage, yeast, or RNA-protein fusion display or other library, immunizing a non-human animal comprising at least some of the human immunoglobulin locus, and preparation of chimeric, CDR-grafted, and humanized antibodies. See, e.g., US Patent Publication No. 20090311253. Variable domains may also be prepared using affinity maturation techniques.

B. Criteria for Selecting Parent Monoclonal Antibodies

An embodiment is provided comprising selecting parent antibodies with at least one or more properties desired in the DVD binding protein molecule. In an embodiment, the desired property is one or more antibody parameters, such as, for example, antigen specificity, affinity to antigen, potency, biological function, epitope recognition, stability, solubility, production efficiency, immunogenicity, pharmacokinetics, bioavailability, tissue cross reactivity, or orthologous antigen binding. See, e.g., US Patent Publication No. 20090311253.

C. Construction of Binding Protein Molecules

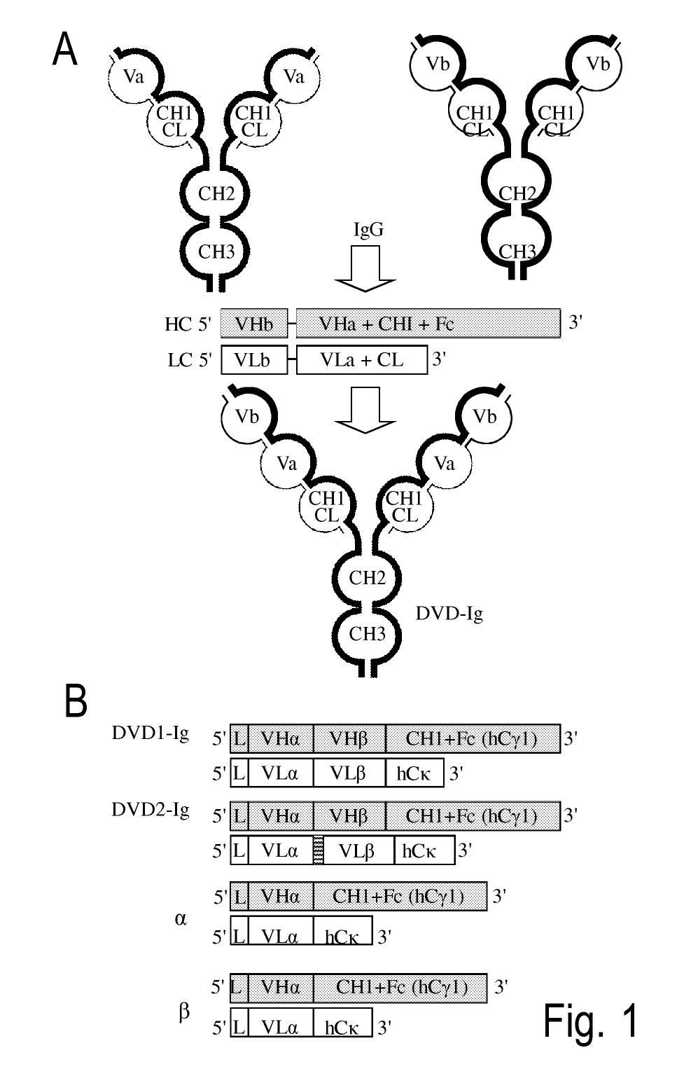

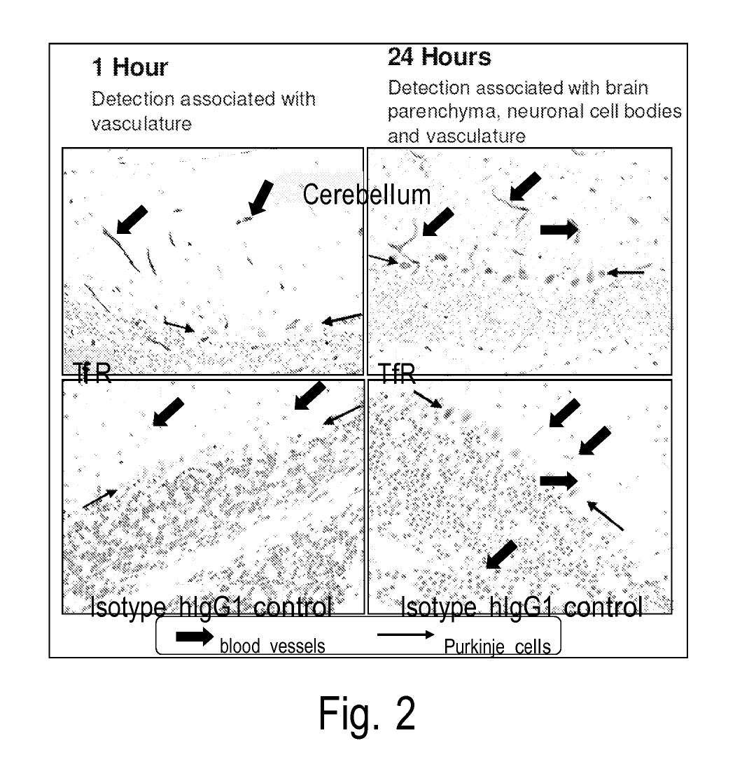

The binding protein may be designed such that two different light chain variable domains (VL) from the two different parent monoclonal antibodies are linked in tandem directly or via a linker by recombinant DNA techniques, followed by the light chain constant domain CL. Similarly, the heavy chain comprises two different heavy chain variable domains (VH) linked in tandem, directly or via a linker, followed by the constant domain CH1 and Fc region (FIG. 1).

The variable domains can be obtained using recombinant DNA techniques from parent antibodies generated by any one of the methods described herein. In an embodiment, the variable domain is a murine heavy or light chain variable domain. In another embodiment, the variable domain is a CDR grafted or a humanized variable heavy or light chain domain. In an embodiment, the variable domain is a human heavy or light chain variable domain.

The linker sequence may be a single amino acid or a polypeptide sequence. In an embodiment, the choice of linker sequences is based on crystal structure analysis of several Fab molecules. There is a natural flexible linkage between the variable domain and the CH1/CL constant domain in Fab or antibody molecular structure. This natural linkage comprises approximately 10-12 amino acid residues, contributed by 4-6 residues from the C-terminus of a V domain and 4-6 residues from the N-terminus of a CL/CH1 domain. DVD binding proteins were generated using N-terminal 5-6 amino acid residues, or 11-12 amino acid residues, of CL or CH1 as a linker in the light chain and heavy chains, respectively. The N-terminal residues of CL or CH1 domains, particularly the first 5-6 amino acid residues, can adopt a loop conformation without strong secondary structures, and therefore can act as flexible linkers between the two variable domains. The N-terminal residues of CL or CH1 domains are natural extension of the variable domains, as they are part of the Ig sequences, and therefore their use minimizes to a large extent any immunogenicity potentially arising from the linkers and junctions.

In a further embodiment, of any of the heavy chain, light chain, two chain, or four chain embodiments, includes at least one linker comprising AKTTPKLEEGEFSEAR (SEQ ID NO: 1); AKTTPKLEEGEFSEARV (SEQ ID NO: 2); AKTTPKLGG (SEQ ID NO: 3); SAKTTPKLGG (SEQ ID NO: 4); SAKTTP (SEQ ID NO: 5); RADAAP (SEQ ID NO: 6); RADAAPTVS (SEQ ID NO: 7); RADAAAAGGPGS (SEQ ID NO: 8); RADAAAA (G₄S)₄ (SEQ ID NO: 9), SAKTTPKLEEGEFSEARV (SEQ ID NO: 10); ADAAP (SEQ ID NO: 11); ADAAPTVSIFPP (SEQ ID NO: 12); TVAAP (SEQ ID NO: 13); TVAAPSVFIFPP (SEQ ID NO: 14); QPKAAP (SEQ ID NO: 15); QPKAAPSVTLFPP (SEQ ID NO: 16); AKTTPP (SEQ ID NO: 17); AKTTPPSVTPLAP (SEQ ID NO: 18); AKTTAP (SEQ ID NO: 19); AKTTAPSVYPLAP (SEQ ID NO: 20); ASTKGP (SEQ ID NO: 21); ASTKGPSVFPLAP (SEQ ID NO: 22), GGGGSGGGGSGGGGS (SEQ ID NO: 23); GENKVEYAPALMALS (SEQ ID NO: 24); GPAKELTPLKEAKVS (SEQ ID NO: 25); or GHEAAAVMQVQYPAS (SEQ ID NO: 26); TVAAPSVFIFPPTVAAPSVFIFPP (SEQ ID NO: 27); ASTKGPSVFPLAPASTKGPSVFPLAP (SEQ ID NO: 28); G/S based sequences (e.g., G4S repeats; SEQ ID NO: 29) GSGSGNGS (SEQ ID NO: 209), GSGSGSGS (SEQ ID NO: 210), GGSGSGSG (SEQ ID NO: 211), GGSGSG (SEQ ID NO: 212), GGSG (SEQ ID NO: 213), GGSGNGSG (SEQ ID: 214), or GSG (SEQ ID NO: 215).

In an embodiment, X2 is an Fc region. In another embodiment, X2 is a variant Fc region.

In various embodiments, the linker comprises GS-H10 (Chain H) GGGGSGGGGS (SEQ ID NO: 178). In various embodiments, the linker comprises GS-L10 (Chain L) GGSGGGGSG (SEQ ID NO: 179). In various embodiments, the linker comprises HG-short (Chain H) ASTKGP (SEQ ID NO: 21). In various embodiments, the linker comprises LK-long (Chain L) TVAAPSVFIFPP (SEQ ID NO: 14). For example SEQ ID NOs: 21 and 178 are located on a variable heavy chain or domain of a DVD-Ig protein. For example SEQ ID NOs: 14 and 179 are located on a variable light chain or domain of a DVD-Ig protein.

Other linker sequences may include any sequence of any length of a CL/CH1 domain but not all residues of a CL/CH1 domain; for example the first 5-12 amino acid residues of a CL/CH1 domain; the light chain linkers can be from Cκ or Cλ; and the heavy chain linkers can be derived from CH1 of any isotype, including Cγ1, Cγ2, Cγ3, Cγ4, Cα1, Cα2, Cδ, Cε, and Cμ. Linker sequences may also be derived from other proteins such as Ig-like proteins (e.g., TCR, FcR, KIR); G/S based sequences (e.g., G4S repeats; SEQ ID NO: 29); hinge region-derived sequences; and other natural sequences from other proteins.

In an embodiment, a constant domain is linked to the two linked variable domains using recombinant DNA techniques. In an embodiment, a sequence comprising linked heavy chain variable domains is linked to a heavy chain constant domain and a sequence comprising linked light chain variable domains is linked to a light chain constant domain. In an embodiment, the constant domains are human heavy chain constant domains and human light chain constant domains respectively. In an embodiment, the DVD heavy chain is further linked to an Fc region. The Fc region may be a native sequence Fc region or a variant Fc region. In another embodiment, the Fc region is a human Fc region. In another embodiment, the Fc region includes Fc region from IgG1, IgG2, IgG3, IgG4, IgA, IgM, IgE, or IgD.

In another embodiment, two heavy chain DVD polypeptides and two light chain DVD polypeptides are combined to form a DVD binding protein. Tables 1-4 list amino acid sequences of VH and VL regions and CDRs of exemplary antibodies useful for treating disease. In an embodiment, a DVD binding protein comprising at least one CDR or at least two of the VH and/or VL regions listed in Table 1-4, in any orientation, is provided. Exemplary DVD binding proteins are provided in Table 3. In some embodiments, VD1 and VD2 are independently chosen. Therefore, in some embodiments, VD1 and VD2 comprise the same SEQ ID NO and, in other embodiments, VD1 and VD2 comprise different SEQ ID NOS. The VH and VL domain sequences provided below comprise CDRs and framework sequences that are either known in the art or readily discernible using methods known in the art. In some embodiments, one or more of these CDRs and/or framework sequences are replaced, without loss of function, by other CDRs and/or framework sequences from binding proteins that are known in the art to bind to the same antigen. Detailed description of specific DVD-Ig binding proteins capable of binding specific targets, and methods of making the same, is provided in the Examples section below.

D. Production of Binding Proteins

The binding proteins provided herein may be produced by any of a number of techniques known in the art. For example, expression from host cells, wherein expression vector(s) encoding the DVD heavy and/or DVD light chains are/is transfected into a host cell by standard techniques. Although it is possible to express the DVD binding proteins provided herein in either prokaryotic or eukaryotic host cells, DVD binding proteins are expressed in eukaryotic cells, for example, mammalian host cells, because such eukaryotic cells (and in particular mammalian cells) are more likely than prokaryotic cells to assemble and secrete a properly folded and immunologically active DVD binding protein.

In an exemplary system for recombinant expression of DVD proteins, a recombinant expression vector encoding both the DVD heavy chain and the DVD light chain is introduced into dhfr-CHO cells by calcium phosphate-mediated transfection. Within the recombinant expression vector, the DVD heavy and light chain genes are each operatively linked to CMV enhancer/AdMLP promoter regulatory elements to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the DVD heavy and light chains and intact DVD protein is recovered from the culture medium. Standard molecular biology techniques are used to prepare the recombinant expression vector, transfect the host cells, select for transformants, culture the host cells and recover the DVD protein from the culture medium. A method of synthesizing a DVD protein provided herein by culturing a host cell provided herein in a suitable culture medium until a DVD protein is synthesized is also provided. The method can further comprise isolating the DVD protein from the culture medium.

An important feature of DVD binding protein is that it can be produced and purified in a way similar to that of a conventional antibody. The production of DVD binding protein results in a homogeneous, single major product with desired dual-specific activity, without the need for sequence modification of the constant region or chemical modifications. Other previously described methods to generate “bi-specific”, “multi-specific”, and “multi-specific multivalent” full length binding proteins can lead to the intracellular or secreted production of a mixture of assembled inactive, mono-specific, multi-specific, multivalent, full length binding proteins, and multivalent full length binding proteins with a combination of different binding sites.

Surprisingly, the design of the “dual-specific multivalent full length binding proteins” provided herein leads to a dual variable domain light chain and a dual variable domain heavy chain that assemble primarily to the desired “dual-specific multivalent full length binding proteins”.

At least 50%, at least 75% and at least 90% of the assembled and expressed DVD-Ig binding molecules are the desired dual-specific tetravalent protein, and therefore possess enhanced commercial utility. Thus, a method for expressing a dual variable domain light chain and a dual variable domain heavy chain in a single cell leading to a single primary product of a “dual-specific tetravalent full length binding protein” is provided.

Methods of expressing a dual variable domain light chain and a dual variable domain heavy chain in a single cell leading to a “primary product” of a “dual-specific tetravalent full length binding protein”, where the “primary product” is more than 50%, such as more than 75% and more than 90%, of all assembled protein, comprising a dual variable domain light chain and a dual variable domain heavy chain are provided.

E. DVD Cassettes

In certain embodiments, cassettes can be used to construct binding proteins that specifically bind to an antigen expressed on brain vascular endothelium of a subject that facilitates uptake of the binding protein into the brain of the subject. In some embodiments, the formula for these binding proteins is

Outer1-(X1)m-Inner1-(X2)n (I)

According to Formula I, Outer1 is a first outer binding domain and Inner1 is a first inner binding domain. In certain embodiments, Inner1 represents a binding domain positioned closer to the Fc region of a DVD-Ig protein than Outer1. In other embodiments, Outer1 is located at or near the N-terminal end of the binding protein while the Inner1 is located at or near the C-terminal end of the binding protein.

According to Formula I, X1 is a linker. According to some embodiments, X1 is any of the linkers defined herein. According to other specific embodiments, X1 has a sequence comprising the amino acid sequences of SEQ ID NO:14 or 21 when Outer1 specifically binds an antigen expressed on brain vascular endothelium of a subject that facilitates uptake of the binding protein into the brain of the subject and Inner1 does not specifically bind the antigen, while X1 has a sequence comprising the amino acid sequence of SEQ ID NO:178 or 179 when Inner1 specifically binds an antigen expressed on brain vascular endothelium of a subject that facilitates uptake of the binding protein into the brain of the subject and Outer1 does not specifically bind the antigen. According to Formula I, X2 is an Fc region. The values of m and n in Formula I are 0 or 1. In certain embodiments, when n is 0, X1 is X1 comprises the amino acid sequence of SEQ ID NO:14 or 179 depending on whether Outer1 or Inner1 specifically binds an antigen expressed on brain vascular endothelium of a subject that facilitates uptake of the binding protein into the brain of the subject. When n is 1, X1 comprises the amino acid sequences of SEQ ID NO: 21 or 178 depending on whether Outer1 or Inner1 specifically binds the antigen.

When Outer1 is used to specifically bind an antigen expressed on the brain vascular endothelium of a subject that facilitates uptake of the binding protein into the brain, it does not need to have as high an affinity as when Inner1 is used. Thus, in certain embodiments, when Outer1 specifically binds an antigen expressed on brain vascular endothelium of a subject that facilitates uptake of the binding protein into the brain of the subject the binding affinity that Outer1 has for the antigen is lower than if Inner1 were to bind the antigen. For example, when Outer1 specifically binds the antigen, the EC50 of the binding is greater than about 1, about 2, about 3, about 4, about 5, about 6, about 7, about 8, about 9 or about 10 nM. In other embodiments, when Outer1 specifically binds the antigen, the EC50 of the binding is between about 1 and about 10 nM, about 2 and about 8 nM, about 3 and about 10 nM, about 3 and about 9 nM, about 3 and about 8 nM, about 3 and about 7 nM, about 3 and about 6 nM, about 3 and about 5 nM, about 3 and about 4 nM, about 4 and about 10 nM or about 5 and about 10 nM.

In other embodiments, when Outer1 specifically binds transferrin receptor (TfR), Outer1 has an affinity for TfR that is lower than if Inner1 were to specifically bind to TfR. For example, when Outer1 specifically binds the antigen, the EC50 of the binding is greater than about 1, 2, 3, 4, 5, 6, 7, 8, 9 or 10 nM. In other embodiments, the EC50 is greater than about 3 nM. In other embodiments, when Outer1 specifically binds TfR, the EC50 of the binding is between about 1 and 10 nM, 2 and 8 nM, 3 and 10 nM, 3 and 9 nM, 3 and 8 nM, 3 and 7 nM, 3 and 6 nM, 3 and 5 nM, 3 and 4 nM, 4 and 10 nM or 5 and 10 nM. In other embodiments, the EC50 is between about 3 and 10 nM, 3 and 9 nM, 3 and 8 nM, 3 and 7 nM, 3 and 6 nM, 3 and 5 nM, or 3 and 4 nM. In certain embodiments, Outer1 comprises the amino acid sequence of SEQ ID NO:56.

In other embodiments, when Outer1 specifically binds an antigen expressed on brain vascular endothelium of a subject that facilitates uptake of the binding protein into the brain, Inner1 binds another antigen. This antigen (e.g., Inner1) can be selected from CGRP, TNFα, RGMA, Substance P, Bradykinin, Nav1.7, LPA, P2X3, NGF, Abeta; APP; BACE1; IL-1β; IGF1, or 2; IL-18; IL-6; RAGE; NGF; EGFR; cMet, Her-2 and CD-20.

When Inner1 is used to specifically bind an antigen expressed on brain vascular endothelium of a subject that facilitates uptake of the binding protein into the brain it needs to have a higher affinity than when Outer1 is used. Thus, in certain embodiments, when Inner1 specifically binds an antigen expressed on brain vascular endothelium of a subject that facilitates uptake of the binding protein into the brain of the subject the binding affinity that Inner1 has for the antigen is higher than if Outer1 were to bind the antigen. For example, when Inner1 specifically binds the antigen, the EC50 of the binding is less than about 1, about 2, about 3, about 4, about 5, about 6, about 7, about 8, about 9 or about 10 nM. In other embodiments, when Outer1 specifically binds the antigen, the EC50 of the binding is between about 1 and about 0.001 nM, about 2 and about 0.001 nM, about 3 and about 0.0001 nM, about 3 and about 0.001 nM, about 3 and about 0.01 nM, about 3 and about 0.1 nM, about 3 and about 1 nM, about 3 and about 5 nM, about 3 and about 10 nM, about 4 and about 10 nM or about 5 and about 10 nM.

In other embodiments, when Outer1 specifically binds transferrin receptor (TfR), Inner1 has an affinity for TfR that is higher than if Outer1 were to specifically bind to TfR. For example, when Inner1 specifically binds the antigen, the EC50 of the binding is less than about 1, about 2, about 3, about 4, about 5, about 6, about 7, about 8, about 9 or about 10 nM. In other embodiments, the EC50 is less than about 3 nM. In other embodiments, when Inner1 specifically binds TfR, the EC50 of the binding is between about 1 and about 0.001 nM, about 2 and about 0.001 nM, about 3 and about 0.0001 nM, about 3 and about 0.001 nM, about 3 and about 0.01 nM, about 3 and about 0.1 nM, about 3 and about 1 nM, about 3 and about 5 nM, about 3 and about 10 nM, about 4 and about 10 nM or about 5 and about 10 nM. In other embodiments, the EC50 is between about 3 and about 0.0001 nM, about 3 and about 0.001 nM, about 3 and about 0.01 nM, about 3 and about 0.1 nM, about 3 and about 1 nM, about 3 and about 5 nM or about 3 and about 10 nM. In certain embodiments, Inner1 comprises the amino acid sequence of a binder described herein, for example in a table or chart. For example, the amino acid sequence comprises SEQ ID NO: 36.

In other embodiments, when Inner1 specifically binds an antigen expressed on brain vascular endothelium of a subject that facilitates uptake of the binding protein into the brain, Outer1 binds another antigen. This antigen can be selected from CGRP, TNFα, RGMA, Substance P, Bradykinin, Nav1.7, LPA, P2X3, NGF, Abeta; APP; BACE1; IL-1β; IGF1, or 2; IL-18; IL-6; RAGE; NGF; EGFR; cMet, Her-2 and CD-20.

In other embodiments, a binding protein may comprise a second binding protein. In some embodiments, the formula for this second binding protein is

Outer2-(X1)m-Inner2-(X2)n (II)

According to Formula II, Outer2 is a second outer binding domain and Inner2 is a second inner binding domain. As explained above, in certain embodiments, the inner binding domain represents a binding domain positioned closer to the Fc region of a DVD-Ig protein than the outer binding domain. In other embodiments, the outer binding domain is located at or near the N-terminal end of the binding protein while the inner binding domain is located at or near the C-terminal end of the binding protein. X1 and X2 are as defined in Formula I, above.

Outer2 and Inner2 operate in the same manner as Outer1 and Inner1 described above. This second binding protein can be associated with a first binding protein to form a binding polypeptide such as a DVD-Ig protein. In these embodiments, in the first binding protein n is 1 and in the second binding protein n is 0. In certain embodiments, both Outer1 and Outer2 bind an antigen expressed on brain vascular endothelium of a subject that facilitates uptake of the binding protein into the brain of the subject. In other embodiments, both Inner1 and Inner2 bind the antigen. According to other embodiments, Outer1 and Inner2 or Outer2 and Inner1 bind the antigen. In certain embodiments, Outer2 comprises the amino acid sequence of SEQ ID NO: 37. In other embodiments, Inner2 comprises the amino acid sequence of SEQ ID NO: 57. See International Application PCT/US2013/073114 and U.S. patent application Ser. No. 14/097,033, the contents of which are hereby incorporated by reference in their entireties.

### II. Uses of Binding Proteins

In an embodiment, the binding proteins provided herein are capable of binding to an antigen in or on the brain and/or neutralizing the activity of antigen targets both in vitro and in vivo. Accordingly, such binding proteins can be used to inhibit antigen activity, e.g., in a cell culture containing the antigens, in human subjects or in other mammalian subjects having the antigens with which a binding protein provided herein cross-reacts. In another embodiment, a method for reducing antigen activity in a subject suffering from a disease or disorder in which the antigen activity is detrimental is provided. A binding protein provided herein can be administered to a human subject for therapeutic purposes.

DVD binding proteins are useful as therapeutic agents to simultaneously block two different targets to enhance efficacy/safety and/or increase patient coverage.

Additionally, DVD binding proteins provided herein can be employed for tissue-specific delivery (e.g., target a tissue marker in the brain and a disease mediator for enhanced local PK thus higher efficacy and/or lower toxicity), including intracellular delivery (targeting an internalizing receptor and an intracellular molecule), delivering to inside brain (targeting transferrin receptor and a CNS disease mediator for crossing the blood-brain barrier). DVD binding protein can also serve as a carrier protein to deliver an antigen to a specific location via binding to a non-neutralizing epitope of that antigen and also to increase the half-life of the antigen. Furthermore, DVD binding protein can be designed to either be physically linked to medical devices implanted into patients or target these medical devices (see Burke et al. (2006) Advanced Drug Deliv. Rev. 58(3): 437-446; Hildebrand et al. (2006) Surface and Coatings Technol. 200(22-23): 6318-6324; Drug/device combinations for local drug therapies and infection prophylaxis, Wu (2006) Biomaterials 27(11):2450-2467; Mediation of the cytokine network in the implantation of orthopedic devices, Marques (2005) Biodegradable Systems in Tissue Engineer. Regen. Med. 377-397). Briefly, directing appropriate types of cell to the site of medical implant may promote healing and restoring normal tissue function. Alternatively, inhibition of mediators (including but not limited to cytokines), released upon device implantation by a DVD binding protein coupled to, or targeted to, a device is also provided.

A. Use of Binding Proteins in Various Neurological Diseases

Neurodegenerative diseases (e.g., AD, PD and MS) are either chronic, in which case they are usually age-dependent, or acute (e.g., stroke, traumatic brain injury, spinal cord injury, etc.). They are characterized by progressive loss of neuronal functions (e.g., neuronal cell death, axon loss, neuritic dystrophy, demyelination), loss of mobility and loss of memory. These chronic neurodegenerative diseases represent a complex interaction between multiple cell types and mediators. Treatment strategies for such diseases are limited and mostly constitute either blocking inflammatory processes with non-specific anti-inflammatory agents (e.g., corticosteroids, COX inhibitors) or agents to prevent neuron loss and/or synaptic functions. These treatments often fail to stop disease progression. Specific therapies targeting more than one disease mediator may provide even better therapeutic efficacy for chronic neurodegenerative diseases than observed with targeting a single disease mechanism (see Deane et al. (2003) Nature Med. 9:907-13; and Masliah et al. (2005) Neuron 46:857).

The binding protein molecules provided herein can allow for transport of therapeutics across the blood brain barrier. In certain embodiments, these therapeutics bind one or more targets involved in chronic neurodegenerative diseases such as Alzheimer's disease. The efficacy of binding protein molecules and its combination with other therapeutics can be validated in pre-clinical animal models such as the transgenic mice that over-express amyloid precursor protein or RAGE and develop Alzheimer's disease-like symptoms. In addition, binding protein molecules can be constructed and tested for efficacy in the animal models and the best therapeutic binding protein can be selected for testing in human patients. Binding protein molecules can also be employed for treatment of other neurodegenerative diseases such as Parkinson's disease. Other pain related targets include CGRP, TNFα, RGMA, Substance P, Bradykinin, Nav1.7, LPA, P2X3, and NGF. In various embodiments, the pharmaceutical composition includes the binding protein, and a detectable agent. In various embodiments, the detectable agent comprises a detectable agent or imaging agent for analysis of the brain. For example, the detectable agent comprises a fluorescent agent, a colorimetric agent, an enzymatic agent, or a radioactive agent.

### III. Pharmaceutical Compositions

Pharmaceutical compositions comprising one or more binding proteins, either alone or in combination with prophylactic agents, therapeutic agents, and/or pharmaceutically acceptable carriers are provided. The pharmaceutical compositions comprising binding proteins provided herein are for use in, but not limited to, diagnosing, detecting, or monitoring a disorder, in preventing, treating, managing, or ameliorating a disorder or one or more symptoms thereof, and/or in research. The formulation of pharmaceutical compositions, either alone or in combination with prophylactic agents, therapeutic agents, and/or pharmaceutically acceptable carriers, is known to one skilled in the art (U.S. Patent Publication No. 20090311253).

Methods of administering a prophylactic or therapeutic agent provided herein include, but are not limited to, parenteral administration (e.g., intradermal, intramuscular, intraperitoneal, intravenous and subcutaneous), epidural administration, intratumoral administration, mucosal administration (e.g., intranasal and oral routes) and pulmonary administration (e.g., aerosolized compounds administered with an inhaler or nebulizer). The formulation of pharmaceutical compositions for specific routes of administration, and the materials and techniques necessary for the various methods of administration are available and known to one skilled in the art (U.S. Patent Publication No. 20090311253).

Dosage regimens may be adjusted to provide the optimum desired response (e.g., a therapeutic or prophylactic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. The term “dosage unit form” refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms provided herein are dictated by and directly dependent on (a) the unique characteristics of the active compound and the particular therapeutic or prophylactic effect to be achieved, and (b) the limitations inherent in the art of compounding such an active compound for the treatment of sensitivity in individuals.

An exemplary, non-limiting range for a therapeutically or prophylactically effective amount of a binding protein provided herein is 0.1-100 mg/kg, for example, 1-40 mg/kg. It is to be noted that dosage values may vary with the type and severity of the condition to be alleviated is to be further understood that for any particular subject, specific dosage regimens may be adjusted over time according to the individual need and the professional judgment of the person administering or supervising the administration of the compositions, and that dosage ranges set forth herein are exemplary only and are not intended to limit the scope or practice of the claimed composition.

### IV. Combination Therapy

A binding protein provided herein also can also be administered with one or more additional medicaments or therapeutic agents useful in the treatment of various diseases, the additional agent being selected by the skilled artisan for its intended purpose. For example, the additional agent can be a therapeutic agent art-recognized as being useful to treat the disease or condition being treated by the antibody provided herein. The combination can also include more than one additional agent, e.g., two or three additional agents.

The binding agent in various embodiments is administered with an agent that is a protein, a peptide, a carbohydrate, a drug, a small molecule, and a genetic material (e.g., DNA or RNA). In various embodiments, the agent is an imaging agent, a cytotoxic agent, an angiogenesis inhibitor, a kinase inhibitor, a co-stimulation molecule blocker, an adhesion molecule blocker, an anti-cytokine antibody or functional fragment thereof, methotrexate, cyclosporin, rapamycin, FK506, a detectable label or reporter, a TNF antagonist, an antirheumatic, a muscle relaxant, a narcotic, a non-steroid anti-inflammatory drug (NSAID), an analgesic, an anesthetic, a sedative, a local anesthetic, a neuromuscular blocker, an antimicrobial, an antipsoriatic, a corticosteroid, an anabolic steroid, an erythropoietin, an immunization, an immunoglobulin, an immunosuppressive, a growth hormone, a hormone replacement drug, a radiopharmaceutical, an antidepressant, an antipsychotic, a stimulant, an asthma medication, a beta agonist, an inhaled steroid, an epinephrine or analog, a cytokine, or a cytokine antagonist.

The additional agent in various embodiments is a therapeutic agent. In various embodiments, the therapeutic agent comprises budenoside, epidermal growth factor, a corticosteroid, cyclosporin, sulfasalazine, an aminosalicylate, 6-mercaptopurine, azathioprine, metronidazole, a lipoxygenase inhibitor, mesalamine, olsalazine, balsalazide, an antioxidant, a thromboxane inhibitor, an IL-1 receptor antagonist, an anti-IL-1β mAbs, an anti-IL-6 or IL-6 receptor mAb, a growth factor, an elastase inhibitor, a pyridinyl-imidazole compound, an antibody specific against or an agonist of TNF, LT, IL-1, IL-2, IL-6, IL-7, IL-8, IL-12, IL-13, IL-15, IL-16, IL-18, IL-23, EMAP-II, GM-CSF, FGF, or PDGF, an antibody to CD2, CD3, CD4, CD8, CD-19, CD25, CD28, CD30, CD40, CD45, CD69, CD90 or a ligand thereof, methotrexate, cyclosporin, FK506, rapamycin, mycophenolate mofetil, leflunomide, an NSAID, ibuprofen, prednisolone, a phosphodiesterase inhibitor, an adenosine agonist, an antithrombotic agent, a complement inhibitor, an adrenergic agent, IRAK, NIK, IKK, p38, a MAP kinase inhibitor, an IL-1β converting enzyme inhibitor, a TNFα-converting enzyme inhibitor, a T-cell signaling inhibitor, a metalloproteinase inhibitor, sulfasalazine, azathioprine, a 6-mercaptopurine, an angiotensin converting enzyme inhibitor, a soluble cytokine receptor, a soluble p55 TNF receptor, a soluble p75 TNF receptor, sIL-1 RI, sIL-1RII, sIL-6R, an anti-inflammatory cytokine, IL-4, IL-10, IL-11, IL-13, or TGFβ.

Combination therapy agents include, but are not limited to, antineoplastic agents, radiotherapy, chemotherapy such as DNA alkylating agents, cisplatin, carboplatin, anti-tubulin agents, paclitaxel, docetaxel, taxol, doxorubicin, gemcitabine, gemzar, anthracyclines, adriamycin, topoisomerase I inhibitors, topoisomerase II inhibitors, 5-fluorouracil (5-FU), leucovorin, irinotecan, receptor tyrosine kinase inhibitors (e.g., erlotinib, gefitinib), COX-2 inhibitors (e.g., celecoxib), kinase inhibitors, and siRNAs.

### V. Diagnostics

The disclosure herein also provides diagnostic applications including, but not limited to, diagnostic assay methods, diagnostic kits containing one or more binding proteins, and adaptation of the methods and kits for use in automated and/or semi-automated systems. The methods, kits, and adaptations provided may be employed in the detection, monitoring, and/or treatment of a disease or disorder in an individual. This is further elucidated below.

A. Method of Assay

The present disclosure also provides a method for determining the presence, amount or concentration of an analyte, or fragment thereof, in a test sample using at least one binding protein as described herein. Any suitable assay as is known in the art can be used in the method. Examples include, but are not limited to, immunoassays and/or methods employing mass spectrometry.

Immunoassays provided by the present disclosure may include sandwich immunoassays, radioimmunoassay (RIA), enzyme immunoassay (EIA), enzyme-linked immunosorbent assay (ELISA), competitive-inhibition immunoassays, fluorescence polarization immunoassay (FPIA), enzyme multiplied immunoassay technique (EMIT), bioluminescence resonance energy transfer (BRET), and homogenous chemiluminescent assays, among others.

A chemiluminescent microparticle immunoassay, in particular one employing the ARCHITECT® automated analyzer (Abbott Laboratories, Abbott Park, Ill.), is an example of an immunoassay.

Methods employing mass spectrometry are provided by the present disclosure and include, but are not limited to MALDI (matrix-assisted laser desorption/ionization) or by SELDI (surface-enhanced laser desorption/ionization).

Methods for collecting, handling, processing, and analyzing biological test samples using immunoassays and mass spectrometry would be well-known to one skilled in the art, are provided for in the practice of the present disclosure (US Patent Publication No. 20090311253).

B. Kit

A kit for assaying a test sample for the presence, amount or concentration of an analyte, or fragment thereof, in a test sample is also provided. The kit comprises at least one component for assaying the test sample for the analyte, or fragment thereof, and instructions for assaying the test sample for the analyte, or fragment thereof. The at least one component for assaying the test sample for the analyte, or fragment thereof, can include a composition comprising a binding protein, as disclosed herein, and/or an anti-analyte binding protein (or a fragment, a variant, or a fragment of a variant thereof), which is optionally immobilized on a solid phase.

Optionally, the kit may comprise a calibrator or control, which may comprise isolated or purified analyte. The kit can comprise at least one component for assaying the test sample for an analyte by immunoassay and/or mass spectrometry. The kit components, including the analyte, binding protein, and/or anti-analyte binding protein, or fragments thereof, may be optionally labeled using any art-known detectable label. The materials and methods for the creation provided for in the practice of the present disclosure would be known to one skilled in the art (U.S. Patent Publication No. 20090311253).

C. Adaptation of Kit and Method

The kit (or components thereof), as well as the method of determining the presence, amount or concentration of an analyte in a test sample by an assay, such as an immunoassay as described herein, can be adapted for use in a variety of automated and semi-automated systems (including those wherein the solid phase comprises a microparticle), as described, for example, in U.S. Pat. Nos. 5,089,424 and 5,006,309, and as commercially marketed, for example, by Abbott Laboratories (Abbott Park, Ill.) as ARCHITECT®.

Other platforms available from Abbott Laboratories include, but are not limited to, AxSYM®, IMx® (see, for example, U.S. Pat. No. 5,294,404, PRISM®, EIA (bead), and Quantum™ II, as well as other platforms. Additionally, the assays, kits and kit components can be employed in other formats, for example, on electrochemical or other hand-held or point-of-care assay systems. The present disclosure is, for example, applicable to the commercial Abbott Point of Care (i-STAT®, Abbott Laboratories) electrochemical immunoassay system that performs sandwich immunoassays. Immunosensors and their methods of manufacture and operation in single-use test devices are described, for example in U.S. Pat. Nos. 5,063,081, 7,419,821, and 7,682,833; and US Publication Nos. 20040018577, 20060160164 and 20090311253.

It will be readily apparent to those skilled in the art that other suitable modifications and adaptations of the methods described herein are obvious and may be made using suitable equivalents without departing from the scope of the embodiments disclosed herein. Having now described certain embodiments in detail, the same will be more clearly understood by reference to the following examples, which are included for purposes of illustration only and are not intended to be limiting.

### EXAMPLES

### Example 1

### Generation and Characterization of Dual Variable Domain Immunogobulin (DVD-Ig) Binding Proteins

Four-chain dual variable domain immunoglobulin (DVD-Ig) binding proteins using parent antibodies with known amino acid sequences were generated by synthesizing polynucleotide fragments encoding DVD-Ig binding protein variable heavy and DVD-Ig binding protein variable light chain sequences and cloning the fragments into a pHybE-D2 vector according to art known methods. The DVD-Ig binding protein constructs were cloned into and expressed in 293 cells and purified according to art known methods. DVD-Ig VH and VL chains for the DVD-Ig binding proteins, as well as selected CDR sequences, are provided below in Tables 1 and 2, respectively. Exemplary DVD-Ig binding protein variable domain sequences are provided in Table 3.

TABLE 1
List of Amino Acid Sequences of VH and VL Regions of Antibodies for
Generating Binding Proteins, Including Multivalent Binding Proteins (CDRs
highlighted)

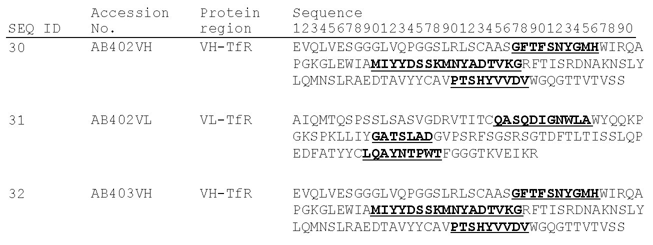

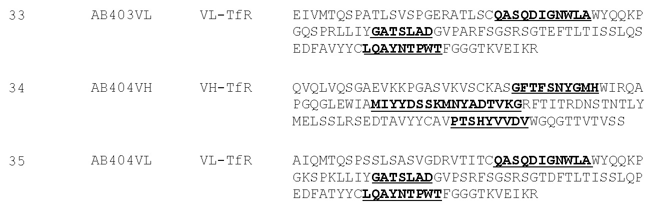

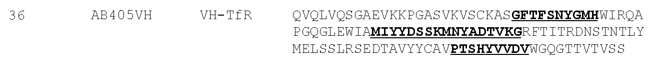

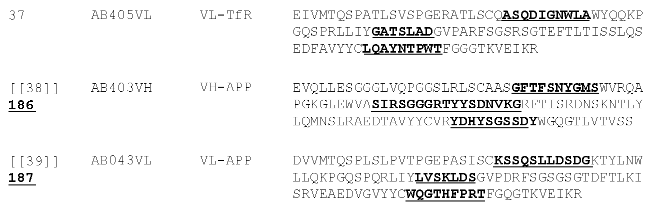

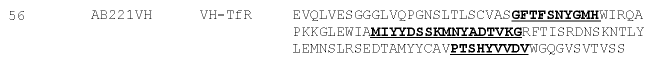

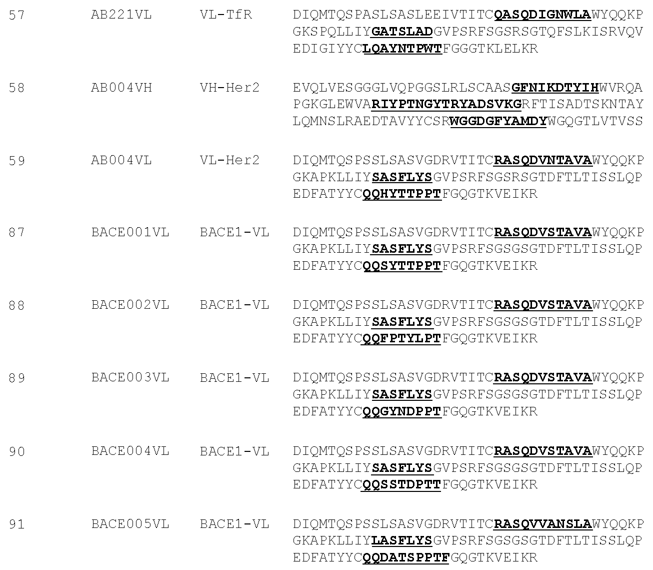

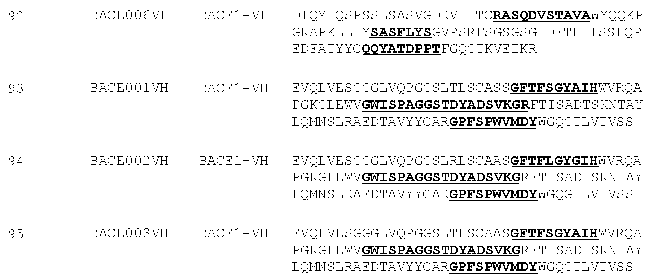

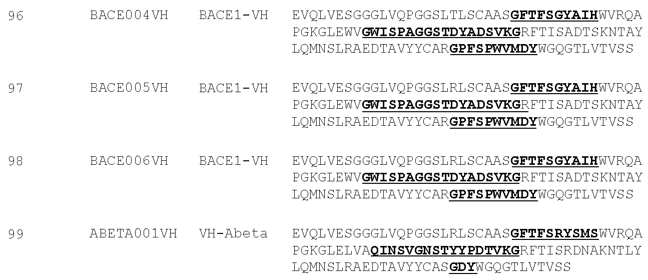

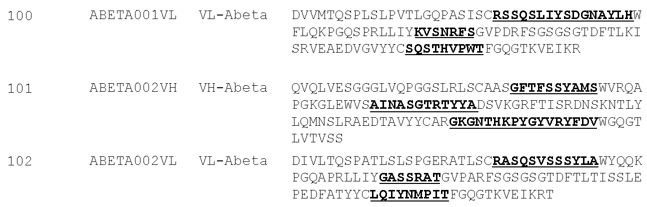

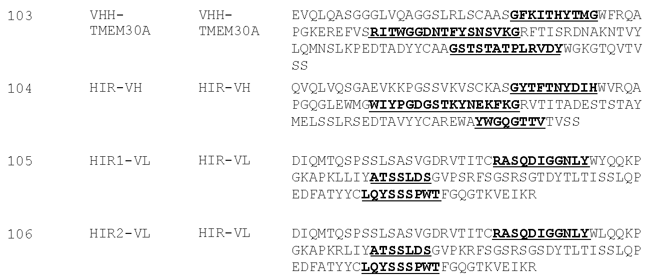

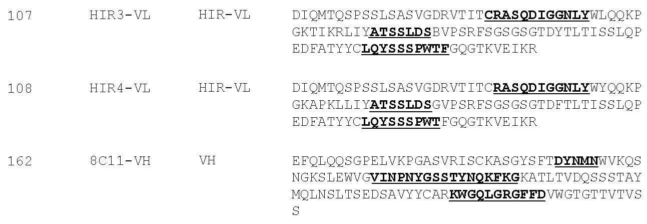

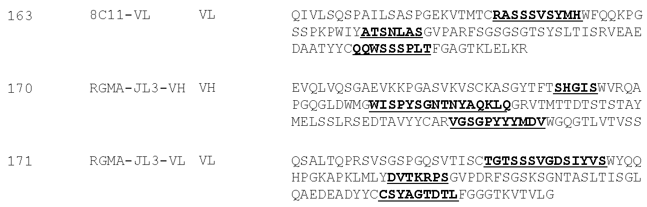

Note that CDRS for the VH and VL sequences are highlighted above and in Table 2 below.

TABLE 2
CDR Sequences of exemplary VH and VL domains

| Accession | | | |
|---|---|---|---|
| No. | CDR | Sequence | SEQ ID NO: |
| AB402VH | CDR1 | GFTFSNYGMH | 76 |
| (TfR) | | | |
| | CDR2 | MIYYDSSKMNYADTVKG | 77 |
| | CDR3 | PTSHYVVDV | 78 |
| AB402VL | CDR1 | QASQDIGNWLA | 79 |
| (TfR) | | | |
| | CDR2 | GATSLAD | 80 |
| | CDR3 | LQAYNTPWT | 81 |
| AB403VH | CDR1 | GFTFSNYGMH | 76 |
| (TfR) | | | |
| | CDR2 | MIYYDSSKMNYADTVKG | 82 |
| | CDR3 | PTSHYVVDV | 83 |
| AB403VL | CDR1 | QASQDIGNWLA | 84 |
| (TfR) | | | |
| | CDR2 | GATSLAD | 85 |
| | CDR3 | LQAYNTPWT | 86 |
| AB404VH | CDR1 | GFTFSNYGMH | 76 |
| (TfR) | | | |
| | CDR2 | MIYYDSSKMNYADTVKG | 77 |
| | CDR3 | PTSHYVVDV | 78 |
| AB405VH | CDR1 | GFTFSNYGMH | 76 |
| (TfR) | | | |
| | CDR2 | MIYYDSSKMNYADTVKG | 77 |
| | CDR3 | PTSHYVVDV | 78 |
| AB405VL | CDR1 | QASQDIGNWLA | 79 |
| (TfR) | | | |
| | CDR2 | GATSLAD | 80 |
| | CDR3 | LQAYNTPWT | 86 |
| AB043VH | CDR1 | GFTFSNYGMS | 109 |
| (APP) | | | |
| | CDR2 | SIRSGGGRTYYSDNVKG | 110 |
| | CDR3 | YDHYSGSSDY | 111 |
| AB043VL | CDR1 | KSSQSLLDSDG | 112 |
| (APP) | | | |
| | CDR2 | LVSKLDS | 113 |
| | CDR3 | WQGTHFPRT | 114 |
| AB221VH | CDR1 | GFTFSNYGMH | 115 |
| (TfR) | | | |
| | CDR2 | MIYYDSSKMNYADTVKG | 116 |
| | CDR3 | PTSHYVVDV | 117 |
| AB221VL | CDR1 | QASQDIGNWLA | 118 |
| (TfR) | | | |
| | CDR2 | GATSLAD | 119 |
| | CDR3 | LQAYNTPWT | 120 |
| AB004VH | CDR1 | GFNIKDTYIH | 121 |
| (Her2 ) | | | |
| | CDR2 | RIYPTNGYTRYADSVKG | 122 |
| | CDR3 | WGGDGFYAMDY | 123 |
| AB004VL | CDR1 | RASQDVNTAVA | 124 |
| (Her2 ) | | | |
| | CDR2 | SASFLYS | 125 |
| | CDR3 | QQHYTTPPT | 126 |
| BACE001VL | CDR1 | RASQDVSTAVA | 127 |
| (BACE1) | | | |
| | CDR2 | SASFLYS | 128 |
| | CDR3 | QQSYTTPPT | 129 |
| BACE002VL | CDR1 | RASQDVSTAVA | 127 |
| (BACE1) | | | |
| | CDR2 | SASFLYS | 128 |
| | CDR3 | QQFPTYLPT | 130 |
| BACE003VL | CDR1 | RASQDVSTAVA | 127 |
| (BACE1) | | | |
| | CDR2 | SASFLYS | 128 |
| | CDR3 | QQGYNDPPT | 131 |
| BACE004VL | CDR1 | RASQDVSTAVA | 127 |
| (BACE1) | | | |
| | CDR2 | SASFLYS | 128 |
| | CDR3 | QQSSTDPTT | 132 |
| BACE005VL | CDR1 | RASQVVANSLA | 133 |
| (BACE1) | | | |
| | CDR2 | SASFLYS | 128 |
| | CDR3 | QQDATSPPTF | 134 |
| BACE006VL | CDR1 | RASQDVSTAVA | 127 |
| (BACE1) | | | |
| | CDR2 | SASFLYS | 128 |
| | CDR3 | QQYATDPPT | 135 |
| BACE001VH | CDR1 | GFTFSGYAIH | 136 |
| (BACE1) | | | |
| | CDR2 | GWISPAGGSTDYADSVKG | 137 |
| | CDR3 | GPFSPWVMDY | 138 |
| BACE002VH | CDR1 | GFTFLGYGIH | 139 |
| (BACE1) | | | |
| | CDR2 | GWISPAGGSTDYADSVKG | 137 |
| | CDR3 | GPFSPWVMDY | 138 |
| BACE003VH | CDR1 | GFTFSGYAIH | 140 |
| (BACE1) | | | |
| | CDR2 | GWISPAGGSTDYADSVKG | 137 |
| | CDR3 | GPFSPWVMDY | 138 |
| BACE004VH | CDR1 | GFTFSGYAIH | 140 |
| (BACE1) | | | |
| | CDR2 | GWISPAGGSTDYADSVKG | 137 |
| | CDR3 | GPFSPWVMDY | 138 |
| BACE005VH | CDR1 | GFTFSGYAIH | 140 |
| (BACE1) | | | |
| | CDR2 | GWISPAGGSTDYADSVKG | 137 |
| | CDR3 | GPFSPWVMDY | 138 |
| BACE005VH | CDR1 | GFTFSGYAIH | 140 |
| (BACE1) | | | |
| | CDR2 | GWISPAGGSTDYADSVKG | 137 |
| | CDR3 | GPFSPWVMDY | 138 |
| ABETA001VH | CDR1 | GFTFSRYSMS | 141 |
| (Abeta) | | | |
| | CDR2 | QINSVGNSTYYPDTVKG | 142 |
| | CDR3 | GDY | 143 |
| ABETA001VL | CDR1 | RSSQSLIYSDGNAYLH | 144 |
| (Abeta) | | | |
| | CDR2 | KVSNRFS | 145 |
| | CDR3 | SQSTHVPWT | 146 |
| ABETA002VH | CDR1 | GFTFSSYAMS | 147 |
| (Abeta) | | | |
| | CDR2 | AINASGTRTYYA | 148 |
| | CDR3 | GKGNTHKPYGYVRYFDV | 149 |
| ABETA002VL | CDR1 | RASQSVSSSYLA | 150 |
| (Abeta) | | | |
| | CDR2 | GASSRAT | 151 |
| | CDR3 | LQIYNMPIT | 152 |
| VHH-TMEM30A | CDR1 | GFKITHYTMG | 153 |
| (TMEM30A) | | | |
| | CDR2 | RITWGGDNTFYSNSVKG | 154 |
| | CDR3 | GSTSTATPLRVDY | 155 |
| HIR-VH | CDR1 | GYTFTNYDIH | 156 |
| (HIR) | | | |
| | CDR2 | WIYPGDGSTKYNEKFKG | 157 |
| | CDR3 | YWGQGTTV | 158 |
| HIR1-VL | CDR1 | RASQDIGGNLY | 159 |
| (HIR) | | | |
| | CDR2 | ATSSLDS | 160 |
| | CDR3 | LQYSSSPWT | 161 |
| HIR2-VL | CDR1 | RASQDIGGNLY | 159 |
| (HIR) | | | |
| | CDR2 | ATSSLDS | 160 |
| | CDR3 | LQYSSSPWT | 161 |
| HIR3-VL | CDR1 | RASQDIGGNLY | 159 |
| (HIR) | | | |
| | CDR2 | ATSSLDS | 160 |
| | CDR3 | LQYSSSPWT | 161 |
| HIR4-VL | CDR1 | RASQDIGGNLY | 159 |
| (HIR) | | | |
| | CDR2 | ATSSLDS | 160 |
| | CDR3 | LQYSSSPWT | 161 |
| 8C11-VH | CDR1 | DYNMN | 164 |
| (TNF) | | | |
| | CDR2 | VINPNYGSSTYNQKFKG | 165 |
| | CDR3 | KWGQLGRGFFD | 166 |
| 8C11-VL | CDR1 | RASSSVSYMH | 167 |
| (TNF) | | | |
| | CDR2 | ATSNLAS | 168 |
| | CDR3 | QQWSSSPLT | 169 |
| RGMA-VH | CDR1 | SHGIS | 172 |
| (RGMA) | | | |
| | CDR2 | WISPYSGNTNYAQKLQ | 173 |
| | CDR3 | VGSGPYYYMDV | 174 |
| RGMA-VL | CDR1 | TGTSSSVGDSIYVS | 175 |
| (RGMA) | | | |
| | CDR2 | DVTKRPS | 176 |
| | CDR3 | CSYAGTDTL | 177 |

TABLE 3
DVD-Ig Protein Sequences

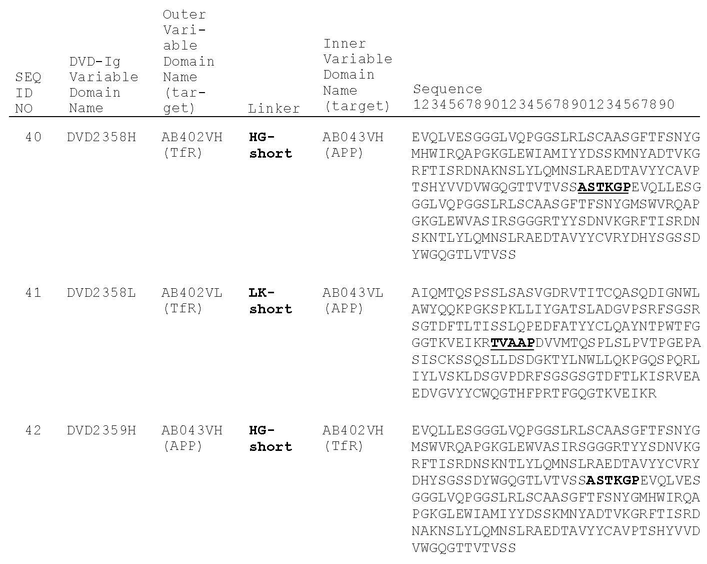

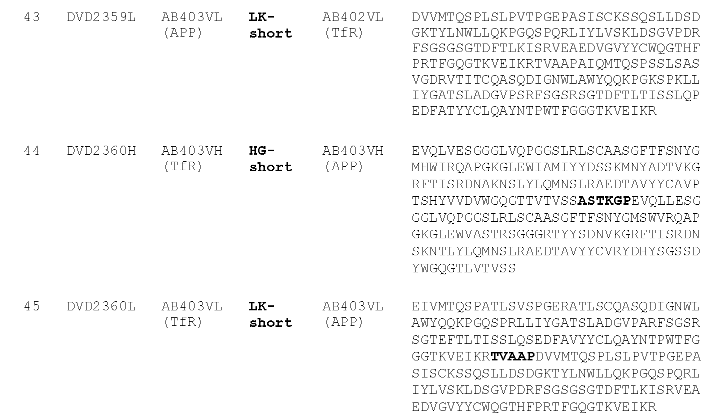

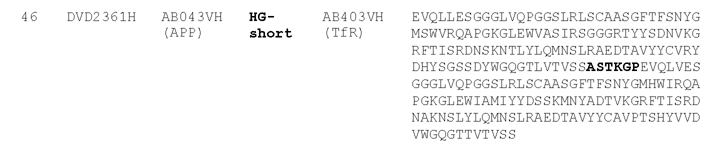

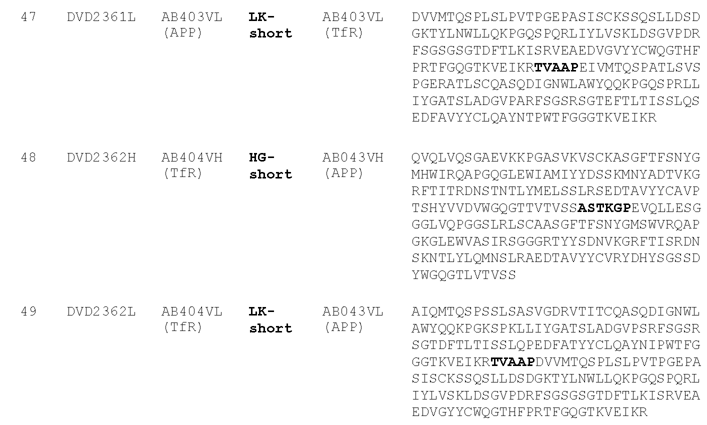

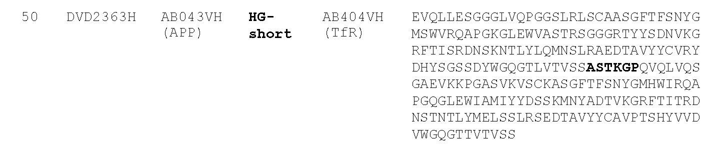

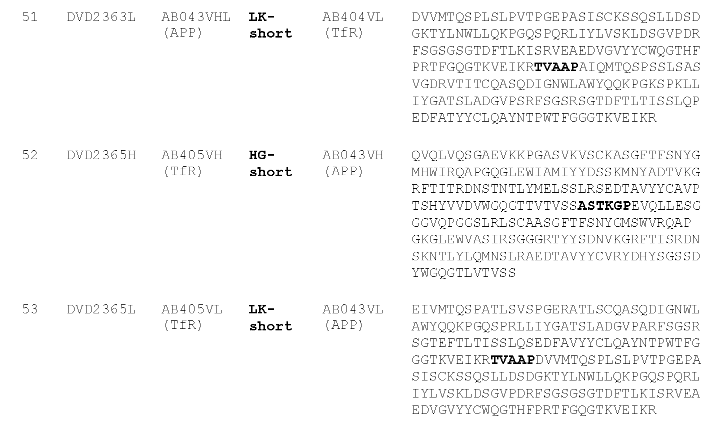

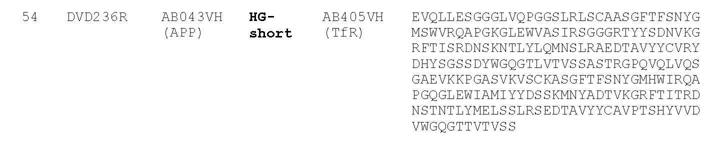

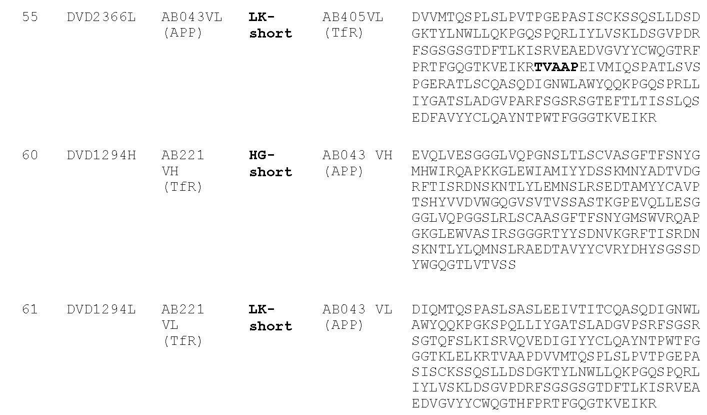

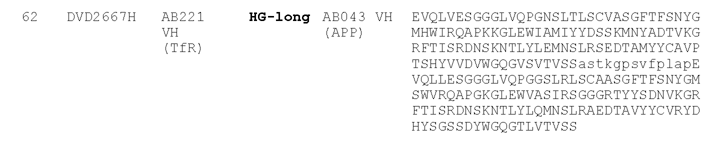

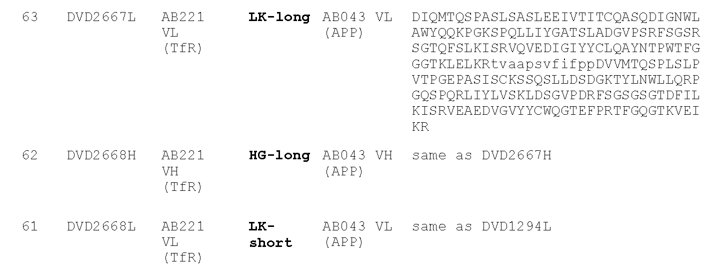

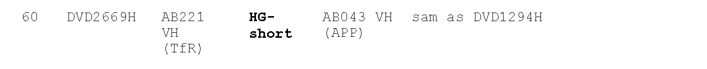

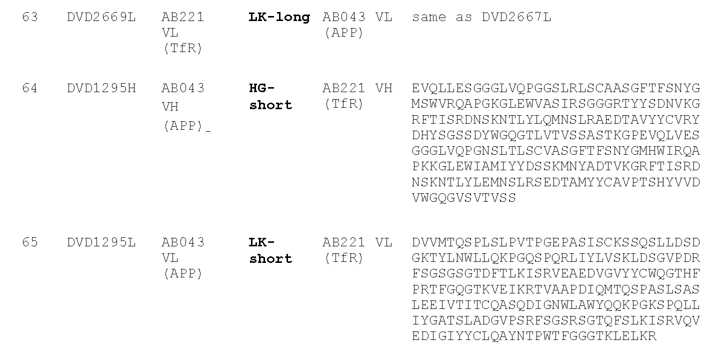

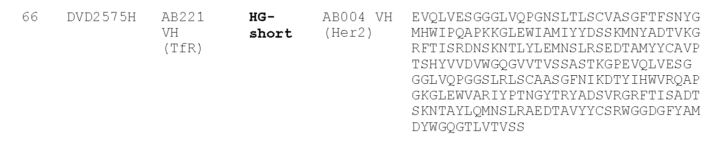

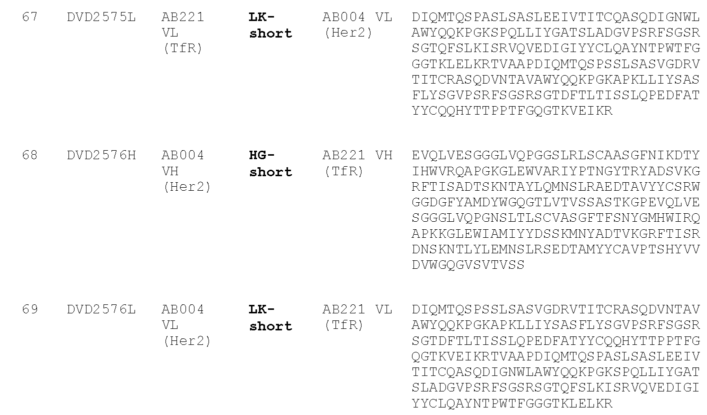

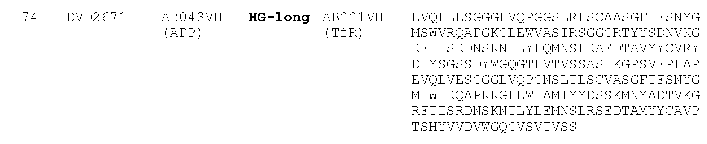

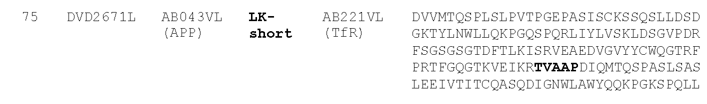

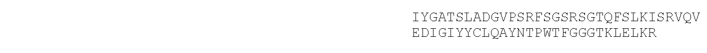

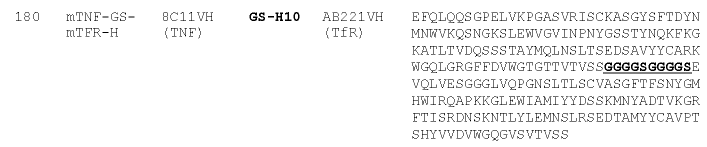

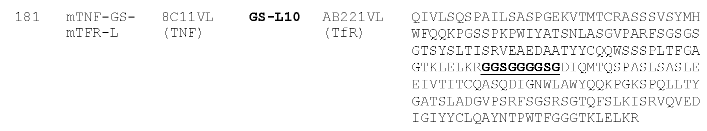

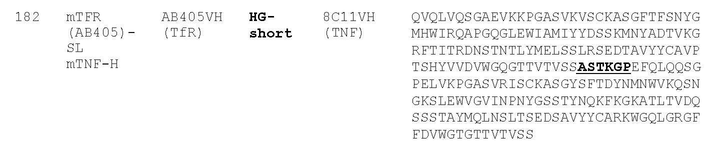

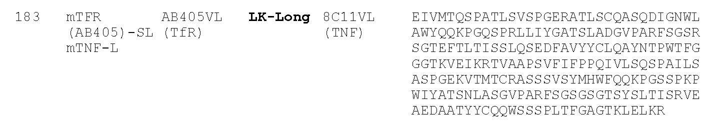

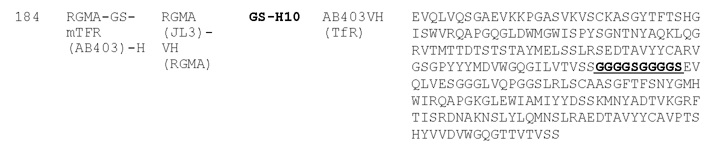

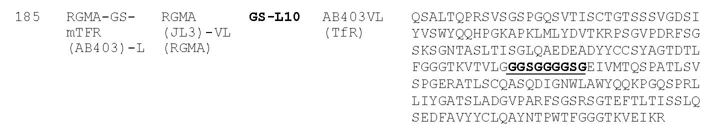

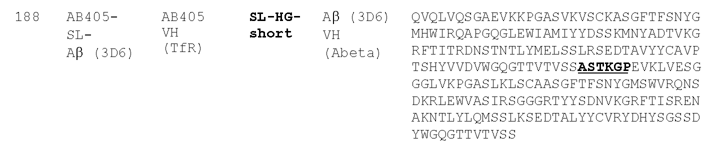

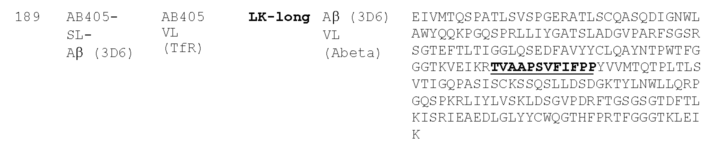

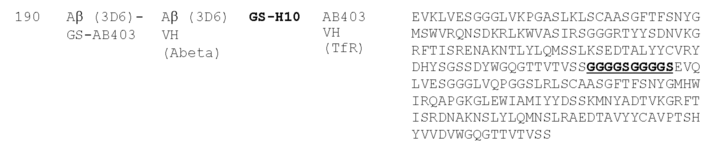

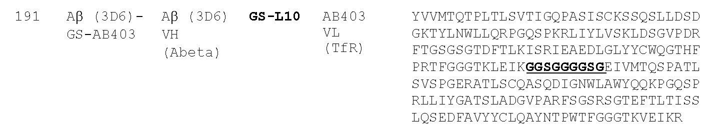

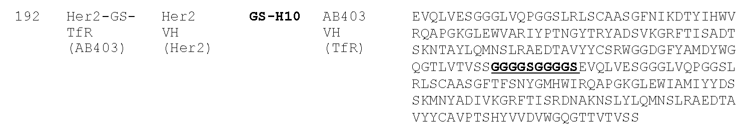

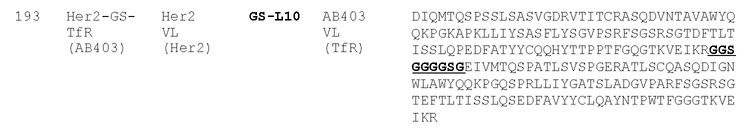

Other sequences are listed below in Table 4 that can be used to construct DVD-Ig proteins or other binding proteins according to any method described herein.

TABLE 4
Additional BBB DVD-Ig Protein Variable Domain Sequences that bind to
Abeta

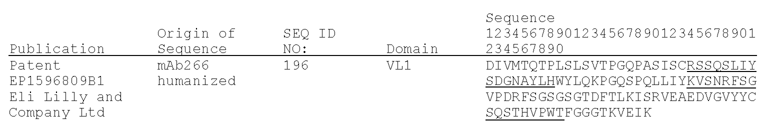

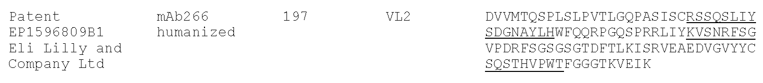

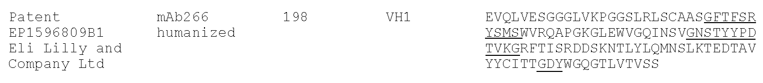

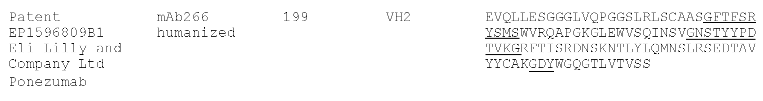

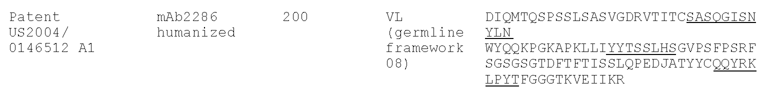

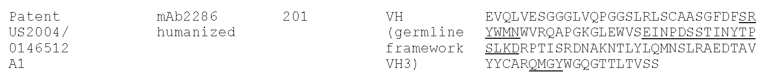

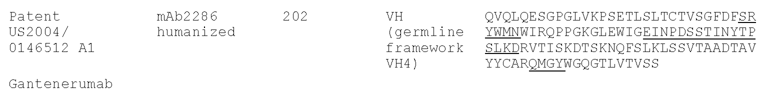

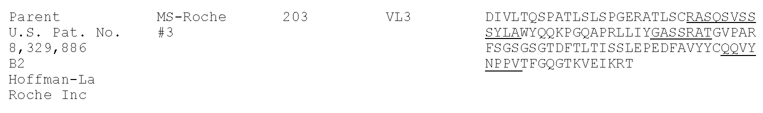

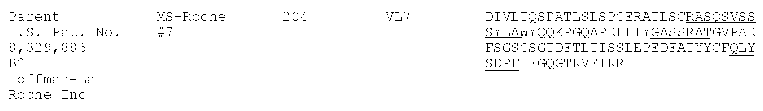

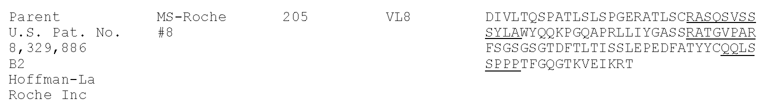

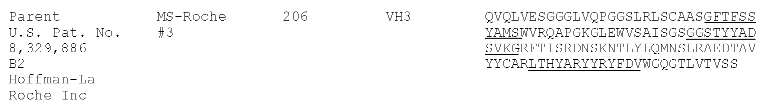

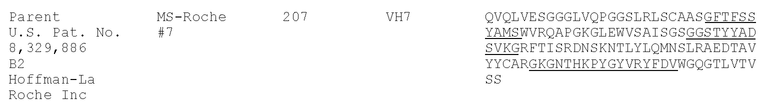

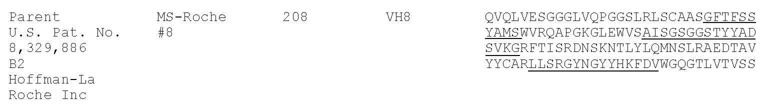

### Example 2

### Design, Construction, and Analysis of a DVD-Ig Protein

1.1: Construction and Expression of Humanized Anti Mouse or Human Parent Antibodies

1.1.A: Selection of Human Antibody Frameworks

Each murine variable heavy and variable light chain gene sequence was separately aligned against 44 human immunoglobulin germline variable heavy chain or 46 germline variable light chain sequences (derived from NCBI Ig Blast website at http://www.ncbi.nlm.nih.gov/igblast/retrieveig.html) using Vector NTI software. Humanization was based on amino acid sequence homology, CDR cluster analysis, frequency of use among expressed human antibodies, and available information on the crystal structures of human antibodies. Taking into account possible effects on antibody binding, VH-VL pairing, and other factors, murine residues were mutated to human residues where murine and human framework residues are different, with a few exceptions. Additional humanization strategies were designed based on an analysis of human germline antibody sequences, or a subgroup thereof, that possessed a high degree of homology, i.e., sequence similarity, to the actual amino acid sequence of the murine antibody variable regions.

Homology modeling was used to identify residues unique to the murine antibody sequences that were predicted to be critical to the structure of the antibody binding site. Homology modeling is a computational method whereby approximate three dimensional coordinates are generated for a protein. The source of initial coordinates and guidance for their further refinement was a second protein, the reference protein, for which the three dimensional coordinates were known and the sequence of which was related to the sequence of the first protein. The relationship among the sequences of the two proteins was used to generate a correspondence between the reference protein and the protein for which coordinates were desired, the target protein. The primary sequences of the reference and target proteins were aligned with coordinates of identical portions of the two proteins transferred directly from the reference protein to the target protein. Coordinates for mismatched portions of the two proteins, e.g., from residue mutations, insertions, or deletions, were constructed from generic structural templates and energy refined to insure consistency with the already transferred model coordinates. This computational protein structure may be further refined or employed directly in modeling studies. The quality of the model structure was determined by the accuracy of the contention that the reference and target proteins were related and the precision with which the sequence alignment is constructed.

For the murine mAbs, a combination of BLAST searching and visual inspection was used to identify suitable reference structures. Sequence identity of 25% between the reference and target amino acid sequences was considered the minimum necessary to attempt a homology modeling exercise. Sequence alignments were constructed manually and model coordinates were generated with the program Jackal (Petrey et al. (2003) Proteins 53 (Suppl. 6):430-435).

The primary sequences of the murine and human framework regions of the selected antibodies share significant identity. Residue positions that differ are candidates for inclusion of the murine residue in the humanized sequence in order to retain the observed binding potency of the murine antibody. A list of framework residues that differ between the human and murine sequences is constructed manually. Table 5 shows the framework sequences chosen for this study.

TABLE 5
Sequence Of Human IgG Heavy Chain Constant Domain And Light Chain
Constant Domain

| | SEQ | Sequence |
|---|---|---|
| Protein | ID NO | 1234567890123456789012345678901234567890 |
| Wild type hIgG1 | 70 | ASTKGPSVFPLAPSSKSTSGGTAALGCLVKDYFPEPVTVS |
| constant region | | WNSGALTSGVHTFPAVLQSSGLYSLSSVVTVPSSSLGTQT |
| | | YICNVNHKPSNTKVDKKVEPKSCDKTHTCPPCPAPELLGG |
| | | PSVFLFPPKPKDTLMISRTPEVTCVVVDVSHEDPEVKFNW |
| | | YVDGVEVHNAKTKPREEQYNSTYRVVSVLTVLHQDWLNGK |
| | | EYKCKVSNKALPAPIEKTISKAKGQPREPQVYTLPPSREE |
| | | MTKNQVSLTCLVKGFYPSDIAVEWESNGQPENNYKTTPPV |
| | | LDSDGSFFLYSKLTVDKSRWQQGNVFSCSVMHEALHNHYT |
| | | QKSLSLSPGK |
| Mutant hIgG1 | 71 | ASTKGPSVFPLAPSSKSTSGGTAALGCLVKDYFPEPVTVS |
| constant region | | WNSGALTSGVHTFPAVLQSSGLYSLSSVVTVPSSSLGTQT |
| | | YICNVNHKPSNTKVDKKVEPKSCDKTHTCPPCPAPEAAGG |
| | | PSVFLFPPKPKDTLMISRTPEVTCVVVDVSHEDPEVKFNW |
| | | YVDGVEVHNAKTKPREEQYNSTYRVVSVLTVLHQDWLNGK |
| | | EYKCKVSNKALPAPIEKTISKAKGQPREPQVYTLPPSREE |
| | | MTKNQVSLTCLVKGFYPSDIAVEWESNGQPENNYKTTPPV |
| | | LDSDGSFFLYSKLTVDKSRWQQGNVFSCSVMHEALHNHYT |
| | | QKSLSLSPGK |
| Ig kappa constant | 72 | TVAAPSVFIFPPSDEQLKSGTASVVCLLNNFYPREAKVQW |
| region | | KVDNALQSGNSQESVTEQDSKDSTYSLSSTLTLSKADYEK |
| | | HKVYACEVTHQGLSSPVTKSFNRGEC |
| Ig Lambda | 73 | QPKAAPSVTLFPPSSEELQANKATLVCLISDFYPGAVTVA |
| constant region | | WKADSSPVKAGVETTTPSKQSNNKYAASSYLSLTPEQWKS |
| | | HRSYSCQVTHEGSTVEKTVAPTECS |

The likelihood that a given framework residue would impact the binding properties of the antibody depends on its proximity to the CDR residues. Therefore, using the model structures, the residues that differ between the murine and human sequences were ranked according to their distance from any atom in the CDRs. Those residues that fell within 4.5 Å of any CDR atom were identified as most important and were recommended to be candidates for retention of the murine residue in the humanized antibody (i.e., a back mutation).

### Example 3

### Assays Used to Identify and Characterize Parent Antibodies and DVD-Ig Proteins

The following assays were used throughout the Examples to identify and characterize parent antibodies and DVD-Ig proteins, unless otherwise stated.

3.1 Size Exclusion Chromatography

Antibodies were diluted to 2.5 mg/mL with water and 20 mL analyzed on a Shimadzu HPLC system using a TSK gel G3000 SWXL column (Tosoh Bioscience, cat# k5539-05k). Samples were eluted from the column with 211 mM sodium sulfate, 92 mM sodium phosphate, pH 7.0, at a flow rate of 0.3 mL/minutes. The HPLC system operating conditions were the following:

Mobile phase: 211 mM Na2SO4, 92 mM Na2HPO4*7H2O, pH 7.0

Gradient: Isocratic

Flow rate: 0.3 mL/minute

Detector wavelength: 280 nm

Autosampler cooler temp: 4° C.

Column oven temperature: Ambient

Run time: 50 minutes

Table 6 contains DVD-Ig constructs expressed as percent monomer (unaggregated protein of the expected molecular weight) as determined by the above protocol.

TABLE 6
Purity of DVD-Ig Constructs as Determined by Size Exclusion
Chromatography

| | N-terminal/Outer | C-terminal/Inner | |
|---|---|---|---|
| | Variable Domain | Variable Domain | |
| DVD-Ig ID | (VD) | (VD) | % Monomer (purity) |
| DVD1294 | TfR | APP | 95 |
| DVD2669 | TfR | APP | 100 |
| DVD2668 | TfR | APP | 98.1 |
| DVD2667 | TfR | APP | 98.4 |
| DVD1295 | APP | TfR | 97 |
| DVD2671 | APP | TfR | 96.5 |
| DVD2358 | TfR(AB402) | APP | 98 |
| DVD2359 | APP | TfR(AB402) | 96 |
| DVD2360 | TfR(AB403) | APP | 98 |
| DVD2361 | APP | TfR(AB403) | 95 |
| DVD2362 | TfR(AB404) | APP | 97.8 |
| DVD2363 | APP | TfR(AB404) | 96.8 |
| DVD2365 | TfR(AB405) | APP | 93 |
| DVD2366 | APP | TfR(AB405) | 100 |
| DVD2575 | TfR | Her2 | 100 |
| DVD2576 | Her2 | TfR | 97 |

DVD-Ig proteins showed an excellent SEC profile with all DVD-Ig proteins showing >90% monomer. This DVD-Ig profile is similar to that observed for parent antibodies.

### Example 4

### Cell-Based Electrochemiluminescence-Meso Scale Discovery (ECL-MSD) Binding Assay

Meso-scale Discovery (MSD) Electrochemiluminescence (ECL) assays to screen for antibodies or DVD-Ig proteins that bind a desired target antigen expressed on the cell surface were performed as follows: Hek 293 cells overexpressing mouse Transferrin Receptor were added onto MSD 96-well plates (MSD Cat# L11 XB-3, lot#2290-EA). The plates which were blocked using blocking buffer (30% FBS Serum (Hyclone) in PBS) at 37° C. for 1 hour. After incubating at room temperature (RT) for 30 minutes with mild agitation, plates were washed with DPBS 3 times and Abs/DVD-Ig proteins (10,000 ng/ml) were added. After incubating for 1 hour at RT, plates were washed 3 times with DPBS and 25 μl of Goat anti-human Sulfo TAG (MSD Cat# R32AC-, Lot# W001162) at 1 μg/ml was added. Following incubation at RT for 1 hour, plates were washed and MSD read buffer was added before reading on a MSD SECTOR Imager 6000. EC50 values were obtained using Xlfit4 software package.

TABLE 7
Transferrin Receptor Binding Assay and Biacore with Transferrin
Receptor Abs and DVD-Ig Proteins

| | Ex- | Hek293/mTfR | | | |
|---|---|---|---|---|---|
| TfR Abs | pression | MSD binding | Biacore, rmTfR (123-763) | | |
| or | yield | assay EC50 | Ka | Kd | KD |
| DVDs | (mg/L) | (nM) | (1/Ms) | (1/s) | (nM) |
| IgG | N/A | No Binding | | | |
| AB221 | 110 | 0.1 | 3.94E04* | 1.81E−04* | 5* |
| AB402 | 47.5 | 0.06 | 1.64E04 | 6.12E−05 | 3.7 |
| AB403 | 35.5 | 0.09 | 2.20E04 | 8.39E−05 | 3.8 |
| AB404 | 63 | 3.0 | | | No |
| | | | | | binding |
| AB405 | 34 | 13.6 | | | No |
| | | | | | Binding |
| DVD1294 | 11.7 | 0.11 | ND | ND | ND |
| DVD2669 | 8.8 | 0.12 | ND | ND | ND |
| DVD2668 | 9.7 | 0.10 | ND | ND | ND |
| DVD2667 | 9.3 | ND | ND | ND | ND |
| DVD1295 | 13.8 | 3.70 | ND | ND | ND |
| DVD2671 | 6.1 | 1.20 | ND | ND | ND |
| DVD2358 | 12.9 | 0.10 | 3.13E04 | 4.06E−05 | 1.3 |
| DVD2359 | 83.5 | 12.0 | Slow | slow | LOD |
| DVD2360 | 50.4 | 0.15 | 2.80E04 | 4.80E−05 | 1.7 |
| DVD2361 | 62.8 | 10.7 | Slow | slow | LOD |
| DVD2362 | 19.6 | 3.0 | Slow | Slow | LOD |
| DVD2363 | 21.8 | No binding | | | |
| DVD2365 | 13.8 | 3.2 | 9.87E03 | 7.71E−04 | 78 |
| DVD2366 | 21.8 | No binding | ND | ND | ND |
| DVD2575 | 62.8 | 0.11 | ND | ND | ND |
| DVD2576 | 80.2 | 3.40 | ND | ND | ND |

*measured using rmTfR (119-763)

### Example 5

### Affinity Determination Using BIACORE Technology

The BIACORE assay (Biacore, Inc, Piscataway, N.J.) was used to determine the affinity of antibodies or DVD-Ig proteins with kinetic measurements of on-rate and off-rate constants. Binding of antibodies or DVD-Ig protein to a target antigen (for example, a purified recombinant target antigen) was determined by surface plasmon resonance-based measurements with a Biacore® 1000 or 3000 instrument (Biacore® AB, Uppsala, Sweden) using running HBS-EP (10 mM HEPES [pH 7.4], 150 mM NaCl, 3 mM EDTA, and 0.005% surfactant P20) at 25° C. All chemicals were obtained from Biacore® AB (Uppsala, Sweden) or otherwise from a different source as described in the text. For example, approximately 5000 RU of goat anti-human IgG, (Fcγ), fragment specific polyclonal antibody (Pierce Biotechnology Inc, Rockford, Ill.) diluted in 10 mM sodium acetate (pH 4.5) was directly immobilized across a CM5 research grade biosensor chip using a standard amine coupling kit according to manufacturer's instructions and procedures at 25 μg/ml. Unreacted moieties on the biosensor surface were blocked with ethanolamine. Modified carboxymethyl dextran surface in flowcell 2 and 4 was used as a reaction surface. Unmodified carboxymethyl dextran without goat anti-human IgG in flow cell 1 and 3 was used as the reference surface. For kinetic analysis, rate equations derived from the 1:1 Langmuir binding model were fitted simultaneously to association and dissociation phases of all eight injections (using global fit analysis) with the use of BIAevaluation 4.0.1 software. Purified antibodies or DVD-Ig proteins were diluted in HEPES-buffered saline for capture across goat anti-human IgG specific reaction surfaces. Antibodies or DVD-Ig proteins to be captured as ligands (25 μg/ml) were injected over reaction matrices at a flow rate of 5 μl/minute. The association and dissociation rate constants, kon (M⁻¹ s⁻¹) and koff (s⁻¹) were determined under a continuous flow rate of 25 μl/minute. Rate constants were derived by making kinetic binding measurements at different antigen concentrations ranging from 10-200 nM. The equilibrium dissociation constant (M) of the reaction between antibodies or DVD-Ig proteins and the target antigen was then calculated from the kinetic rate constants by the following formula: KD=koff/kon. Binding was recorded as a function of time and kinetic rate constants were calculated. In this assay, on-rates as fast as 106 M⁻¹ s⁻¹ and off-rates as slow as 10⁻⁶ s⁻¹ were measured (Table 7).

Lower affinity variants of AB221, which are AB404 and AB405, as determined by cell based MSD binding assay did not show significant binding by Biacore. DVD-Ig proteins containing lower affinity variants of AB221 showed reduced binding or no significant binding as measured by cell-based MSD binding assay or Biacore.

### Example 6

### In Vivo Biodistribution of Antibodies and DVD-Ig Proteins

A: Measuring Antibody and DVD-Ig Protein Concentrations in Mouse Brain and Serum

Wild type female C57B/6 mice, age 6 to 8 weeks, were intravenously injected with anti-TfR variants, control IgG, anti-TfR containing DVD-Ig proteins (20 mg/kg). After the indicated time, mice were perfused with D-PBS at a rate of 2 ml/minute for 10 minutes. Brains were extracted and homogenized using Bullet Blender Blue (NextAdvance BBX24B) in 1% NP-40 (Calbiochem) in PBS containing CompleteMini EDTA-free protease inhibitor cocktail tablets (Roche Diagnostics). Homogenized brain samples were rotated at 4° C. for 1 hour before spinning at 14,000 rpm for 20 minutes. Supernatant was isolated for brain antibody measurement. Whole blood was collected before perfusion in serum separator microcontainer tubes (BDDiagnostics), allowed to clot for at least 30 minutes, and spun down at 5000 g for 90 seconds. Supernatant was isolated for antibody measurement in serum. Antibody or DVD-Ig protein concentrations in mouse serum and brain samples were measured with an ECL-MSD assay. MSD high bind 96-well plates (MSD cat # L11XB-3) were coated with a F(ab′)2 fragment of donkey anti-human IgG, Fc fragment-specific polyclonal antibody (Jackson ImmunoResearch) overnight at 4° C. Plates were blocked with 3% MSD Block buffer for 1 hour at 25° C. Each antibody or DVD-Ig protein was used as an internal standard to quantify respective antibody or DVD-Ig protein concentrations. Plates were washed with wash buffer and standards and samples diluted in 0.1% serum containing 1% MSD assay buffer were added and incubated for 2 hours at 25° C. Bound antibody was detected with goat anti-human Sulfo-TAG, MSD and read on MSD SECTOR Imager 6000. Concentrations were determined from the standard curve with a five-parameter nonlinear regression program using Excel Fit software.

B. Immunohistochemistry

To determine antibody distribution in the brain, wild-type mice were intravenously injected with an indicated antibody or DVD-Ig protein (20 mg/kg or as indicated). After the indicated time, mice were perfused as described above, and brains were fixed in 4% paraformaldehyde for 8 hours. Following fixation, tissues were processed through a graded series of alcohol to xylene and then paraffin embedded. For histological evaluation, 5-μm brain sections were stained for the detection of anti-Human IgG.

First, the sections were de-paraffinized and rehydrated to water and placed into Tris wash buffer. IHC staining was completed on a Dako autostainer links 48 system. The sections were blocked with 3% hydrogen peroxide for 30 minutes, washed with wash buffer then incubated for 8 minutes with protease. Sections were blocked with a streptavidin and biotin blocking kit (Vector Laboratories, Burlingame, Calif.) for 8 minutes each, followed by Dako protein block for 30 minutes. Next, the sections were incubated for 1 hour at room temperature with a biotinylated-Donkey anti-human IgG (F(ab′) fragmented antibody at 15 μg/ml followed by a streptavidin peroxidase reagent for 30 minutes. Following the streptavidin step the sections were reacted with DAB chromogen for 3 minutes to form a brown precipitate. The sections were then washed with water, counterstained, dehydrated and mounted for microscopic observation.

Image analysis: A semi-quantitative analysis of mean parenchymal intensity per section was developed. The cerebellum and cortex sections were analyzed morphometrically using Image Pro Plus software. The analysis was performed on three images of parenchymal staining at a magnification of 20×. The average intensities of four representative human IgG positive areas were selected per animal. All settings (filters and light levels) for each image were kept constant throughout the experiment. Measurements were analyzed as mean intensity measurements and exported to Microsoft Excel.

Table 8 provides the in vivo biodistribution characteristics of TfR Abs with 20 mpk dosing.

An inverse relationship between brain uptake and affinity was observed with anti-TfR Abs listed on Table 8. Two lower affinity TfR Abs (AB404 and AB405) showed improved uptake, in some cases >8 fold increase in % injected dose over control IgG was measured. Twenty four hours after injection, strong parenchymal and neuronal staining was observed.

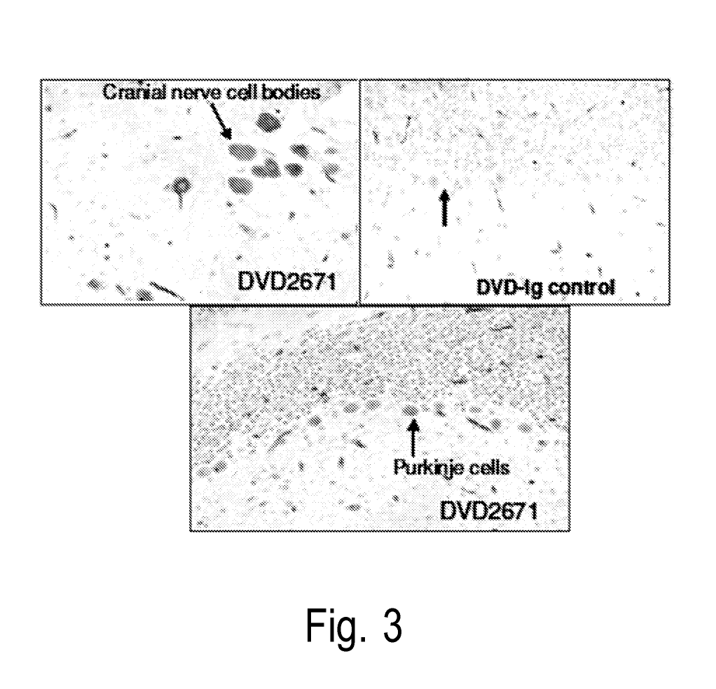

Elevated DVD-Ig protein levels were detected in brain by two orthogonal methods: localization to neuronal cells and parenchyma by IHC (FIG. 3 and Table 9) and higher than control DVD-Ig protein brain concentration as measured by MSD-ECL (Table 9), demonstrating transport across the BBB after therapeutic dosing via IV administration. For some of the DVD-Ig proteins, up to 10 fold higher brain uptake, which was measured by percentage (%) injected dose/gram of brain, was observed compared to control DVD-Ig protein.

TABLE 8
In Vivo Biodistribution Characteristics Of Transferrin Receptor Abs With 20 MPK IV Dosing

| TfR Abs/in vivo | TfR binding | | | Brain | Serum | Parenchymal | Neuronal |
|---|---|---|---|---|---|---|---|
| collection time/IV | EC50 | % ID/g | Fold increase | conc. | conc. | staining | staining intensity |
| injection dose | (nM) | brain | over IgG | (nM) | (nM) | intensity Max = 4 | Max = 4 |
| IgG, 24 h, 20 mg/kg | NB | 0.3 +/− 0.03 | N/A | 1.14 +/− 0.17 | 1693 | 0 | 0 |
| AB221, 1 h, 20 mg/kg | 0.1 | 0.06 +/− 0.03 | | 2.14 +/− 0.13 | 2266 | 0 | 0 |
| AB221, 24 hr, 20 mg/kg | | 0.46 +/− 0.15 | 1.5 | 1.51 +/− 0.59 | 466 | 1.5 | 1.9 |
| AB221, 48 hr, 20 mg/kg | | 0.53 +/− 0.11 | | 1.73 +/− 0.43 | 46 | 2 | 2.3 |
| AB404, 1 h, 20 mg/kg | 3.0 | 0.84 +/− 0.28 | | 2.78 +/− 0.59 | 2580 | 0 | 0 |
| AB404, 24 hr, 20 mg/kg | | 1.97 +/− 0.11 | 6.6 | 6.48 +/− 1.09 | 1606 | 2.5 | 2.3 |
| AB404, 48 hr, 20 mg/kg | | 1.59 +/− 0.21 | | 4.83 +/− 0.27 | 993 | 2.5 | 1.5 |
| AB405, 1 h, 20 mg/kg | 13.6 | 1.05 +/− 0.15 | | 3.73 +/− 0.92 | 2566 | 0 | 0 |
| AB405, 24 hr, 20 mg/kg | | 1.97 +/− 0.33 | 6.6 | 6.97 +/− 0.44 | 1966 | 2 | 2 |
| AB405, 48 hr, 20 mg/kg | | 1.62 +/− 0.18 | | 5.96 +/− 0.35 | 933 | 2 | 2.3 |

TABLE 9
In Vivo Biodistribution Characteristics Of Transferrin Receptor DVD-Ig
Proteins At 24 Hours With 20 mg/kg IV Injection (*10 MPK Injection)

| | | Fold increase | | | |
|---|---|---|---|---|---|
| | | over control | | Serum | Parenchymal/ |
| DVD-Ig | | DVD-Ig | Brain conc. | conc. | Neuronal |
| protein | % ID/g brain | protein | (nM) | (nM) | staining |
| Isotype | 0.38 +/− 0.12 | N/A | 0.95 +/− 0.30 | 1420 | −/− |
| control | | | | | |
| DVD-Ig | | | | | |
| protein | | | | | |
| DVD2668 | 0.51 +/− 0.10 | 1.3 | 1.82 +/− 0.38 | 129.6 | −/− |
| DVD2667 | 3.87 +/− 0.85 | 10.2 | 4.79 +/− 0.41 | 855.1 | +/+ |
| DVD1295 | 1.87 +/− 0.19 | 5.0 | 7.74 +/− 0.64 | 763.3 | +/+ |
| DVD2671 | 0.97 +/− 0.26 | 2.6 | 2.90 +/− 0.44 | 588.2 | +/+ |
| DVD2359 | 0.84 +/− 0.23 | 2.2 | 3.04 +/− 0.34 | 1494.2 | +/+ |
| DVD2361 | 0.79 +/− 0.12 | 2.1 | 3.09 +/− 0.28 | 887.8 | +/+ |
| DVD2362 | 4.08 +/− 0.27 | 10.7 | 17.42 +/− 2.09 | 459.3 | +/+ |
| DVD2365 | 3.87 +/− 0.85 | 10.2 | 15.83 +/− 2.48 | 432.8 | +/+ |
| DVD2575 | 0.53 +/− 0.15 | 1.4 | 1.4 +/− 0.4 | 188.2 | +/+ |
| DVD2576 | 1.14 +/− 0.21 | 3.0 | 2.4 +/− 0.7 | 18.3 | +/+ |

Elevated DVD-Ig protein levels were detected in brain by two orthogonal methods: localization to neuronal cells and parenchyma by IHC (FIG. 3 and Table 9) and higher than control DVD-Ig protein brain concentration as measured by MSD-ECL (Table 9), demonstrating transport across the BBB after therapeutic dosing via IV administration. For some of the DVD-Ig proteins, up to 10 fold higher brain uptake, which was measured by percentage (%) injected dose/gram of brain, was observed compared to control DVD-Ig protein.

### Example 7

### Brain Uptake of a DVD-Ig Protein Via Different Routes of Systemic Administration

Similar brain uptake was observed with IV and intraperitoneal (IP) administration (20 mg/kg) using DVD2671. Subcutaneous (SC) administration yielded lower brain and serum concentrations in comparison (Table 10).

TABLE 10
Brain Uptake Of 20 MPK DVD2671 After 24 Hours Via Different
Routes Of Administration

| Route of administration | Brain conc. (nM) | Serum conc. (nM) |
|---|---|---|
| IV | 3.15 +/− 0.26 | 860 |
| IP | 3.72 +/− 0.82 | 730 |
| SC | 2.27+/1.04 | 490 |

### Example 8

### Pharmacokinetic Study Using DVD-Ig Proteins

Mice were injected SC with indicated DVD-Ig proteins at 20 mpk or 50 mpk and processed after 96 hours as described above. Another group was injected SC twice (at 0 and 48 hours) at 20 mpk and processed after 96 hours as described above. Brain serum concentration of 1.58+/−0.20 nM was retained at 96 hours after single 20 mpk SC administration. Brain and serum concentrations measured after 24 hours of IV injections of 20 mpk or 50 mpk of TfR Abs or DVD2671 from different studies are shown for comparison in Table 11.

TABLE 11
Brain And Serum Data For Antibody Or DVD-Ig Binding Proteins

| | | Time of sample | | |
|---|---|---|---|---|
| Ab/DVD | Treatment | collection (H) | Serum (nM) | Brain (nM) |
| DVD2671 | SC - single dose | 1 | 3.7 +/− 3.1 | |
| | at 0 h, 20 mpk | 24 | 142.5 +/− 3.2 | |
| | | 48 | 33.8 +/− 12.5 | |
| | | 72 | 8.8 +/− 2.5 | |
| | | 96 | 1.7 +/− 0.2 | 1.58 +/− 0.20 |
| | SC - single dose | 1 | 11.25 +/− 7.5 | |
| | at 0 h, 50 mpk | 24 | 680.0 +/− 143.6 | |
| | | 48 | 185.5 +/− 65.6 | |
| | | 72 | 93.8 +/− 36.4 | |
| | | 96 | 6.1 +/− 1.7 | 6.86 +/− 2.6 |
| | SC - multiple | 1 | 3.24 +/− 2.5 | 9.03 +/− 1.4 |
| | dose at | 24 | 110.0 +/− 16.83* | |
| | 0 and 48 h, | 48 | 20.04 +/− 1.6 | |
| | 40 mpk | 72 | 365.0 +/− 189.12 | |
| | | 96 | 95.64 +/− 70.24 | |
| IgG** | IV - single dose | 24 | 1791 +/− 127.3 | 1.5 +/− 0.3 |
| | 20 mpk | | | |
| AB221** | IV - single dose | 24 | 643.3 +/− 61.27 | 1.6 +/− 0.22 |
| | 20 mpk | | | |
| AB404* | IV - single dose | 24 | 1093 +/− 724 | 62.8 +/− 0.26 |
| | 20 mpk | | | |
| AB405* | IV - single dose | 24 | 1227 +/− 628.5 | 7.00 +/− 0.04 |
| | 20 mpk | | | |
| Control | IV - single dose | 24 | 1420 +/− 150 | 0.94 +/− 0.30 |
| DVD-Ig | 20 mpk | | | |
| DVD2671 | IV - single dose | 24 | 588.2 +/− 136.7 | 2.90 +/− 0.44 |
| | 20 mpk*** | | | |
| | IV - single dose | 48 | 118.75 +/− 62.90 | 3.07 +/− 0.28 |
| | 20 mpk | | | |
| | IV - single dose | 24 | 2396 +/− 929.4 | 5.08 +/− 1.25 |
| | 50 mpk* | | | |
| | IV - single dose | 48 | 1281.57 +/− 195 | 5.27 +/− 1.40 |
| | 50 mpk | | | |

*n = 2;
**n = 3;
***n = 4

### Example 9

### DVD-Ig Protein Generation and In Vitro/In Vivo Screening

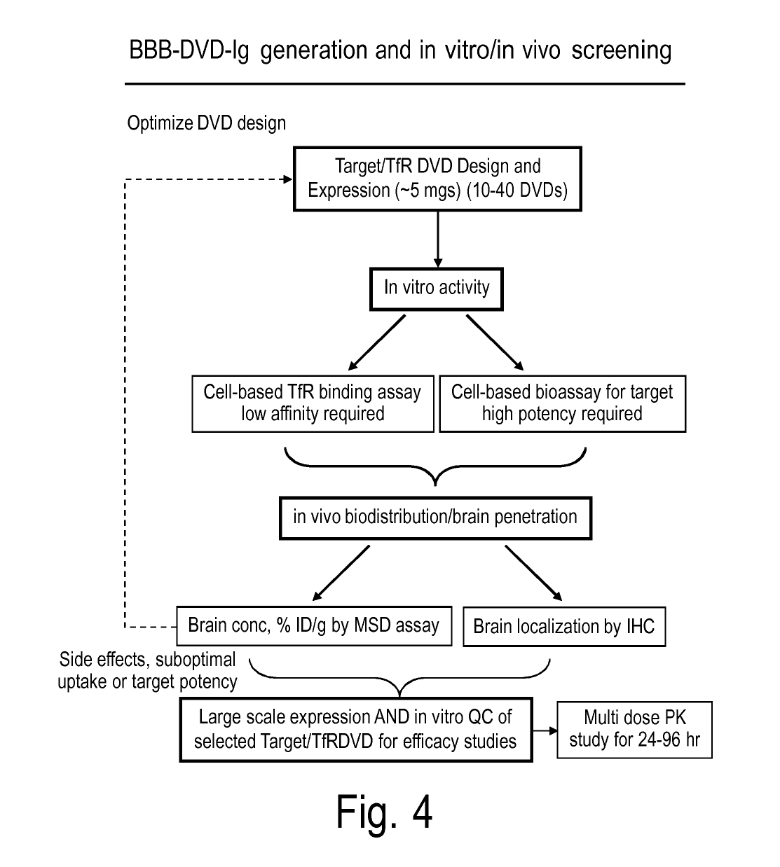

DVD-Ig binding proteins were generated using recombinant methods, and were screened using in vitro and in vivo systems described herein (FIG. 4). Methods and systems described in previous examples (e.g., section describing Assays Used to Identify and Characterize Parent Antibodies and DVD-Ig Protein) were used in the following examples unless indicated otherwise.

DVD-Ig binding proteins (approximately 10-40 DVD-Ig proteins) having domains that specifically bind a target and a TfR (target/TfR DVD-Ig protein) were designed and expressed at a concentration of about five milligrams (mg) using methods and material described herein. In vitro analysis was performed on the target/TfR DVD-Ig proteins using a cell-based TfR binding assay (low affinity required) and a cell-based bioassay target (high potency required). The target/TfR DVD-Ig proteins were then analyzed and compared in an in vivo biodistribution/brain penetration system using murine subjects. Specimens/samples (e.g., cells and tissues) from the subjects were analyzed to determine the presence of the target/TfR DVD-Ig protein in the subjects. A portion of the specimen/samples were analyzed to determine concentration of target/TfR DVD-Ig protein in the brain, and to calculate percent injected dose per gram tissue (% ID/g). Another portion of the specimens/samples was analyzed immunohistochemically to determine localization of the target/TfR DVD-Ig proteins in the brain. Data for indicia of side effects, suboptimal uptake or target potency were analyzed, such that DVD-Ig design could be optimized by again designing and expressing target/TfR DVD-Ig proteins and analyzing using the assays and methods described above (e.g., in vitro activity and in vivo biodistribution/brain penetration).

The target/TfR DVD-Ig proteins having been tested and/or optimized were then expressed in a large scale. In vitro quality control (QC) methods and conditions were performed on the material used in efficacy studies. The resulting DVD-Ig proteins were then used in a multi-dose pharmacokinetics (PK) study over a period of 24-96 hours.

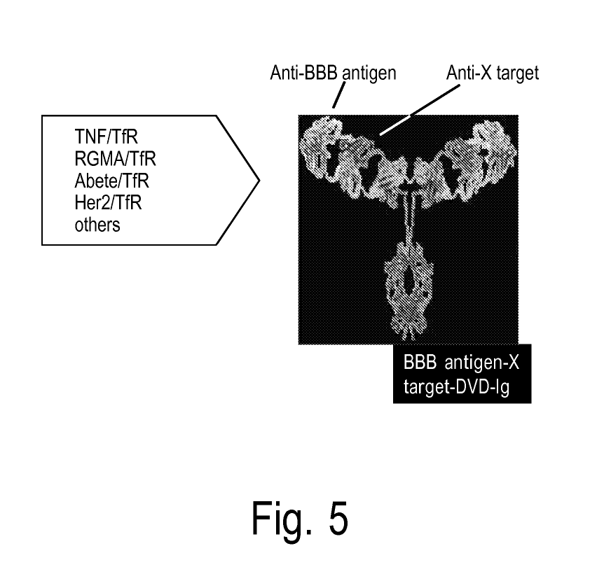

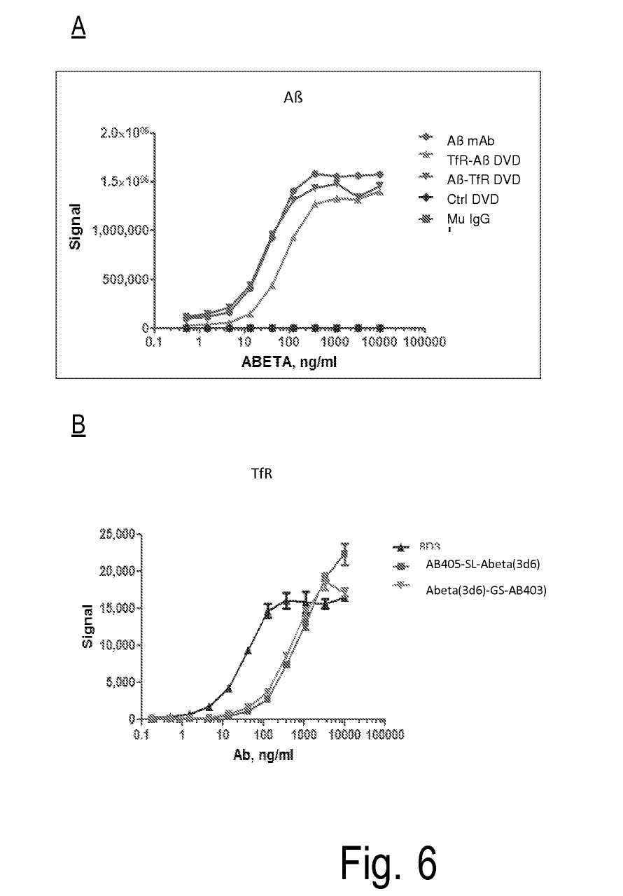

FIG. 5 shows an exemplary DVD-Ig binding protein. The DVD-Ig protein includes at least one heavy chain variable domain that specifically binds a BBB antigen (anti-BBB antigen), and at least one heavy chain variable domain that specifically binds target X. FIG. 5 shows one embodiment of the DVD-Ig binding protein (i.e., anti-BBB antigen binding portion in the outer position and anti-X target portion in the inner position), however in various embodiments the anti-X target portion is in the outer position and the anti-BBB antigen is in the inner position. Without being limited by any particular theory or mechanism of action, the DVD-Ig proteins engineered and analyzed by these methods efficiently penetrate the BBB and bind to target on or in the brain. A DVD-Ig binding protein combining a variable domain (VD) that binds TfR with a VD that binds AB was engineered/constructed and expressed. In vitro studies were performed and data show that the TfR-Aβ DVD-Ig binding protein showed favorable in vitro properties. High affinity binding to Aβ was observed for the TfR-Aβ DVD-Ig binding protein (FIG. 6, panel A). The affinities to Aβ were comparable to the parent antibody. Low affinity binding to TfR was observed for the TfR-Aβ DVD-Ig binding protein (FIG. 6, panel B). The TfR affinities were about 30-fold lower than parent antibody. Similar patterns of binding properties (i.e., high affinity for Aβ and low affinity for TfR) were observed for TfR-Aβ DVD-Ig binding proteins with the TfR binder and Aβ binder in either the VD1 or VD2 positions. Thus, the different orientations (i.e., TfR VD1 and Aβ VD2 DVD-Ig binding protein, as compared to Aβ VD1 and TfR VD2 DVD-Ig binding protein) showed similar binding affinity patterns.

These initial in vitro data were supplemented with additional in vivo analyses described in Examples herein.

### Example 10

### In Vivo Tissue Distribution Analysis of DVD-Ig Proteins

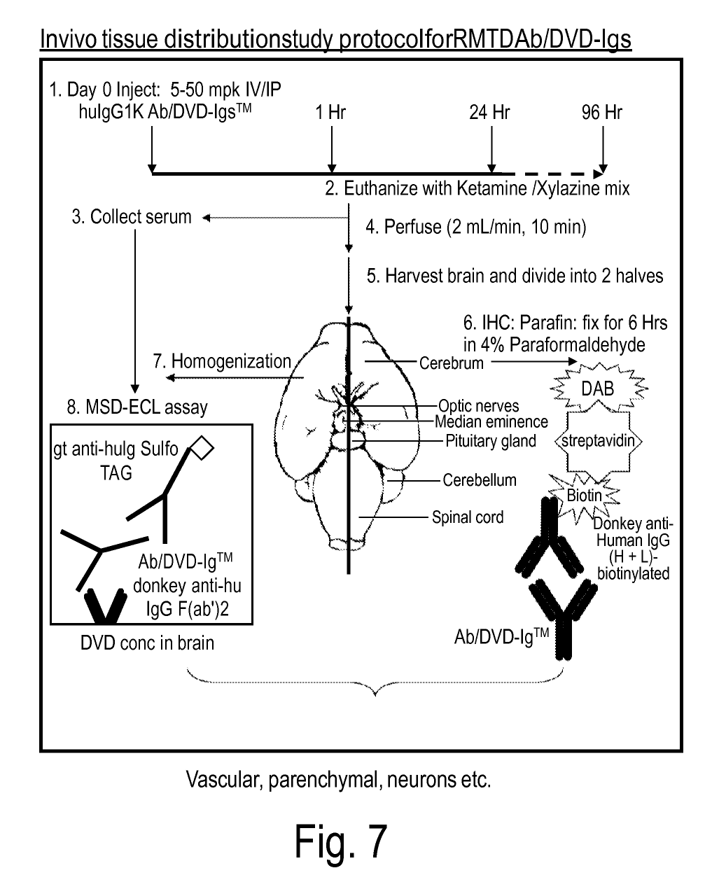

Analysis of in vivo tissue distribution for antibodies or DVD-Ig proteins was performed as shown in FIG. 7. At day 0, murine subjects were intravenously injected or intraperitoneally injected with 5-50 mg/kg (mpk) of a DVD-Ig protein, or a control human IgG1k antibody. The DVD-Ig protein contained a binding region specific for a BBB antigen and another binding region specific for a target. The subjects were injected at hour zero, 1 hour, 24 hours or 96 hours. Subjects were sacrificed at the indicated time using a mixture of ketamine and xylazine.

Serum from each subject was collected and analyzed for concentration of control antibody or DVD-Ig protein using MSD-ESL assays. The assay procedure involved using plates coated with F(ab′)2 fragment of donkey anti-human IgG which specifically bound both the human IgG1k antibody and a DVD-Ig protein. Plates were then contacted with a full length anti-human immunoglobulin (Ig) having a sulfo detection tag, and presence of antibody was detected.

After serum collection, subjects were perfused with D-PBS at a rate of 2 ml/minute for 10 minutes. Brains were harvested and vertically sectioned/divided into equal halves (including equal portions of the cerebrum, optic nerves, pituitary gland, cerebellum and spinal cord). One half of the brain was homogenized and analyzed using an MSD-ECL assay. The other half of the brain was analyzed by immunohistochemical methods.

For the immunohistochemical analysis, tissues were treated with paraformaldehyde and then embedded in paraffin. The embedded material was stained for the detection of anti-human IgG using a biotinylated donkey anti-human IgG (H+L). The material was then contacted with biotin, streptavidin and diaminobenzidine (DAB).

Data from the MSD-ECL assays were used to identify presence of the DVD-Ig proteins in serum and in different tissues/cells of the brain, e.g., vascular, parenchymal and neuronal. Data for the DVD-Ig proteins were compared to data obtained from subjects administered the control human IgG1k antibody.

### Example 11

### Analysis of Anti-Mouse TfR (AB221) Antibody and Humanized Variants

Examples herein analyzed the binding and BBB penetration characteristics of antibody AB221 (IgG2a antibody that specifically recognizes murine TfR) and humanized variants (Table 12). The presence of TfR antibody AB221 and its humanized variants were analyzed using assays and methods described in FIG. 7. Data show higher presence of TfR antibody AB221 and its humanized variants in the serum and brain compared to the human isotype IgG1 control. Immunohistochemical staining showed greater staining for AB221 anti-mouse TfR antibody in the blood vessels and Purkinje cells in the cerebellum compared to control isotype human IgG1 control antibody.

TABLE 12
TfR Binding And In Vivo Biodistribution Data For Anti-Mouse TfR (AB221)
Antibody And Humanized Variants

| | mTfR | | | | | | |
|---|---|---|---|---|---|---|---|
| | binding | | | | | | |
| | cell-based | Brain conc. | | | | | |
| Name of | assay | (20 mpK | | Fold | Serum | Parenchymal | Neuronal |
| Antibody | EC50, | dose) | % ID/g | increase | conc. | staining | staining |
| (Ab) | (nM) | (nM) | brain | over IgG | (nM) | Max = 4 | Max = 4 |
| Control | NB | 1.14 ± 0.17 | 0.3 ± 0.03 | N/A | | 0 | 0 |
| Ig | | | | | | | |
| AB221 | 0.12 | 3.20 ± 0.35 | 0.96 ± 0.09 | 3.2 | 600.0 | 1.5 | 1.9 |
| AB402 | 0.06 | | | | | | |
| AB403 | 0.09 | | | | | | |
| AB404 | 3.0 | 6.0 ± 0.50 | 1.5 | 5 | 580 | 2.5 | 2.3 |
| AB405 | 13.55 | 7.0 ± 0.71 | 1.5 | 5 | 800 | 5 | 2 |

Without being limited by any particular theory or mechanism of action, it is here envisioned that lower affinity TfR antibodies may be in some circumstances more efficiently penetrate the BBB than higher affinity TfR antibodies on the outer position of the same DVD-Ig protein. Alternatively, it is envisioned also that higher affinity TfR antibodies (as determined by binding assays or similar methods) on the inner position of a DVD-Ig protein may in many circumstances more efficiently penetrate the BBB than lower affinity TfR antibodies on the inner position of the same DVD-Ig protein.

### Example 12

### Elevated Levels of DVD-Ig Proteins Detected by Two Orthogonal Methods (IHC and MSD-ECL) and Ability to Transport Across the BBB after a Therapeutic Dosing

Orthogonal methods (MSD-ECL assays and IHC staining) were used in examples herein to determine whether DVD-Ig proteins having a portion that binds TfR effectively crossed the BBB and localized to the brain, e.g., neuronal cells and parenchyma (Table 13).

TABLE 13
MSD-ECL Assay Data And Immunohistochemical Staining Data
For DVD-Ig BB

| | | | Fold | | |
|---|---|---|---|---|---|
| | | | increase | | |
| | | % Injected | over | Parenchymal | Neuronal |
| | EC50 | dose/g brain | control | staining at | staining at |
| DVD-Igs | (nM) | at 24 h | IgG | 24 h | 24 h |
| Isotype | NB | 0.013 ± 0.003 | N/A | No | NO |
| control | | | | | |
| DVD-Ig | | | | | |
| TfRLSAbeta | 0.1 | 0.06 ± 0.01 | 4.6 | No | No |
| DVD2668 | | | | | |
| AbetaLSTfR | 1.2 | 0.14 ± 0.01 | 10.8 | Yes | Yes |
| DVD2671 | | | | | |
| TfRSSHer2 | 0.1 | 0.07 ± 0.01 | 5.4 | Yes | Yes |
| DVD2575 | | | | | |
| Her2ssTfR | 3.4 | 0.14 ± 0.01 | 10.8 | Yes | Yes |
| DVD2576 | | | | | |

The DVD-Ig proteins containing a portion that specifically bound TfR with a lower affinity were present at a higher concentration in the brain than control DVD-Ig protein (Table 13). The MSD-ECL and immunohistochemical data demonstrate that the DVD-Ig proteins were effectively transported across the BBB after therapeutic dosing. FIG. 3 is a set of exemplary micrographs showing that the DVD-Ig proteins were more localized in the parenchymal tissues and neuronal cells of the brain than subjects administered the control (non-TfR) DVD-Ig protein.

### Example 13

### Selection of TNF/TfR DVD-Ig Proteins for Treating Pain

Table 14 contains a list of TNF/TfR DVD-Ig proteins treating pain. The criteria for selection of DVD-Ig proteins for subsequent pain efficacy analysis included data showing low TfR binding affinity, as based on the data in examples herein lower affinity TfR antibodies in the outer position of a DVD-Ig protein more efficiently penetrate the BBB. DVD-Ig proteins were further selected based on the highest concentration in the serum and brain, penetration through the BBB, and highest anti-TNF potency.

Data show that anti-TNF antibody 8C11 strongly bound and inhibited TNF. The anti-TNF antibody 8C11 has the following binding VH and VL regions:

>VH
(SEQ ID NO: 162)
EFQLQQSGPELVKPGASVRISCKASGYSFTDYNMNINVKQSNGKSLEVV
VGVINPNYGSSTYNQKFKGKATLTVDQSSSTAYMQLNSLTSEDSAVYYC
ARKWGQLGRGFFDVWGTGTTVTVSS
>VL
(SEQ ID NO: 163)
QIVLSQSPAILSASPGEKVTMTCRASSSVSYMHWFQQKPGSSPKPWIYA
TSNLASGVPARFSGSGSGTSYSLTISRVEAEDAATYYCQQWSSSPLTFG
AGTKLELKR

TABLE 14
TNF-TfR DVD-Ig Proteins Analyzed For Suitability For Pain Efficacy
Examples

| | | | L929 TNF | | | |
|---|---|---|---|---|---|---|
| | | 293/mTfR | Neutralization | | | |
| | % | cell-based | assay | % ID/g brain | | Serum |
| DVD-Ig protein | Monomer | assay | IC50 | (10mpk/20mpk/ | Brain conc. | conc. |
| pair | (purity) | EC50 (nM) | (nM) | 30mpk) | (nM) | (nM) |
| DVD control | 97.9 | NA | NA | 0.33 +/− 0.08 | 2.5 +/− 0.63 | 2268 |
| TNF (8C11) | 99 | NA | 5.0 | 0.42 +/− 0.13 | 3.1 +/− 1.8 | 1166 |
| muTNF(8C11)- | 93.6 | 4.2 | 4.29 | 0.79 +/− 0.25 | 2.77 +/− 0.46 | 355.2 |
| SS- | | | | | | |
| muTfR(AB221) | | | | | | |
| muTfR(AB221)- | 94 | 0.28 | 24.3 | 0.46 +/− 0.05 | 1.96 +/− 0.30 | 121.25 |
| SL- | | | | | | |
| muTNF(8C11) | | | | | | |
| muTNF(8C11)- | 97.3 | 0.97 | 0.25 | 0.67 +/− 0.10 | 2.57 +/− 0.44 | 415.4 |
| SL- | | | | | | |
| muTfR(AB221) | | | | | | |
| muTNF(8C11)- | 95.3 | 1.16 | 0.42 | 0.44 +/− 0.05 | 1.57 +/− 0.17 | 284.5 |
| LS- | | | | | | |
| muTfR(AB221) | | | | | | |
| muTfR(AB221)- | 93.4 | 0.43 | 5.6 | 0.57 +/− 0.10 | 1.80 +/− 0.07 | 187.1 |
| LL- | | | | | | |
| muTNF(8C11) | | | | | | |
| muTNF(8C11)- | 96.6 | 0.99 | 0.09 | 0.52 +/− 0.56 | 2.87 +/− 0.22 | 214.1 |
| LL- | | | | | | |
| muTfR(AB221) | | | | | | |
| muTfR(AB221)- | 97 | 0.16 | 9.54 | 0.39 +/− 0.02 | 1.57 +/− 0.12 | 458.2 |
| GS- | | | | | | |
| muTNF(8C11) | | | | | | |
| muTNF(8C11)- | 97.1 | 1.68 | 0.59 | 1.91 +/− 0.21 | 10.2 +/− 0.81 | 364.5 |
| GS- | | | | | | |
| muTfR(AB221) | | | | | | |
| muTfR(AB405)- | 93.2 | 3.76 | 4.94 | 3.2 +/− 0.15 | 17.4 +/− 4.8 | 249.8 |
| SL- | | | | | | |
| muTNF(8C11) | | | | | | |
| muTfR(AB405)- | 97.5 | 8.64 | 262 | 5.36 +/− 0.56 | 10.05 +/− 0.88 | 202.64 |
| LS- | | | | | | |
| muTNF(8C11) | | | | | | |
| muTfR(AB405)- | 98.6 | 8.79 | 41.41 | 5.31 +/− 0.83 | 11.46 +/− 2.69 | 130.8 |
| LL- | | | | | | |
| muTNF(8C11) | | | | | | |
| muTfR(AB405)- | 98.9 | 5.33 | 5870 | 4.12 +/− 0.96 | 8.16 +/− 1.61 | 201.8 |
| SS- | | | | | | |
| muTNF(8C11) | | | | | | |

Monoclonal antibody AB221 was used in examples herein and data using this antibody were compared to data using antibody 8C11. The six CDRs for antibody 8C11 (CDR-L1, -L2, and -L3 of light chain and CDR-H1, -H2, and -H3 of heavy chain) are highlighted/bolded portions in SEQ ID NOs: 162-163 above. The CDRs of the heavy chain are DYNMN (SEQ ID NO: 164), VINPNYGSSTYNQKFKG (SEQ ID NO: 165), and KWGQLGRGFFD (SEQ ID NO: 166). The CDRs of the light chain are RASSSVSYMH (SEQ ID NO: 167), ATSNLAS (SEQ ID NO: 168), and QQWSSSPLT (SEQ ID NO: 169)

### Example 14

### Brain Concentration and Location Analysis of Selected DVD-Ig Proteins

DVD-Ig proteins having an antigen binding portion comprising antibody AB221 or humanized variant AB405 were constructed and analyzed in examples herein. Control DVD-Ig proteins were also analyzed.

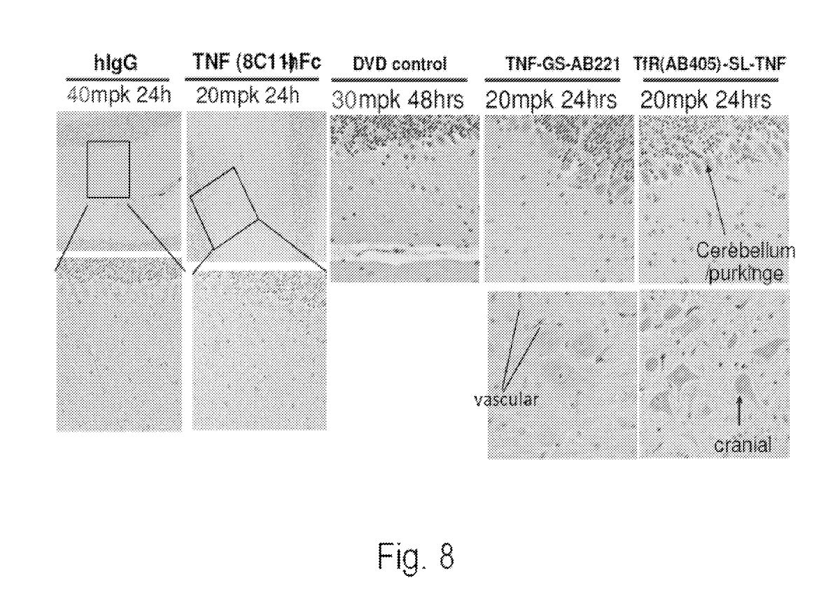

The 8C11-hFc protein comprised 8C11 antibody, which specifically binds TNF, and has a human Immunoglobulin G (hFc) constant region. The TNF-GS-AB221 DVD-Ig protein contained an Ab variable domain that binds TNF (8C11), a GS linker and Ab AB221 (IgG2a) that specifically recognizes murine TfR. The TfR (AB405)-SL-TNF DVD-Ig protein contained the variable domain of AB405 (a humanized variant of AB221 antibody), a SL linker, and an antibody that binds TNF (8C11). Exemplary tissue staining data is shown in FIG. 8. Table 15 shows the concentration and localization data in the brain for subjects administered TfR(AB405)-SL-TNF DVD-Ig protein, TNF-GS-AB221 DVD-Ig protein, 8C11-hFc protein, human IgG or the DVD-Ig protein control. Data show higher concentration of TNF-GS-AB221 DVD-Ig protein and TfR(AB405)-SL-TNF DVD-Ig protein in the brain compared to the control antibody, DVD control and TNF antibody. Furthermore, the TfR(AB405)-SL-TNF DVD-Ig protein had higher concentrations in the brain compared to the TNF-GS-AB221 DVD-Ig protein.

TABLE 15
Protein Concentration And Localization In The Brain

| | | TNF | | | |
|---|---|---|---|---|---|
| | | (8C11)- | DVD | | |
| | HIgG | hFc | control | TNF-GS- | TfR(AB405)- |
| | (40 mpk | (20 mpk | (30 mpk | AB221 | SL-TNF |
| | 24 hr) | 24 hr) | 48 hr) | (20 mpk 24 hrs) | (20 mpk 24 hrs) |
| Brain (nM) | 5.4 +/− 1.0* | 3.1 +/− 1.8 | 2.5 +/− 0.63* | 5.58 +/− 0.88 | 16.2 +/− 1.3 |
| Spinal Cord (nM) | ND | ND | ND | ND | 10.0 +/− 0.5 |
| IHC Staining | 0/0.12 | 0/0.12 | 0/0 | 0.5/1 | 1.5/1.9 |
| max = 4 (Ave) | | | | | |
| Parenchyma/neuron | | | | | |

### Example 15

### Intrathecal Administration of TNF/Tfr DVD-Ig Proteins Crossed the BBB and Effectively Reduced Pain

Examples herein analyzed the effectiveness of intrathecal administration of TNF/TfR DVD-Ig proteins in a pain efficacy model. Partial nerve injuries, such as unilateral loose ligation or chronic constriction injury (CCI) of the sciatic nerve, result in the animal persistently holding the ipsilateral hind paw in a guarded position. Depending on the tightness of ligation, the allodynia and hyperalgesia can persist for hours or days. The Bennett model as it is known involves a surgery to induce a nerve injury and is a well-known pharmacokinetics (PK) and pain efficacy model.

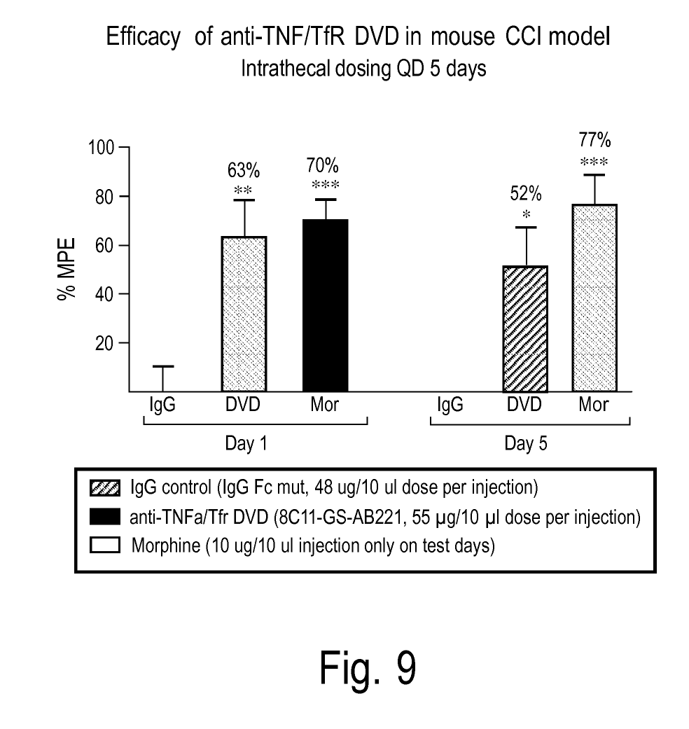

BALB/c murine subjects underwent a Bennett surgery and were intrathecally injected daily with either control IgG specific for mouse Fc (48 μg/10 μl dose per injection); 8C11-GS-AB221 DVD-Ig protein (anti-TNFα/anti-TfR; 55 μg/10 μl dose per injection); or morphine (10 μg/10 μl dose per injection). Subjects were injected daily. Mechanical allodynia was assessed in the above Bennett model 120 minutes post-injection administration at day 1 and day 5 (FIG. 9).

Intrathecal injection of the 8C11-GS-AB221 DVD-Ig protein (anti-TNFα/anti-TfR) in the Bennett model reduced more pain in subjects than intrathecal injection of the control IgG protein. Data for subjects intrathecally injected with the 8C11-GS-AB221 DVD-Ig protein were comparable to data observed for subjects intrathecally injected morphine. Data show that 17 nM of 8C11-GS-AB221 DVD-Ig protein was detected in the brain, and 52 nM of 8C11-GS-AB221 DVD-Ig protein was detected in the spinal cord. The amount of 8C11-GS-AB221 DVD-Ig protein present in the brain following an intravenous injection was observed to be similar (16 nM; Table 14), which shows that efficacious amounts were obtained in the brain by administering a 20 mpk intravenous injection.

### Example 16

### Intravenous Administration of TNF/TfR DVD-Ig Proteins was Effective to Cross the BBB and Reduce Pain

Examples herein analyzed the effectiveness of intravenous administration of TNF/TfR DVD-Ig proteins in the Bennett PK/pain efficacy model described above.

BALB/c murine subjects underwent a Bennett surgery and were intravenously injected with: control IgG specific for mouse Fc (48 μg/10 μl dose per injection); 8C11-GS-AB221 DVD-Ig protein (anti-TNFα/anti-TfR; 55 μg/10 μl dose per injection); or an acute post-operation dose of gabapentin (10 μg/10 μl dose per injection). Subjects were injected daily (20 mg/kg). Mechanical allodynia was assessed in the above Bennett model 120 minutes post-injection at day 1 and at day 5.

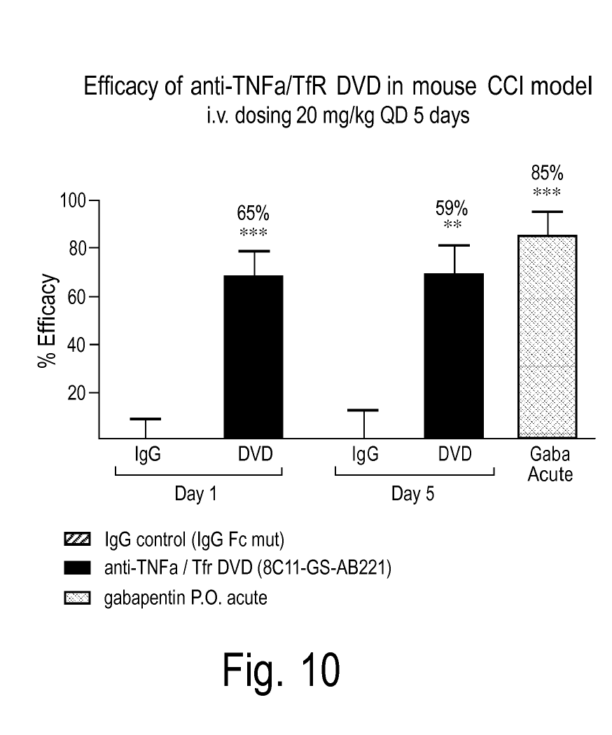

Data show that intravenous injection of the 8C11-GS-AB221 DVD-Ig protein (anti-TNFα/anti-TfR) reduced pain in subjects in the Bennett model better than intravenous injection of the control IgG (FIG. 10). Most importantly, data for intravenous injection of the 8C11-GS-AB221 DVD-Ig protein show that it was only slightly less effective in obtaining pain reduction that intravenous injection of an acute dose of gabapentin. Data herein show that administration of 8C11-GS-AB221 DVD-Ig protein by either by an intrathecal administration or an intravenous administration was effective to reduce pain.

### Example 17

### TNF/TfR DVD-Ig Proteins Concentration and Localization in the Serum and Brain

This example analyzed the concentration and localization of TfR((AB405)-SL-TNF DVD-Ig proteins administered to subjects in a PK study described above. Subjects were administered different doses (single or multiple doses of 20-40 mpk) of TfR (AB405)-SL-TNF DVD-Ig proteins either subcutaneously (SC), intravenously (IV), or intraperitoneally (IP).

Data in Table 16 show comparable serum and brain concentration for TfR(AB405)-SL-TNF DVD-Ig proteins using either intravenous and intraperitoneal routes of administration. Data show that TfR/TNF DVD-Ig binding proteins effectively entered the brain. Without being limited by any particular theory or mechanism of action, it is envisioned that these TfR/TNF DVD-Ig are potential therapeutic agents for crossing the BBB and treating different neurological diseases and conditions.

TABLE 16
TfR(AB405)-SL-TNF DVD-Ig Protein Concentrations And Localization In The Brain

| | Time | | | | | IHC Staining Max = 4 | |
|---|---|---|---|---|---|---|---|
| | of | | | | | (ave) | |
| | collection | Serum | | Brain | Spinal cord | Parenchyma | |
| Treatment | (hr) | (ug/mL) | (nM) | (nM) | (nM) | Neuron | |
| SC - single | 1 | 0.63 +/− 0.25 | 3.1 +/− 1.2 | 8.4 +/− 1.6 | 5.7 +/− 1.5 | 0.87 | 1.0 |
| dose 20mpk | 24 | 38.9 +/− 11.5 | 194.9 +/− 57.5 | | | | |
| IP - single | 1 | 34.7 +/− 8.6 | 173.6 +/− 43.2 | 16.2 +/− 1.3 | 10.0 +/− 0.5 | 1.5 | 1.9 |
| dose 20mpk | 24 | 109.4 +/− 16.2 | 547.2 +/− 81.3 | | | | |
| IV - single | 1 | 83.8 +/− 21.1 | 419.5 +/− 105.9 | 16.8 +/− 1.8 | 9.3 +/− 1.5 | 1.9 | 1.4 |
| dose 20mpk | 24 | 109.0 +/− 20.9 | 545.0 +/− 104.7 | | | | |
| SC - single | 1 | 1.0 +/− 0.69 | 5.4 +/− 3.4 | 16.2 +/− 1.7 | 8.0 +/− 1.3 | 1.1 | 0.87 |
| dose 40mpk | 24 | 88.0 +/− 7.3 | 440.0 +/− 36.9 | | | | |
| IP - single | 1 | 45.0 +/− 11.5 | 225.5 +/− 57.8 | 22.4 +/− 4.3 | 9.6 +/− 2.2 | 1.5 | 0.87 |
| dose 40mpk | 24 | 218.5 +/− 15.1 | 1092. +/− 75.4 | | | | |
| IV - single | 1 | 168.7 +/− 44.6 | 843.3 +/− 223.4 | 20.7 +/− 2.6 | 10.2 +/− 2.6 | 2.2 | 1.7 |
| dose 40mpk | 24 | 172.6 +/− 9.9 | 863.2 +/− 49.9 | | | | |
| IV - multiple | 48 | 860.4 +/− 65.3 | 4302 +/− 326.9 | 23.3 +/− 2.7 | | | |
| dose | | | | | | | |
| 20mpk | | | | | | | |

### Example 18

### RGMA-TfR DVD-Ig Proteins in a Multiple Sclerosis Model

The early stages of many neurodegenerative diseases are characterized by neurite damage and compromised synaptic function. Neurite degeneration often leads to neuronal cell death and impairs the conduction of signals in the affected nerves, causing impairment in sensation, movement, cognition, or other functions depending on which nerves are involved. Neurite degeneration is also a pathological indicator of the autoimmune disease multiple sclerosis (MS). Repulsive guidance molecule A (RGMA) is plays an important role in axonal guidance, inhibition of axonal outgrowth, and the formation of scar tissue after brain injury. RGMA-TfR DVD-Ig proteins were constructed and tested in a mouse model of MS. The DVD-Ig proteins were engineered to have the anti-BBB antigen binding portion or anti-target portion in the outer position (N-terminus) or the inner position (C-terminus). Depending on the position of the anti-BBB antigen binding portion, one would either select a lower affinity antibody (outer position) or a higher affinity antibody (inner position). As this Example was using an anti-BBB antigen binding portion in the outer position, the criteria for selection of DVD-Ig proteins for use in the MS efficacy model was low TfR binding affinity, the highest anti-RGMA potency, the highest serum and brain concentrations, and greatest BBB penetration. Accordingly, RGMA (AE12-1)-GS-TfR(AB403) DVD-Ig binding proteins and TfR(AB405)-SL-RGMA(AE12-1) DVD-Ig binding proteins were expressed in a larger scale and analyzed for efficacy in a MS model described herein.

RGMA-JL3-VH (AE12-1) (SEQ ID NO: 170) binding
protein amino acid sequence is
EVQLVQSGAEVKKPGASVKVSCKASGYTFTSHGISWVRQAPGQGLDWMGW
ISPYSGNTNYAQKLQGRVTMTTDTSTSTAYMELSSLRSEDTAVYYCARVG
SGPYYYMDVWGQGTLVTVSS
RGMA-JL3-VL (AE12-1) ) (SEQ ID NO: 171) binding
protein amino acid sequence is
QSALTQPRSVSGSPGQSVTISCTGTSSSVGDSIYVSWYQQHPGKAPKLML
YDVTKRPSGVPDRFSGSKSGNTASLTISGLQAEDEADYYCCSYAGTDTLF
GGGTKVTVLG

The six CDRs (CDR-L1, -L2, and -L3 of light chain and CDR-H1, -H2, and -H3 of heavy chain are highlighted/bolded portions in SEQ ID NOs: 170-171 above. The CDRs of the heavy chain are SHGIS (SEQ ID NO: 172), WISPYSGNTNYAQKLQ (SEQ ID NO: 173), and VGSGPYYYMDV (SEQ ID NO: 174). The CDRs of the light chain are TGTSSSVGDSIYVS (SEQ ID NO: 175), DVTKRPS (SEQ ID NO: 176), and CSYAGTDTL (SEQ ID NO: 177). See U.S. Pat. No. 8,822,645 and International Publication No. WO/2013/112922, each of which is incorporated by reference herein in its entirety.

TABLE 17
RGMA-TfR DVD-Ig Proteins Analyzed For Suitability For MS Efficacy Examples

| | | 293/mTfR | | % ID/g brain | | |
|---|---|---|---|---|---|---|
| | % Monomer | cell-based | BMP Assay | (10mpk/20mpk/ | Brain cons | Serum Conc |
| Name of DVD pair | (purity) | assay EC50 | IC50 (nM) | 30mpk) | (nM) | (nM) |
| DVD control | 97.9 | NA | NA | 0.33 +/− 0.08 | 2.5 +/− 0.63 | 2268 +/− 188.3 |
| RGMA (AE12-1) | 100% | NA | 0.25 | 0.4 +/− 0.16 | 5.9 +/− 2.1 | 4512 |
| RGMA(AE12-1)-LS-TfR(AB402) | 99.3 | 127.4 | 0.213 | 3.0 +/− 0.60 | 10.7 +/− 1.2 | 1183.75 |
| RGMA(AE12-1)-SL-TfR(AB402) | 90.5 | 2.13 | 0.367 | 1.7 +/− 0.34 | 6.5 +/− 0.89 | 818.75 |
| RGMA(AE12-1)-LL-TfR(AB402) | 91.8 | 1.67 | 1.78 | 1.4 +/− 0.21 | 6.2 +/− 0.89 | 533.75 |
| RGMA(AE12-1)-SS-TfR(AB402) | 96.4 | 30.85 | 0.436 | 3.9 +/− 0.62 | 7.8 +/− 0.95 | 669.5 |
| RGMA(AE12-1)-GS-TfR(AB402) | 91.4 | 5.36 | 0.014 | 6.3 +/− 0.75 | 9.9 +/− 1.2 | 187.04 |
| RGMA(AE12-1)-LS-TfR(AB403) | 99.1 | 25.73 | <0.025 nM | 10.1 +/− 1.5 | 18.4 +/− 5.0 | 481.4 |
| RGMA(AE12-1)-SS-TfR(AB403) | 98.2 | 71.03 | 1.18 | 4.4 +/− 0.49 | 9.3 +/− 1.11 | 486.9 |
| RGMA(AE12-1)-GS-TfR(AB403) | 98.8* | 4.3 | <0.025 nM | 3.1 +/− 0.36 | 14.8 +/− 0.65 | 396.6 |
| TfR(AB405)-SL-RGMA(AE12-1) | 95.2** | 4.41 | 0.73 | 2.4 +/− 0.42 | 13.1 +/− 2.8 | 642.8 |
| TfR(AB405)-LS-RGMA(AE12-1) | 99.2 | 3.9 | No inhibition | 11.4 +/− 3.99 | 18.3 +/− 1.7 | 462.2 |
| TfR(AB405)-GS-RGMA(AE12-1) | 90.1 | 28.07 | 3.12 | 8.9 +/− 0.48 | 19.1 +/− 1.88 | 611.9 |
| TfR(AB405)-LL-RGMA(AE12-1) | 98.9 | 4.44 | 0.701 | 10.2 +/− 0.48 | 19.0 +/− 2.94 | 754.15 |
| TfR(AB404)-LL-RGMA(AE12-1) | 94.1 | 3.61 | 13.25 | 11.9 +/− 2.63 | 24.8 +/− 3.77 | 464.4 |

*scaled for efficacy studies;
**used for efficacy studies

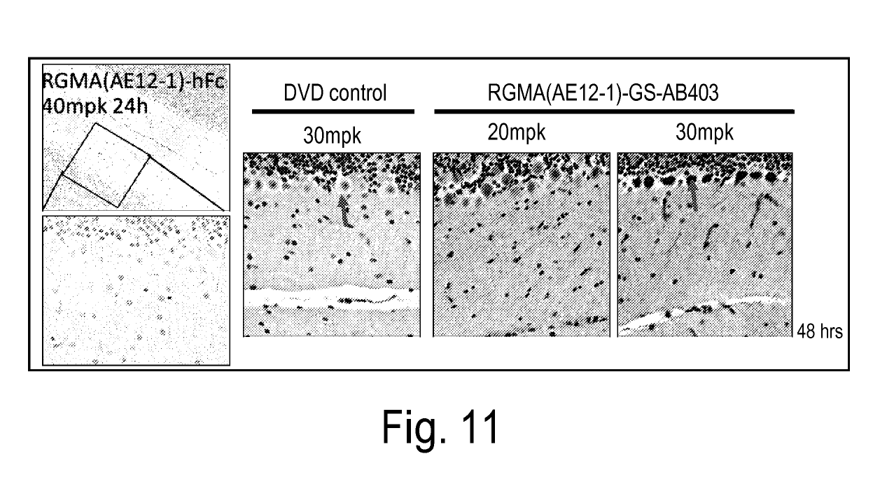

FIG. 11, panel A is a set of exemplary micrographs of stained brain tissues from subjects administered either (1) 40 mpk of RGMA (AE12-1)-hFc at 24 hours; (2) 30 mpk human IgG control; (3) 20 mpk of RGMA (AE12-1)-GS-AB403 DVD-Ig protein, or (4) 30 mpk of RGMA (AE12-1)-GS-AB403 DVD-Ig protein. Data show effective staining of brain tissues from subjects administered with RGMA (AE12-1)-GS-AB403 DVD-Ig protein, indicating that the DVD-Ig protein effectively crossed the BBB and bound to RGMA on neuronal cells/tissue in the brain. Table 18 A lists additional exemplary binding protein concentration data in brain issues from subjects after intraveneous administration of RGMA/Tfr DVD-Ig protein. Table 18A shows data for subjects administered (40 mg/kg) of anti-RGMA (AE12-1) antibody or anti-RGMA/anti-TFR DVD-Ig binding protein [TfR(AB405)-SL-RGMA(AE12-1)]. Control subjects were administered an IgG control antibody. Data show substantially higher levels of anti-RGMA/anti-TFR DVD-Ig binding protein in the brain tissue compared to levels of anti-RGMA antibody in the brain tissue of that treatment group.

TABLE 18
Binding Protein Brain Concentration Data

| | | | Anti-RGMA-SL-Tfr |
|---|---|---|---|
| | | | DVD-Ig binding |
| Test article | Anti-RGMA IgG | IgG control | protein |
| Dose, route, time | 40 mg/kg, | 40 mg/kg, | 40 mg/kg, |
| | IV, 24 h | IV, 24 h | IV, 24 h |
| Brian | 5.3 ± 1.8 | 5.4 ± 1.0 | 25.7 ± 4.0 |
| concentration | | | |
| (nM) ± SD | | | |

Analysis: 1way ANOVA, Bonferroni posthoc test** P ≦ 0.01; *** P ≦ 0.001

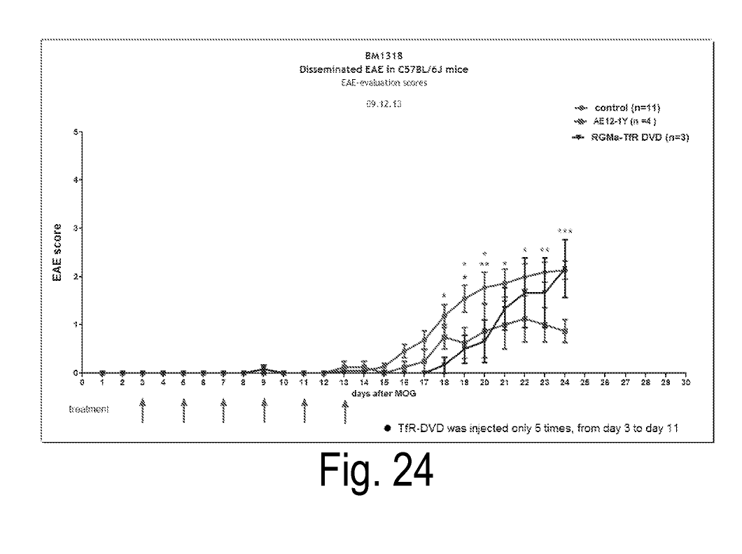

Data show that TfR/RGMA DVD-Ig treatment significantly increased myelination compared to the parent antibody. For example, a murine model system for disseminated encephalomyelitis (EAE) was utilized (FIG. 24). Autoimmune encephalomyelitis (EAE) scores were obtained for murine C57Bl/6J subjects administered myelin oligodendrocyte glycoprotein (MOG) and then injected (5 mpk) either RGMA antibody (AE12-1Y; not the parent Ab for RGMA/TfR DVD-Ig binding protein), or RGMA/TfR (AE12-1/AB403; 20 mpk) DVD-Ig binding protein, or control human immunoglobulin. The subjects were administered RGMA antibody or control immunoglobulin six times on days 3, 5, 7, 9, 11 and 13 after MOG administration. RGMA/TfR DVD-Ig binding protein was administered only five times on days 3, 5, 7, 9, and 11 after MOG treatment. For the control subjects (six injections of 5 mpk), data show that 11 days post last dosing the serum level detected was 321.1+/−224.2 nM. For the RGMA antibody injected subjects (six injections of 5 mpk), data show that 11 days post last dosing the serum level detected was 84.5+/−219.6 nM. For the RGMA/TfR DVD-Ig binding protein treated subjects (five injections of 20 mpk), data show that 13 days post last dosing the serum level was below the detection limit. The data indicate that over a period of time that there was a greater efficacy of the RGMA/TfR DVD-Ig binding protein compared to the RGMA antibody.

### Example 19

### TfR/RGMA DVD-Ig Protein Concentration and Localization in the Serum and Brain

The concentration and localization of AB405-SL-RGMA DVD-Ig proteins administered to male BALB/c murine subjects were analyzed in a PK study. Subjects were administered AB405-SL-RGMA DVD-Ig proteins at different doses (single or multiple doses of 20-40 mpk) either subcutaneously (SC), intravenously (IV), or intraperitoneally (IP). Table 19 shows concentration and localization data in serum and the brain for subjects administered AB405-SL-RGMA DVD-Ig proteins at different doses.

The DVD-Ig proteins were detected by IHC staining in parenchyma and neuronal cells two hours following a single systemic injection (20 mpk, intravenous). Increased brain uptake was observed 24 hours after injection. The DVD-Ig proteins accumulated in the brain by using multiple injections, for example using two 20 mpk intravenous injections.

TABLE 19
AB405-SL-RGMA DVD-Ig Protein Concentration And Localization In Serum And The Brain In A PK Study

| | | Time of | | | | | | |
|---|---|---|---|---|---|---|---|---|
| | | tissue | | | | | | |
| | | collection | Serum | | Brain | Spinal Cord | IHC Staining | |
| Ab/DVD | Treatment | (h) | (ug/mL) | (nM) | (nM) | (nM) | max = 4 (Ave) | |
| AB405-SL- | IV - single | 1 | 115.4 +/− 25.5 | 576.9 +/− 127.8 | | | | |
| RGMA | dose 20mpk | 24 | 98.6 +/− 7.7 | 601.8 +/− 419.0 | 21.6 +/− 4.6 | 8.3 +/− 3.9 | 0.75 | 0.75 |
| | | 48 | 40.9 +/− 6.1 | 204.9 +/− 30.7 | 17.9 +/− 1.6 | 8.2 +/− 1.5 | 1.1 | 1.1 |
| | IV - single | 1 | 289.3 +/− 33.2 | 1446 +/− 166.6 | | | | |
| | dose 40mpk | 24 | 207.1 +/− 21.7 | 1036 +/− 108.7 | 25.7 +/− 4.0 | 13.4 +/− 1.8 | 1.5 | 0.87 |
| | | 48 | 79.7 +/− 15.8 | 398.5 +/− 79.4 | 23.3 +/− 2.1 | 11.43 +/− 1.2 | 1.7 | 0.87 |
| | IV - multiple | 48 | 538.9 +/− 26.9 | 2273 +/− 848.8 | 30.4 +/− 4.6 | ND | | |
| | dose 20mpk | | | | | | | |
| | @ 0, 24, 48 hr | | | | | | | |
| RGMA hFc | IV - single | 24 | 653.0 +/− 151.6 | 4354 +/− 1011 | 5.3 +/− 1.8 | ND | 0.25 | 0 |
| | dose 40mpk | | | | | | | |
| IgG Fc Mut | IV - single | 24 | 449.8 +/− 77.3 | 2999 +/− 515.6 | 5.4 +/− 1.0 | ND | 0 | 0.12 |
| | dose 40mpk | | | | | | | |

### Example 20

### RGMA(AE12-1)-GS-TfR(AB403) Provides Increase in Remyelination and Decrease in Demyelination in Cuprizone Treated Mice

A cuprizone mouse model of demyelination was used to show that a bifunctional DVD-Ig protein that specifically binds transferrin receptor and RGMa reduces demyelination and promotes remyelination of depleted tissue in the brain.

The murine cuprizone model is described in greater detail in Skripuletz et al. (2008) Am. J. Phys. 172(4):1053-61), incorporated by reference herein in its entirety. In this model, mice are fed with the copper chelator cuprizone, leading to oligodendrocyte death and a subsequent reversible demyelination. The cuprizone model correlates with newer histopathological data in multiple sclerosis (MS) and is a valuable tool for studies on de- and remyelination. It was previously shown that mice treated with cuprizone have an intact blood brain barrier as measured by Evan's Blue. Neither transferrin receptor nor RGMa levels were altered by cuprizone treatment. Assessment of myelin by IHC staining has been established. Percent myelin basic protein (MBP) positive area in the hippocampus, cortex and total brain of mouse subjects were analyzed. Lower levels of MBP indicate greater incidence of brain demyelination, which is indicative of MS. Mice treated with cuprizone alone showed the lowest levels of MBP.

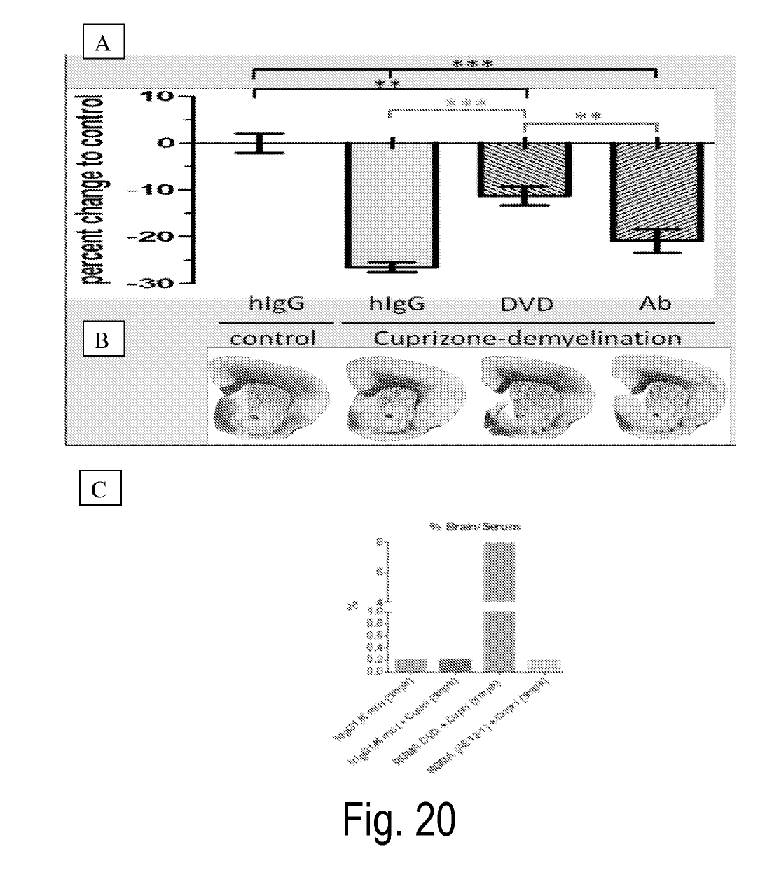

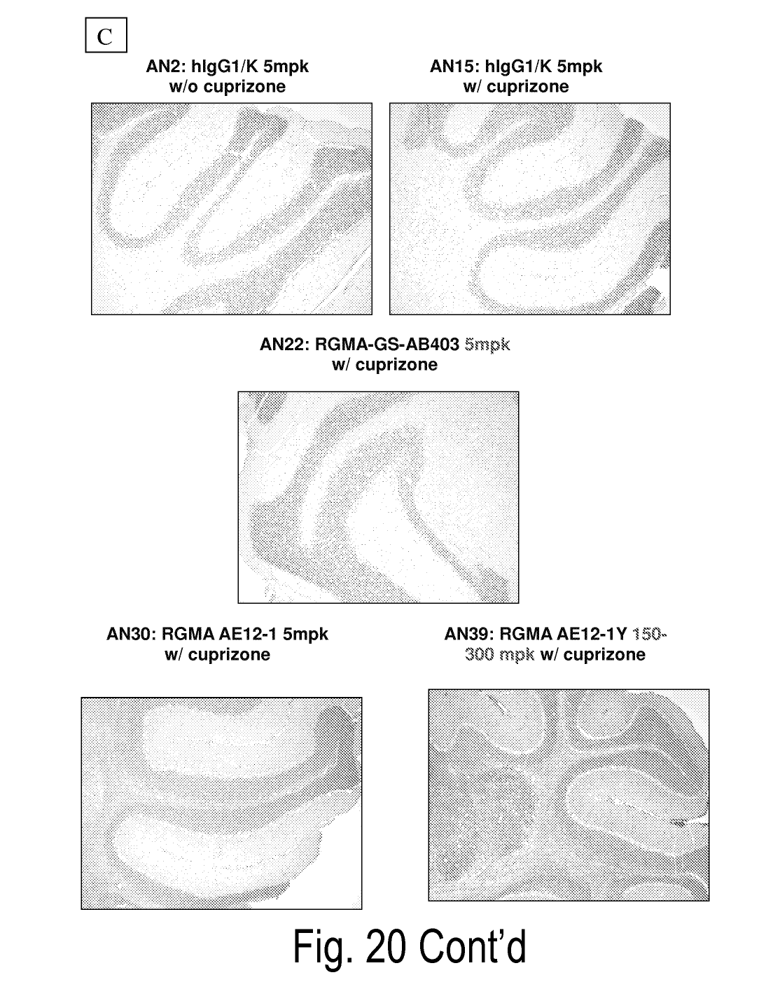

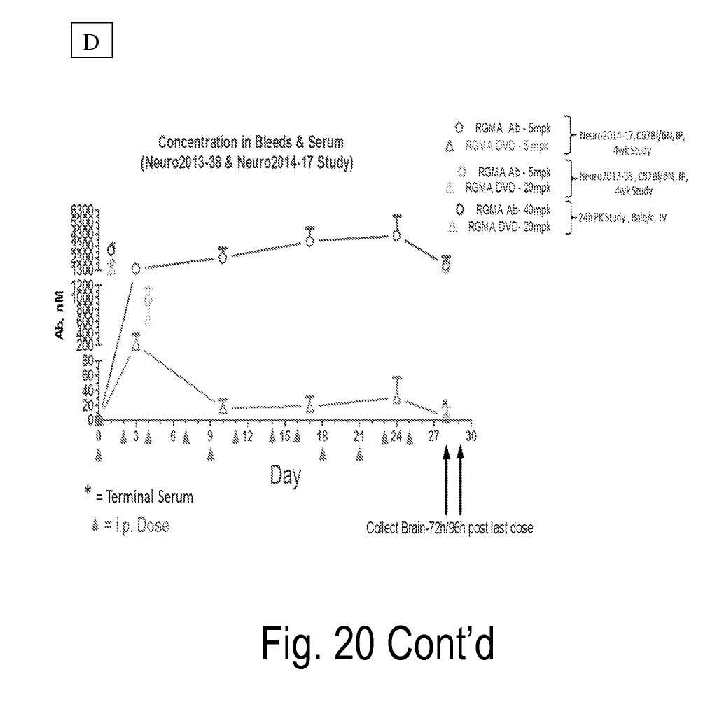

Mice were administered cuprizone for four weeks, twice per week in freshly prepared food. Mice administered cuprizone showed lower levels of remyelination than mice treated with control. The mice were treated three times a week (Monday, Wednesday and Friday) with intraperitoneal injections of either RGMA(AE12-1)-GS-TfR(AB403) DVD-Ig protein (SEQ ID NOs: 184 and 185) (20 mg/kg), AE12.1, an RGMA specific Ab at 5 mg/kg, or a control human IgG at 5 mg/kg (See FIG. 20 panels A-C). Analysis of multiple studies show that RGMA DVD-Ig binding proteins were present in less amounts in tail bleed and serum samples (FIG. 20 panel D).

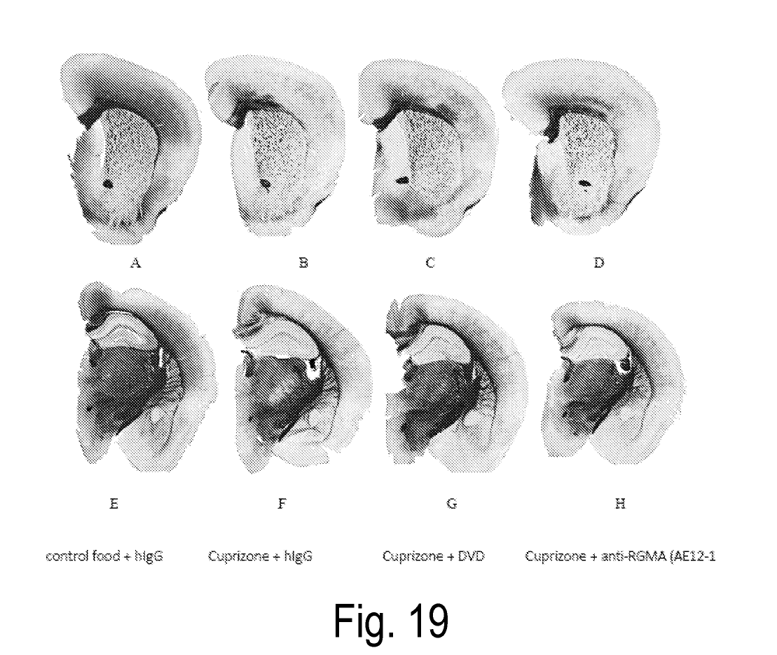

FIG. 19, panels A-H show photographs of MBP staining in mice treated with control, cuprizone, cuprizone and RGMA(AE12-1)-GS-TfR(AB403) DVD-Ig protein as well as cuprizone and RGMa antibody. Brains of mice administered cuprizone and RGMA(AE12-1)-GS-TfR(AB403) DVD-Ig protein showed greater myelin integrity than the brains of mice administered cuprizone alone. Cuprizone treatment significantly reduced MBP positive area as shown in FIG. 20, panels A-D (27% reduction shown in some embodiments). Mice administered RGMA (AE12-1)-GS-TfR (AB403) DVD-Ig protein showed only an 11% reduction in MBP, while mice administered RGMa antibody showed a 21% reduction compared to a 26% reduction for control IgG. Comparing the treatment groups to the cuprizone treated group, MBP+ area in the RGMa antibody treatment group was not significantly increased, whereas the RGMA/TfR DVD-Ig protein group showed a significant increase in MBP+ area from the cuprizone group. Importantly, a non-specific DVD-Ig control that does not bind a BBB specific antigen, accordingly data show that it does not get transported across the BBB. Thus, the RGMA/TfR DVD-Ig binding protein by specifically binding a BBB antigen is able to pass through to the BBB.

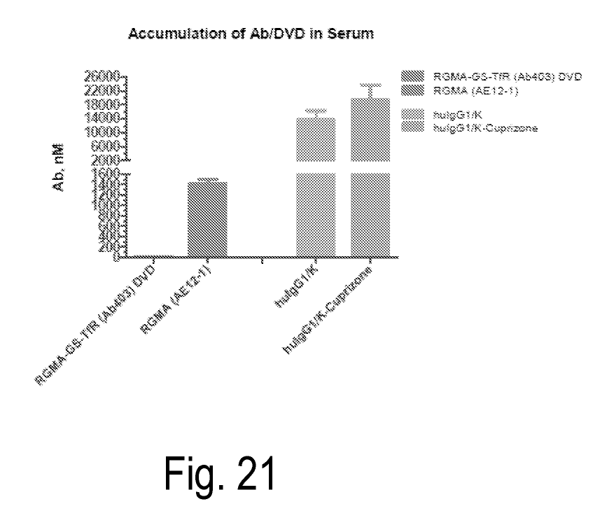

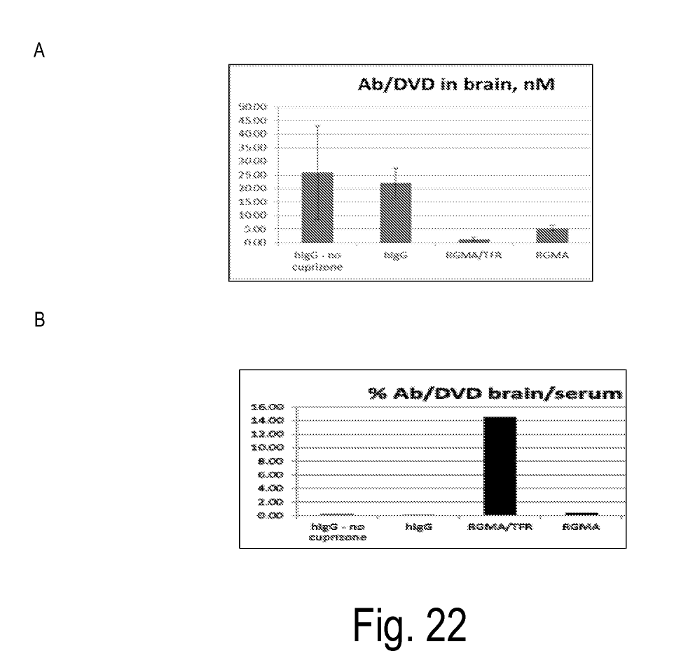

FIG. 21 shows the relatively low concentration of RGMA (AE12-1)-GS-TfR(AB403) DVD-Ig protein in serum relative to RGMa antibody or human IgG. FIG. 22, panel A shows the concentrations of RGMA (AE12-1)-GS-TfR(AB403) DVD-Ig protein, RGMa antibody and human IgG in the brain of mouse subjects. FIG. 22, panel B shows that 15% of the RGMA(AE12-1)-GS-TfR(AB403) DVD-Ig protein transcytosed from serum to brain while RGMa antibody and human IgG were present at much lower percentages.

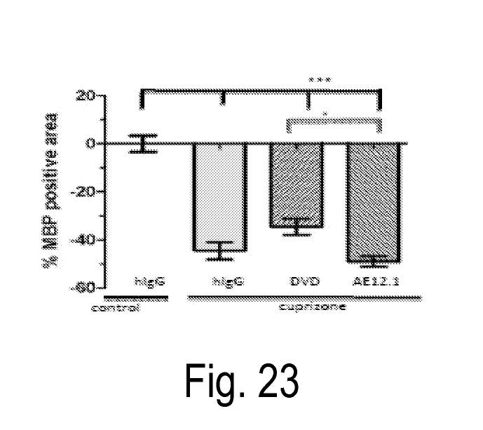

FIG. 23 is a bar graph showing the relative decrease in myelin basic protein positive areas in mouse brain when equal amounts of (5 mg/kg) administered control, RGMA(AE12-1)-GS-TfR(AB403) DVD-Ig protein or RGMa Ab after cuprizone challenge. In the DVD-Ig protein treatment group the MBP+ area is significantly increased when compared to data for the RGMa Ab treatment group. In summary, these data indicate that the RGMA/TfR DVD-Ig binding proteins crossed the BBB, entered the brain, prevented demyelination and were an effective treatment in a model of MS.

### Example 21

### Preparation and Analysis of Amyloid-Beta/Tfr DVD-Ig Proteins

Bapineuzumab is a humanized Ab that targets the neurotoxic amyloid-beta peptide. Amyloid-beta (Aβ) is an early biomarker of Alzheimer's disease and other neurodegenerative pathologies. Murine antibody 3D6 is the parent of the humanized monoclonal antibody Bapineuzumab. The 3D6 antibody is a monoclonal antibody that specifically binds to an N-terminal epitope located in the human β-amyloid peptide, specifically, residues 1-5. See U.S. Pat. No. 7,189,819, incorporated by reference herein in its entirety. A cell line producing the 3D6 monoclonal antibody (RB96 3D6.32.2.4) was deposited with the American Type Culture Collection (ATCC), Manassas, Va. 20108, USA on Apr. 8, 2003 under the terms of the Budapest Treaty and has deposit number PTA-5130. Shown below are the heavy chain variable domain (SEQ ID NO: 38) and light chain variable domain (SEQ ID NO: 39) of 3D6 Ab.

SEQ ID NO: 38
EVKLVESGGGLVKPGASLKLSCAASGFTFSNYGMSWVRQNSDKRLEWV
ASIRSGGGRTYYSDNVKGRFTISRENAKNTLYLQMSSLKSEDTALYYC
VRYDHYSGSSDYWGQGTTVTVSS
SEQ ID NO: 39
YVVMTQTPLTLSVTIGQPASISCKSSQSLLDSDGKTYLNWLLQRPGQS
PKRLIYLVSKLDSGVPDRFTGSGSGTDFTLKISRIEAEDLGLYYCWQG
THFPRTFGGGTKLEIK121

DVD-Ig proteins were designed and constructed that contained a portion that specifically bound to Aβ, and another portion that bound TfR. Examples of DVD-Ig protein and portions that bind Aβ are shown herein (e.g., Tables 1-4). Examples herein (see for example Table 3) show a list of Aβ/TfR DVD-Ig proteins that were engineered and analyzed in assays and a model system for binding to TfR and Aβ, for concentration in serum and the brain, and for IHC staining in the parenchyma and neurons.

Eight week old C57Bl/6N female murine subjects were intravenously administered 20 mpk of the DVD-Ig proteins. The AB405-SL-Aβ (3D6) DVD-Ig protein, Aβ (3D6)-GS-AB403 DVD-Ig protein, and Aβ (3D6)-SS-AB402 DVD-Ig protein-, which had high affinity for Abeta but low affinity for TfR, were present/localized in greater amount in brain tissue than subjects administered the control IgG. Table 20 provides data showing that a single IP injection (20 mpk) of anti-TfR/Aβ DVD-Ig resulted in significantly increased intracellular Aβ38, Aβ40, Aβ42 and membrane-bound Aβ40 levels in Tg2576 mice brain homogenates in 24 hours compared to mice administered 3D6 anti-Aβ Ab. The 3D6 Abeta binding affinity was 34.2 ng/ml (Table 20).

TABLE 20
Aβ/TfR DVD-Ig Protein Concentrations And Localization In The Brain

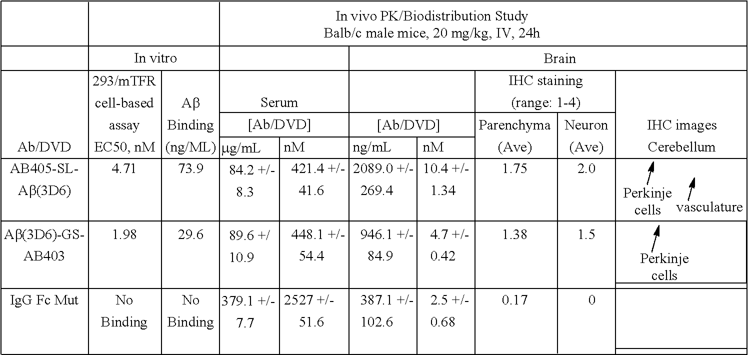

### Example 22

### TfR-SL-Aβ(3D6) DVD-Ig Protein Pharmacodynamic (PD) Study Using Tg2576 Murine Subjects

Tg2576 mice overexpress a mutant form of amyloid precursor protein (APP), APPK670/671L, linked to early-onset familial AD. These mice develop amyloid plaques and progressive cognitive deficits. Tg2576 murine subjects, which were 4.5 months old, were obtained (Taconic Biosciences) and were administered an IV injection of 5 mpk 3D6 Abeta antibody or 20 mpk of AB405-SL-AB(3D6), a TfR/Abeta DVD-Ig protein. After 4 hours, 24 hours or 14 days, Abeta (Aβ) levels and Ab or DVD-Ig protein levels were measured in serum and brain/cerebellum extracts (extracellular/PBS extract, intracellular/NP-40 extract and membrane-bound/SDS extract fractions) using different methods described below.

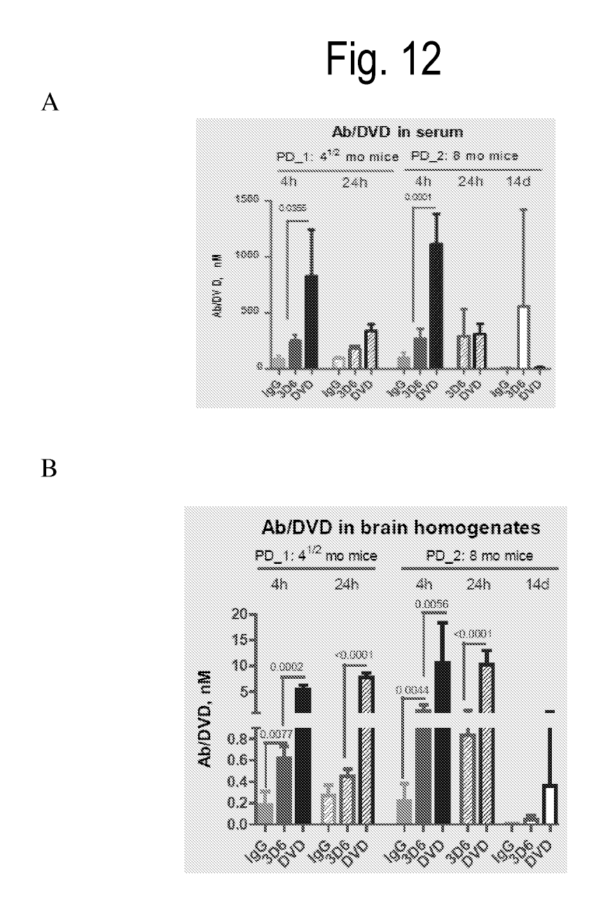

An hFc capture MSD assay was used to determine the 3D6 antibody or AB405-SL-AB(3D6) DVD-Ig in serum samples and brain (cerebellum) samples from the subjects (see Example 6A). The amounts of TfR/Abeta DVD-Ig protein detected in serum were about three- or four-fold higher/greater at 4 hours after injection compared to samples from subject administered 3D6 antibody only. However, the amounts of TfR/Abeta DVD-Ig protein detected in serum samples were only about two-fold higher at 24 hours compared to samples for subjects administered 3D6 Ab (FIG. 12, panel A). This may be due to the target mediated disposition (TMD) of the TfR/Abeta DVD-Ig protein due to the TfR binding domain which binds to most tissues/cells in the body as they all express TfR. Due to this well-known and well established TMD effect, TfR targeting DVD-Ig proteins clear from serum much more quickly than non-TfR targeted parent Abs (e.g., Abeta Ab). For example, at 14 days after administration, the serum concentration data for mice subjects administered 3D6 antibody only was much higher than the serum concentration for the TfR/Abeta DVD-Ig protein (FIG. 12, panel A and FIG. 12, panel B). Therefore, to achieve similar exposure in 24 hours for both Abeta Ab and TfR DVD-Ig proteins, TfR DVD-Ig proteins were injected at 4× higher dose as compared to the antibody only. Most importantly, the amount of TfR/Abeta DVD-Ig protein was about three-fold or four fold, or even eight-fold or nine-fold greater in brain cerebellum at 4 hours after injection compared to 3D6 antibody. Furthermore, the amount of TfR/Abeta DVD-Ig protein was about 12-fold to 18-fold greater at 24 hours after injection compared to 3D6 antibody (FIG. 12, panel B).

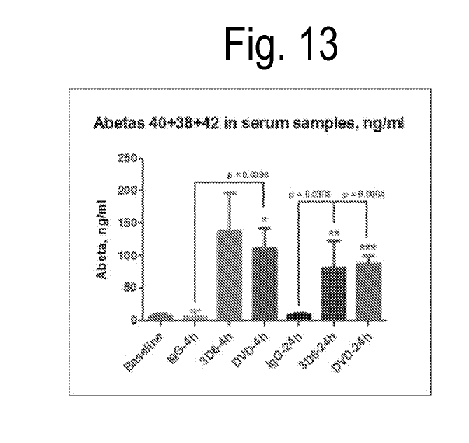

Analysis was performed to determine levels of Abeta in serum and in brain. A MSD Rodent/Human MultiPlex Panel 4G8 Abeta 3-Plex Ultra-Sensitive assay was used to measure individual amounts of Aβ38, Aβ40, or Aβ42 species in serum or brain. An increase in Abeta concentration was observed in serum samples for subjects treated with 3D6 antibody or AB405-SL-AB(3D6) (FIG. 13). The Abeta concentration in serum increased several fold in subjects receiving 3D6 or DVD-Ig protein treatment compared to untreated subjects (baseline) or subjects receiving control IgG. A number of antibodies have been described which increase total peripheral Abeta levels upon administration to APP transgenic animal models or AD patients. This effect may be due to stabilization of circulating antibody-bound Abeta interfering with the physiological systemic clearance of peripheral Abeta (i.e., by decreasing the turnover rate of Abeta) (Golde et al. (2009) CNS Neurol. Disord. Drug Targets 8:31-49). Free Abeta in the plasma has a half-life of approximately 5 minutes. When bound to an Ab the half-life is dramatically prolonged; in some cases the half-life of the anti-Abeta mAb:Abeta complex appears to be roughly equivalent to the half-life of the monoclonal antibody (approximately 5-7 days) (See Levites et al. (2006) FASEB J. 20(14):2576-2578). A decrease in turnover rate in serum in turn leads to increased Abeta levels in serum.

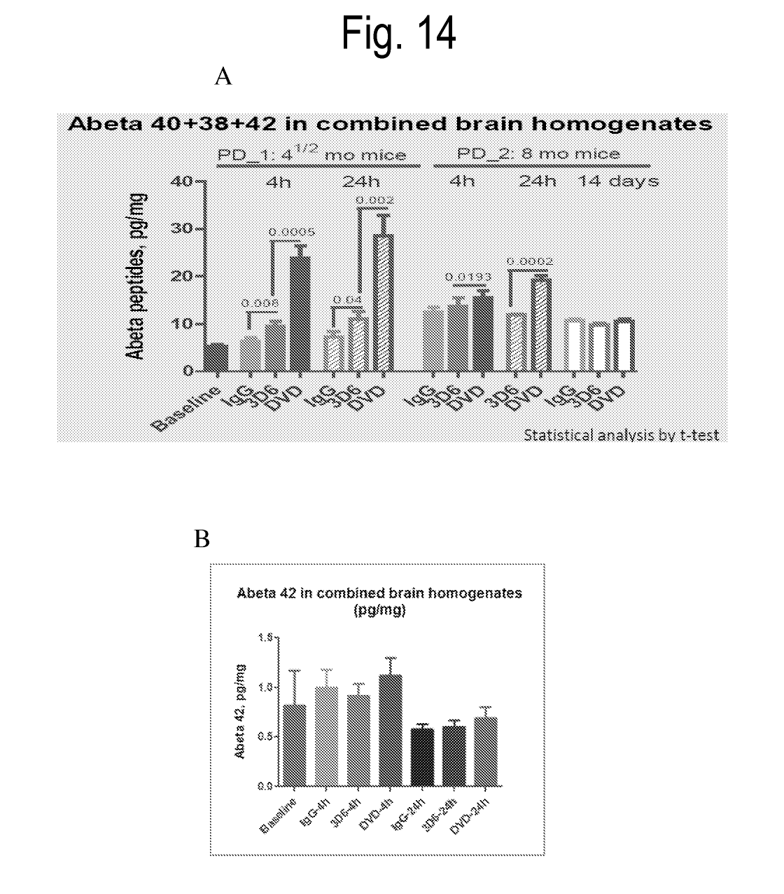

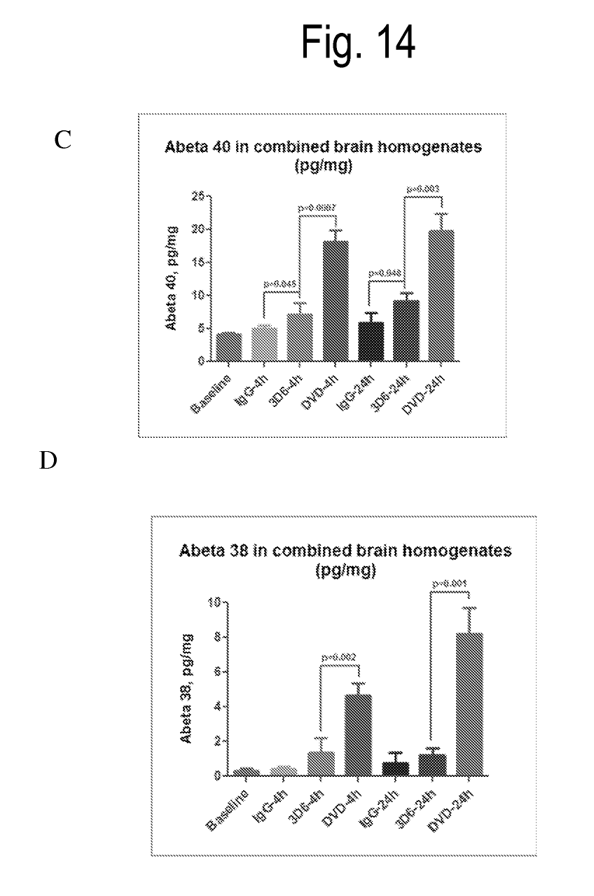

An increase in the concentration of Abeta was observed in brain samples for subjects treated with TfR/Abeta DVD-Ig protein. The total amount of Aβ40, Aβ38 and Aβ42 in brain samples from subjects intravenous or IV injected 4-24 hours previously with AB405-SL-AB(3D6) was greater compared to brain samples from subjects IV injected with 3D6 Ab (FIG. 14, panel A). Intracellular Aβ38, Aβ40, Aβ42 and membrane-bound Aβ40 levels in brain homogenates increased significantly by DVD treatment in 4-24 hours. Serum Aβ increased both by 3D6 or DVD-Ig treatment compared to IgG treatment. Individual amounts of each of Aβ40, Aβ38 and Aβ42 were also greater in brain samples from subjects IV injected with TfR/Abeta DVD-Ig protein compared to brain samples from subjects IV injected with 3D6 antibody only (FIG. 14, panel B, FIG. 14, panel C, and FIG. 14, panel D).

Homogenates were prepared using different mixtures/buffers (i.e., PBS, NP-40, or SDS) and were used to analyzed for concentration of different Abeta peptides. PBS-buffer brain homogenization provides a system for detecting/identifying extracellular soluble Abeta. NP-40 buffer brain homogenization provides a system for detecting/identifying intracellular soluble Abeta. SDS buffer-brain homogenization is a system for membrane-associated Abeta. A formic acid or GuHCL homogenization system may be used for detecting/identifying insoluble Abeta. Thus, many different systems may be effectively used to obtain Abeta data.

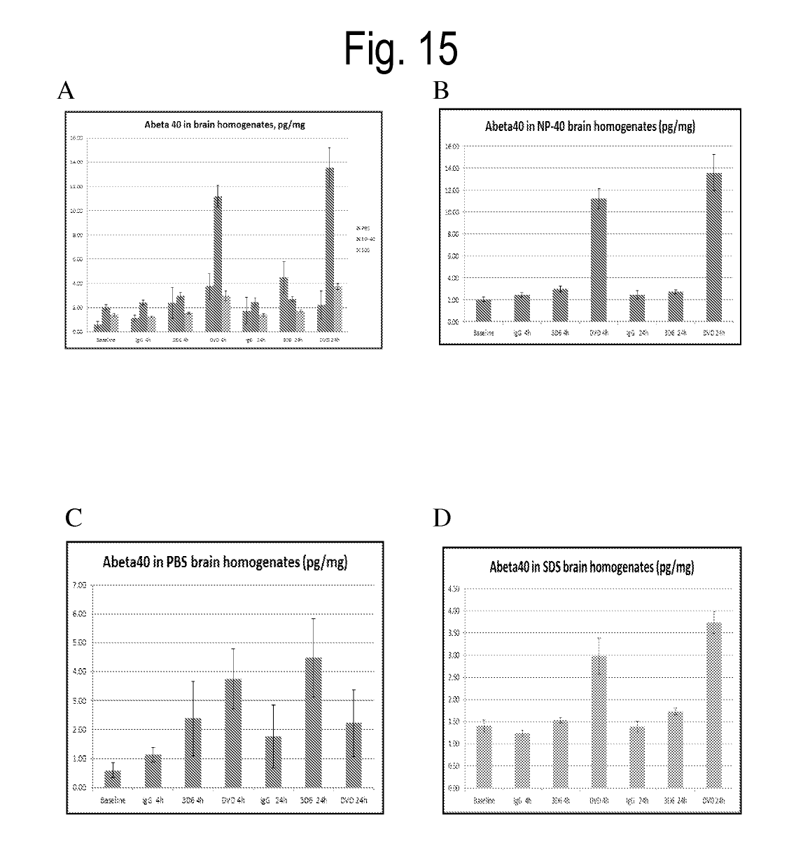

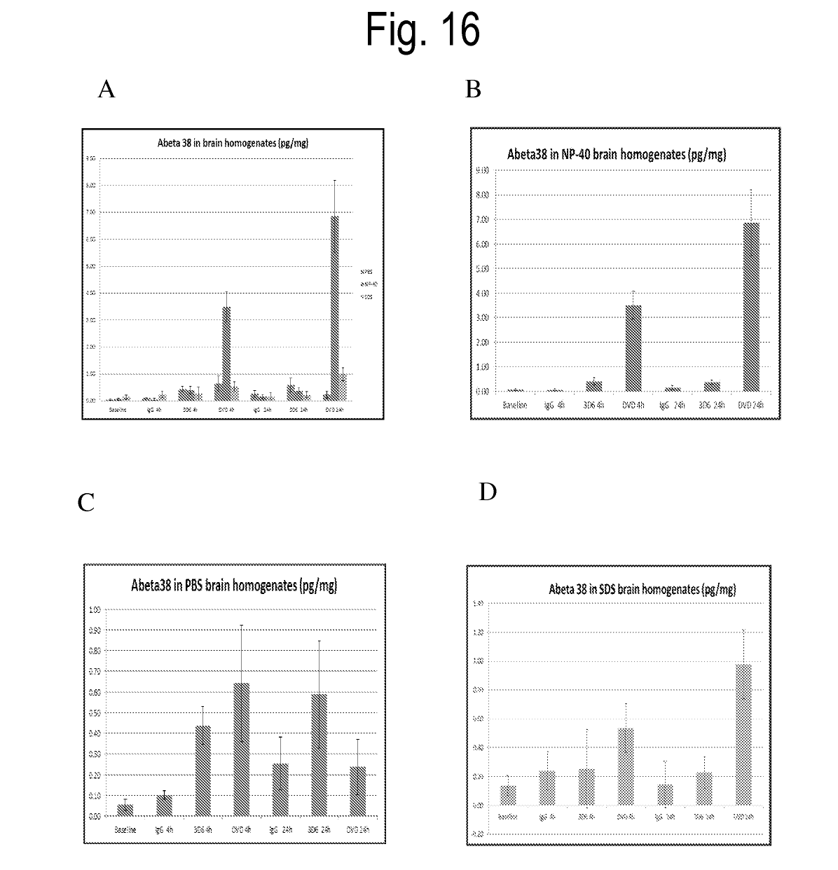

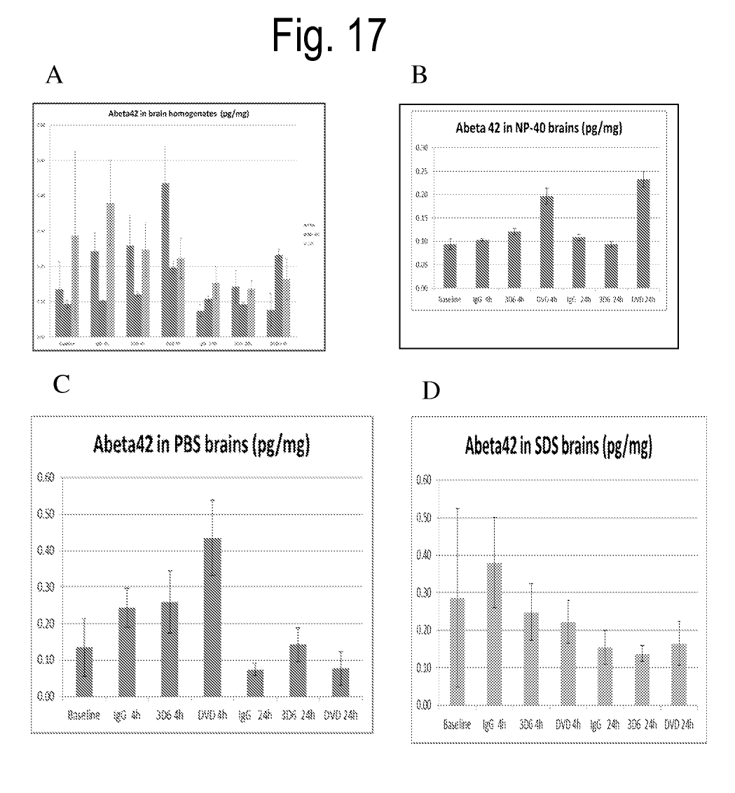

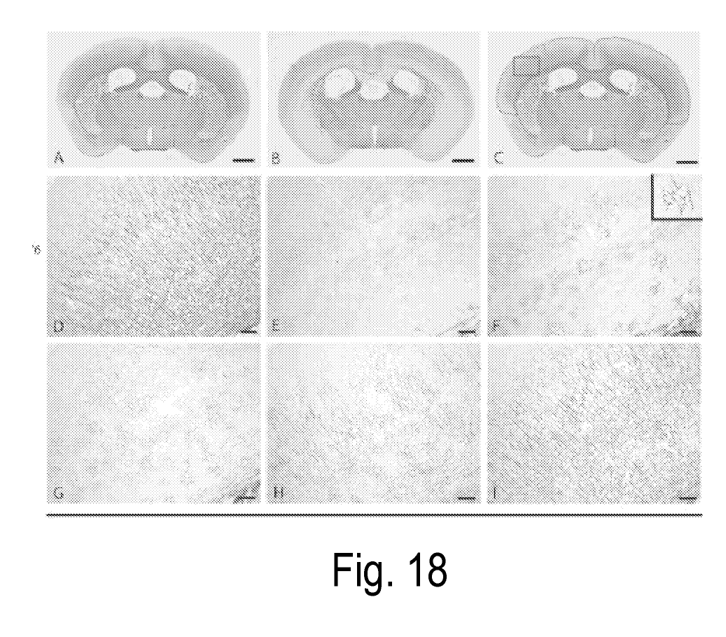

Intracellular and membrane-bound Aβ38 levels increased several fold in homogenate samples from subjects treated with the TfR/Abeta DVD-Ig protein compared to samples from subjects treated with 3D6 antibody alone (FIG. 16, panels A-D). Similar dramatic results were seen for Aβ40, as multiple-fold increases in Aβ40 levels were observed in homogenates from subjects treated with the TfR/Abeta DVD-Ig protein compared to samples from subjects treated with 3D6 antibody (FIG. 15, panels A-D). Some intracellular and membrane-bound Aβ38 levels increased several fold in NP-40 and PBS homogenate samples from subjects treated with the AB405-SL-AB(3D6) compared to samples from subjects treated with 3D6 antibody (FIG. 16, panels A-D). Similar concentration trends were shown for Aβ42 (FIG. 17, panels A-D).

Increased DVD-Ig protein binding to Abeta in brain may decrease turnover rate of Abeta and may lead to accumulation of DVD-Ig protein-bound Abeta, similar to what is observed in serum. AB405-SL-AB(3D6), a TfR/Abeta DVD-Ig protein, was found at higher levels than 3D6 antibody in subjects IV injected with either the DVD-Ig protein or the monoclonal antibody respectively. The monoclonal antibody was not observed to enter the brain in an appreciable amount compared to the DVD-Ig protein.

In summary, data in Examples herein shown enhanced brain uptake into mouse brain and PD/efficacy in various mouse models achieved using BBB-penetrating mouse TfR DVD-Ig binding proteins.

Without being limited by any particular theory or mechanism of action, DVD-Ig proteins engineered and analyzed by these methods efficiently penetrate the BBB and bind to Abeta targets in the brain. Furthermore, once bound, the DVD-Ig protein may be effective in clearing Abeta from the brain. Further analysis will determine whether this observed effect requires multiple injections of Abeta/TfR DVD-Ig protein for a longer period of time (e.g. >3 months).

### Example 23

### Her2/TfR DVD-Ig Binding Protein 2 Week PK Study in SCID Mice

A two weeks PK study was performed using SCID mice to determine, among other things, brain uptake and serum exposure of Her2-GS-TfR (AB403) DVD-Ig binding protein (20 mpk; VH SEQ ID NO: 192 and VL SEQ ID NO:193) compared to Her2 antibody (5 mpk). The model use 9 wk CB-17/Icr-Fox Chase SCID female mice (Charles River Labs).

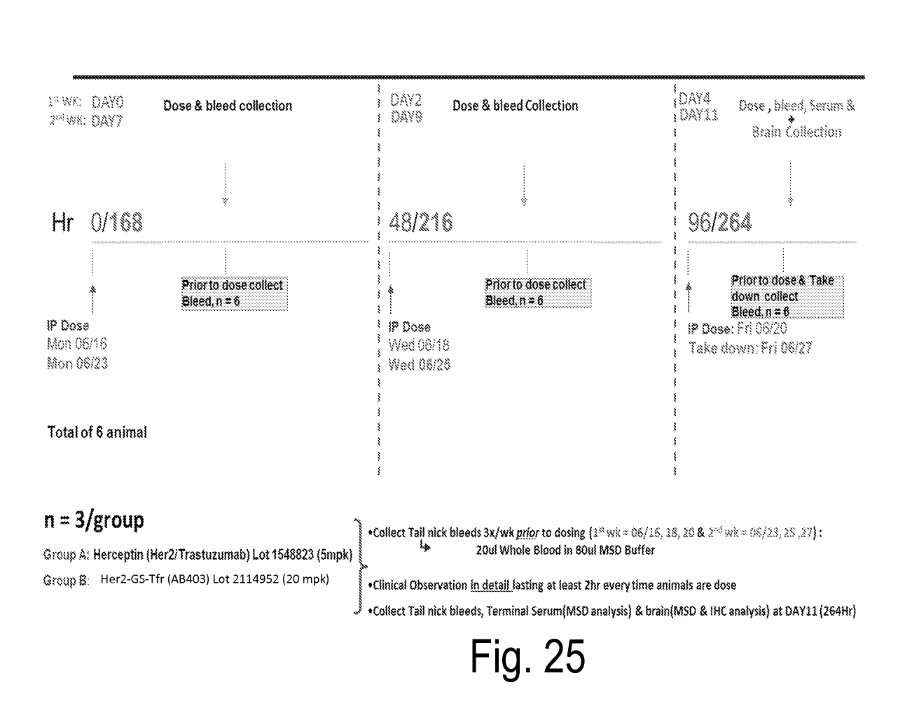

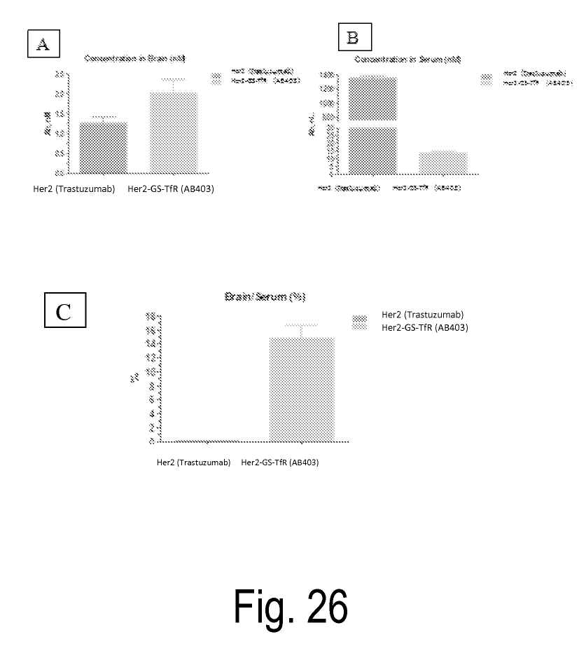

The subjects (total of 6 subjects; n=3 for each group) on day zero were administered an intraperitoneal dose of either Herceptin or Her2-GS-TfRDVD-Ig binding protein. Subjects were administered a total of five doses over two weeks and samples were collected and analyzed using tail nick bleeds. Samples were also analyzed for terminal serum (using MSD analysis) and brain exposure (using MSD and IHC analysis) at day 11. FIG. 25 shows a schematic of the administration and collection regimen and Table 21 shows data for Herceptin and Her2-Gs-Tfr (AB403) DVD-Ig binding protein in brain homogenates (see also FIG. 26 panels A-C). Data show that the concentration of Her2-Gs-Tfr DVD-Ig binding protein in the brain was higher compared to Herceptin. Herceptin concentration was higher in the serum.

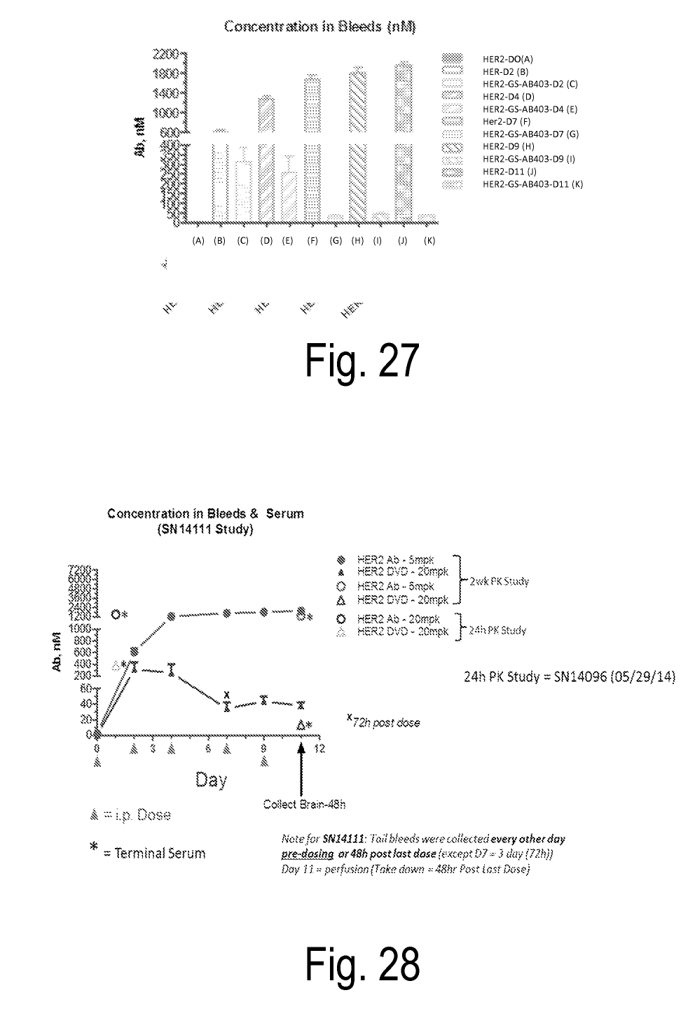

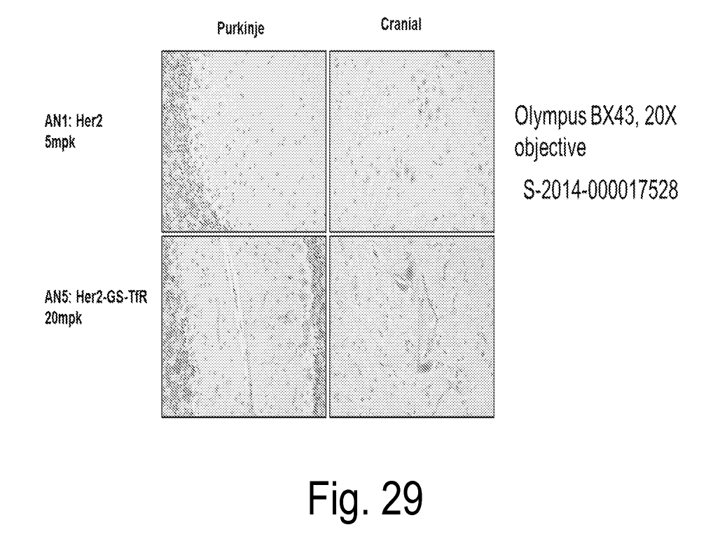

Further analysis of tail bleed samples at days zero, two, four, seven, nine and eleven show that the Her2-Gs-Tfr (AB403) DVD-Ig binding protein entered the blood and over a period of time decreased in concentration (see Table 22 and FIG. 27). This two week PK study was also compared to data for a similar 24 hour PK study (FIG. 28). Similar results were observed, in which lower concentrations of Her2-Gs-Tfr DVD-Ig binding protein were observed in tail bleeds and serum samples compared to samples from Herceptin treated subjects. Staining of brain samples was also performed (see FIG. 29 and Table 23). Data show higher staining of the brain vasculature, parenchyma and neurons for subjects treated with Her2/TfR DVD-Ig binding protein than for subjects administered Herceptin. Specific staining of the cranial and purkinje regions was observed for the subjects administered Her2/TfR DVD-Ig binding protein.

In conclusion, data indicate that the Her2-Gs-Tfr DVD-Ig binding proteins were able to cross the BBB and target the brain.

TABLE 21
Concentrations in Brain Homogenate & Serum from SCID mice

| | | Treatment | | | | | | | | |
|---|---|---|---|---|---|---|---|---|---|---|
| | | i.p. 2x/wk (total | | | | | | | | |
| | | 5 doses in | Brain Conc. | | Serum Conc. | | Ab/Protein | Ab/Serum | Brain Mean | Serum Mean |
| ANIMAL # | Test Article | 2 wks) | ng/mL | nM | ug/mL | nM | nM/ug | % | nM | nM |
| 1 | Herceptin | 264 h (5mpk) | 212.02 | 1.41 | 198.82 | 1325.49 | 0.100 | 0.10663888 | | |
| 2 | (HER2/Trastuzumab) | | 216.54 | 1.44 | 212.72 | 1418.11 | 0.098 | 0.10179502 | | |
| 3 | Lot 1548823 | | 136.32 | 0.91 | 197.40 | 1316.01 | 0.068 | 0.06905934 | 1.26 | 1353.20 |
| 4 | Her2-GS- | 264 h (20mpk) | 270.56 | 1.80 | 2.97 | 14.87 | 0.138 | 12.1298182 | | |
| 5 | TfR(AB403) Lot | | 403.74 | 2.69 | 2.90 | 14.50 | 0.202 | 18.5687502 | | |
| 6 | 2114952 | | 235.81 | 1.57 | 2.29 | 11.47 | 0.111 | 13.7069479 | 2.02 | 13.61 |

TABLE 22
Concentrations in Tail Bleeds (DAY 0, 2, 4, 7, 9, 11) from SCID mice (20 ul Whole Blood in 80 ul MSD Assay Buffer w/EDTA) - by hFc
capture

| | | Bleeds Conc, ug/ml | | | | | | Bleeds Conc, nM | | | | | |
|---|---|---|---|---|---|---|---|---|---|---|---|---|---|
| | | D0 | D2 | D4 | D7 | D9 | D11 | D0 | D2 | D4 | D7 | D9 | D11 |
| # | Test Article | (06/16) | (06/18) | (06/20) | (06/23) | (06/25) | (06/27) | (06/16) | (06/18) | (06/20) | (06/23) | (06/25) | (06/27) |
| 1 | Herceptin | 1.08 | 96.75 | 172.29 | 230.18 | 288.35 | 273.07 | 7.23 | 644.97 | 1148.60 | 1534.54 | 1922.36 | 1820.49 |
| 2 | (HER2/ | 0.05 | 97.23 | 194.33 | 250.43 | 236.02 | 303.92 | 0.31 | 648.20 | 1295.52 | 1669.51 | 1573.44 | 2026.11 |
| | Trastuzumab) | | | | | | | | | | | | |
| 3 | Lot 1548823 | 0.04 | 80.55 | 203.89 | 272.46 | 284.51 | 301.91 | 0.27 | 536.99 | 1359.25 | 1816.41 | 1896.76 | 2012.71 |
| 4 | Her2-GS- | BDL | 73.22 | 70.29 | 6.95 | 9.59 | 6.62 | BDL | 366.10 | 351.44 | 34.77 | 47.93 | 33.10 |
| 5 | TfR(AB403) Lot | BDL | 80.98 | 66.37 | 8.30 | 9.24 | 7.84 | BDL | 404.92 | 331.83 | 41.52 | 46.21 | 39.19 |
| 6 | 2114952 | BDL | 34.79 | 18.20 | 5.20 | 7.31 | 8.31 | BDL | 173.97 | 90.99 | 25.99 | 36.53 | 41.55 |

Bleeds Mean, nM

| | | D0 | D2 | D4 | D7 | D9 | D11 |
|---|---|---|---|---|---|---|---|
| # | Test Article | (06/16) | (06/18) | (06/20) | (06/23) | (06/25) | (06/27) |
| 1 | Herceptin | | | | | | |
| 2 | (HER2/Trastuzumab) | | | | | | |
| 3 | Lot 1548823 | 2.60 | 610.05 | 1267.79 | 1673.49 | 1797.52 | 1953.11 |
| 4 | Her2-GS- | | | | | | |
| 5 | TfR(AB403) Lot | | | | | | |
| 6 | 2114952 | BDL | 315.00 | 258.09 | 34.09 | 43.56 | 37.95 |

TABLE 23
Staining of brains for 2 week PK study
SN14111 every other day IP dosing, 5 doses in 2 weeks, samples collected 48 h post last dose

| | | | | Vasculature | Parenchyma | Neuron | Note |
|---|---|---|---|---|---|---|---|
| A: 5mpk | 1 | Herceptin (Her2/Trastuzumab) A-903699/ | IP | 0.5 | 0 | 0 | chloroid plexus |
| | 2 | Lot2169936(From Jon Hickson) | IP | 1 | 0 | 0 | chloroid plexus |
| | 3 | | IP | 0.5 | 0 | 0 | chloroid plexus |
| B: 20mpk | 4 | Her2-GS-TfR(AB403) Lot 2114952 | IP | 3 | 0.5 | 0.5 | cranial & purkinje |
| | 5 | | IP | 3 | 1 | 1 | cranial & purkinje |
| | 6 | | IP | 2 | 0.5 | 0.5 | cranial & purkinje |

### INCORPORATION BY REFERENCE

The contents of all cited references (including literature references, patents, patent applications, and websites) that maybe cited throughout this application are hereby expressly incorporated by reference in their entirety for any purpose, as are the references cited therein. The disclosure will employ, unless otherwise indicated, conventional techniques of immunology, molecular biology and cell biology, and pathology, which are well known in the art.

The present disclosure also incorporates by reference in their entirety techniques well known in the field of molecular biology and drug delivery. These techniques include, but are not limited to, techniques described in the following publications: Ausubel et al. (eds.) (1993) CURRENT PROTOCOLS IN MOLECULAR BIOLOGY, John Wiley & Sons, NY; Ausubel et al. (eds.) (4th ed.; 1999) SHORT PROTOCOLS IN MOLECULAR BIOLOGY, John Wiley & Sons, NY; Bergman et al. (1998) J. Pharmacol. Exp. Ther. 284(1):111-5; Braen et al. (2010) Int. J. Toxicol. 29(3):259-67; Smolen and Ball (eds.) (1984) CONTROLLED DRUG BIOAVAILABILITY, DRUG PRODUCT DESIGN AND PERFORMANCE, John Wiley & Sons, NY; Garg and Balthasar (2009) AAPS J. 11(3):553-7; Giege and Ducruix (2nd ed.; 2009) CRYSTALLIZATION OF NUCLEIC ACIDS AND PROTEINS, a Practical Approach, pp. 201-16, Oxford University Press, NY; Goodson (1981) in: MEDICAL APPLICATIONS OF CONTROLLED RELEASE, 2:115-138 (CRC Press, Florida); Hammerling et al. (1981) in: MONOCLONAL ANTIBODIES AND T-CELL HYBRIDOMAS, pp. 563-681, Elsevier, NY; Harlow et al. (1988) ANTIBODIES: A LABORATORY MANUAL, Cold Spring Harbor Laboratory Press, 2nd ed. (Cold Water Press, New York); Kabat et al. (1987 and 1991) SEQUENCES OF PROTEINS OF IMMUNOLOGICAL INTEREST, National Institutes of Health, Bethesda, Md.; Kabat et al. (5^{th} ed.; 1991) SEQUENCES OF PROTEINS OF IMMUNOLOGICAL INTEREST, U.S. Department of Health and Human Services, NIH Publication No. 91-3242; Kontermann and Dubel (eds.) (2001) ANTIBODY ENGINEERING, Springer-Verlag, NY (ISBN 3-540-41354-5); Kriegler (1990) Gene Transfer and Expression, A Laboratory Manual, Stockton Press, NY; Levites et al. (Epub 2006) FASEB J. 20(14):2576-8; Lu and Weiner (eds.) (2001) CLONING AND EXPRESSION VECTORS FOR GENE FUNCTION ANALYSIS, BioTechniques Press, MA; Langer and Wise (eds.) (1974) MEDICAL APPLICATIONS OF CONTROLLED RELEASE, CRC Press, FLA; Old and Primrose (3d ed.; 1985) PRINCIPLES OF GENE MANIPULATION: AN INTRODUCTION TO GENETIC ENGINEERING Blackwell Scientific Publications, MA 2:409; Robinson (ed.) (1978) SUSTAINED AND CONTROLLED RELEASE DRUG DELIVERY SYSTEMS, Marcel Dekker, Inc., NY; Sambrook et al. (eds.) (2nd ed.; 1989) MOLECULAR CLONING: A LABORATORY MANUAL Cold Spring Harbor Laboratory Press, NY; Shen et al. (2004) Adv. Drug Deliv. Rev. 14:56(12):1825-57; Skripuletz et al. (2008) Am. J. Phys. 172(4):1053-61; Winnacker (1987) FROM GENES TO CLONES: INTRODUCTION TO GENE TECHNOLOGY, VCH Publishers, NY;

### EQUIVALENTS

The disclosure may be embodied in other specific forms without departing from the spirit or essential characteristics thereof. The foregoing embodiments are therefore to be considered in all respects illustrative rather than limiting of the disclosure. Scope of the disclosure is thus indicated by the appended claims rather than by the foregoing description, and all changes that come within the meaning and range of equivalency of the claims are therefore intended to be embraced herein.

1. A dual variable domain (DVD) binding protein comprising at least a first and a second binding domain, wherein the first binding domain specifically binds a target that facilitates entrance of the binding protein into a brain, and the second binding domain specifically binds to TNF, APP, BACE1, HER2, APP, Abeta or RGMa, wherein the binding protein is effective for modulating the concentration of TNF, APP, BACE1, HER2, APP, Abeta or RGMa within the brain and/or serum of the subject.

2. The DVD binding protein of claim 1, wherein the target that facilitates entrance into or passage across the barrier comprises a receptor expressed on vascular endothelial cells.

3. The DVD binding protein of claim 1, wherein the first binding domain specifically binds a receptor selected from the group consisting of the insulin receptor, the transferrin receptor, LRP, the melanocortin receptor, the nicotinic acetylcholine receptor, the VACM-1 receptor, IGFR, EPCR, EGFR, TNFR, the leptin receptor, M6PR, the lipoprotein receptor, NCAM, LIFR, LfR, MRP1, AchR, DTr, the glutathione transporter, SR-B1, MYOF, TFRC, ECE1, LDLR, PVR, CDC50A, SCARF1, MRC1, HLA-DRA, RAMP2, VLDLR, STAB1, TLR9, CXCL16, NTRK1, CD74, DPP4, endothelial growth factor receptors 1, 2 and 3, the glucocorticoid receptor, the ionotropic glutamate receptor, the M3 receptor, the aryl hydrocarbon receptor, the GLUT-1, inositol-1,4,5-trisphosphate (IP3) receptor, the N-methyl-D-aspartate receptor, S1P1, the P2Y receptor, TMEM30A, and RAGE.

4-7. (canceled)

8. The DVD binding protein of claim 1, wherein the DVD-Ig comprises a polypeptide chain comprising the formula VD1-(X1)n-VD2-C-(X2)n, wherein
VD1 is a first heavy chain variable domain;
VD2 is a second heavy chain variable domain;
C is a heavy chain constant domain;
X1 is a linker with the proviso that it is not CH1;
X2 is an Fc region;
(X1)n is (X1)0 or (X1)1;
(X2)n is (X2)0 or (X2)1.

9. The DVD binding protein of claim 8, wherein
(a) VD1 or VD2 comprises three CDRs, wherein at least one CDR comprises an amino acid sequence selected from the group consisting of SEQ ID NOs: 141, 142, 143, 147, 148, 149, 172, 173, and 174,
(b) VD1 or VD2 comprises three CDRs, wherein at least one CDR comprises an amino acid sequence selected from the group consisting of SEQ ID NOs: 76, 77, 78, 82, 83, 115, 116, and 117;
(c) VD1 or VD2 comprises three CDRs, wherein at least one CDR comprises an amino acid sequence selected from the group consisting of SEQ ID NOs: 109, 110, and 111;
(d) VD1 or VD2 comprises three CDRs, wherein at least one CDR comprises an amino acid sequence selected from the group consisting of SEQ ID NOs: 121, 122, and 123;
(e) VD1 or VD2 comprises three CDRs, wherein at least one CDR comprises an amino acid sequence selected from the group consisting of SEQ ID NOs: 136, 137, 138, 139, and 140;
(f) VD1 or VD2 comprises three CDRs, wherein at least one CDR comprises an amino acid sequence selected from the group consisting of SEQ ID NOs: 153, 154, and 155;
(g) VD1 or VD2 comprises three CDRs, wherein at least one CDR comprises an amino acid sequence selected from the group consisting of SEQ ID NOs: 156, 157, and 158;
(h) VD1 or VD2 comprises three CDRs, wherein at least one CDR comprises an amino acid sequence selected from the group consisting of SEQ ID NOs: 164, 165, and 166;
(i) VD1 or VD2 comprises three CDRs, wherein at least one CDR comprises an amino acid sequence selected from the group consisting of SEQ ID NOs: 172, 173, and 174;
(j) VD1 or VD2 independently comprises an amino acid sequence selected from the group consisting of SEQ ID NOs: 38, 99 and 101;
(k) VD1 or VD2 independently comprises an amino acid sequence of SEQ ID NO: 186;
(l) VD1 or VD2 independently comprises an amino acid sequence of SEQ ID NO: 58;
(m) VD1 or VD2 independently comprises an amino acid sequence selected from the group consisting of SEQ ID NOs: 93, 94, 95, 96, 97 and 98;
(n) VD1 or VD2 independently-comprises an amino acid sequence of SEQ ID NO: 103;
(o) VD1 or VD2 independently comprises an amino acid sequence of SEQ ID NO: 104;
(p) VD1 or VD2 independently comprises an amino acid sequence of SEQ ID NO: 162;
(q) VD1 or VD2 independently comprises an amino acid sequence of SEQ ID NO: 170
(r) VD1 or VD2 independently comprises an amino acid sequence selected from the group consisting of SEQ ID NOs: 30, 32, 34, 36, and 56;
(s) VD1 comprises an amino acid sequence selected from the group consisting of SEQ ID NOs: 30, 32, 34, 38, 56, 103, and 104, and VD2 comprises an amino acid sequence selected from the group consisting of SEQ ID NOs: 38, 58, 93, 94, 95, 96, 97, 98, 99, 101, 162, 170, and 186;
(t) VD1 comprises an amino acid sequence selected from the group consisting of SEQ ID NOs: 38, 58, 93, 94, 95, 96, 97, 98, 99, 101, 162, 170, and 186, and VD2 comprises an amino acid sequence selected from the group consisting of SEQ ID NOs: 30, 32, 34, 36, 38, 56, 103, and 104;
(u) VD1, linker and VD2 of the polypeptide chain in combination comprise an amino acid sequence selected from SEQ ID NOs: 40, 42, 44, 46, 48, 50, 52, 54, 60, 62, 64, 74, 188, and 190;
(v) VD1, linker and VD2 of the polypeptide chain in combination comprise an amino acid sequence selected from SEQ ID NOs: 66, 68, and 192;
(w) VD1, linker and VD2 of the polypeptide chain in combination comprise an amino acid sequence selected from SEQ ID NOs: 180, and 182; or
(x) VD1, linker and VD2 of the polypeptide chain in combination comprise 184.

10. The DVD binding protein of claim 1, wherein the DVD-Ig comprises a polypeptide chain comprising the formula VD1-(X1)n-VD2-C-(X2)n, wherein
VD1 is a first light chain variable domain;
VD2 is a second light chain variable domain;
C is a light chain constant domain;
X1 is a linker with the proviso that it is not CH1;
X2 is an Fc region;
(X1)n is (X1)0 or (X1)1;
(X2)n is (X2)0 or (X2)1.

11. The DVD binding protein of claim 10, wherein
(a) VD1 or VD2 comprises three CDRs, wherein at least one CDR comprises an amino acid sequence selected from the group consisting of SEQ ID NOs: 144, 145, 146, 150, 151, and 152;
(b) VD1 or VD2 comprises three CDRs, wherein at least one CDR comprises an amino acid sequence selected from the group consisting of SEQ ID NOs: 79, 80, 81, 84, 85, 86, 112, 113, 114, 118, 119, and 120;
(c) VD1 or VD2 comprises three CDRs, wherein at least one CDR comprises an amino acid sequence selected from the group consisting of SEQ ID NOs: 112, 113, and 114;
(d) VD1 or VD2 comprises three CDRs, wherein at least one CDR comprises an amino acid sequence selected from the group consisting of SEQ ID NOs: 124, 125, and 126;
(e) VD1 or VD2 comprises three CDRs, wherein at least one CDR comprises an amino acid sequence selected from the group consisting of SEQ ID NOs: 127, 128, 129, 130, 131, 132, 133, 134, and 135;
(f) VD1 or VD2 comprises three CDRs, wherein at least one CDR comprises an amino acid sequence selected from the group consisting of SEQ ID NOs: 153, 154, and 155;
(g) VD1 or VD2 comprises three CDRs, wherein at least one CDR comprises an amino acid sequence selected from the group consisting of SEQ ID NOs: 159, 160, and 161;
(h) VD1 or VD2 comprises three CDRs, wherein at least one CDR comprises an amino acid sequence selected from the group consisting of SEQ ID NOs: 167, 168, and 169;
(i) VD1 or VD2 comprises three CDRs, wherein at least one CDR comprises an amino acid sequence selected from the group consisting of SEQ ID NOs: 175, 176, and 177;
(j) VD1 or VD2 independently comprises an amino acid sequence selected from the group consisting of SEQ ID NOs: 39, 100 and 102;
(k) VD1 or VD2 independently comprises an amino acid sequence of SEQ ID NO: 187;
(l) VD1 or VD2 independently comprises an amino acid sequence of SEQ ID NO: 59;
(m) VD1 or VD2 independently comprises an amino acid sequence selected from the group consisting of SEQ ID NOs: 87, 88, 89, 90, 91, and 92;
(m) VD1 or VD2 independently comprises an amino acid sequence of SEQ ID NO: 103;
(n) VD1 or VD2 independently comprises an amino acid sequence selected from the group consisting of SEQ ID NOs: 105, 106, 107, and 108;
(o) VD1 or VD2 independently comprises an amino acid sequence of SEQ ID NO: 163;
(p) VD1 or VD2 independently comprises an amino acid sequence of SEQ ID NO: 171;
(q) VD1 or VD2 independently comprises an amino acid sequence selected from the group consisting of SEQ ID NOs: 31, 33, 35, 37, 39, and 57;
(r) VD1 comprises an amino acid sequence selected from the group consisting of SEQ ID NOs: 31, 33, 35, 37, 39, 57, 59, 103, 105, 106, 107, and 108, and VD2 comprises an amino acid sequence selected from the group consisting of SEQ ID NOs: 39, 87, 88, 89, 90, 91, 92, 100, 102, 163, and 171;
(s) VD1 comprises an amino acid sequence selected from the group consisting of SEQ ID NOs: 39, 87, 88, 89, 90, 91, 92, 100, 102, 163 and 171, and VD2 comprises an amino acid sequence selected from the group consisting of SEQ ID NO: 31, 33, 35, 37, 39, 57, 59, 103, 105, 106, 107, and 108; and/or
(t) VD1, linker and VD2 in combination comprise an amino acid sequence selected from SEQ ID NOs: 41, 43, 45, 47, 49, 51, 53, 55, 61, 63, 65, 75, 189, and 191;
(u) VD1, linker and VD2 in combination comprise an amino acid sequence selected from SEQ ID NOs:67, 69, and 193;
(v) VD1, linker and VD2 in combination comprise an amino acid sequence selected from SEQ ID NOs: 181, and 183;
(w) VD1, linker and VD2 in combination comprise an amino acid sequence of SEQ ID NO: 185.

12. The DVD binding protein of claim 8, wherein (X1)n is (X1)0; or
wherein the linker comprises an amino acid sequence selected from the group consisting of AKTTPKLEEGEFSEAR (SEQ ID NO: 1); AKTTPKLEEGEFSEARV (SEQ ID NO: 2); AKTTPKLGG (SEQ ID NO: 3); SAKTTPKLGG (SEQ ID NO: 4); SAKTTP (SEQ ID NO: 5); RADAAP (SEQ ID NO: 6); RADAAPTVS (SEQ ID NO: 7); RADAAAAGGPGS (SEQ ID NO: 8); RADAAAA (G₄S)₄ (SEQ ID NO: 9) SAKTTPKLEEGEFSEARV (SEQ ID NO: 10); ADAAP (SEQ ID NO: 11); ADAAPTVSIFPP (SEQ ID NO: 12); TVAAP (SEQ ID NO: 13); TVAAPSVFIFPP (SEQ ID NO: 14); QPKAAP (SEQ ID NO: 15); QPKAAPSVTLFPP (SEQ ID NO: 16); AKTTPP (SEQ ID NO: 17); AKTTPPSVTPLAP (SEQ ID NO: 18); AKTTAP (SEQ ID NO: 19); AKTTAPSVYPLAP (SEQ ID NO: 20); ASTKGP (SEQ ID NO: 21); ASTKGPSVFPLAP (SEQ ID NO: 22), GGGGSGGGGSGGGGS (SEQ ID NO: 23); GENKVEYAPALMALS (SEQ ID NO: 24); GPAKELTPLKEAKVS (SEQ ID NO: 25); or GHEAAAVMQVQYPAS (SEQ ID NO: 26); TVAAPSVFIFPPTVAAPSVFIFPP (SEQ ID NO: 27); ASTKGPSVFPLAPASTKGPSVFPLAP (SEQ ID NO: 28); G/S based sequences (e.g., G4S repeats; SEQ ID NO: 29); GGGGSGGGGS (SEQ ID NO:178)ⱼ GGSGGGGSG (SEQ ID NO:179), GSGSGNGS (SEQ ID NO: 209), GSGSGSGS (SEQ ID NO: 210 GGSGSGSG (SEQ ID NO: 211), GGSGSG (SEQ ID NO: 212), GGSG (SEQ ID NO: 213), GGSGNGSG (SEQ ID:214), and GSG (SEQ ID NO: 215); or
wherein the binding protein comprises at least one disulfide bond; or
wherein the binding protein comprises at least one Fc constant region.

13-16. (canceled)

17. An isolated nucleic acid encoding the DVD binding protein of claim 1.

18. A vector comprising an isolated nucleic acid of claim 17.

19. A host cell comprising a vector of claim 18.

20-21. (canceled)

22. A method of producing a DVD binding protein, the method comprising the step of culturing a host cell of claim 19 in culture medium under conditions sufficient to produce the DVD binding protein.

23. A protein produced by the method of claim 22.

24. A pharmaceutical composition comprising the DVD binding protein of claim 1, and a pharmaceutically acceptable carrier.

25. The pharmaceutical composition of claim 24, further comprising at least one additional therapeutic agent.

26. The pharmaceutical composition of claim 25, wherein the additional therapeutic agent is selected from the group consisting of an imaging agent, a cytotoxic agent, an angiogenesis inhibitor, a kinase inhibitor, a co-stimulation molecule blocker, an adhesion molecule blocker, an anti-cytokine antibody or functional fragment thereof, a detectable label or reporter, an antirheumatic, a muscle relaxant, a narcotic, a non-steroid anti-inflammatory drug (NSAID), an analgesic, an anesthetic, a sedative, a neuromuscular blocker, an antimicrobial, an antipsoriatic, a corticosteriod, an anabolic steroid, an erythropoietin, an immunoglobulin, an immunosuppressive, a growth hormone, a hormone replacement drug, a radiopharmaceutical, an antidepressant, an antipsychotic, a stimulant, an asthma medication, a beta agonist, an inhaled steroid, an epinephrine or analog, a cytokine, and a cytokine antagonist;
or wherein the additional therapeutic agent is selected from the group consisting of budenoside, epidermal growth factor, a corticosteroid, cyclosporin, sulfasalazine, an amino salicylate, 6-mercaptopurine, azathioprine, metronidazole, a lipoxygenase inhibitor mesalamine olsalazine balsalazide, an antioxidant, a thromboxane inhibitor, a growth factor, an elastase inhibitor, a pyridinyl-imidazole compound, an antibody, antagonist or agonist of TNF, LT, IL-1, IL-1R, IL-2, IL-4, IL-6, IL-6R, IL-7, IL-8, IL-10, IL-11, IL-12, IL-13, IL-15, IL-16, IL-18, IL-23, TGF-β, EMAP-II, GM-CSF, FGF, PDGF, CD2, CD3, CD4, CD8, CD-19, CD25, CD28, CD30, CD40, CD45, CD69, CD90 or a ligand thereof, methotrexate, FK506, rapamycin, mycophenolate mofetil, leflunomide, ibuprofen, prednisolone, a phosphodiesterase inhibitor, an adenosine agonist, an antithrombotic agent, a complement inhibitor, an adrenergic agent, IRAK, NIK, IKK, p38, a MAP kinase inhibitor, an IL-1β converting enzyme inhibitor, a TNFα-converting enzyme inhibitor, a T-cell signaling inhibitor, a metalloproteinase inhibitor, an angiotensin converting enzyme inhibitor, a soluble cytokine receptor, a soluble p55 TNF receptor, a soluble p75 TNF receptor, sIL-1RI, sIL-1RII, sIL-6R and combinations thereof.

27-30. (canceled)

31. A method for treating Alzheimer's disease in a subject and/or for modulating Abeta levels within a cell in a subject's central nervous system, the method comprising administering a binding protein, wherein the binding protein comprises a dual variable domain (DVD) binding protein comprising first and second polypeptide chains, wherein said first polypeptide chain comprises a first VD1-(X1)n-VD2-C-(X2)n, wherein
VD1 is a first heavy chain variable domain;
VD2 is a second heavy chain variable domain;
C is a heavy chain constant domain;
X1 is a linker with the proviso that it is not CH1;
X2 is an Fc region;
n is 0 or 1; and
wherein said second polypeptide chain comprises a second VD1-(X1)n-VD2-C-(X2)n, wherein
VD1 is a first light chain variable domain;
VD2 is a second light chain variable domain;
C is a light chain constant domain;
X1 is a linker with the proviso that it is not CH1;
X2 does not comprise an Fc region;
n is 0 or 1; and
wherein the binding protein comprises at least a first and a second binding domain, wherein the first binding domain specifically binds a target that facilitates entrance of the binding protein into a brain, and the second binding domain specifically binds to Abeta, and wherein the binding protein is effective for modulating the Abeta levels within the brain of the subject.

32-34. (canceled)

35. The method of claim 31, wherein the Abeta is intracellular.

36. The method of claim 31, wherein the Abeta is cell membrane bound.

37. (canceled)

38. A method for treating multiple sclerosis in a subject and/or for modulating myelin basic protein levels within a cell in a subject's central nervous system, the method comprising administering a binding protein, wherein the binding protein comprises a dual variable domain (DVD) binding protein comprising first and second polypeptide chains, wherein said first polypeptide chain comprises a first VD1-(X1)n-VD2-C-(X2)n, wherein
VD1 is a first heavy chain variable domain;
VD2 is a second heavy chain variable domain;
C is a heavy chain constant domain;
X1 is a linker with the proviso that it is not CH1;
X2 is an Fc region;
n is 0 or 1; and
wherein said second polypeptide chain comprises a second VD1-(X1)n-VD2-C-(X2)n, wherein
VD1 is a first light chain variable domain;
VD2 is a second light chain variable domain;
C is a light chain constant domain;
X1 is a linker with the proviso that it is not CH1;
X2 does not comprise an Fc region;
n is 0 or 1; and
wherein the binding protein comprises at least a first and a second binding domain, wherein the first binding domain specifically binds a target that facilitates entrance of the binding protein into a brain, and the second binding domain specifically binds to RGMa, and wherein the binding protein is effective for modulating the RGMa levels within the brain of the subject.

39-41. (canceled)

42. A method for treating Parkinson's disease in a subject comprising administering a binding protein, wherein the binding protein comprises a dual variable domain (DVD) binding protein comprising first and second polypeptide chains, wherein said first polypeptide chain comprises a first VD1-(X1)n-VD2-C-(X2)n, wherein
VD1 is a first heavy chain variable domain;
VD2 is a second heavy chain variable domain;
C is a heavy chain constant domain;
X1 is a linker with the proviso that it is not CH1;
X2 is an Fc region;
n is 0 or 1; and
wherein said second polypeptide chain comprises a second VD1-(X1)n-VD2-C-(X2)n, wherein
VD1 is a first light chain variable domain;
VD2 is a second light chain variable domain;
C is a light chain constant domain;
X1 is a linker with the proviso that it is not CH1;
X2 does not comprise an Fc region;
n is 0 or 1; and
comprising at least a first and a second binding domain, wherein the first binding domain specifically binds a target that facilitates entrance of TNF into the brain, and wherein the binding protein reduces the loss of dopaminergic neurons or is effective for modulating the concentration of TNF within the brain and/or cerebrospinal fluid of the subject.

43. (canceled)

44. A method for treating a disease or condition in a subject in need thereof, the method comprising administering a binding protein, wherein the binding protein comprises a dual variable domain (DVD) binding protein comprising first and second polypeptide chains, wherein said first polypeptide chain comprises a first VD1-(X1)n-VD2-C-(X2)n, wherein
VD1 is a first heavy chain variable domain;
VD2 is a second heavy chain variable domain;
C is a heavy chain constant domain;
X1 is a linker with the proviso that it is not CH1;
X2 is an Fc region;
n is 0 or 1; and
wherein said second polypeptide chain comprises a second VD1-(X1)n-VD2-C-(X2)n, wherein
VD1 is a first light chain variable domain;
VD2 is a second light chain variable domain;
C is a light chain constant domain;
X1 is a linker with the proviso that it is not CH1;
X2 does not comprise an Fc region;
n is 0 or 1; and
wherein the binding protein comprises at least a first and a second binding domain, wherein the first binding domain specifically binds a target that facilitates entrance into the brain, and the second binding domain specifically binds to BACE1, HER2, or APP, and wherein administration of the binding protein to a subject is effective for modulating the concentration of BACE1, HER2, or APP in the brain or cerebrospinal fluid of the subject.

45. The method of claim 44, wherein the disease or condition is neurological or associated with cancer.

46. The method of claim 44, wherein the disease or condition is Alzheimer's disease, Parkinson's disease, or multiple sclerosis.

47. (canceled)

48. The binding protein of claim 31, wherein the
(a) VD1 or VD2 of the first polypeptide chain comprises three CDRs, wherein at least one CDR comprises an amino acid sequence selected from the group consisting of SEQ ID NOs: 141, 142, 143, 147, 148, 149, 172, 173, and 174;
(b) VD1 or VD2 of the first polypeptide chain comprises an amino acid sequence selected from the group consisting of 38, 99, 101, 198, 199, 201, 202, 206, 207, and 208;
(c) VD1 or VD2 of the second polypeptide chain comprises three CDRs, wherein at least one CDR comprises an amino acid sequence selected from the group consisting of SEQ ID NOs: 144, 145, 146, 150, 151, and 152;
(d) VD1 or VD2 of the second polypeptide chain in combination comprise an amino acid sequence selected from the group consisting of SEQ ID NOs: 39, 100, 102, 196, 197, 200, 203, 204, and 205;
(e) VD1, linker, and VD2 of the first polypeptide chain in combination comprise the amino acid sequence selected from the group consisting of SEQ ID NOs: 188 and 190; or
(f) VD1, linker, and VD2 of the second polypeptide chain in combination comprise the amino acid sequence selected from the group consisting of SEQ ID NOs: 189 and 191.

49. The binding protein of claim 38, wherein the
(a) VD1 or VD2 of the first polypeptide chain comprises three CDRs, wherein at least one CDR comprises an amino acid sequence selected from the group consisting of SEQ ID NOs: 172, 173, and 174;
(b) VD1 or VD2 of the first polypeptide chain comprises the amino acid sequence of SEQ ID NO: 170;
(c) VD1 or VD2 of the second polypeptide chain comprises three CDRs, wherein at least one CDR comprises an amino acid sequence selected from the group consisting of SEQ ID NO: 175, 176, and 177;
(d) VD1 or VD2 of the second polypeptide chain comprises the amino acid sequence of SEQ ID NO: 171;
(e) VD1, linker, and VD2 of the first polypeptide chain in combination comprise the amino acid sequence of SEQ ID NO: 184; or
(f) VD1, linker, and VD2 of the second polypeptide chain in combination comprise the amino acid sequence selected from the group consisting of 185; or

50. The binding protein of claim 42, wherein the
(a) VD1 or VD2 of the first polypeptide chain comprises three CDRs, wherein at least one CDR comprises an amino acid sequence selected from the group consisting of SEQ ID NOs: 164, 165, and 165;
(b) VD1 or VD2 of the first polypeptide chain comprises the amino acid sequence of SEQ ID NO: 162;
(c) VD1 or VD2 of the second polypeptide chain comprises three CDRs, wherein at least one CDR comprises an amino acid sequence selected from the group consisting of SEQ ID NO: 167, 168, and 169;
(d) VD1 or VD2 of the second polypeptide chain comprises the amino acid sequence of SEQ ID NO: 163;
(e) VD1, linker, and VD2 of the first polypeptide chain in combination comprise the amino acid sequence of SEQ ID NO: 182; or
(f) VD1, linker, and VD2 of the second polypeptide chain in combination comprise the amino acid sequence selected from the group consisting of SEQ ID NO: 184.

51. The binding protein of claim 44, wherein the
(a) VD1 or VD2 of the first polypeptide comprises three CDRs, wherein at least one CDR comprises an amino acid sequence selected from the group consisting of SEQ ID NOs: 121, 122, and 123;
(b) VD1 or VD2 of the first polypeptide chain comprises the amino acid sequence of SEQ ID NO: 58;
(c) VD1 or VD2 of the second polypeptide comprises three CDRs, wherein at least one CDR comprises an amino acid sequence selected from the group consisting of SEQ ID NOs: 124, 125, and 126;
(d) VD1 or VD2 of the first polypeptide chain comprises the amino acid sequence of SEQ ID NO: 59;
(e) VD1, linker and VD2 of the first polypeptide chain in combination comprise an amino acid sequence selected from the group consisting of SEQ ID NOs: 66, 68, and 192; or
(f) VD1, linker and VD2 of the second polypeptide chain in combination comprise an amino acid sequence selected from the group consisting of SEQ ID NOs: 67, 69, and 193.

52. The binding protein of claim 44, wherein the
(a) VD1 or VD2 of the first polypeptide chain comprises three CDRs, wherein at least one CDR comprises an amino acid sequence selected from the group consisting of SEQ ID NOs: 136, 137, 138, 139, and 140;
(b) VD1 or VD2 of the first polypeptide chain comprises an amino acid sequence selected from the group consisting of SEQ ID NOs: 93, 94, 95, 97, and 98;
(c) VD1 or VD2 of the second polypeptide chain comprises three CDRs, wherein at least one CDR comprises an amino acid sequence selected from the group consisting of SEQ ID NOs: 127, 128, 129, 130, 131, 132, 133, 134, and 140; or
(d) VD1 or VD2 comprises of the second polypeptide chain comprises an amino acid sequence selected from the group consisting of SEQ ID NOs: 87, 88, 89, 90, 91, and 92;

53. The binding protein of claim 44, wherein the
(a) VD1 or VD2 of the first polypeptide chain comprises three CDRs, wherein at least one CDR comprises an amino acid sequence selected from the group consisting from the group of SEQ ID NOs: 109, 110, and 111;
(b) VD1 or VD2 of the second polypeptide chain comprises an amino acid sequence of SEQ ID NOs: 186;
(c) VD1 or VD2 of the second polypeptide chain comprises three CDRs, wherein at least one CDR comprises an amino acid sequence selected from the group consisting of SEQ ID NOs: 112, 113, and 114;
(d) VD1 or VD2 of the second polypeptide chain comprises an amino acid sequence selected from the group from the group consisting of 187;
(e) VD1, linker, and VD2 of the first polypeptide chain in combination comprises an amino acid sequence selected from the group of SEQ ID NOs: 40, 42, 44, 46, 48, 50, 52, 54, 60, 62, 64, and 74; or
(f) VD1, linker, and VD2 of the second polypeptide chain in combination comprise an amino acid sequence selected from the group of SEQ ID NOs: 41, 43, 45, 47, 49, 51, 53, 55, 61, 63, 65, and 75.


SEQUENCE LISTING (215 sequences)

SEQ ID NO: 1 - protein, 16 aa, Artificial Sequence
  Description of Artificial Sequence: Synthetic peptide
AKTTPKLEEGEFSEAR

SEQ ID NO: 2 - protein, 17 aa, Artificial Sequence
  Description of Artificial Sequence: Synthetic peptide
AKTTPKLEEGEFSEARV

SEQ ID NO: 3 - protein, 9 aa, Artificial Sequence
  Description of Artificial Sequence: Synthetic peptide
AKTTPKLGG

SEQ ID NO: 4 - protein, 10 aa, Artificial Sequence
  Description of Artificial Sequence: Synthetic peptide
SAKTTPKLGG

SEQ ID NO: 5 - protein, 6 aa, Artificial Sequence
  Description of Artificial Sequence: Synthetic peptide
SAKTTP

SEQ ID NO: 6 - protein, 6 aa, Artificial Sequence
  Description of Artificial Sequence: Synthetic peptide
RADAAP

SEQ ID NO: 7 - protein, 9 aa, Artificial Sequence
  Description of Artificial Sequence: Synthetic peptide
RADAAPTVS

SEQ ID NO: 8 - protein, 12 aa, Artificial Sequence
  Description of Artificial Sequence: Synthetic peptide
RADAAAAGGPGS

SEQ ID NO: 9 - protein, 27 aa, Artificial Sequence
  Description of Artificial Sequence: Synthetic peptide
RADAAAAGGGGSGGGGSGGGGSGGGGS

SEQ ID NO: 10 - protein, 18 aa, Artificial Sequence
  Description of Artificial Sequence: Synthetic peptide
SAKTTPKLEEGEFSEARV

SEQ ID NO: 11 - protein, 5 aa, Artificial Sequence
  Description of Artificial Sequence: Synthetic peptide
ADAAP

SEQ ID NO: 12 - protein, 12 aa, Artificial Sequence
  Description of Artificial Sequence: Synthetic peptide
ADAAPTVSIFPP

SEQ ID NO: 13 - protein, 5 aa, Artificial Sequence
  Description of Artificial Sequence: Synthetic peptide
TVAAP

SEQ ID NO: 14 - protein, 12 aa, Artificial Sequence
  Description of Artificial Sequence: Synthetic peptide
TVAAPSVFIFPP

SEQ ID NO: 15 - protein, 6 aa, Artificial Sequence
  Description of Artificial Sequence: Synthetic peptide
QPKAAP

SEQ ID NO: 16 - protein, 13 aa, Artificial Sequence
  Description of Artificial Sequence: Synthetic peptide
QPKAAPSVTLFPP

SEQ ID NO: 17 - protein, 6 aa, Artificial Sequence
  Description of Artificial Sequence: Synthetic peptide
AKTTPP

SEQ ID NO: 18 - protein, 13 aa, Artificial Sequence
  Description of Artificial Sequence: Synthetic peptide
AKTTPPSVTPLAP

SEQ ID NO: 19 - protein, 6 aa, Artificial Sequence
  Description of Artificial Sequence: Synthetic peptide
AKTTAP

SEQ ID NO: 20 - protein, 13 aa, Artificial Sequence
  Description of Artificial Sequence: Synthetic peptide
AKTTAPSVYPLAP

SEQ ID NO: 21 - protein, 6 aa, Artificial Sequence
  Description of Artificial Sequence: Synthetic peptide
ASTKGP

SEQ ID NO: 22 - protein, 13 aa, Artificial Sequence
  Description of Artificial Sequence: Synthetic peptide
ASTKGPSVFPLAP

SEQ ID NO: 23 - protein, 15 aa, Artificial Sequence
  Description of Artificial Sequence: Synthetic peptide
GGGGSGGGGSGGGGS

SEQ ID NO: 24 - protein, 15 aa, Artificial Sequence
  Description of Artificial Sequence: Synthetic peptide
GENKVEYAPALMALS

SEQ ID NO: 25 - protein, 15 aa, Artificial Sequence
  Description of Artificial Sequence: Synthetic peptide
GPAKELTPLKEAKVS

SEQ ID NO: 26 - protein, 15 aa, Artificial Sequence
  Description of Artificial Sequence: Synthetic peptide
GHEAAAVMQVQYPAS

SEQ ID NO: 27 - protein, 24 aa, Artificial Sequence
  Description of Artificial Sequence: Synthetic peptide
TVAAPSVFIFPPTVAAPSVFIFPP

SEQ ID NO: 28 - protein, 26 aa, Artificial Sequence
  Description of Artificial Sequence: Synthetic peptide
ASTKGPSVFPLAPASTKGPSVFPLAP

SEQ ID NO: 29 - protein, 5 aa, Artificial Sequence
  Description of Artificial Sequence: Synthetic peptide
GGGGS

SEQ ID NO: 30 - protein, 118 aa, Artificial Sequence
  Description of Artificial Sequence: Synthetic polypeptide
EVQLVESGGGLVQPGGSLRLSCAASGFTFSNYGMHWIRQAPGKGLEWIAMIYYDSSKMNYADTVKGRFTISRDNAKNSLYLQMNSLRAEDTAVYYCAVPTSHYVVDVWGQGTTVTVSS

SEQ ID NO: 31 - protein, 108 aa, Artificial Sequence
  Description of Artificial Sequence: Synthetic polypeptide
AIQMTQSPSSLSASVGDRVTITCQASQDIGNWLAWYQQKPGKSPKLLIYGATSLADGVPSRFSGSRSGTDFTLTISSLQPEDFATYYCLQAYNTPWTFGGGTKVEIKR

SEQ ID NO: 32 - protein, 118 aa, Artificial Sequence; identical to SEQ ID NO: 30
  Description of Artificial Sequence: Synthetic polypeptide

SEQ ID NO: 33 - protein, 108 aa, Artificial Sequence
  Description of Artificial Sequence: Synthetic polypeptide
EIVMTQSPATLSVSPGERATLSCQASQDIGNWLAWYQQKPGQSPRLLIYGATSLADGVPARFSGSRSGTEFTLTISSLQSEDFAVYYCLQAYNTPWTFGGGTKVEIKR

SEQ ID NO: 34 - protein, 118 aa, Artificial Sequence
  Description of Artificial Sequence: Synthetic polypeptide
QVQLVQSGAEVKKPGASVKVSCKASGFTFSNYGMHWIRQAPGQGLEWIAMIYYDSSKMNYADTVKGRFTITRDNSTNTLYMELSSLRSEDTAVYYCAVPTSHYVVDVWGQGTTVTVSS

SEQ ID NO: 35 - protein, 108 aa, Artificial Sequence; identical to SEQ ID NO: 31
  Description of Artificial Sequence: Synthetic polypeptide

SEQ ID NO: 36 - protein, 118 aa, Artificial Sequence; identical to SEQ ID NO: 34
  Description of Artificial Sequence: Synthetic polypeptide

SEQ ID NO: 37 - protein, 108 aa, Artificial Sequence; identical to SEQ ID NO: 33
  Description of Artificial Sequence: Synthetic polypeptide

SEQ ID NO: 38 - protein, 119 aa, Artificial Sequence
  Description of Artificial Sequence: Synthetic polypeptide
EVKLVESGGGLVKPGASLKLSCAASGFTFSNYGMSWVRQNSDKRLEWVASIRSGGGRTYYSDNVKGRFTISRENAKNTLYLQMSSLKSEDTALYYCVRYDHYSGSSDYWGQGTTVTVSS

SEQ ID NO: 39 - protein, 112 aa, Artificial Sequence
  Description of Artificial Sequence: Synthetic polypeptide
YVVMTQTPLTLSVTIGQPASISCKSSQSLLDSDGKTYLNWLLQRPGQSPKRLIYLVSKLDSGVPDRFTGSGSGTDFTLKISRIEAEDLGLYYCWQGTHFPRTFGGGTKLEIK

SEQ ID NO: 40 - protein, 243 aa, Artificial Sequence
  Description of Artificial Sequence: Synthetic polypeptide
EVQLVESGGGLVQPGGSLRLSCAASGFTFSNYGMHWIRQAPGKGLEWIAMIYYDSSKMNYADTVKGRFTISRDNAKNSLYLQMNSLRAEDTAVYYCAVPTSHYVVDVWGQGTTVTVSSASTKGPEVQLLESGGGLVQPGGSLRLSCAASGFTFSNYGMSWVRQAPGKGLEWVASIRSGGGRTYYSDNVKGRFTISRDNSKNTLYLQMNSLRAEDTAVYYCVRYDHYSGSSDYWGQGTLVTVSS

SEQ ID NO: 41 - protein, 226 aa, Artificial Sequence
  Description of Artificial Sequence: Synthetic polypeptide
AIQMTQSPSSLSASVGDRVTITCQASQDIGNWLAWYQQKPGKSPKLLIYGATSLADGVPSRFSGSRSGTDFTLTISSLQPEDFATYYCLQAYNTPWTFGGGTKVEIKRTVAAPDVVMTQSPLSLPVTPGEPASISCKSSQSLLDSDGKTYLNWLLQKPGQSPQRLIYLVSKLDSGVPDRFSGSGSGTDFTLKISRVEAEDVGVYYCWQGTHFPRTFGQGTKVEIKR

SEQ ID NO: 42 - protein, 243 aa, Artificial Sequence
  Description of Artificial Sequence: Synthetic polypeptide
EVQLLESGGGLVQPGGSLRLSCAASGFTFSNYGMSWVRQAPGKGLEWVASIRSGGGRTYYSDNVKGRFTISRDNSKNTLYLQMNSLRAEDTAVYYCVRYDHYSGSSDYWGQGTLVTVSSASTKGPEVQLVESGGGLVQPGGSLRLSCAASGFTFSNYGMHWIRQAPGKGLEWIAMIYYDSSKMNYADTVKGRFTISRDNAKNSLYLQMNSLRAEDTAVYYCAVPTSHYVVDVWGQGTTVTVSS

SEQ ID NO: 43 - protein, 226 aa, Artificial Sequence
  Description of Artificial Sequence: Synthetic polypeptide
DVVMTQSPLSLPVTPGEPASISCKSSQSLLDSDGKTYLNWLLQKPGQSPQRLIYLVSKLDSGVPDRFSGSGSGTDFTLKISRVEAEDVGVYYCWQGTHFPRTFGQGTKVEIKRTVAAPAIQMTQSPSSLSASVGDRVTITCQASQDIGNWLAWYQQKPGKSPKLLIYGATSLADGVPSRFSGSRSGTDFTLTISSLQPEDFATYYCLQAYNTPWTFGGGTKVEIKR

SEQ ID NO: 44 - protein, 243 aa, Artificial Sequence; identical to SEQ ID NO: 40
  Description of Artificial Sequence: Synthetic polypeptide

SEQ ID NO: 45 - protein, 226 aa, Artificial Sequence
  Description of Artificial Sequence: Synthetic polypeptide
EIVMTQSPATLSVSPGERATLSCQASQDIGNWLAWYQQKPGQSPRLLIYGATSLADGVPARFSGSRSGTEFTLTISSLQSEDFAVYYCLQAYNTPWTFGGGTKVEIKRTVAAPDVVMTQSPLSLPVTPGEPASISCKSSQSLLDSDGKTYLNWLLQKPGQSPQRLIYLVSKLDSGVPDRFSGSGSGTDFTLKISRVEAEDVGVYYCWQGTHFPRTFGQGTKVEIKR

SEQ ID NO: 46 - protein, 243 aa, Artificial Sequence; identical to SEQ ID NO: 42
  Description of Artificial Sequence: Synthetic polypeptide

SEQ ID NO: 47 - protein, 226 aa, Artificial Sequence
  Description of Artificial Sequence: Synthetic polypeptide
DVVMTQSPLSLPVTPGEPASISCKSSQSLLDSDGKTYLNWLLQKPGQSPQRLIYLVSKLDSGVPDRFSGSGSGTDFTLKISRVEAEDVGVYYCWQGTHFPRTFGQGTKVEIKRTVAAPEIVMTQSPATLSVSPGERATLSCQASQDIGNWLAWYQQKPGQSPRLLIYGATSLADGVPARFSGSRSGTEFTLTISSLQSEDFAVYYCLQAYNTPWTFGGGTKVEIKR

SEQ ID NO: 48 - protein, 243 aa, Artificial Sequence
  Description of Artificial Sequence: Synthetic polypeptide
QVQLVQSGAEVKKPGASVKVSCKASGFTFSNYGMHWIRQAPGQGLEWIAMIYYDSSKMNYADTVKGRFTITRDNSTNTLYMELSSLRSEDTAVYYCAVPTSHYVVDVWGQGTTVTVSSASTKGPEVQLLESGGGLVQPGGSLRLSCAASGFTFSNYGMSWVRQAPGKGLEWVASIRSGGGRTYYSDNVKGRFTISRDNSKNTLYLQMNSLRAEDTAVYYCVRYDHYSGSSDYWGQGTLVTVSS

SEQ ID NO: 49 - protein, 226 aa, Artificial Sequence; identical to SEQ ID NO: 41
  Description of Artificial Sequence: Synthetic polypeptide

SEQ ID NO: 50 - protein, 243 aa, Artificial Sequence
  Description of Artificial Sequence: Synthetic polypeptide
EVQLLESGGGLVQPGGSLRLSCAASGFTFSNYGMSWVRQAPGKGLEWVASIRSGGGRTYYSDNVKGRFTISRDNSKNTLYLQMNSLRAEDTAVYYCVRYDHYSGSSDYWGQGTLVTVSSASTKGPQVQLVQSGAEVKKPGASVKVSCKASGFTFSNYGMHWIRQAPGQGLEWIAMIYYDSSKMNYADTVKGRFTITRDNSTNTLYMELSSLRSEDTAVYYCAVPTSHYVVDVWGQGTTVTVSS

SEQ ID NO: 51 - protein, 226 aa, Artificial Sequence; identical to SEQ ID NO: 43
  Description of Artificial Sequence: Synthetic polypeptide

SEQ ID NO: 52 - protein, 243 aa, Artificial Sequence; identical to SEQ ID NO: 48
  Description of Artificial Sequence: Synthetic polypeptide

SEQ ID NO: 53 - protein, 226 aa, Artificial Sequence; identical to SEQ ID NO: 45
  Description of Artificial Sequence: Synthetic polypeptide

SEQ ID NO: 54 - protein, 243 aa, Artificial Sequence; identical to SEQ ID NO: 50
  Description of Artificial Sequence: Synthetic polypeptide

SEQ ID NO: 55 - protein, 226 aa, Artificial Sequence; identical to SEQ ID NO: 47
  Description of Artificial Sequence: Synthetic polypeptide

SEQ ID NO: 56 - protein, 118 aa, Artificial Sequence
  Description of Artificial Sequence: Synthetic polypeptide
EVQLVESGGGLVQPGNSLTLSCVASGFTFSNYGMHWIRQAPKKGLEWIAMIYYDSSKMNYADTVKGRFTISRDNSKNTLYLEMNSLRSEDTAMYYCAVPTSHYVVDVWGQGVSVTVSS

SEQ ID NO: 57 - protein, 108 aa, Artificial Sequence
  Description of Artificial Sequence: Synthetic polypeptide
DIQMTQSPASLSASLEEIVTITCQASQDIGNWLAWYQQKPGKSPQLLIYGATSLADGVPSRFSGSRSGTQFSLKISRVQVEDIGIYYCLQAYNTPWTFGGGTKLELKR

SEQ ID NO: 58 - protein, 120 aa, Artificial Sequence [sequence omitted]

SEQ ID NO: 59 - protein, 108 aa, Artificial Sequence
  Description of Artificial Sequence: Synthetic polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKR

SEQ ID NO: 60 - protein, 243 aa, Artificial Sequence
  Description of Artificial Sequence: Synthetic polypeptide
EVQLVESGGGLVQPGNSLTLSCVASGFTFSNYGMHWIRQAPKKGLEWIAMIYYDSSKMNYADTVKGRFTISRDNSKNTLYLEMNSLRSEDTAMYYCAVPTSHYVVDVWGQGVSVTVSSASTKGPEVQLLESGGGLVQPGGSLRLSCAASGFTFSNYGMSWVRQAPGKGLEWVASIRSGGGRTYYSDNVKGRFTISRDNSKNTLYLQMNSLRAEDTAVYYCVRYDHYSGSSDYWGQGTLVTVSS

SEQ ID NO: 61 - protein, 226 aa, Artificial Sequence
  Description of Artificial Sequence: Synthetic polypeptide
DIQMTQSPASLSASLEEIVTITCQASQDIGNWLAWYQQKPGKSPQLLIYGATSLADGVPSRFSGSRSGTQFSLKISRVQVEDIGIYYCLQAYNTPWTFGGGTKLELKRTVAAPDVVMTQSPLSLPVTPGEPASISCKSSQSLLDSDGKTYLNWLLQKPGQSPQRLIYLVSKLDSGVPDRFSGSGSGTDFTLKISRVEAEDVGVYYCWQGTHFPRTFGQGTKVEIKR

SEQ ID NO: 62 - protein, 250 aa, Artificial Sequence
  Description of Artificial Sequence: Synthetic polypeptide
EVQLVESGGGLVQPGNSLTLSCVASGFTFSNYGMHWIRQAPKKGLEWIAMIYYDSSKMNYADTVKGRFTISRDNSKNTLYLEMNSLRSEDTAMYYCAVPTSHYVVDVWGQGVSVTVSSASTKGPSVFPLAPEVQLLESGGGLVQPGGSLRLSCAASGFTFSNYGMSWVRQAPGKGLEWVASIRSGGGRTYYSDNVKGRFTISRDNSKNTLYLQMNSLRAEDTAVYYCVRYDHYSGSSDYWGQGTLVTVSS

SEQ ID NO: 63 - protein, 233 aa, Artificial Sequence
  Description of Artificial Sequence: Synthetic polypeptide
DIQMTQSPASLSASLEEIVTITCQASQDIGNWLAWYQQKPGKSPQLLIYGATSLADGVPSRFSGSRSGTQFSLKISRVQVEDIGIYYCLQAYNTPWTFGGGTKLELKRTVAAPSVFIFPPDVVMTQSPLSLPVTPGEPASISCKSSQSLLDSDGKTYLNWLLQKPGQSPQRLIYLVSKLDSGVPDRFSGSGSGTDFTLKISRVEAEDVGVYYCWQGTHFPRTFGQGTKVEIKR

SEQ ID NO: 64 - protein, 243 aa, Artificial Sequence
  Description of Artificial Sequence: Synthetic polypeptide
EVQLLESGGGLVQPGGSLRLSCAASGFTFSNYGMSWVRQAPGKGLEWVASIRSGGGRTYYSDNVKGRFTISRDNSKNTLYLQMNSLRAEDTAVYYCVRYDHYSGSSDYWGQGTLVTVSSASTKGPEVQLVESGGGLVQPGNSLTLSCVASGFTFSNYGMHWIRQAPKKGLEWIAMIYYDSSKMNYADTVKGRFTISRDNSKNTLYLEMNSLRSEDTAMYYCAVPTSHYVVDVWGQGVSVTVSS

SEQ ID NO: 65 - protein, 226 aa, Artificial Sequence
  Description of Artificial Sequence: Synthetic polypeptide
DVVMTQSPLSLPVTPGEPASISCKSSQSLLDSDGKTYLNWLLQKPGQSPQRLIYLVSKLDSGVPDRFSGSGSGTDFTLKISRVEAEDVGVYYCWQGTHFPRTFGQGTKVEIKRTVAAPDIQMTQSPASLSASLEEIVTITCQASQDIGNWLAWYQQKPGKSPQLLIYGATSLADGVPSRFSGSRSGTQFSLKISRVQVEDIGIYYCLQAYNTPWTFGGGTKLELKR

SEQ ID NO: 66 - protein, 244 aa, Artificial Sequence
  Description of Artificial Sequence: Synthetic polypeptide
EVQLVESGGGLVQPGNSLTLSCVASGFTFSNYGMHWIRQAPKKGLEWIAMIYYDSSKMNYADTVKGRFTISRDNSKNTLYLEMNSLRSEDTAMYYCAVPTSHYVVDVWGQGVSVTVSSASTKGPEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSS

SEQ ID NO: 67 - protein, 221 aa, Artificial Sequence
  Description of Artificial Sequence: Synthetic polypeptide
DIQMTQSPASLSASLEEIVTITCQASQDIGNWLAWYQQKPGKSPQLLIYGATSLADGVPSRFSGSRSGTQFSLKISRVQVEDIGIYYCLQAYNTPWTFGGGTKLELKRTVAAPDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKR

SEQ ID NO: 68 - protein, 244 aa, Artificial Sequence
  Description of Artificial Sequence: Synthetic polypeptide
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPEVQLVESGGGLVQPGNSLTLSCVASGFTFSNYGMHWIRQAPKKGLEWIAMIYYDSSKMNYADTVKGRFTISRDNSKNTLYLEMNSLRSEDTAMYYCAVPTSHYVVDVWGQGVSVTVSS

SEQ ID NO: 69 - protein, 221 aa, Artificial Sequence
  Description of Artificial Sequence: Synthetic polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPDIQMTQSPASLSASLEEIVTITCQASQDIGNWLAWYQQKPGKSPQLLIYGATSLADGVPSRFSGSRSGTQFSLKISRVQVEDIGIYYCLQAYNTPWTFGGGTKLELKR

SEQ ID NO: 70 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 71 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 72 - protein, 106 aa, Homo sapiens
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 73 - protein, 105 aa, Homo sapiens
QPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 74 - protein, 250 aa, Artificial Sequence
  Description of Artificial Sequence: Synthetic polypeptide
EVQLLESGGGLVQPGGSLRLSCAASGFTFSNYGMSWVRQAPGKGLEWVASIRSGGGRTYYSDNVKGRFTISRDNSKNTLYLQMNSLRAEDTAVYYCVRYDHYSGSSDYWGQGTLVTVSSASTKGPSVFPLAPEVQLVESGGGLVQPGNSLTLSCVASGFTFSNYGMHWIRQAPKKGLEWIAMIYYDSSKMNYADTVKGRFTISRDNSKNTLYLEMNSLRSEDTAMYYCAVPTSHYVVDVWGQGVSVTVSS

SEQ ID NO: 75 - protein, 226 aa, Artificial Sequence; identical to SEQ ID NO: 65
  Description of Artificial Sequence: Synthetic polypeptide

SEQ ID NO: 76 - protein, 10 aa, Artificial Sequence
  Description of Artificial Sequence: Synthetic peptide
GFTFSNYGMH

SEQ ID NO: 77 - protein, 17 aa, Artificial Sequence
  Description of Artificial Sequence: Synthetic peptide
MIYYDSSKMNYADTVKG

SEQ ID NO: 78 - protein, 9 aa, Artificial Sequence
  Description of Artificial Sequence: Synthetic peptide
PTSHYVVDV

SEQ ID NO: 79 - protein, 11 aa, Artificial Sequence
  Description of Artificial Sequence: Synthetic peptide
QASQDIGNWLA

SEQ ID NO: 80 - protein, 7 aa, Artificial Sequence
  Description of Artificial Sequence: Synthetic peptide
GATSLAD

SEQ ID NO: 81 - protein, 9 aa, Artificial Sequence
  Description of Artificial Sequence: Synthetic peptide
LQAYNTPWT

SEQ ID NO: 82 - protein, 17 aa, Artificial Sequence
  Description of Artificial Sequence: Synthetic peptide
MIYYDSSKMNYADTVKG

SEQ ID NO: 83 - protein, 9 aa, Artificial Sequence
  Description of Artificial Sequence: Synthetic peptide
PTSHYVVDV

SEQ ID NO: 84 - protein, 11 aa, Artificial Sequence
  Description of Artificial Sequence: Synthetic peptide
QASQDIGNWLA

SEQ ID NO: 85 - protein, 7 aa, Artificial Sequence
  Description of Artificial Sequence: Synthetic peptide
GATSLAD

SEQ ID NO: 86 - protein, 9 aa, Artificial Sequence
  Description of Artificial Sequence: Synthetic peptide
LQAYNTPWT

SEQ ID NO: 87 - protein, 108 aa, Artificial Sequence
  Description of Artificial Sequence: Synthetic polypeptide
DIQMTQSPSSLSASVGDRVTITCRASQDVSTAVAWYQQKPGKAPKLLIYSASFLYSGVPSRFSGSGSGTDFTLTISSLQPEDFATYYCQQSYTTPPTFGQGTKVEIKR

SEQ ID NO: 88 - protein, 108 aa, Artificial Sequence
  Description of Artificial Sequence: Synthetic polypeptide
DIQMTQSPSSLSASVGDRVTITCRASQDVSTAVAWYQQKPGKAPKLLIYSASFLYSGVPSRFSGSGSGTDFTLTISSLQPEDFATYYCQQFPTYLPTFGQGTKVEIKR

SEQ ID NO: 89 - protein, 108 aa, Artificial Sequence
  Description of Artificial Sequence: Synthetic polypeptide
DIQMTQSPSSLSASVGDRVTITCRASQDVSTAVAWYQQKPGKAPKLLIYSASFLYSGVPSRFSGSGSGTDFTLTISSLQPEDFATYYCQQGYNDPPTFGQGTKVEIKR

SEQ ID NO: 90 - protein, 108 aa, Artificial Sequence
  Description of Artificial Sequence: Synthetic polypeptide
DIQMTQSPSSLSASVGDRVTITCRASQDVSTAVAWYQQKPGKAPKLLIYSASFLYSGVPSRFSGSGSGTDFTLTISSLQPEDFATYYCQQSSTDPTTFGQGTKVEIKR

SEQ ID NO: 91 - protein, 108 aa, Artificial Sequence
  Description of Artificial Sequence: Synthetic polypeptide
DIQMTQSPSSLSASVGDRVTITCRASQVVANSLAWYQQKPGKAPKLLIYLASFLYSGVPSRFSGSGSGTDFTLTISSLQPEDFATYYCQQDATSPPTFGQGTKVEIKR

SEQ ID NO: 92 - protein, 108 aa, Artificial Sequence
  Description of Artificial Sequence: Synthetic polypeptide
DIQMTQSPSSLSASVGDRVTITCRASQDVSTAVAWYQQKPGKAPKLLIYSASFLYSGVPSRFSGSGSGTDFTLTISSLQPEDFATYYCQQYATDPPTFGQGTKVEIKR

SEQ ID NO: 93 - protein, 119 aa, Artificial Sequence
  Description of Artificial Sequence: Synthetic polypeptide
EVQLVESGGGLVQPGGSLRLSCAASGFTFSGYAIHWVRQAPGKGLEWVGWISPAGGSTDYADSVKGRFTISADTSKNTAYLQMNSLRAEDTAVYYCARGPFSPWVMDYWGQGTLVTVSS

SEQ ID NO: 94 - protein, 119 aa, Artificial Sequence
  Description of Artificial Sequence: Synthetic polypeptide
EVQLVESGGGLVQPGGSLRLSCAASGFTFLGYGIHWVRQAPGKGLEWVGWISPAGGSTDYADSVKGRFTISADTSKNTAYLQMNSLRAEDTAVYYCARGPFSPWVMDYWGQGTLVTVSS

SEQ ID NO: 95 - protein, 119 aa, Artificial Sequence; identical to SEQ ID NO: 93
  Description of Artificial Sequence: Synthetic polypeptide

SEQ ID NO: 96 - protein, 119 aa, Artificial Sequence; identical to SEQ ID NO: 93
  Description of Artificial Sequence: Synthetic polypeptide

SEQ ID NO: 97 - protein, 119 aa, Artificial Sequence; identical to SEQ ID NO: 93
  Description of Artificial Sequence: Synthetic polypeptide

SEQ ID NO: 98 - protein, 119 aa, Artificial Sequence; identical to SEQ ID NO: 93
  Description of Artificial Sequence: Synthetic polypeptide

SEQ ID NO: 99 - protein, 112 aa, Artificial Sequence
  Description of Artificial Sequence: Synthetic polypeptide
EVQLVESGGGLVQPGGSLRLSCAASGFTFSRYSMSWVRQAPGKGLELVAQINSVGNSTYYPDTVKGRFTISRDNAKNTLYLQMNSLRAEDTAVYYCASGDYWGQGTLVTVSS

SEQ ID NO: 100 - protein, 113 aa, Artificial Sequence
  Description of Artificial Sequence: Synthetic polypeptide
DVVMTQSPLSLPVTLGQPASISCRSSQSLIYSDGNAYLHWFLQKPGQSPRLLIYKVSNRFSGVPDRFSGSGSGTDFTLKISRVEAEDVGVYYCSQSTHVPWTFGQGTKVEIKR

SEQ ID NO: 101 - protein, 126 aa, Artificial Sequence
  Description of Artificial Sequence: Synthetic polypeptide
QVQLVESGGGLVQPGGSLRLSCAASGFTFSSYAMSWVRQAPGKGLEWVSAINASGTRTYYADSVKGRFTISRDNSKNTLYLQMNSLRAEDTAVYYCARGKGNTHKPYGYVRYFDVWGQGTLVTVSS

SEQ ID NO: 102 - protein, 110 aa, Artificial Sequence
  Description of Artificial Sequence: Synthetic polypeptide
DIVLTQSPATLSLSPGERATLSCRASQSVSSSYLAWYQQKPGQAPRLLIYGASSRATGVPARFSGSGSGTDFTLTISSLEPEDFATYYCLQIYNMPITFGQGTKVEIKRT

SEQ ID NO: 103 - protein, 122 aa, Artificial Sequence
  Description of Artificial Sequence: Synthetic polypeptide
EVQLQASGGGLVQAGGSLRLSCAASGFKITHYTMGWFRQAPGKEREFVSRITWGGDNTFYSNSVKGRFTISRDNAKNTVYLQMNSLKPEDTADYYCAAGSTSTATPLRVDYWGKGTQVTVSS

SEQ ID NO: 104 - protein, 113 aa, Artificial Sequence
  Description of Artificial Sequence: Synthetic polypeptide
QVQLVQSGAEVKKPGSSVKVSCKASGYTFTNYDIHWVRQAPGQGLEWMGWIYPGDGSTKYNEKFKGRVTITADESTSTAYMELSSLRSEDTAVYYCAREWAYWGQGTTVTVSS

SEQ ID NO: 105 - protein, 108 aa, Artificial Sequence
  Description of Artificial Sequence: Synthetic polypeptide
DIQMTQSPSSLSASVGDRVTITCRASQDIGGNLYWYQQKPGKAPKLLIYATSSLDSGVPSRFSGSRSGTDYTLTISSLQPEDFATYYCLQYSSSPWTFGQGTKVEIKR

SEQ ID NO: 106 - protein, 108 aa, Artificial Sequence
  Description of Artificial Sequence: Synthetic polypeptide
DIQMTQSPSSLSASVGDRVTITCRASQDIGGNLYWLQQKPGKAPKRLIYATSSLDSGVPKRFSGSRSGSDYTLTISSLQPEDFATYYCLQYSSSPWTFGQGTKVEIKR

SEQ ID NO: 107 - protein, 108 aa, Artificial Sequence
  Description of Artificial Sequence: Synthetic polypeptide
DIQMTQSPSSLSASVGDRVTITCRASQDIGGNLYWLQQKPGKTIKRLIYATSSLDSGVPSRFSGSGSGTDYTLTISSLQPEDFATYYCLQYSSSPWTFGQGTKVEIKR

SEQ ID NO: 108 - protein, 108 aa, Artificial Sequence
  Description of Artificial Sequence: Synthetic polypeptide
DIQMTQSPSSLSASVGDRVTITCRASQDIGGNLYWYQQKPGKAPKLLIYATSSLDSGVPSRFSGSGSGTDFTLTISSLQPEDFATYYCLQYSSSPWTFGQGTKVEIKR

SEQ ID NO: 109 - protein, 10 aa, Artificial Sequence
  Description of Artificial Sequence: Synthetic peptide
GFTFSNYGMS

SEQ ID NO: 110 - protein, 17 aa, Artificial Sequence
  Description of Artificial Sequence: Synthetic peptide
SIRSGGGRTYYSDNVKG

SEQ ID NO: 111 - protein, 10 aa, Artificial Sequence
  Description of Artificial Sequence: Synthetic peptide
YDHYSGSSDY

SEQ ID NO: 112 - protein, 11 aa, Artificial Sequence
  Description of Artificial Sequence: Synthetic peptide
KSSQSLLDSDG

SEQ ID NO: 113 - protein, 7 aa, Artificial Sequence
  Description of Artificial Sequence: Synthetic peptide
LVSKLDS

SEQ ID NO: 114 - protein, 9 aa, Artificial Sequence
  Description of Artificial Sequence: Synthetic peptide
WQGTHFPRT

SEQ ID NO: 115 - protein, 10 aa, Artificial Sequence
  Description of Artificial Sequence: Synthetic peptide
GFTFSNYGMH

SEQ ID NO: 116 - protein, 17 aa, Artificial Sequence
  Description of Artificial Sequence: Synthetic peptide
MIYYDSSKMNYADTVKG

SEQ ID NO: 117 - protein, 9 aa, Artificial Sequence
  Description of Artificial Sequence: Synthetic peptide
PTSHYVVDV

SEQ ID NO: 118 - protein, 11 aa, Artificial Sequence
  Description of Artificial Sequence: Synthetic peptide
QASQDIGNWLA

SEQ ID NO: 119 - protein, 7 aa, Artificial Sequence
  Description of Artificial Sequence: Synthetic peptide
GATSLAD

SEQ ID NO: 120 - protein, 9 aa, Artificial Sequence
  Description of Artificial Sequence: Synthetic peptide
LQAYNTPWT

SEQ ID NO: 121 - protein, 10 aa, Artificial Sequence
  Description of Artificial Sequence: Synthetic peptide
GFNIKDTYIH

SEQ ID NO: 122 - protein, 17 aa, Artificial Sequence
  Description of Artificial Sequence: Synthetic peptide
RIYPTNGYTRYADSVKG

SEQ ID NO: 123 - protein, 11 aa, Artificial Sequence
  Description of Artificial Sequence: Synthetic peptide
WGGDGFYAMDY

SEQ ID NO: 124 - protein, 11 aa, Artificial Sequence
  Description of Artificial Sequence: Synthetic peptide
RASQDVNTAVA

SEQ ID NO: 125 - protein, 7 aa, Artificial Sequence
  Description of Artificial Sequence: Synthetic peptide
SASFLYS

SEQ ID NO: 126 - protein, 9 aa, Artificial Sequence
  Description of Artificial Sequence: Synthetic peptide
QQHYTTPPT

SEQ ID NO: 127 - protein, 11 aa, Artificial Sequence
  Description of Artificial Sequence: Synthetic peptide
RASQDVSTAVA

SEQ ID NO: 128 - protein, 7 aa, Artificial Sequence
  Description of Artificial Sequence: Synthetic peptide
SASFLYS

SEQ ID NO: 129 - protein, 9 aa, Artificial Sequence
  Description of Artificial Sequence: Synthetic peptide
QQSYTTPPT

SEQ ID NO: 130 - protein, 9 aa, Artificial Sequence
  Description of Artificial Sequence: Synthetic peptide
QQFPTYLPT

SEQ ID NO: 131 - protein, 9 aa, Artificial Sequence
  Description of Artificial Sequence: Synthetic peptide
QQGYNDPPT

SEQ ID NO: 132 - protein, 9 aa, Artificial Sequence
  Description of Artificial Sequence: Synthetic peptide
QQSSTDPTT

SEQ ID NO: 133 - protein, 11 aa, Artificial Sequence
  Description of Artificial Sequence: Synthetic peptide
RASQVVANSLA

SEQ ID NO: 134 - protein, 10 aa, Artificial Sequence
  Description of Artificial Sequence: Synthetic peptide
QQDATSPPTF

SEQ ID NO: 135 - protein, 9 aa, Artificial Sequence
  Description of Artificial Sequence: Synthetic peptide
QQYATDPPT

SEQ ID NO: 136 - protein, 10 aa, Artificial Sequence
  Description of Artificial Sequence: Synthetic peptide
GFTFSGYAIH

SEQ ID NO: 137 - protein, 18 aa, Artificial Sequence
  Description of Artificial Sequence: Synthetic peptide
GWISPAGGSTDYADSVKG

SEQ ID NO: 138 - protein, 10 aa, Artificial Sequence
  Description of Artificial Sequence: Synthetic peptide
GPFSPWVMDY

SEQ ID NO: 139 - protein, 10 aa, Artificial Sequence
  Description of Artificial Sequence: Synthetic peptide
GFTFLGYGIH

SEQ ID NO: 140 - protein, 10 aa, Artificial Sequence
  Description of Artificial Sequence: Synthetic peptide
GFTFSGYAIH

SEQ ID NO: 141 - protein, 10 aa, Artificial Sequence
  Description of Artificial Sequence: Synthetic peptide
GFTFSRYSMS

SEQ ID NO: 142 - protein, 17 aa, Artificial Sequence
  Description of Artificial Sequence: Synthetic peptide
QINSVGNSTYYPDTVKG

SEQ ID NO: 143 - protein, 3 aa, Artificial Sequence
  Description of Artificial Sequence: Synthetic peptide
GDY

SEQ ID NO: 144 - protein, 16 aa, Artificial Sequence
  Description of Artificial Sequence: Synthetic peptide
RSSQSLIYSDGNAYLH

SEQ ID NO: 145 - protein, 7 aa, Artificial Sequence
  Description of Artificial Sequence: Synthetic peptide
KVSNRFS

SEQ ID NO: 146 - protein, 9 aa, Artificial Sequence
  Description of Artificial Sequence: Synthetic peptide
SQSTHVPWT

SEQ ID NO: 147 - protein, 10 aa, Artificial Sequence
  Description of Artificial Sequence: Synthetic peptide
GFTFSSYAMS

SEQ ID NO: 148 - protein, 12 aa, Artificial Sequence
  Description of Artificial Sequence: Synthetic peptide
AINASGTRTYYA

SEQ ID NO: 149 - protein, 17 aa, Artificial Sequence
  Description of Artificial Sequence: Synthetic peptide
GKGNTHKPYGYVRYFDV

SEQ ID NO: 150 - protein, 12 aa, Artificial Sequence
  Description of Artificial Sequence: Synthetic peptide
RASQSVSSSYLA

SEQ ID NO: 151 - protein, 7 aa, Artificial Sequence
  Description of Artificial Sequence: Synthetic peptide
GASSRAT

SEQ ID NO: 152 - protein, 9 aa, Artificial Sequence
  Description of Artificial Sequence: Synthetic peptide
LQIYNMPIT

SEQ ID NO: 153 - protein, 10 aa, Artificial Sequence
  Description of Artificial Sequence: Synthetic peptide
GFKITHYTMG

SEQ ID NO: 154 - protein, 17 aa, Artificial Sequence
  Description of Artificial Sequence: Synthetic peptide
RITWGGDNTFYSNSVKG

SEQ ID NO: 155 - protein, 13 aa, Artificial Sequence
  Description of Artificial Sequence: Synthetic peptide
GSTSTATPLRVDY

SEQ ID NO: 156 - protein, 10 aa, Artificial Sequence
  Description of Artificial Sequence: Synthetic peptide
GYTFTNYDIH

SEQ ID NO: 157 - protein, 17 aa, Artificial Sequence
  Description of Artificial Sequence: Synthetic peptide
WIYPGDGSTKYNEKFKG

SEQ ID NO: 158 - protein, 8 aa, Artificial Sequence
  Description of Artificial Sequence: Synthetic peptide
YWGQGTTV

SEQ ID NO: 159 - protein, 11 aa, Artificial Sequence
  Description of Artificial Sequence: Synthetic peptide
RASQDIGGNLY

SEQ ID NO: 160 - protein, 7 aa, Artificial Sequence
  Description of Artificial Sequence: Synthetic peptide
ATSSLDS

SEQ ID NO: 161 - protein, 9 aa, Artificial Sequence
  Description of Artificial Sequence: Synthetic peptide
LQYSSSPWT

SEQ ID NO: 162 - protein, 121 aa, Artificial Sequence
  Description of Artificial Sequence: Synthetic polypeptide
EFQLQQSGPELVKPGASVRISCKASGYSFTDYNMNWVKQSNGKSLEWVGVINPNYGSSTYNQKFKGKATLTVDQSSSTAYMQLNSLTSEDSAVYYCARKWGQLGRGFFDVWGTGTTVTVSS

SEQ ID NO: 163 - protein, 107 aa, Artificial Sequence
  Description of Artificial Sequence: Synthetic polypeptide
QIVLSQSPAILSASPGEKVTMTCRASSSVSYMHWFQQKPGSSPKPWIYATSNLASGVPARFSGSGSGTSYSLTISRVEAEDAATYYCQQWSSSPLTFGAGTKLELKR

SEQ ID NO: 164 - protein, 5 aa, Artificial Sequence
  Description of Artificial Sequence: Synthetic peptide
DYNMN

SEQ ID NO: 165 - protein, 17 aa, Artificial Sequence
  Description of Artificial Sequence: Synthetic peptide
VINPNYGSSTYNQKFKG

SEQ ID NO: 166 - protein, 11 aa, Artificial Sequence
  Description of Artificial Sequence: Synthetic peptide
KWGQLGRGFFD

SEQ ID NO: 167 - protein, 10 aa, Artificial Sequence
  Description of Artificial Sequence: Synthetic peptide
RASSSVSYMH

SEQ ID NO: 168 - protein, 7 aa, Artificial Sequence
  Description of Artificial Sequence: Synthetic peptide
ATSNLAS

SEQ ID NO: 169 - protein, 9 aa, Artificial Sequence
  Description of Artificial Sequence: Synthetic peptide
QQWSSSPLT

SEQ ID NO: 170 - protein, 120 aa, Artificial Sequence
  Description of Artificial Sequence: Synthetic polypeptide
EVQLVQSGAEVKKPGASVKVSCKASGYTFTSHGISWVRQAPGQGLDWMGWISPYSGNTNYAQKLQGRVTMTTDTSTSTAYMELSSLRSEDTAVYYCARVGSGPYYYMDVWGQGTLVTVSS

SEQ ID NO: 171 - protein, 110 aa, Artificial Sequence
  Description of Artificial Sequence: Synthetic polypeptide
QSALTQPRSVSGSPGQSVTISCTGTSSSVGDSIYVSWYQQHPGKAPKLMLYDVTKRPSGVPDRFSGSKSGNTASLTISGLQAEDEADYYCCSYAGTDTLFGGGTKVTVLG

SEQ ID NO: 172 - protein, 5 aa, Artificial Sequence
  Description of Artificial Sequence: Synthetic peptide
SHGIS

SEQ ID NO: 173 - protein, 16 aa, Artificial Sequence
  Description of Artificial Sequence: Synthetic peptide
WISPYSGNTNYAQKLQ

SEQ ID NO: 174 - protein, 11 aa, Artificial Sequence
  Description of Artificial Sequence: Synthetic peptide
VGSGPYYYMDV

SEQ ID NO: 175 - protein, 14 aa, Artificial Sequence
  Description of Artificial Sequence: Synthetic peptide
TGTSSSVGDSIYVS

SEQ ID NO: 176 - protein, 7 aa, Artificial Sequence
  Description of Artificial Sequence: Synthetic peptide
DVTKRPS

SEQ ID NO: 177 - protein, 9 aa, Artificial Sequence
  Description of Artificial Sequence: Synthetic peptide
CSYAGTDTL

SEQ ID NO: 178 - protein, 10 aa, Artificial Sequence
  Description of Artificial Sequence: Synthetic peptide
GGGGSGGGGS

SEQ ID NO: 179 - protein, 9 aa, Artificial Sequence
  Description of Artificial Sequence: Synthetic peptide
GGSGGGGSG

SEQ ID NO: 180 - protein, 249 aa, Artificial Sequence
  Description of Artificial Sequence: Synthetic polypeptide
EFQLQQSGPELVKPGASVRISCKASGYSFTDYNMNWVKQSNGKSLEWVGVINPNYGSSTYNQKFKGKATLTVDQSSSTAYMQLNSLTSEDSAVYYCARKWGQLGRGFFDVWGTGTTVTVSSGGGGSGGGGSEVQLVESGGGLVQPGNSLTLSCVASGFTFSNYGMHWIRQAPKKGLEWIAMIYYDSSKMNYADTVKGRFTISRDNSKNTLYLEMNSLRSEDTAMYYCAVPTSHYVVDVWGQGVSVTVSS

SEQ ID NO: 181 - protein, 224 aa, Artificial Sequence
  Description of Artificial Sequence: Synthetic polypeptide
QIVLSQSPAILSASPGEKVTMTCRASSSVSYMHWFQQKPGSSPKPWIYATSNLASGVPARFSGSGSGTSYSLTISRVEAEDAATYYCQQWSSSPLTFGAGTKLELKRGGSGGGGSGDIQMTQSPASLSASLEEIVTITCQASQDIGNWLAWYQQKPGKSPQLLIYGATSLADGVPSRFSGSRSGTQFSLKISRVQVEDIGIYYCLQAYNTPWTFGGGTKLELKR

SEQ ID NO: 182 - protein, 245 aa, Artificial Sequence
  Description of Artificial Sequence: Synthetic polypeptide
QVQLVQSGAEVKKPGASVKVSCKASGFTFSNYGMHWIRQAPGQGLEWIAMIYYDSSKMNYADTVKGRFTITRDNSTNTLYMELSSLRSEDTAVYYCAVPTSHYVVDVWGQGTTVTVSSASTKGPEFQLQQSGPELVKPGASVRISCKASGYSFTDYNMNWVKQSNGKSLEWVGVINPNYGSSTYNQKFKGKATLTVDQSSSTAYMQLNSLTSEDSAVYYCARKWGQLGRGFFDVWGTGTTVTVSS

SEQ ID NO: 183 - protein, 227 aa, Artificial Sequence
  Description of Artificial Sequence: Synthetic polypeptide
EIVMTQSPATLSVSPGERATLSCQASQDIGNWLAWYQQKPGQSPRLLIYGATSLADGVPARFSGSRSGTEFTLTISSLQSEDFAVYYCLQAYNTPWTFGGGTKVEIKRTVAAPSVFIFPPQIVLSQSPAILSASPGEKVTMTCRASSSVSYMHWFQQKPGSSPKPWIYATSNLASGVPARFSGSGSGTSYSLTISRVEAEDAATYYCQQWSSSPLTFGAGTKLELKR

SEQ ID NO: 184 - protein, 248 aa, Artificial Sequence
  Description of Artificial Sequence: Synthetic polypeptide
EVQLVQSGAEVKKPGASVKVSCKASGYTFTSHGISWVRQAPGQGLDWMGWISPYSGNTNYAQKLQGRVTMTTDTSTSTAYMELSSLRSEDTAVYYCARVGSGPYYYMDVWGQGTLVTVSSGGGGSGGGGSEVQLVESGGGLVQPGGSLRLSCAASGFTFSNYGMHWIRQAPGKGLEWIAMIYYDSSKMNYADTVKGRFTISRDNAKNSLYLQMNSLRAEDTAVYYCAVPTSHYVVDVWGQGTTVTVSS

SEQ ID NO: 185 - protein, 227 aa, Artificial Sequence
  Description of Artificial Sequence: Synthetic polypeptide
QSALTQPRSVSGSPGQSVTISCTGTSSSVGDSIYVSWYQQHPGKAPKLMLYDVTKRPSGVPDRFSGSKSGNTASLTISGLQAEDEADYYCCSYAGTDTLFGGGTKVTVLGGGSGGGGSGEIVMTQSPATLSVSPGERATLSCQASQDIGNWLAWYQQKPGQSPRLLIYGATSLADGVPARFSGSRSGTEFTLTISSLQSEDFAVYYCLQAYNTPWTFGGGTKVEIKR

SEQ ID NO: 186 - protein, 119 aa, Artificial Sequence [sequence omitted]

SEQ ID NO: 187 - protein, 113 aa, Artificial Sequence
  Description of Artificial Sequence: Synthetic polypeptide
DVVMTQSPLSLPVTPGEPASISCKSSQSLLDSDGKTYLNWLLQKPGQSPQRLIYLVSKLDSGVPDRFSGSGSGTDFTLKISRVEAEDVGVYYCWQGTHFPRTFGQGTKVEIKR

SEQ ID NO: 188 - protein, 243 aa, Artificial Sequence; identical to SEQ ID NO: 48
  Description of Artificial Sequence: Synthetic polypeptide

SEQ ID NO: 189 - protein, 233 aa, Artificial Sequence
  Description of Artificial Sequence: Synthetic polypeptide
EIVMTQSPATLSVSPGERATLSCQASQDIGNWLAWYQQKPGQSPRLLIYGATSLADGVPARFSGSRSGTEFTLTISSLQSEDFAVYYCLQAYNTPWTFGGGTKVEIKRTVAAPSVFIFPPDVVMTQSPLSLPVTPGEPASISCKSSQSLLDSDGKTYLNWLLQKPGQSPQRLIYLVSKLDSGVPDRFSGSGSGTDFTLKISRVEAEDVGVYYCWQGTHFPRTFGQGTKVEIKR

SEQ ID NO: 190 - protein, 247 aa, Artificial Sequence
  Description of Artificial Sequence: Synthetic polypeptide
EVQLLESGGGLVQPGGSLRLSCAASGFTFSNYGMSWVRQAPGKGLEWVASIRSGGGRTYYSDNVKGRFTISRDNSKNTLYLQMNSLRAEDTAVYYCVRYDHYSGSSDYWGQGTLVTVSSGGGGSGGGGSEVQLVESGGGLVQPGGSLRLSCAASGFTFSNYGMHWIRQAPGKGLEWIAMIYYDSSKMNYADTVKGRFTISRDNAKNSLYLQMNSLRAEDTAVYYCAVPTSHYVVDVWGQGTTVTVSS

SEQ ID NO: 191 - protein, 230 aa, Artificial Sequence
  Description of Artificial Sequence: Synthetic polypeptide
DVVMTQSPLSLPVTPGEPASISCKSSQSLLDSDGKTYLNWLLQKPGQSPQRLIYLVSKLDSGVPDRFSGSGSGTDFTLKISRVEAEDVGVYYCWQGTHFPRTFGQGTKVEIKRGGSGGGGSGEIVMTQSPATLSVSPGERATLSCQASQDIGNWLAWYQQKPGQSPRLLIYGATSLADGVPARFSGSRSGTEFTLTISSLQSEDFAVYYCLQAYNTPWTFGGGTKVEIKR

SEQ ID NO: 192 - protein, 248 aa, Artificial Sequence
  Description of Artificial Sequence: Synthetic polypeptide
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSEVQLVESGGGLVQPGGSLRLSCAASGFTFSNYGMHWIRQAPGKGLEWIAMIYYDSSKMNYADTVKGRFTISRDNAKNSLYLQMNSLRAEDTAVYYCAVPTSHYVVDVWGQGTTVTVSS

SEQ ID NO: 193 - protein, 225 aa, Artificial Sequence
  Description of Artificial Sequence: Synthetic polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRGGSGGGGSGEIVMTQSPATLSVSPGERATLSCQASQDIGNWLAWYQQKPGQSPRLLIYGATSLADGVPARFSGSRSGTEFTLTISSLQSEDFAVYYCLQAYNTPWTFGGGTKVEIKR

SEQ ID NO: 194-195 - skipped (2 entries)

SEQ ID NO: 196 - protein, 112 aa, Artificial Sequence
  Description of Artificial Sequence: Synthetic polypeptide
DIVMTQTPLSLSVTPGQPASISCRSSQSLIYSDGNAYLHWYLQKPGQSPQLLIYKVSNRFSGVPDRFSGSGSGTDFTLKISRVEAEDVGVYYCSQSTHVPWTFGGGTKVEIK

SEQ ID NO: 197 - protein, 112 aa, Artificial Sequence
  Description of Artificial Sequence: Synthetic polypeptide
DVVMTQSPLSLPVTLGQPASISCRSSQSLIYSDGNAYLHWFQQRPGQSPRRLIYKVSNRFSGVPDRFSGSGSGTDFTLKISRVEAEDVGVYYCSQSTHVPWTFGGGTKVEIK

SEQ ID NO: 198 - protein, 113 aa, Artificial Sequence
  Description of Artificial Sequence: Synthetic polypeptide
EVQLVESGGGLVKPGGSLRLSCAASGFTFSRYSMSWVRQAPGKGLEWVGQINSVGNSTYYPDTVKGRFTISRDDSKNTLYLQMNSLKTEDTAVYYCITTGDYWGQGTLVTVSS

SEQ ID NO: 199 - protein, 112 aa, Artificial Sequence
  Description of Artificial Sequence: Synthetic polypeptide
EVQLLESGGGLVQPGGSLRLSCAASGFTFSRYSMSWVRQAPGKGLEWVSQINSVGNSTYYPDTVKGRFTISRDNSKNTLYLQMNSLRSEDTAVYYCAKGDYWGQGTLVTVSS

SEQ ID NO: 200 - protein, 112 aa, Artificial Sequence
  Description of Artificial Sequence: Synthetic polypeptide
  MOD_RES (86)..(86): Ile or Leu
DIQMTQSPSSLSASVGDRVTITCSASQGISNYLNWYQQKPGKAPKLLIYYTSSLHSGVPSFPSRFSGSGSGTDFTFTISSLQPEDXATYYCQQYRKLPYTFGGGTKVEIIKR

SEQ ID NO: 201 - protein, 113 aa, Artificial Sequence
  Description of Artificial Sequence: Synthetic polypeptide
EVQLVESGGGLVQPGGSLRLSCAASGFDFSRYWMNWVRQAPGKGLEWVSEINPDSSTINYTPSLKDRPTISRDNAKNTLYLQMNSLRAEDTAVYYCARQMGYWGQGTTLTVSS

SEQ ID NO: 202 - protein, 113 aa, Artificial Sequence
  Description of Artificial Sequence: Synthetic polypeptide
QVQLQESGPGLVKPSETLSLTCTVSGFDFSRYWMNWIRQPPGKGLEWIGEINPDSSTINYTPSLKDRVTISKDTSKNQFSLKLSSVTAADTAVYYCARQMGYWGQGTLVTVSS

SEQ ID NO: 203 - protein, 110 aa, Artificial Sequence
  Description of Artificial Sequence: Synthetic polypeptide
DIVLTQSPATLSLSPGERATLSCRASQSVSSSYLAWYQQKPGQAPRLLIYGASSRATGVPARFSGSGSGTDFTLTISSLEPEDFAVYYCQQVYNPPVTFGQGTKVEIKRT

SEQ ID NO: 204 - protein, 110 aa, Artificial Sequence
  Description of Artificial Sequence: Synthetic polypeptide
DIVLTQSPATLSLSPGERATLSCRASQSVSSSYLAWYQQKPGQAPRLLIYGASSRATGVPARFSGSGSGTDFTLTISSLEPEDFATYYCFQLYSDPFTFGQGTKVEIKRT

SEQ ID NO: 205 - protein, 110 aa, Artificial Sequence
  Description of Artificial Sequence: Synthetic polypeptide
DIVLTQSPATLSLSPGERATLSCRASQSVSSSYLAWYQQKPGQAPRLLIYGASSRATGVPARFSGSGSGTDFTLTISSLEPEDFATYYCQQLSSPPPTFGQGTKVEIKRT

SEQ ID NO: 206 - protein, 122 aa, Artificial Sequence
  Description of Artificial Sequence: Synthetic polypeptide
QVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCARLTHYARYYRYFDVWGQGTLVTVSS

SEQ ID NO: 207 - protein, 126 aa, Artificial Sequence
  Description of Artificial Sequence: Synthetic polypeptide
QVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCARGKGNTHKPYGYVRYFDVWGQGTLVTVSS

SEQ ID NO: 208 - protein, 124 aa, Artificial Sequence
  Description of Artificial Sequence: Synthetic polypeptide
QVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCARLLSRGYNGYYHKFDVWGQGTLVTVSS

SEQ ID NO: 209 - protein, 8 aa, Artificial Sequence
  Description of Artificial Sequence: Synthetic peptide
GSGSGNGS

SEQ ID NO: 210 - protein, 8 aa, Artificial Sequence
  Description of Artificial Sequence: Synthetic peptide
GSGSGSGS

SEQ ID NO: 211 - protein, 8 aa, Artificial Sequence
  Description of Artificial Sequence: Synthetic peptide
GGSGSGSG

SEQ ID NO: 212 - protein, 6 aa, Artificial Sequence
  Description of Artificial Sequence: Synthetic peptide
GGSGSG

SEQ ID NO: 213 - protein, 4 aa, Artificial Sequence
  Description of Artificial Sequence: Synthetic peptide
GGSG

SEQ ID NO: 214 - protein, 8 aa, Artificial Sequence
  Description of Artificial Sequence: Synthetic peptide
GGSGNGSG

SEQ ID NO: 215 - protein, 3 aa, Artificial Sequence
  Description of Artificial Sequence: Synthetic peptide
GSG